(19) United States
(12) Patent Application Publication
(10) Pub. No.: US 2005/0192215 A1
(43) Pub. Date: Sep. 1, 2005

(54) Methods and materials relating to novel polypeptides and polynucleotides

(76) Inventors: Malabika Ghosh, Sunnyvale, CA (US); Y Tom Tang, San Jose, CA (US); Jian-Rui Wang, San Jose, CA (US); Zhiwei Wang, Athens, GA (US); Qing Zhao, San Jose, CA (US); Chongjun Xu, San Jose, CA (US); Julio J Mulero, Sunnyvale, CA (US); Bryan J Boyle, San Francisco, CA (US)

Correspondence Address: NUVELO, INC, 675 ALMANOR AVE., SUNNYVALE, CA, 94085, US

(21) Appl. No.: 10/496,905

(22) PCT Filed: Dec. 2, 2002
(86) PCT No.: PCT/US02/38526

§ 371 Date: Jun. 16, 2004

Related U.S. Application Data
(63) Continuation-in-part of application No. 10/005,499, filed on Dec. 3, 2001, now abandoned.
Application No. 10/005,499 is a continuation-in-part of application No. PCT/US00/35017, filed on Dec. 22, 2000.
Application No. PCT/US00/35017 is a continuation-in-part of application No. 09/552,317, filed on Apr. 25, 2000, now abandoned.
Application No. 09/552,317 is a continuation-in-part of application No. 09/488,725, filed on Jan. 21, 2000, now abandoned.

(51) Int. Cl. C12Q 1/68; C07H 21/04; A61K 38/17; C07K 16/28; C07K 14/705

(52) U.S. Cl. 514/12; 530/350; 435/6; 435/69.1; 435/320.1; 435/325; 536/23.5

(57) ABSTRACT

The invention provides novel polynucleotides and polypeptides encoded by such polynucleotides and mutants or variants thereof that correspond to the novel polynucleotides and polypeptides. Other aspects of the invention include vectors containing processes for producing novel polypeptides, and antibodies specific for such polypeptides.

Related subject matter is disclosed in the following co-owned, co-pending applications:
1) U.S. application Ser. No. 10/005,499, filed Dec. 3, 2001, entitled “Methods and Materials Relating to Novel Secreted Adiponectin-like Polypeptides and Polynucleotides”, Attorney Docket No. HYS-46, which is a continuation-in-part application of PCT Application Serial No. PCT/US00/35017 filed Dec. 22, 2000 entitled “Novel Contigs Obtained from Various Libraries”, Attorney Docket No. 784CIP3A/PCT, which in turn is a continuation-in-part application of U.S. application Ser. No. 09/552,317 filed Apr. 25, 2000 entitled “Novel Contigs Obtained from Various Libraries”, Attorney Docket No. 784CIP, which in turn is a continuation-in-part application of U.S. application Ser. No. 09/488'725 filed Jan. 21, 2000 entitled “Novel Contigs Obtained from Various Libraries”, Attorney Docket No. 784; PCT application Serial No. PCT/US00/34263, filed Dec. 22, 2000 entitled “Novel Nucleic Acids and Polypeptides”, Attorney Docket No. 784CIP2-2F/PCT, which in turn is a continuation-in-part application of U.S. application Ser. No. 09/620,312 filed Jul. 19, 2000 entitled “Novel Nucleic Acids and Polypeptides”, Attorney Docket No. 784CIP2B; PCT Application Serial No. PCT/US01/03800 filed Feb. 5, 2001 entitled “Novel Contigs Obtained from Various Libraries”, Attorney Docket No. 787CIP3/PCT, which in turn is a continuation-in-part application of U.S. application Ser. No. 09/560,875 filed Apr. 27, 2000 entitled “Novel Contigs Obtained from Various Libraries”, Attorney Docket No. 787CIP, which in turn is a continuation-in-part application of U.S. application Ser. No. 09/496,914 filed Feb. 3, 2000 entitled “Novel Contigs Obtained from Various Libraries”, Attorney Docket No. 787; PCT application Serial No. PCT/US01/04098, filed Feb. 5, 2001 entitled “Novel Nucleic Acids and Polypeptides”, Attorney Docket No. 787CIP2-2G/PCT, which in turn is a continuation-in-part application of U.S. application Ser. No. 09/598,075 filed Jun. 20, 2000 entitled “Novel Nucleic Acids and Polypeptides”, Attoreny Docket No. 787CIP2G; PCT Application Serial No. PCT/US01/08631 filed Mar. 30, 2001 entitled “Novel Contigs Obtained from Various Libraries”, Attorney Docket No. 790CIP3/PCT, which in turn is a continuation-in-part application of U.S. application Ser. No. 09/649,167 filed Aug. 23, 2000 entitled “Novel Contigs Obtained from Various Libraries”, Attorney Docket No. 790CIP, which in turn is a continuation-in-part application of U.S. application Ser. No. 09/540,217 filed Mar. 31, 2000 entitled “Novel Contigs Obtained from Various Libraries”, Attorney Docket No. 790; U.S. application Ser. No. 09/728,952 filed Nov. 30, 2000 entitled “Novel Nucleic Acids and Polypeptides”, Attorney Docket No. 799; and U.S. Provisional application Ser. No. 60/306,971 filed Jul. 21, 2001 entitled “Novel Nucleic Acids and Polypeptides”, Attorney Docket No. 805;
2) U.S. Application Ser. No. 60/341,362, filed Dec. 17, 2001, entitled “Methods and Materials Relating to Novel Serpin-like Polypeptides and Polynucleotides,” Attorney Docket No. HYS-47, which is related to PCT Application Serial No. PCT/US01/08631, filed Mar. 30, 2001, entitled “Novel Contigs Obtained from Various Libraries,” Attorney Docket No. 790CIP3/PCT, which in turn is a continuation-in-part application of U.S. application Ser. No. 09/649,167, filed Aug. 23, 2000, entitled “Novel Contigs Obtained from Various Libraries,” Attorney Docket No. 790CIP, which in turn is a continuation-in-part application of U.S. application Ser. No. 09/540,217 filed Mar. 31, 2000 entitled “Novel Contigs Obtained from Various Libraries,” Attorney Docket No. 790;
3) U.S. Application Ser. No. 60/379,875 filed May 10, 2002 entitled “Novel Nogo-Receptor-like Protein Materials and Methods,” Attorney Docket No. HYS-52;
4) U.S. Application Ser. No. 60/379,834 filed May 10, 2002 entitled “Methods and Materials Relating to Scavenger Receptor-like Polypeptides and Polynucleotides,” Attorney Docket No. HYS-54, which is related to U.S. application Ser. No. 09/687,535 filed Oct. 13, 2000 entitled “Methods and Materials Relating to Scavenger Receptor-like Polypeptides and Polynucleotides,” Attorney Docket No. HYS-32;
5) U.S. Application Ser. No. 60/384,450 filed May 31, 2002 entitled “Methods and Materials Relating to Neural Immunoglobulin Cell Adhesion Molecule-like Polypeptides and Polynucleotides,” Attorney Docket No. HYS-55;
6) U.S. Application Ser. No. 60/384,665 filed May 31, 2002 entitled “Methods and Materials Relating to Growth Hormone-like Polypeptides and Polynucleotides,” Attorney Docket No. HYS-57;
7) U.S. Application Ser. No. 60/389,715 filed Jun. 17, 2002 entitled “Methods and Materials Relating to Neutrophil Gelatinase-associated Lipocalin-like Polypeptides and Polynucleotides,” Attorney Docket No. HYS-58, which is related to U.S. Application Ser. No. 60/365,384 filed on Mar. 14, 2002 entitled “Novel Nucleic Acids and Secreted Polypeptides,” Attorney Docket No. 814, which is a continuation-in-part application of PCT Application Serial No. PCT/US00/35017 filed Dec. 22, 2000 entitled “Novel Contigs Obtained from Various Libraries,” Attorney Docket No. 784CIP3/PCT, which in turn is a continuation-in-part application of U.S. application Ser. No. 09/552,317 filed Apr. 25, 2000 entitled “Novel Contigs Obtained from Various Libraries,” Attorney Docket No. 784CIP, which in turn is a continuation-in-part application of U.S. application Ser. No. 09/488,725 filed Jan. 21, 2000 entitled “Novel Contigs Obtained from Various Libraries,” Attorney Docket No. 784; and is a continuation-in-part application of PCT Application Serial No. PCT/US00/34263 filed Dec. 26, 2000 entitled “Novel Nucleic Acids and Polypeptides,” Attorney Docket No. 784CIP2-2F/PCT, which is a continuation-in-part application of U.S. application Ser. No. 09/620,312 filed Jul. 19, 2000, entitled “Novel Nucleic Acids and Polypeptides,” Attorney Docket No. 784CIP2B, which in turn is a continuation-in-part application of U.S. application Ser. No. 09/552,317 filed Apr. 25, 2000, entitled “Novel Contigs Obtained from Various Libraries,” Attorney Docket No. 784CIP, which in turn is a continuation-in-part application of U.S. application Ser. No. 09/488,725 filed Jan. 21, 2000, entitled “Novel Contigs Obtained from Various Libraries,” Attorney Docket No. 784;
8) U.S. Application Ser. No. 60/393,722 filed Jul. 2, 2002 entitled “Methods and Materials Relating to Novel Mucolipin-like Polypeptides and Polynucleotides,” Attorney Docket No. HYS-60;
9) U.S. Application Ser. No. 60/390,531 filed Jun. 21, 2002 entitled “Methods and Materials Relating to Peroxidasin-like Polypepides and Polynucleotides,” Attorney Docket No. HYS-61;
10) U.S. Application Ser. No. 60/391,326 filed Jun. 24, 2002 entitled “Methods and Materials Relating to Synaptic Associated Protein 90/Postsynaptic Density Protein 95 kDa-associated Protein-like Polypeptides and Polynucleotides,” Attorney Docket No. HYS-62; all of which are herein incorporated by reference in their entirety.

### 1. BACKGROUND

1.1 Technical Field

The present invention provides novel polynucleotides and proteins encoded by such polynucleotides, along with uses for these polynucleotides and proteins, for example in therapeutic, diagnostic and research methods.

1.2 Background Art

Technology aimed at the discovery of protein factors (including e.g., cytokines, such as lymphokines, interferons, CSFs, chemokines, and interleukins) has matured rapidly over the past decade. The now routine hybridization cloning and expression cloning techniques clone novel polynucleotides “directly” in the sense that they rely on information directly related to the discovered protein (i.e., partial DNA/amino acid sequence of the protein in the case of hybridization cloning; activity of the protein in the case of expression cloning). More recent “indirect” cloning techniques such as signal sequence cloning, which isolates DNA sequences based on the presence of a now well-recognized secretory leader sequence motif, as well as various PCR-based or low stringency hybridization-based cloning techniques, have advanced the state of the art by making available large numbers of DNA/amino acid sequences for proteins that are known to have biological activity, for example, by virtue of their secreted nature in the case of leader sequence cloning, by virtue of their cell or tissue source in the case of PCR-based techniques, or by virtue of structural similarity to other genes of known biological activity.

Identified polynucleotide and polypeptide sequences have numerous applications in, for example, diagnostics, forensics, gene mapping, identification of mutations responsible for genetic disorders or other traits, to assess biodiversity, and to produce many other types of data and products dependent on DNA and amino acid sequences. Proteins are known to have biological activity, for example, by virtue of their secreted nature in the case of leader sequence cloning, by virtue of their cell or tissue source in the case of PCR-based techniques, or by virtue of structural similarity to other genes of known biological activity. It is to these polypeptides and the polynucleotides encoding them that the present invention is directed.

### 2. SUMMARY OF THE INVENTION

This invention is based on the discovery of novel polypeptides, novel isolated polynucleotides encoding such polypeptides, including recombinant DNA molecules, cloned genes or degenerate variants thereof, especially naturally occurring variants such as allelic variants, antisense polynucleotide molecules, and antibodies that specifically recognize one or more epitopes present on such polypeptides, as well as hybridomas producing such antibodies. The compositions of the present invention additionally include vectors such as expression vectors containing the polynucleotides of the invention, cells genetically engineered to contain such polynucleotides, and cells genetically engineered to express such polynucleotides.

The compositions of the invention provide isolated polynucleotides that include, but are not limited to, a polynucleotide comprising the nucleotide sequence set forth in SEQ ID NO 1-4, 6, 14, 16, 25-27, 29, 157-159, 161, 183-185, 187, 214, 216, 240, 242, 271, 273, 300-301, 303, 322, 324, 345-347, 349, 353-354, 356, 377, 379, 405-407, 409, 418-419, 421, 441-443, 485-486, 488, 503, 504, 506, 514-515, 517, 526-527, 529, 547, 549, 556, 558, 570-571, 573, 577-578, 580, 587, 589, 601, 603, 606, 608, 611, 613, 617, 619, 621, 623, 625, 627, 629, or 631; or a fragment thereof that retains a desired biological activity, a polynucleotide comprising the full length protein coding sequence of SEQ ID NO: 1-4, 6, 14, 16, 25-27, 29, 157-159, 161, 183-185, 187, 214, 216, 240, 242, 271, 273, 300-301, 303, 322, 324, 345-347, 349, 353-354, 356, 377, 379, 405-407, 409, 418-419, 421, 441-443, 485-486, 488, 503, 504, 506, 514-515, 517, 526-527, 529, 547, 549, 556, 558, 570-571, 573, 577-578, 580, 587, 589, 601, 603, 606, 608, 611, 613, 617, 619, 621, 623, 625, 627, 629, or 631 (for example, the open reading frame of SEQ ID NO: 5, 15, 28, 160, 186, 215, 241, 272, 302, 323, 348, 355, 378, 408, 420, 444, 487, 505, 516, 528, 542, 548, 557, 572, 579, 588, 602, 607, 612, 618, 622, 626, or 630); and a polynucleotide comprising the nucleotide sequence of the mature protein coding sequence of any of SEQ ID NO: 1-4,6, 14, 16, 25-27, 29, 157-159, 161, 183-185, 187, 214, 216, 240, 242, 271, 273, 300-301, 303, 322, 324, 345-347, 349, 353-354, 356, 377, 379, 405-407, 409, 418-419, 421, 441-443, 485486, 488, 503, 504, 506, 514-515, 517, 526-527, 529, 547, 549, 556, 558, 570-571, 573, 577-578, 580, 587, 589, 601, 603, 606, 608, 611, 613, 617, 619, 621, 623, 625, 627, 629, or 631. The polynucleotides of the present invention also include, but are not limited to, a polynucleotide that hybridizes under stringent hybridization conditions to (a) the complement of any of the nucleotide sequences set forth in SEQ ID NO: 1-4, 6, 14, 16, 25-27, 29, 157-159, 161, 183-185, 187, 214, 216, 240, 242, 271, 273, 300-301, 303, 322, 324, 345-347, 349, 353-354, 356, 377, 379, 405-407, 409, 418-419, 421, 441-443, 485-486, 488, 503, 504, 506, 514-515, 517, 526-527, 529, 547, 549, 556, 558, 570-571, 573, 577-578, 580, 587, 589, 601, 603, 606, 608, 611, 613, 617, 619, 621, 623, 625, 627, 629, or 631; (b) a nucleotide sequence encoding any of the amino acid sequences set forth in SEQ D NO: 5, 7-13, 15, 17-24, 28, 30-156, 160, 162-182, 186, 188-213, 215, 217-239, 241, 243-270, 272, 274-299, 302, 304-321, 323, 325-344, 348, 350-352, 355, 357-376, 378, 380-401, 408, 410-414, 415, 420, 422-439, 444-480, 482-484,487,489-501, 505, 507-512, 516, 518-524, 528, 530-539, 542, 544-546, 548, 550-553, 557, 559-567, 572, 574, 576, 579, 581-584, 588, 590, 596, 602, 604-605, 607, 609-610, 612, 614-615, 618, 620, 622, 624, 626, 628, 630, 632, or 634-653; a polynucleotide which is an allelic variant of any polynucleotides recited above having at least 70% polynucleotide sequence identity to the polynucleotides; a polynucleotide which encodes a species homolog (e.g. orthologs) of any of the peptides recited above; or a polynucleotide that encodes a polypeptide comprising a specific domain or truncation of the polypeptide of SEQ ID NO: 5, 7-13, 15, 17-24, 28, 30-156, 160, 162-182, 186, 188-213, 215, 217-239, 241, 243-270, 272, 274-299, 302, 304-321, 323, 325-344, 348, 350-352, 355, 357-376, 378, 380-401, 408, 410-414, 415, 420, 422-439, 444-480, 482-484, 487, 489-501, 505, 507-512, 516, 518-524, 528, 530-539, 542, 544-546, 548, 550-553, 557, 559-567, 572, 574, 576, 579, 581-584, 588, 590, 596, 602, 604-605, 607, 609-610, 612, 614-615, 618, 620, 622, 624, 626, 628, 630, 632, or 634-653.

A collection as used in this application can be a collection of only one polynucleotide. The collection of sequence information or unique identifying information of each sequence can be provided on a nucleic acid array. In one embodiment, segments of sequence information are provided on a nucleic acid array to detect the polynucleotide that contains the segment. The array can be designed to detect full-match or mismatch to the polynucleotide that contains the segment. The collection can also be provided in a computer-readable format.

This invention further provides cloning or expression vectors comprising at least a fragment of the polynucleotides set forth above and host cells or organisms transformed with these expression vectors. Useful vectors include plasmids, cosmids, lambda phage derivatives, phagemids, and the like, that are well known in the art. Accordingly, the invention also provides a vector including a polynucleotide of the invention and a host cell containing the polynucleotide. In general, the vector contains an origin of replication functional in at least one organism, convenient restriction endonuclease sites, and a selectable marker for the host cell. Vectors according to the invention include expression vectors, replication vectors, probe generation vectors, and sequencing vectors. A host cell according to the invention can be a prokaryotic or eukaryotic cell and can be a unicellular organism or part of a multicellular organism.

The compositions of the present invention include polypeptides comprising, but not limited to, an isolated polypeptide selected from the group comprising the amino acid sequence of SEQ ID NO: 5, 7-13, 15, 17-24, 28, 30-156, 160, 162-182, 186, 188-213, 215, 217-239, 241, 243-270, 272, 274-299, 302, 304-321, 323, 325-344, 348, 350-352, 355, 357-376, 378, 380-401, 408, 410-414, 415, 420, 422-439, 444-480, 482-484, 487, 489-501, 505, 507-512, 516, 518-524, 528, 530-539, 542, 544-546, 548, 550-553, 557, 559-567, 572, 574, 576, 579, 581-584, 588, 590, 596, 602, 604-605, 607, 609-610, 612, 614-615, 618, 620, 622, 624, 626, 628, 630, 632, or 634-653; or the corresponding full length or mature protein. Polypeptides of the invention also include polypeptides with biological activity that are encoded by (a) any of the polynucleotides having a nucleotide sequence set forth in SEQ ID NO: 1-4, 6, 14, 16, 25-27, 29, 157-159, 161, 183-185, 187, 214, 216, 240, 242, 271, 273, 300-301, 303, 322, 324, 345-347, 349, 353-354, 356, 377, 379, 405-407, 409, 418419, 421, 441443, 485-486, 488, 503, 504, 506, 514-515, 517, 526-527, 529, 547, 549, 556, 558, 570-571, 573, 577-578, 580, 587, 589, 601, 603, 606, 608, 611, 613, 617, 619, 621, 623, 625, 627, 629, or 631; or (b) polynucleotides that hybridize to the complement of the polynucleotides of (a) under stringent hybridization conditions. Biologically or immunologically active variants of any of the protein sequences listed as SEQ ID NO: 5, 7-13, 15, 17-24, 28, 30-156, 160, 162-182, 186, 188-213, 215, 217-239, 241, 243-270, 272, 274-299, 302, 304-321, 323, 325-344, 348, 350-352, 355, 357-376, 378, 380-401, 408, 410-414, 415, 420, 422-439, 444480, 482-484, 487, 489-501, 505, 507-512, 516, 518-524, 528, 530-539, 542, 544-546, 548, 550-553, 557, 559-567, 572, 574, 576, 579, 581-584, 588, 590, 596, 602, 604-605,607,609-610, 612, 614-615, 618, 620, 622, 624, 626, 628, 630, 632, or 634-653 and substantial equivalents thereof that retain biological or immunological activity are also contemplated. The polypeptides of the invention may be wholly or partially chemically synthesized but are preferably produced by recombinant means using the genetically engineered cells (e.g. host cells) of the invention.

The invention also provides compositions comprising a polypeptide of the invention. Pharmaceutical compositions of the invention may comprise a polypeptide of the invention and an acceptable carrier, such as a hydrophilic, e.g., pharmaceutically acceptable, carrier.

The invention also relates to methods for producing a polypeptide of the invention comprising culturing host cells comprising an expression vector containing at least a fragment of a polynucleotide encoding the polypeptide of the invention in a suitable culture medium under conditions permitting expression of the desired polypeptide, and purifying the protein or peptide from the culture or from the host cells. Preferred embodiments include those in which the protein produced by such a process is a mature form of the protein.

Polynucleotides according to the invention have numerous applications in a variety of techniques known to those skilled in the art of molecular biology. These techniques include use as hybridization probes, use as oligomers, or primers, for PCR, use in an array, use in computer-readable media, use for chromosome and gene mapping, use in the recombinant production of protein, and use in generation of antisense DNA or RNA, their chemical analogs and the like. For example, when the expression of an mRNA is largely restricted to a particular cell or tissue type, polynucleotides of the invention can be used as hybridization probes to detect the presence of the particular cell or tissue mRNA in a sample using, e.g., in situ hybridization.

In other exemplary embodiments, the polynucleotides are used in diagnostics as expressed sequence tags for identifying expressed genes or, as well known in the art and exemplified by Vollrath et al., Science 258:52-59 (1992), as expressed sequence tags for physical mapping of the human genome.

The polypeptides according to the invention can be used in a variety of conventional procedures and methods that are currently applied to other proteins. For example, a polypeptide of the invention can be used to generate an antibody that specifically binds the polypeptide. Such antibodies, particularly monoclonal antibodies, are useful for detecting or quantitating the polypeptide in tissue. The polypeptides of the invention can also be used as molecular weight markers, and as a food supplement.

Methods are also provided for preventing, treating, or ameliorating a medical condition which comprises the step of administering to a mammalian subject a therapeutically effective amount of a composition comprising a peptide of the present invention and a pharmaceutically acceptable carrier.

The methods of the invention also provide methods for the treatment of disorders as recited herein which comprise the administration of a therapeutically effective amount of a composition comprising a polynucleotide or polypeptide of the invention and a pharmaceutically acceptable carrier to a mammalian subject exhibiting symptoms or tendencies related to disorders as recited herein. In addition, the invention encompasses methods for treating diseases or disorders as recited herein comprising the step of administering a composition comprising compounds and other substances that modulate the overall activity of the target gene products and a pharmaceutically acceptable carrier. Compounds and other substances can effect such modulation either on the level of target gene/protein expression or target protein activity. Specifically, methods are provided for preventing, treating or ameliorating a medical condition, including viral diseases, which comprises administering to a mammalian subject, including but not limited to humans, a therapeutically effective amount of a composition comprising a polypeptide of the invention or a therapeutically effective amount of a composition comprising a binding partner of (e.g., antibody specifically reactive for) the polypeptides of the invention. The mechanics of the particular condition or pathology will dictate whether the polypeptides of the invention or binding partners (or inhibitors) of these would be beneficial to the individual in need of treatment.

According to this method, polypeptides of the invention can be administered to produce an in vitro or in vivo inhibition of cellular function. A polypeptide of the invention can be administered in vivo alone or as an adjunct to other therapies. Conversely, protein or other active ingredients of the present invention may be included in formulations of a particular agent to minimize side effects of such an agent.

The invention further provides methods for manufacturing medicaments useful in the above-described methods.

The present invention further relates to methods for detecting the presence of the polynucleotides or polypeptides of the invention in a sample (e.g., tissue or sample). Such methods can, for example, be utilized as part of prognostic and diagnostic evaluation of disorders as recited herein and for the identification of subjects exhibiting a predisposition to such conditions.

The invention provides a method for detecting a polypeptide of the invention in a sample comprising contacting the sample with a compound that binds to and forms a complex with the polypeptide under conditions and for a period sufficient to form the complex and detecting formation of the complex, so that if a complex is formed, the polypeptide is detected.

The invention also provides kits comprising polynucleotide probes and/or monoclonal antibodies, and optionally quantitative standards, for carrying out methods of the invention. Furthermore, the invention provides methods for evaluating the efficacy of drugs, and monitoring the progress of patients, involved in clinical trials for the treatment of disorders as recited above.

The invention also provides methods for the identification of compounds that modulate (i.e., increase or decrease) the expression or activity of the polynucleotides and/or polypeptides of the invention. Such methods can be utilized, for example, for the identification of compounds that can ameliorate symptoms of disorders as recited herein. Such methods can include, but are not limited to, assays for identifying compounds and other substances that interact with (e.g., bind to) the polypeptides of the invention.

The invention provides a method for identifying a compound that binds to the polypeptide of the present invention comprising contacting the compound with the polypeptide under conditions and for a time sufficient to form a polypeptide/compound complex and detecting the complex, so that if the polypeptide/compound complex is detected, a compound that binds to the polypeptide of the invention is identified.

Also provided is a method for identifying a compound that binds to a polypeptide of the invention comprising contacting the compound with a polypeptide of the invention in a cell for a time sufficient to form a polypeptide/compound complex wherein the complex drives expression of a reporter gene sequence in the cell and detecting the complex by detecting reporter gene sequence expression so that if the polypeptide/compound complex is detected a compound that binds to the polypeptide of the invention is identified.

### 3. BRIEF DESCRIPTION OF THE DRAWINGS

FIG. 1 shows the BLASTP amino acid sequence alignment between adiponectin-like polypeptide SEQ ID NO: 5 and adiponectin SEQ ID NO: 402 (Hotta et al., Diabetes 50:1126-1133 (2001)).

FIG. 2 shows the BLASTP amino acid sequence alignment between adiponectin-like polypeptide SEQ ID NO: 5 and human adiponectin SEQ ID NO: 404 (Patent No. JP3018186-B1).

FIG. 3 shows the BLASTP amino acid sequence alignment between adiponectin-like polypeptide SEQ ID NO: 15 and adiponectin SEQ ID NO: 402 (Hotta et al., Diabetes 50:1126-1133 (2001)).

FIG. 4 shows the BLASTP amino acid sequence alignment between adiponectin-like polypeptide SEQ ID NO: 15 and human adiponectin SEQ ID NO: 404 (Patent No. JP3018186-B1).

FIG. 5 shows the BLASTP amino acid sequence alignment between adiponectin-like polypeptide SEQ ID NO: 28 and adiponectin SEQ ID NO: 402 (Hotta et al., Diabetes 50:1126-1133 (2001)).

FIG. 6 shows the BLASTP amino acid sequence alignment between adiponectin-like polypeptide SEQ ID NO: 28 and human adiponectin SEQ ID NO: 404 (Patent No. JP3018186-B1).

FIG. 7 shows the BLASTP amino acid sequence alignment between adiponectin-like polypeptide SEQ ID NO: 160 and adiponectin SEQ ID NO: 402 (Hotta et al., Diabetes 50:1126-1133 (2001)).

FIG. 8 shows the BLASTP amino acid sequence alignment between adiponectin-like polypeptide SEQ ID NO: 160 and human adiponectin SEQ ID NO: 404 (Patent No. JP3018186-B1).

FIG. 9 shows the BLASTP amino acid sequence alignment between adiponectin-like polypeptide SEQ ID NO: 186 and adiponectin SEQ ID NO: 402 (Hotta et al., Diabetes 50:1126-1133 (2001)).

FIG. 10 shows the BLASTP amino acid sequence alignment between adiponectin-like polypeptide SEQ ID NO: 186 and human adiponectin SEQ ID NO: 404 (Patent No. JP3018186-B1).

FIG. 11 shows the BLASTP amino acid sequence alignment between adiponectin-like polypeptide SEQ ID NO: 215 and adiponectin SEQ ID NO: 402 (Hotta et al., Diabetes 50:1126-1133 (2001)).

FIG. 12 shows the BLASTP amino acid sequence alignment between adiponectin-like polypeptide SEQ ID NO: 215 and human adiponectin SEQ ID NO: 404 (Patent No. JP3018186-B1).

FIG. 13 shows the BLASTP amino acid sequence alignment between adiponectin-like polypeptide SEQ ID NO: 241 and adiponectin SEQ ID NO: 402 (Hotta et al., Diabetes 50:1126-1133 (2001)).

FIG. 14 shows the BLASTP amino acid sequence alignment between adiponectin-like polypeptide SEQ ID NO: 241 and human adiponectin SEQ ID NO: 404 (Patent No. JP3018186-B1).

FIG. 15 shows the BLASTP amino acid sequence alignment between adiponectin-like polypeptide SEQ ID NO: 272 and adipose tissue-specific protein AdipoQ SEQ ID NO: 403 (Sato et al., J. Biol. Chem. 276:28849-28856 (2001)).

FIG. 16 shows the BLASTP amino acid sequence alignment between adiponectin-like polypeptide SEQ ID NO: 272 and adipose tissue-specific protein AdipoQ SEQ ID NO: 403 (Sato et al., J. Biol. Chem. 276:28849-28856 (2001)).

FIG. 17 shows the BLASTP amino acid sequence alignment between adiponectin-like polypeptide SEQ ID NO: 272 and human adiponectin SEQ ID NO: 404 (Patent No. JP3018186-B1).

FIG. 18 shows the BLASTP amino acid sequence alignment between adiponectin-like polypeptide SEQ ID NO: 302 and adiponectin SEQ ID NO: 402 (Hotta et al., Diabetes 50:1126-1133 (2001)).

FIG. 19 shows the BLASTP amino acid sequence alignment between adiponectin-like polypeptide SEQ ID NO: 302 and human adiponectin SEQ ID NO: 404 (Patent No. JP3018186-B1).

FIG. 20 shows the BLASTP amino acid sequence alignment between adiponectin-like polypeptide SEQ ID NO: 323 and adiponectin SEQ ID NO: 402 (Hotta et al., Diabetes 50:1126-1133 (2001)).

FIG. 21 shows the BLASTP amino acid sequence alignment between adiponectin-like polypeptide SEQ ID NO: 323 and human adiponectin SEQ ID NO: 404 (Patent No. JP3018186-B1).

FIG. 22 shows the BLASTP amino acid sequence alignment between adiponectin-like polypeptide SEQ ID NO: 348 and adiponectin SEQ ID NO: 402 (Hotta et al., Diabetes 50:1126-1133 (2001)).

FIG. 23 shows the BLASTP amino acid sequence alignment between adiponectin-like polypeptide SEQ ID NO: 348 and human adiponectin SEQ ID NO: 404 (Patent No. JP3018186-B1).

FIG. 24 shows the BLASTP amino acid sequence alignment between adiponectin-like polypeptide SEQ ID NO: 355 and adiponectin SEQ ID NO: 402 (Hotta et al., Diabetes 50:1126-1133 (2001)).

FIG. 25 shows the BLASTP amino acid sequence alignment between adiponectin-like polypeptide SEQ ID NO: 355 and human adiponectin SEQ ID NO: 404 (Patent No. JP3018186-B1).

FIG. 26 shows the BLASTP amino acid sequence alignment between adiponectin-like polypeptide SEQ ID NO: 378 and adiponectin SEQ ID NO: 402 (Hotta et al., Diabetes 50:1126-1133 (2001)).

FIG. 27 shows the BLASTP amino acid sequence alignment between adiponectin-like polypeptide SEQ ID NO: 378 and human adiponectin SEQ ID NO: 404 (Patent No. JP3018186-B1).

FIG. 28 shows the BLASTP amino acid sequence alignment of the first high scoring pair (HSP) between Serpin-like polypeptide SEQ ID NO: 408 and SERPINB12 SEQ ID NO: 416 (Askew et al., J. Biol. Chem. 276:49320-49330 (2001), herein incorporated by reference in its entirety).

FIG. 29 shows the BLASTP amino acid sequence alignment of the second high scoring pair (HSP) between Serpin-like polypeptide SEQ ID NO: 408 and SERPINB12 SEQ ID NO: 416 (Askew et al., J. Biol. Chem. 276:49320-49330 (2001), herein incorporated by reference in its entirety).

FIG. 30 shows the BLASTP amino acid sequence alignment of the first high scoring pair (HSP) between Serpin-like polypeptide SEQ ID NO: 408 and human SCCA2 protein SEQ ID NO: 417 (Patent No. DE19742725-A1, herein incorporated by reference in its entirety).

FIG. 31 shows the BLASTP amino acid sequence alignment of the second high scoring pair (HSP) between Serpin-like polypeptide SEQ ID NO: 408 and human SCCA2 protein SEQ ID NO: 417 (Patent No. DE19742725-A1, herein incorporated by reference in its entirety).

FIG. 32 shows a schematic diagram illustrating the major structural features of the Nogo receptor, NgR, and the Nogo receptor homolog, NgRHy.

FIG. 33 shows the BLASTP amino acid sequence alignment between the protein encoded by SEQ ID NO: 419 (i.e. SEQ ID NO: 420), NgRHy, and the human NgR (SEQ ID NO: 440).

FIG. 34 shows the BLASTX amino acid sequence alignment between the protein encoded by SEQ ID NO: 443 (i.e. SEQ ID NO: 444), scavenger receptor-like polypeptide and mouse macrophage scavenger receptor type I (SEQ ID NO: 481).

FIG. 35 (A, B) shows a BLASTP amino acid sequence alignment between neural IgCAM-like polypeptide (SEQ ID NO: 487) and another member of the family, mouse PANG (SEQ ID NO: 502).

FIG. 36 shows a BLASTP amino acid sequence alignment between neural IgCAM-like polypeptide (SEQ ID NO: 505) and bovine NCAM-140 (SEQ ID NO: 513).

FIG. 37 shows a multiple amino acid sequence alignment between neural IgCAM-like polypeptide (SEQ ID NO: 505), neural IgCAM-like polypeptide (SEQ ID NO: 542) and bovine NCAM-140 (SEQ ID NO: 513).

FIG. 38 shows a BLASTP amino acid sequence alignment between neural IgCAM-like polypeptide (SEQ ID NO: 516) and another member of the family, mouse DDM36 (SEQ ID NO: 52).

FIG. 39 (A, B) shows a BLASTP amino acid sequence alignment between neural IgCAM-like polypeptide (SEQ ID NO: 530) and another member of the family, rat BIG-2 (SEQ ID NO: 540).

FIG. 40 shows a BLASTP amino acid sequence alignment between growth hormone-like polypeptide (SEQ ID NO: 548) and human chorionic somatomammotropin hormone-like 1, isoform 3 precursor (SEQ ID NO: 554).

FIG. 41 shows a BLASTP amino acid sequence alignment between growth hormone-like polypeptide (SEQ ID NO: 548) and human chorionic somatomammotropin hormone-like 1, isoform 5 precursor (SEQ ID NO: 555).

FIG. 42 shows a BLASTP amino acid sequence alignment between growth hormone-like polypeptide (SEQ ID NO: 557) and human chorionic somatomammotropin hormone 1, isoform 2 precursor (SEQ ID NO: 568).

FIG. 43 shows a BLASTP amino acid sequence alignment between growth hormone-like polypeptide (SEQ ID NO: 557) and human growth hormone 2, isoform 2 precursor (SEQ ID NO: 569).

FIG. 44 shows a multiple sequence alignment between NGAL-like polypeptides (SEQ ID NO: 572 and 579) and other members of the family: (SEQ ID NO: 585 and 586, respectively).

FIG. 45 shows a BLASTP amino acid sequence alignment of mucolipin-like polypeptide (SEQ ID NO: 588) and human mucolipin 1 (SEQ ID NO: 592).

FIG. 46 (A, B) shows a multiple amino acid sequence alignment of mucolipin-like polypeptide (SEQ ID NO: 588) and other members of the family: mouse mucolipin 2 (SEQ ID NO: 591), human mucolipin 1 (SEQ ID NO: 592), human mucolipin 3 (SEQ ID NO: 593), C. elegans CUP-5 (SEQ ID NO: 595).

FIG. 47 shows an alignment of the conserved serine lipase active site between mucolipin-like polypeptide (SEQ ID NO: 596) and mucolipin 1 (SEQ ID NO: 597), as well as other lipolytic enzymes: H. liph triacylglycerol lipase, hepatic precursor (SEQ ID NO: 598), H. liph lipoprotein lipase precursor (SEQ ID NO: 599), and H. lcat phosphatidylcholine-sterol acyltransferase precursor (SEQ ID NO: 600).

FIG. 48 (A, B) shows a BLASTP amino acid sequence alignment between a peroxidasin-like polypeptide (SEQ ID NO: 602) and another member of the family, human peroxidasin-like protein MG50 (SEQ ID NO: 616).

FIG. 49 (A, B, C) shows a multiple sequence alignment between peroxidasin-like polypeptides SEQ ID NO: 602, 618, 622, and 626.

FIG. 50 (A, B) shows a BLASTP amino acid sequence alignment between a second peroxidasin-like polypeptide (SEQ ID NO: 607) and another member of the family, human peroxidasin-like protein MG50 (SEQ ID NO: 616).

FIG. 51 (A, B) shows a BLASTP amino acid sequence alignment between a third peroxidasin-like polypeptide (SEQ ID NO: 612) and another member of the family, human peroxidasin-like protein MG50 (SEQ ID NO: 616).

FIG. 52 (A, B) shows a BLASTP amino acid sequence alignment between SAPAP-like polypeptide (SEQ ID NO: 630) and rat SAPAP3 (SEQ ID NO: 633).

### 4. DETAILED DESCRIPTION OF THE INVENTION

Table 1 is a correlation table of the novel polynucleotide sequences (14, 6, 14, 16, 25-27, 29, 157-159, 161, 183-185, 187, 214, 216, 240, 242, 271, 273, 300-301, 303, 322, 324, 345-347, 349, 353-354, 356, 377, 379, 405-407, 409, 418-419, 421, 441-443, 485-486, 488, 503, 504, 506, 514-515, 517, 526-527, 529, 547, 549, 556, 558, 570-571, 573, 577-578, 580, 587, 589, 601, 603, 606, 608, 611, 613, 617, 619, 621, 623, 625, 627, 629, and 631) and the novel polypeptides (5, 7-13, 15, 17-24, 28, 30-156, 160, 162-182, 186, 188-213, 215, 217-239, 241, 243-270, 272, 274-299, 302, 304-321, 323, 325-344, 348, 350-352, 355, 357-376, 378, 380-401, 408, 410-414, 415, 420, 422-439, 444-480, 482-484, 487, 489-501, 505, 507-512, 516, 518-524, 528, 530-539, 542, 544-546, 548, 550-553, 557, 559-567, 572, 574, 576, 579, 581-584, 588, 590, 596, 602, 604-605, 607, 609-610, 612, 614-615, 618, 620, 622, 624, 626, 628, 630, 632, and 634-653) and the corresponding SEQ ID NO: in which the sequence was filed in the following priority U.S. patent Applications bearing the serial numbers of: Ser. No. 10/005,499 filed on Dec. 3, 2001, 60/341,362 filed on Dec. 17, 2001, 60/379,875 filed on May 10, 2002, 60/379,834 filed May 10, 2002, 60/384,450 filed on May 31, 2002, 60/384,665 filed on May 31, 2002, 60/389,715 filed on Jun. 17, 2002, 60/393,722, filed on Jul. 2, 2002, 60/390,531 filed on Jun. 21, 2002, and 60/391,326 filed on Jun. 24, 2002.

TABLE 1

| | Identification of Priority Application |
|---|---|
| | that sequence was filed (Attorney |
| SEQ ID NO: | Docket No._SEQ ID NO.) * |
| 1 | HYS-46_1 |
| 2 | HYS-46_2 |
| 3 | HYS-46_3 |
| 4 | HYS-46_4 |
| 5 | HYS-46_5 |
| 6 | HYS-46_6 |
| 7 | HYS-46_7 |
| 8 | HYS-46_8 |
| 9 | HYS-46_9 |
| 10 | HYS-46_10 |
| 11 | HYS-46_11 |
| 12 | HYS-46_12 |
| 13 | HYS-46_13 |
| 14 | HYS-46_14 |
| 15 | HYS-46_15 |
| 16 | HYS-46_16 |
| 17 | HYS-46_17 |
| 18 | HYS-46_18 |
| 19 | HYS-46_19 |
| 20 | HYS-46_20 |
| 21 | HYS-46_21 |
| 22 | HYS-46_22 |
| 23 | HYS-46_23 |
| 24 | HYS-46_24 |
| 25 | HYS-46_25 |
| 26 | HYS-46_26 |
| 27 | HYS-46_27 |
| 28 | HYS-46_28 |
| 29 | HYS-46_29 |
| 30 | HYS-46_30 |
| 31 | HYS-46_31 |
| 32 | HYS-46_32 |
| 33 | HYS-46_33 |
| 34 | HYS-46_34 |
| 35 | HYS-46_35 |
| 36 | HYS-46_36 |
| 37 | HYS-46_37 |
| 38 | HYS-46_38 |
| 39 | HYS-46_39 |
| 40 | HYS-46_40 |
| 41 | HYS-46_41 |
| 42 | HYS-46_42 |
| 43 | HYS-46_43 |
| 44 | HYS-46_44 |
| 45 | HYS-46_45 |
| 46 | HYS-46_46 |
| 47 | HYS-46_47 |
| 48 | HYS-46_48 |
| 49 | HYS-46_49 |
| 50 | HYS-46_50 |
| 51 | HYS-46_51 |
| 52 | HYS-46_52 |
| 53 | HYS-46_53 |
| 54 | HYS-46_54 |
| 55 | HYS-46_55 |
| 56 | HYS-46_56 |
| 57 | HYS-46_57 |
| 58 | HYS-46_58 |
| 59 | HYS-46_59 |
| 60 | HYS-46_60 |
| 61 | HYS-46_61 |
| 62 | HYS-46_62 |
| 63 | HYS-46_63 |
| 64 | HYS-46_64 |
| 65 | HYS-46_65 |
| 66 | HYS-46_66 |
| 67 | HYS-46_67 |
| 68 | HYS-46_68 |
| 69 | HYS-46_69 |
| 70 | HYS-46_70 |
| 71 | HYS-46_71 |
| 72 | HYS-46_72 |
| 73 | HYS-46_74 |
| 75 | HYS-46_75 |
| 76 | HYS-46_76 |
| 77 | HYS-46_77 |
| 78 | HYS-46_78 |
| 79 | HYS-46_79 |
| 80 | HYS-46_80 |
| 81 | HYS-46_81 |
| 82 | HYS-46_82 |
| 83 | HYS-46_83 |
| 84 | HYS-46_84 |
| 85 | HYS-46_85 |
| 86 | HYS-46_86 |
| 87 | HYS-46_87 |
| 88 | HYS-46_88 |
| 89 | HYS-46_89 |
| 90 | HYS-46_90 |
| 91 | HYS-46_91 |
| 92 | HYS-46_92 |
| 93 | HYS-46_93 |
| 94 | HYS-46_94 |
| 95 | HYS-46_95 |
| 96 | HYS-46_96 |
| 97 | HYS-46_97 |
| 98 | HYS-46_98 |
| 99 | HYS-46_99 |
| 100 | HYS-46_100 |
| 101 | HYS-46_101 |
| 102 | HYS-46_102 |
| 103 | HYS-46_103 |
| 104 | HYS-46_104 |
| 105 | HYS-46_105 |
| 106 | HYS-46_106 |
| 107 | HYS-46_107 |
| 108 | HYS-46_108 |
| 109 | HYS-46_109 |
| 110 | HYS-46_110 |
| 111 | HYS-46_111 |
| 112 | HYS-46_112 |
| 113 | HYS-46_113 |
| 114 | HYS-46_114 |
| 115 | HYS-46_115 |
| 116 | HYS-46_116 |
| 117 | HYS-46_117 |
| 118 | HYS-46_118 |
| 119 | HYS-46_119 |
| 120 | HYS-46_120 |
| 121 | HYS-46_121 |
| 122 | HYS-46_122 |
| 123 | HYS-46_123 |
| 124 | HYS-46_124 |
| 125 | HYS-46_125 |
| 126 | HYS-46_126 |
| 127 | HYS-46_127 |
| 128 | HYS-46_128 |
| 129 | HYS-46_129 |
| 130 | HYS-46_130 |
| 131 | HYS-46_131 |
| 132 | HYS-46_132 |
| 133 | HYS-46_133 |
| 134 | HYS-46_134 |
| 135 | HYS-46_135 |
| 136 | HYS-46_136 |
| 137 | HYS-46_137 |
| 138 | HYS-46_138 |
| 139 | HYS-46_139 |
| 140 | HYS-46_140 |
| 141 | HYS-46_141 |
| 142 | HYS-46_142 |
| 143 | HYS-46_143 |
| 144 | HYS-46_144 |
| 145 | HYS-46_145 |
| 146 | HYS-46_146 |
| 147 | HYS-46_147 |
| 148 | HYS-46_148 |
| 149 | HYS-46_149 |
| 150 | HYS-46_150 |
| 151 | HYS-46_151 |
| 152 | HYS-46_152 |
| 153 | HYS-46_153 |
| 154 | HYS-46_154 |
| 155 | HYS-46_155 |
| 156 | HYS-46_156 |
| 157 | HYS-46_157 |
| 158 | HYS-46_158 |
| 159 | HYS-46_159 |
| 160 | HYS-46_160 |
| 161 | HYS-46_161 |
| 162 | HYS-46_162 |
| 163 | HYS-46_163 |
| 164 | HYS-46_164 |
| 165 | HYS-46_165 |
| 166 | HYS-46_166 |
| 167 | HYS-46_167 |
| 168 | HYS-46_168 |
| 169 | HYS-46_169 |
| 170 | HYS-46_170 |
| 171 | HYS-46_171 |
| 172 | HYS-46_172 |
| 173 | HYS-46_173 |
| 174 | HYS-46_174 |
| 175 | HYS-46_175 |
| 176 | HYS-46_176 |
| 177 | HYS-46_177 |
| 178 | HYS-46_178 |
| 179 | HYS-46_179 |
| 180 | HYS-46_180 |
| 181 | HYS-46_181 |
| 182 | HYS-46_182 |
| 183 | HYS-46_183 |
| 184 | HYS-46_184 |
| 185 | HYS-46_185 |
| 186 | HYS-46_186 |
| 187 | HYS-46_187 |
| 188 | HYS-46_188 |
| 189 | HYS-46_189 |
| 190 | HYS-46_190 |
| 191 | HYS-46_191 |
| 192 | HYS-46_192 |
| 193 | HYS-46_193 |
| 194 | HYS-46_194 |
| 195 | HYS-46_195 |
| 196 | HYS-46_196 |
| 197 | HYS-46_197 |
| 198 | HYS-46_198 |
| 199 | HYS-46_199 |
| 200 | HYS-46_200 |
| 201 | HYS-46_201 |
| 202 | HYS-46_202 |
| 203 | HYS-46_203 |
| 204 | HYS-46_204 |
| 205 | HYS-46_205 |
| 206 | HYS-46_206 |
| 207 | HYS-46_207 |
| 208 | HYS-46_208 |
| 209 | HYS-46_209 |
| 210 | HYS-46_210 |
| 211 | HYS-46_211 |
| 212 | HYS-46_212 |
| 213 | HYS-46_213 |
| 214 | HYS-46_214 |
| 215 | HYS-46_215 |
| 216 | HYS-46_216 |
| 217 | HYS-46_217 |
| 218 | HYS-46_218 |
| 219 | HYS-46_219 |
| 220 | HYS-46_220 |
| 221 | HYS-46_221 |
| 222 | HYS-46_222 |
| 223 | HYS-46_223 |
| 224 | HYS-46_224 |
| 225 | HYS-46_225 |
| 226 | HYS-46_226 |
| 227 | HYS-46_227 |
| 228 | HYS-46_228 |
| 229 | HYS-46_229 |
| 230 | HYS-46_230 |
| 231 | HYS-46_231 |
| 232 | HYS-46_232 |
| 233 | HYS-46_233 |
| 234 | HYS-46_234 |
| 235 | HYS-46_235 |
| 236 | HYS-46_236 |
| 237 | HYS-46_237 |
| 238 | HYS-46_238 |
| 239 | HYS-46_239 |
| 240 | HYS-46_240 |
| 241 | HYS-46_241 |
| 242 | HYS-46_242 |
| 243 | HYS-46_243 |
| 244 | HYS-46_244 |
| 245 | HYS-46_245 |
| 246 | HYS-46_246 |
| 247 | HYS-46_247 |
| 248 | HYS-46_248 |
| 249 | HYS-46_249 |
| 250 | HYS-46_250 |
| 251 | HYS-46_251 |
| 252 | HYS-46_252 |
| 253 | HYS-46_253 |
| 254 | HYS-46_254 |
| 255 | HYS-46_255 |
| 256 | HYS-46_256 |
| 257 | HYS-46_257 |
| 258 | HYS-46_258 |
| 259 | HYS-46_259 |
| 260 | HYS-46_260 |
| 261 | HYS-46_261 |
| 262 | HYS-46_262 |
| 263 | HYS-46_263 |
| 264 | HYS-46_264 |
| 265 | HYS-46_265 |
| 266 | HYS-46_266 |
| 267 | HYS-46_267 |
| 268 | HYS-46_268 |
| 269 | HYS-46_269 |
| 270 | HYS-46_270 |
| 271 | HYS-46_271 |
| 272 | HYS-46_272 |
| 273 | HYS-46_273 |
| 274 | HYS-46_274 |
| 275 | HYS-46_275 |
| 276 | HYS-46_276 |
| 277 | HYS-46_277 |
| 278 | HYS-46_278 |
| 279 | HYS-46_279 |
| 280 | HYS-46_280 |
| 281 | HYS-46_281 |
| 282 | HYS-46_282 |
| 283 | HYS-46_283 |
| 284 | HYS-46_284 |
| 285 | HYS-46_285 |
| 286 | HYS-46_286 |
| 287 | HYS-46_287 |
| 288 | HYS-46_288 |
| 289 | HYS-46_289 |
| 290 | HYS-46_290 |
| 291 | HYS-46_291 |
| 292 | HYS-46_292 |
| 293 | HYS-46_293 |
| 294 | HYS-46_294 |
| 295 | HYS-46_295 |
| 296 | HYS-46_296 |
| 297 | HYS-46_297 |
| 198 | HYS-46_298 |
| 299 | HYS-46_299 |
| 300 | HYS-46_300 |
| 301 | HYS-46_301 |
| 302 | HYS-46_302 |
| 303 | HYS-46_303 |
| 304 | HYS-46_304 |
| 305 | HYS-46_305 |
| 306 | HYS-46_306 |
| 307 | HYS-46_307 |
| 308 | HYS-46_308 |
| 309 | HYS-46_309 |
| 310 | HYS-46_310 |
| 311 | HYS-46_311 |
| 312 | HYS-46_312 |
| 313 | HYS-46_313 |
| 314 | HYS-46_314 |
| 315 | HYS-46_315 |
| 316 | HYS-46_316 |
| 317 | HYS-46_317 |
| 318 | HYS-46_318 |
| 319 | HYS-46_319 |
| 320 | HYS-46_320 |
| 321 | HYS-46_321 |
| 322 | HYS-46_322 |
| 323 | HYS-46_323 |
| 324 | HYS-46_324 |
| 325 | HYS-46_325 |
| 326 | HYS-46_326 |
| 327 | HYS-46_327 |
| 328 | HYS-46_328 |
| 329 | HYS-46_329 |
| 330 | HYS-46_330 |
| 331 | HYS-46_331 |
| 332 | HYS-46_332 |
| 333 | HYS-46_333 |
| 334 | HYS-46_334 |
| 335 | HYS-46_335 |
| 336 | HYS-46_336 |
| 337 | HYS-46_337 |
| 338 | HYS-46_338 |
| 339 | HYS-46_339 |
| 340 | HYS-46_340 |
| 341 | HYS-46_341 |
| 342 | HYS-46_342 |
| 343 | HYS-46_343 |
| 344 | HYS-46_344 |
| 345 | HYS-46_345 |
| 346 | HYS-46_346 |
| 347 | HYS-46_347 |
| 348 | HYS-46_348 |
| 349 | HYS-46_349 |
| 350 | HYS-46_350 |
| 351 | HYS-46_351 |
| 352 | HYS-46_352 |
| 353 | HYS-46_353 |
| 354 | HYS-46_354 |
| 355 | HYS-46_355 |
| 356 | HYS-46_356 |
| 357 | HYS-46_357 |
| 358 | HYS-46_358 |
| 359 | HYS-46_359 |
| 360 | HYS-46_360 |
| 361 | HYS-46_361 |
| 362 | HYS-46_362 |
| 363 | HYS-46_363 |
| 364 | HYS-46_364 |
| 365 | HYS-46_365 |
| 366 | HYS-46_366 |
| 367 | HYS-46_367 |
| 368 | HYS-46_368 |
| 369 | HYS-46_369 |
| 370 | HYS-46_370 |
| 371 | HYS-46_371 |
| 372 | HYS-46_372 |
| 373 | HYS-46_373 |
| 374 | HYS-46_374 |
| 375 | HYS-46_375 |
| 376 | HYS-46_376 |
| 377 | HYS-46_377 |
| 378 | HYS-46_378 |
| 379 | HYS-46_379 |
| 380 | HYS-46_380 |
| 381 | HYS-46_381 |
| 382 | HYS-46_382 |
| 383 | HYS-46_383 |
| 384 | HYS-46_384 |
| 385 | HYS-46_385 |
| 386 | HYS-46_386 |
| 387 | HYS-46_387 |
| 388 | HYS-46_388 |
| 389 | HYS-46_389 |
| 390 | HYS-46_390 |
| 391 | HYS-46_391 |
| 392 | HYS-46_392 |
| 393 | HYS-46_393 |
| 394 | HYS-46_394 |
| 395 | HYS-46_395 |
| 396 | HYS-46_396 |
| 397 | HYS-46_397 |
| 398 | HYS-46_398 |
| 399 | HYS-46_399 |
| 400 | HYS-46_400 |
| 401 | HYS-46_401 |
| 402 | HYS-46_402 |
| 403 | HYS-46_403 |
| 404 | HYS-46_404 |
| 405 | HYS-47_1 |
| 406 | HYS-47_2 |
| 407 | HYS-47_3 |
| 408 | HYS-47_4 |
| 409 | HYS-47_5 |
| 410 | HYS-47_6 |
| 411 | HYS-47_7 |
| 412 | HYS-47_8 |
| 413 | HYS-47_9 |
| 414 | HYS-47_10 |
| 415 | HYS-47_11 |
| 416 | HYS-47_12 |
| 417 | HYS-47_13 |
| 418 | HYS-52_1 |
| 419 | HYS-52_2 |
| 420 | HYS-52_3 |
| 421 | HYS-52_4 |
| 422 | HYS-52_5 |
| 423 | HYS-52_6 |
| 424 | HYS-52_7 |
| 425 | HYS-52_8 |
| 426 | HYS-52_9 |
| 427 | HYS-52_10 |
| 428 | HYS-52_11 |
| 429 | HYS-52_12 |
| 430 | HYS-52_13 |
| 431 | HYS-52_14 |
| 432 | HYS-52_15 |
| 433 | HYS-52_16 |
| 434 | HYS-52_17 |
| 435 | HYS-52_18 |
| 436 | HYS-52_19 |
| 437 | HYS-52_20 |
| 438 | HYS-52_21 |
| 439 | HYS-52_22 |
| 440 | HYS-52_23 |
| 441 | HYS-54_1 |
| 442 | HYS-54_2 |
| 443 | HYS-54_3 |
| 444 | HYS-54_4 |
| 445 | HYS-54_5 |
| 446 | HYS-54_6 |
| 447 | HYS-54_7 |
| 448 | HYS-54_8 |
| 449 | HYS-54_9 |
| 450 | HYS-54_10 |
| 451 | HYS-54_11 |
| 452 | HYS-54_12 |
| 453 | HYS-54_13 |
| 454 | HYS-54_14 |
| 455 | HYS-54_15 |
| 456 | HYS-54_16 |
| 457 | HYS-54_17 |
| 458 | HYS-54_18 |
| 459 | HYS-54_19 |
| 460 | HYS-54_20 |
| 461 | HYS-54_21 |
| 462 | HYS-54_22 |
| 463 | HYS-54_23 |
| 464 | HYS-54_24 |
| 465 | HYS-54_25 |
| 466 | HYS-54_26 |
| 467 | HYS-54_27 |
| 468 | HYS-54_28 |
| 469 | HYS-54_29 |
| 470 | HYS-54_30 |
| 471 | HYS-54_31 |
| 472 | HYS-54_32 |
| 473 | HYS-54_33 |
| 474 | HYS-54_34 |
| 475 | HYS-54_35 |
| 476 | HYS-54_36 |
| 477 | HYS-54_37 |
| 478 | HYS-54_38 |
| 479 | HYS-54_39 |
| 480 | HYS-54_40 |
| 481 | HYS-54_41 |
| 482 | HYS-54_42 |
| 483 | HYS-54_43 |
| 484 | HYS-54_44 |
| 485 | HYS-55_1 |
| 486 | HYS-55_2 |
| 487 | HYS-55_3 |
| 488 | HYS-55_4 |
| 489 | HYS-55_5 |
| 490 | HYS-55_6 |
| 491 | HYS-55_7 |
| 492 | HYS-55_8 |
| 493 | HYS-55_9 |
| 494 | HYS-55_10 |
| 495 | HYS-55_11 |
| 496 | HYS-55_12 |
| 497 | HYS-55_13 |
| 498 | HYS-55_14 |
| 499 | HYS-55_15 |
| 500 | HYS-55_16 |
| 501 | HYS-55_17 |
| 502 | HYS-55_18 |
| 503 | HYS-55_19 |
| 504 | HYS-55_20 |
| 505 | HYS-55_21 |
| 506 | HYS-55_22 |
| 507 | HYS-55_23 |
| 508 | HYS-55_24 |
| 509 | HYS-55_25 |
| 510 | HYS-55_26 |
| 511 | HYS-55_27 |
| 512 | HYS-55_28 |
| 513 | HYS-55_29 |
| 514 | HYS-55_30 |
| 515 | HYS-55_31 |
| 516 | HYS-55_32 |
| 517 | HYS-55_33 |
| 518 | HYS-55_34 |
| 519 | HYS-55_35 |
| 520 | HYS-55_36 |
| 521 | HYS-55_37 |
| 522 | HYS-55_38 |
| 523 | HYS-55_39 |
| 524 | HYS-55_40 |
| 525 | HYS-55_41 |
| 526 | HYS-55_42 |
| 527 | HYS-55_43 |
| 528 | HYS-55_44 |
| 529 | HYS-55_45 |
| 530 | HYS-55_46 |
| 531 | HYS-55_47 |
| 532 | HYS-55_48 |
| 533 | HYS-55_49 |
| 534 | HYS-55_50 |
| 535 | HYS-55_51 |
| 536 | HYS-55_52 |
| 537 | HYS-55_53 |
| 538 | HYS-55_54 |
| 539 | HYS-55_55 |
| 540 | HYS-55_56 |
| 547 | HYS-57_1 |
| 548 | HYS-57_2 |
| 549 | HYS-57_3 |
| 550 | HYS-57_4 |
| 551 | HYS-57_5 |
| 552 | HYS-57_6 |
| 553 | HYS-57_7 |
| 554 | HYS-57_8 |
| 555 | HYS-57_9 |
| 556 | HYS-57_10 |
| 557 | HYS-57_11 |
| 558 | HYS-57_12 |
| 559 | HYS-57_13 |
| 560 | HYS-57_14 |
| 561 | HYS-57_15 |
| 562 | HYS-57_16 |
| 563 | HYS-57_17 |
| 564 | HYS-57_18 |
| 565 | HYS-57_19 |
| 566 | HYS-57_20 |
| 567 | HYS-57_21 |
| 568 | HYS-57_22 |
| 569 | HYS-57_23 |
| 570 | HYS-58_1 |
| 571 | HYS-58_2 |
| 572 | HYS-58_3 |
| 573 | HYS-58_4 |
| 574 | HYS-58_5 |
| 575 | HYS-58_6 |
| 576 | HYS-58_7 |
| 577 | HYS-58_8 |
| 578 | HYS-58_9 |
| 579 | HYS-58_10 |
| 580 | HYS-58_11 |
| 581 | HYS-58_12 |
| 582 | HYS-58_13 |
| 583 | HYS-58_14 |
| 584 | HYS-58_15 |
| 585 | HYS-58_16 |
| 586 | HYS-58_17 |
| 587 | HYS-60_1 |
| 588 | HYS-60_2 |
| 589 | HYS-60_3 |
| 590 | HYS-60_4 |
| 591 | HYS-60_5 |
| 592 | HYS-60_6 |
| 593 | HYS-60_7 |
| 594 | HYS-60_8 |
| 595 | HYS-60_9 |
| 596 | HYS-60_10 |
| 597 | HYS-60_11 |
| 598 | HYS-60_12 |
| 599 | HYS-60_13 |
| 600 | HYS-60_14 |
| 601 | HYS-61_1 |
| 602 | HYS-61_2 |
| 603 | HYS-61_3 |
| 604 | HYS-61_4 |
| 605 | HYS-61_5 |
| 606 | HYS-61_6 |
| 607 | HYS-61_7 |
| 608 | HYS-61_8 |
| 609 | HYS-61_9 |
| 61 | HYS-61_10 |
| 629 | HYS-62_1 |
| 630 | HYS-62_2 |
| 631 | HYS-62_3 |
| 632 | HYS-62_4 |
| 633 | HYS-62_5 |

*HYS-46_XXX = SEQ ID NO: XXX of Attorney Docket No. HYS-46, U.S. Ser. No. 10/005,499 filed 12/03/2001, the entire disclosure of which, including sequence listing, is incorporated herein by reference.
HYS-47_XXX = SEQ ID NO: XXX of Attorney Docket No. HYS-47, U.S. Ser. No. 60/341,362 filed 12/17/2001, the entire disclosure of which, including sequence listing, is incorporated herein by reference.
HYS-52_XXX = SEQ ID NO: XXX of Attorney Docket No. HYS-52, U.S. Ser. No. 60/379,875 filed 05/10/2002, the entire disclosure of which, including sequence listing, is incorporated herein by reference.
HYS-54_XXX = SEQ ID NO: XXX of Attorney Docket No. HYS-54, U.S. Ser. No. 60/379,834 filed 05/10/2002, the entire disclosure of which, including sequence listing, is incorporated herein by reference.
HYS-55_XXX = SEQ ID NO: XXX of Attorney Docket No. HYS-55, U.S. Ser. No. 60/384,450 filed 05/31/2002, the entire disclosure of which, including sequence listing, is incorporated herein by reference.
HYS-57_XXX = SEQ ID NO: XXX of Attorney Docket No. HYS-57, U.S. Ser. No. 60/384,665 filed 05/31/2002, the entire disclosure of which, including sequence listing, is incorporated herein by reference.
HYS-58_XXX = SEQ ID NO: XXX of Attorney Docket No. HYS-58, U.S. Ser. No. 60/389,715 filed 06/17/2002, the entire disclosure of which, including sequence listing, is incorporated herein by reference.
HYS-60_XXX = SEQ ID NO: XXX of Attorney Docket No. HYS-60, U.S. Ser. No. 60/393,722 filed 07/02/2002, the entire disclosure of which, including sequence listing, is incorporated herein by reference.
HYS-61_XXX = SEQ ID NO: XXX of Attorney Docket No. HYS-61, U.S. Ser. No. 60/390,531 filed 06/21/2002, the entire disclosure of which, including sequence listing, is incorporated herein by reference.
HYS-62_XXX = SEQ ID NO: XXX of Attorney Docket No. HYS-62, U.S. Ser. No. 60/391,326 filed /06/24/2002, the entire disclosure of which, including sequence listing, is incorporated herein by reference.

4.1 Adiponectin-Like Polypeptides And Polynucleotides

Adipose tissue primarily serves as an energy reservoir by storing fat and is involved in regulating available energy to the body. However, it has only recently become apparent that adipocytes synthesize and secrete many important proteins, including leptin, adipsin, complement components such as C3a and properdin, tumor necrosis factor (TNF)-α, plasminogen-activator inhibitor type 1 (PAI-1), and resistin. These adipocyte proteins are collectively called adipocytokines (Yamauchi et al., Nature Med. 7:941-946 (2001), herein incorporated by reference).

Adiponectin (also known as adipocyte complement-related protein, Acrp30, gelatin-binding protein (GBP28), or APM1) is such an adipocytokine that was identified by differential display cloning of preadipocytes and adipocytes in mouse cells. In humans, it was identified as an adipocyte-specific gene. There appears to be a large family of related proteins that share both sequence and structural homology including C1q, human type VIII and X collagens, precerebellin, and the hibernation-regulated proteins, hib 20, hib 25, and hib 27. Adiponectin (AdipoQ) has a modular design: a cleaved amino-terminal sequence, a region without homology to known proteins, a collagen-like region, and a C-terminal complement factor C1Q-like globular domain (Fruebis et al., Proc. Natl. Acad. Sci. USA 98:2005-2010 (2001), herein incorporated by reference). The globular domain forms homotrimers like TNF-α, and the collagen-like domains can further form higher order structures.

Functionally, adiponectin was found to suppress TNF-α-induced monocyte adhesion to human aortic endothelial cells (Ouchi et al., Circulation 100:2473-2476 (1999), herein incorporated by reference). They also reported that adiponectin suppressed the increased expression of VCAM-1, ICAM-1, and E-selectin, suggesting that adiponectin may attenuate the inflammatory responses associated with atherosclerosis. More recently, authors also reported that plasma levels of adiponectin were significantly lower in patients with coronary artery disease than in age and body mass index-matched normal subjects (Ouchi et al., Circulation 102:1296-1301 (2000), herein incorporated by reference). It was further shown that adiponectin suppressed TNF-α-induced nuclear factor Kappa B (NF-κB) activation accompanied by cAMP accumulation. Adiponectin also inhibited myelomonocytic progenitor cell proliferation, at least in part due to apoptotic mechanisms in hematopoietic colony formation assays. In macrophages, adiponectin suppressed the expression of class A macrophage scavenger receptors (MSR) and altered cholesterol metabolism. In particular, adiponectin reduced intracellular cholesteryl ester content of the macrophages (Ouchi et al., Circulation 103:1057-63 (2001), herein incorporated by reference). The findings suggested that adiponectin protein suppressed the transformation of macrophages to foam cells.

Insulin resistance induced by high-fat diet and associated with obesity is a major risk factor for diabetes and cardiovascular diseases. It has been shown that adipocytokines play a crucial role in these processes. TNF-α overproduced in adipose tissue contributes to insulin resistance. Leptin, another adipocytokine, which contributes to the regulation of food intake and energy expenditure, also affects insulin sensitivity and may lead to hypertension. Similarly, serum adiponectin concentrations are decreased in homozygous obese (ob/ob) mice, obese humans, diabetic patients, and patients with coronary artery diseases (Hotta et al. Arterioscler. Thromb. Vasc. Biol. 20:1595-1599 (2000), herein incorporated by reference).

In mouse models, it was shown that acute treatment with a proteolytically generated globular domain of Acrp30 (gAcrp30) could lead to altered lipid metabolism. In particular, the gAcrp30 reduced plasma fatty acid levels caused by administration of a high-fat test meal (Freubis et al., Proc. Natl. Acad. Sci. USA 98:2005-2010 (2001), herein incorporated by reference). This effect was in part due to increased fatty acid oxidation by muscle. Low doses of gAcrp30 given to mice that were on high-fat/sucrose diet caused profound and sustainable weight reduction without affecting food intake. These data indicated that adiponectin as well as other adiponectin family members may be involved in energy homeostasis and their dysregulation may lead to pathological conditions.

Recently, Yamauchi et al. showed that decreased expression of adiponectin correlates with insulin resistance in mouse models of altered insulin sensitivity (Yamauchi et al., Nature Med. 7:941-946 (2001), herein incorporated by reference). Adiponectin decreased the levels of triglycerides in muscle and liver in obese mice. These effects were due to increased fatty acid combustion and energy dissipation in muscle. The authors further showed that insulin resistance was completely reversed in lipoatrophic mice by administering combination of physiological doses of adiponectin and leptin, but only partially with either adiponectin or leptin alone.

The role of adiponectin was further studied in the adiponectin knock-out (KO) mice by Matsuda et al. (J. Biol. Chem. 277:37487-37491 (2002)) and Kubota et al. (J. Biol. Chem. 277:25863-25866 (2002), both herein incorporated by reference). The adiponectin-deficient mice in each study showed severe neointimal thickening and increased proliferation of vascular smooth muscle cells in mechanically injured arteries. Adenovirus-mediated supplement of adiponectin attenuated the neotintimal proliferation, suggesting that adiponectin plays a direct role in neointimal thickening of arteries, a key feature of the restenosis phenomenon observed after balloon angioplasty. In cultured smooth muscle cells, adiponectin attenuated DNA synthesis induced a variety of growth factors such as PDGF, HB-EGF, bFGF and EGF and cell proliferation and migration induced by HB-EGF. In cultured endothelial cells, adiponectin attenuated HB-EGF expression stimulated by TNFα (Matsuda et al., J. Biol. Chem. 277:37487-37491 (2002), herein incorporated by reference). Kubota et al. further showed that the levels of FFAs, triglycerides and total cholesterol of adipoenctin-deficient mice were significantly elevated indicating that the lipid metabolism of these mice was severely disrupted and the mice were hyperlipidemic (Kubota et al., J. Biol. Chem. 277:25863-25866 (2002), herein incorporated by reference). Adiponectin therefore has antiatherogenic properties.

In a separate study of adiponectin-KO mice, Maeda et al found that there was delayed clearance of FFA in plasma, low levels of fatty acid transport protein 1 (FATP1) mRNA in muscle, high levels of TNFα mRNA in adipose tissue and high plasma TNFα concentrations. These KO mice exhibited severe diet-induced insulin resistence with reduced insulin-receptor substrate 1 (IRS-1)-associated phosphatidyl inositol 3 (PI3)-kinase activity in the muscles. Adenovirus-mediated adiponectin expression in the KO mice reversed the increase of adipose TNFα mRNA and the diet-induced insulin resistance. In cultured myocytes, TNFα decreased FATP1 mRNA, IRS1-associated PI3-kinase activity and glucose uptake whereas adiponectin increased these parameters supporting the similar observations in mice (Maeda et al., Nature Med. 8:731-737, (2002), herein incorporated by reference).

Hotta et al have shown that plasma levels of adiponectin are decreased in Type 2 diabetes patients with coronary artery disease (CAD) complications and may cause the develoment of insulin resistance in these patients. In addition, the plasma adiponectin levels independently negatively correlated with serum triglyceridemia levels suggesting decreased adiponectin is associated with hypertriglyceridemia which is known to play a significant role in the deveopment of atherosclerosis. In addition, sex differences were observed in adiponectin concentrations in the diabetic subjects without CAD with higher levels in clinically normal women as well as in diabetic women suggesting that sex hormones including estrogen, progesterone and androgen may affect plasma adiponectin levels (Hotta et al., Arterioscler. Thromb. Vasc. Biol. 20:1595-1599 (2000), herein incorporated by reference). The plasma levels of adiponectin are also reduced in cardiovascular patients with end stage renal disease and the incidence of cardiovascular death is higher in renal failure patients with low plasma adiponectins compared with those with higher plasma adiponectin levels (Zoccali et al., J Am Soc Nephrol. 13:134-41 (2002), herein incorporated by reference). These data clearly show that adiponectin is involved in metabolic disorders including diabetes cardiovascular disease with and without renal complications.

Based on these studies and others, therapeutics that increase plasma adiponectin should be useful in preventing metabolic disorders, diabetes, cardiovascular and other related disorders such as atherogenesis, hypertriglyceridemia, vascular stenosis after angioplasty. Thus, the adiponectin-like polypeptides and polynucleotides of the invention may be used to treat obesity, diabetes, lipoatrophy, coronary artery diseases, atherosclerosis, and other obesity and diabetes-related cardiovascular pathologies. Adiponectin-like polypeptides and polynucleotides of the invention may also be used in treatment of autoimmune diseases and inflammation, to modulate immune responses, and to treat transplant patients. Adiponectin-like polypetides may also be used in the treatment of tumors such as solid tumors and leukemia.

Thirteen exemplary adiponectin-like sequences of the invention are described below: amino acid SEQ ID NO: 5 (and encoding nucleotide sequence SEQ ID NO: 4), amino aicid SEQ ID NO: 15 (and encoding nucleotide sequence SEQ ID NO: 14), amino acid SEQ ID NO: 28 (and encoding nucleotide SEQ ID NO: 27), amino acid SEQ ID NO: 160 (and encoding nucleotide sequence 159), amino acid SEQ ID NO: 186 (and encoding nucleotide sequence SEQ ID NO: 185), amino acid SEQ ID NO: 215 (and encoding nucleotide sequence SEQ ID NO: 214), amino acid sequence SEQ ID NO: 241 (and encoding nucleotide sequence SEQ ID NO: 240), amino acid SEQ ID NO: 272 (and encoding nucleotide sequence SEQ ID NO: 271), amino acid SEQ ID NO: 302 (and encoding nucleotide sequence SEQ ID NO: 301), amino acid SEQ ID NO: 323 (and encoding nucleotide sequence SEQ ID NO: 322), amino acid SEQ ID NO: 348 (and encoding nucleotide sequence SEQ ID NO: 347), amino acid SEQ ID NO: 355 (and encoding nucleotide sequence SEQ ID NO: 354), and amino acid SEQ ID NO: 378 (and encoding nucleotide sequence SEQ ID NO: 377).

The first adiponectin-like polypeptide of SEQ ID NO: 5 is an approximately 800-amino acid protein with a predicted molecular mass of approximately 90-kDa unglycosylated. The initial methionine starts at position 511 of SEQ ID NO: 4 and the putative stop codon begins at positions 2911 of SEQ ID NO: 4. Protein database searches with the BLASTP algorithm (Altschul S. F. et al., J. Mol. Evol. 36:290-300 (1993) and Altschul S. F. et al., J. Mol. Biol. 21:403-10 (1990), herein incorporated by reference) indicate that SEQ ID NO: 5 is homologous to adiponectin. Using the Pfam software program (Sonnhammer et al., Nucleic Acids Res., 26:320-322 (1998) herein incorporated by reference), adiponectin-like polypeptide of SEQ ID NO: 5 revealed its structural homology to C1q domain. Further description of the Pfam models can be found at http://pfam.wustl.edu/.

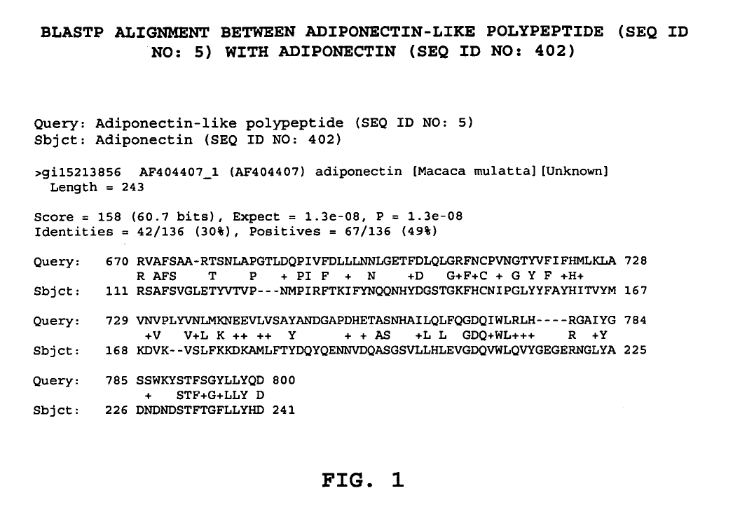

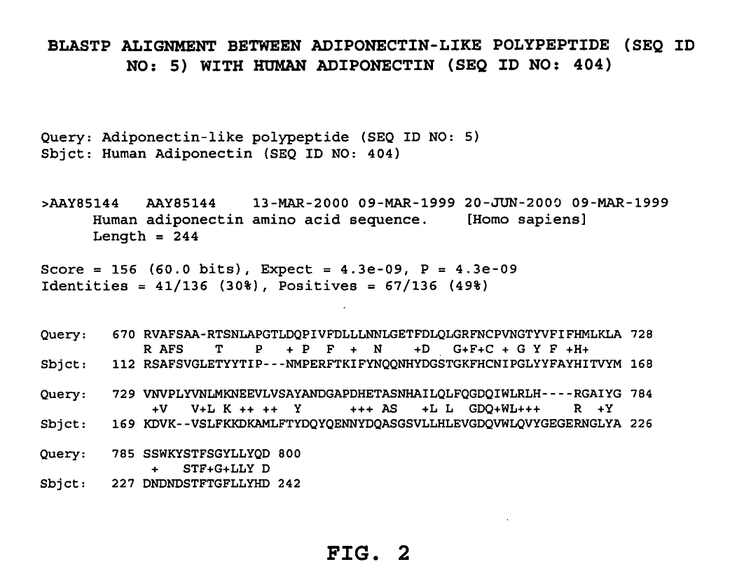

FIG. 1 shows the BLASTP amino acid sequence alignment between adiponectin-like polypeptide SEQ ID NO: 5 and adiponectin SEQ ID NO: 402 (Hotta et al., Diabetes 50:1126-1133 (2001)), indicating that the two sequences share 49% similarity over 136 amino acid residues and 30% identity over the same 136 amino acid residues, wherein A=Alanine, C=Cysteine, D=Aspartic Acid, E=Glutamic Acid, F=Phenylalanine, G-Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine. Gaps are presented as dashes.

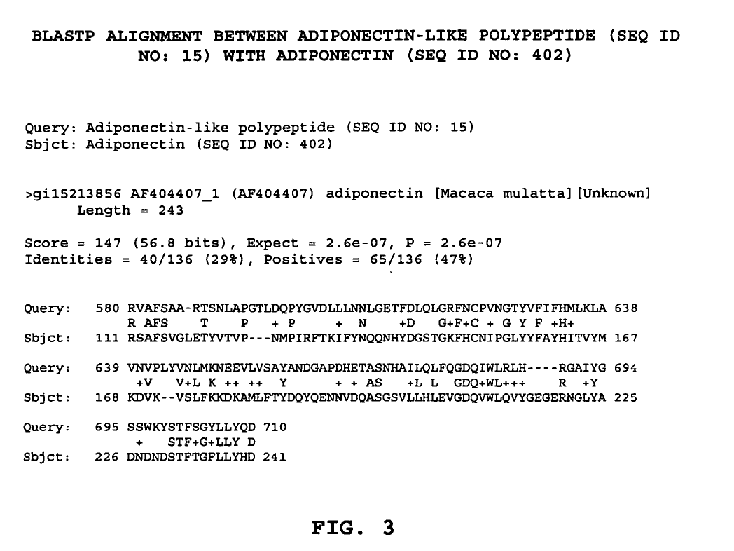

FIG. 2 shows the BLASTP amino acid sequence alignment between adiponectin-like polypeptide SEQ ID NO: 5 and human adiponectin SEQ ID NO: 404 (Patent No. JP3018186-B1), indicating that the two sequences share 49% similarity over 136 amino acid residues and 30% identity over the same 136 amino acid residues, wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine. Gaps are presented as dashes.

Using eMATRIX software package (Stanford University, Stanford, Calif.) (Wu et al., J. Comp. Biol. 6:219-235 (1999), herein incorporated by reference), adiponectin-like polypeptide of SEQ ID NO: 5 was determined to have following eMATRIX domain hits. The results describe: corresponding SEQ ID NO: in sequence listing, e-value, subtype, Accession number, name, position of the domain in the full-length protein, and the amino acid sequence and are displayed in Table 2 below, wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y'Tyrosine.

TABLE 2

| | |
|---|---|
| SEQ | Amino acid |

| | | |
|---|---|---|
| ID | Accession | sequence (start |

| | | | | | |
|---|---|---|---|---|---|
| NO; | e−value | Subtype | No. | Name | and end position) |

| | | | | | |
|---|---|---|---|---|---|
| 7 | 9.294e−19 | 18.26 | BL01113B | C1q domain | PIVFDLLLNNLGETFDLQ |
| | | | | proteins | LGRFNCPVNGTYVFIFHM |
| | | | | | (689-725) |
| 8 | 8.235e−12 | 15.60 | PR00007C | Complement | ETASNHAILQLFQGDQIW |
| | | | | C1Q domain | LRLH (757-779) |
| | | | | signature | |
| 9 | 4.857e−11 | 13.18 | BL01113C | C1q domain | ETASNHAILQLFQGDQIW |
| | | | | proteins | LR (757-777) |
| 10 | 1.250e−10 | 9.64 | PR00007D | Complement | KYSTFSGYLLY |
| | | | | C1Q domain | (788-799) |
| | | | | signature | |
| 11 | 2.161e−10 | 7.47 | BL01113D | C1q domain | STFSGYLLYQ |
| | | | | proteins | (790-800) |
| 12 | 7.107e−10 | 14.16 | PR00007B | Complement | FNCPVNGTYVFIFHMLKL |
| | | | | C1Q domain | AV (710-730) |
| | | | | signature | |
| 13 | 7.517e−10 | 19.33 | PR00007A | Complement | PGTLDQPIVFDLLLNNLG |
| | | | | C1Q domain | ETFDLQLGR |
| | | | | signature | (683-710) |

The second adiponectin-like polypeptide of SEQ ID NO: 15 is an approximately 710-amino acid protein with a predicted molecular mass of approximately 80-kDa unglycosylated. The initial methionine starts at position 511 of SEQ ID NO: 14 and the putative stop codon begins at positions 2641 of SEQ ID NO: 14. Protein database searches with the BLASTP algorithm (Altschul S. F. et al., J. Mol. Evol. 36:290-300 (1993) and Altschul S. F. et al., J. Mol. Biol. 21:403-10 (1990), herein incorporated by reference) indicate that SEQ ID NO: 15 is homologous to adiponectin. Using the Pfam software program (Sonnhammer et al., Nucleic Acids Res., 26:320-322 (1998) herein incorporated by reference), adiponectin-like polypeptide of SEQ ID NO: 15 revealed its structural homology to C1q domain. Further description of the Pfam models can be found at http://pfam.wustl.edu/.

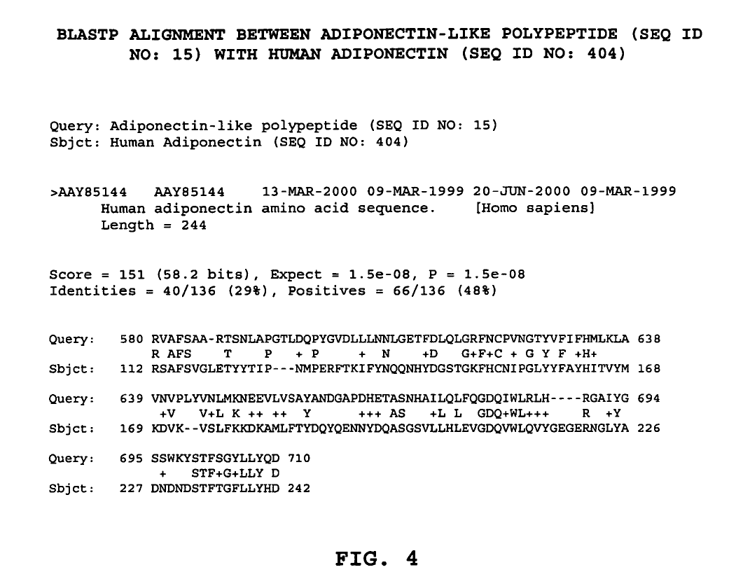

FIG. 3 shows the BLASTP amino acid sequence alignment between adiponectin-like polypeptide SEQ ID NO: 15 and adiponectin SEQ ID NO: 402 (Hotta et al., Diabetes 50:1126-1133 (2001)), indicating that the two sequences share 47% similarity over 136 amino acid residues and 29% identity over the same 136 amino acid residues, wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine. Gaps are presented as dashes.

FIG. 4 shows the BLASTP amino acid sequence alignment between adiponectin-like polypeptide SEQ ID NO: 15 and human adiponectin SEQ ID NO: 404 (Patent No. JP3018186-B 1), indicating that the two sequences share 48% similarity over 136 amino acid residues and 29% identity over the same 136 amino acid residues, wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine. Gaps are presented as dashes.

Using eMATRIX software package (Stanford University, Stanford, Calif.) (Wu et al., J. Comp. Biol., 6:219-235 (1999), herein incorporated by reference), adiponectin-like polypeptide of SEQ ID NO: 15 was determined to have following eMATRIX domain hits. The results describe: corresponding SEQ ID NO: in sequence listing, e-value, subtype, Accession number, name, position of the domain in the full-length protein, and the amino acid sequence and are shown in Table 3 below, wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine.

TABLE 3

| | |
|---|---|
| SEQ | Amino acid |

| | | |
|---|---|---|
| ID | Accession | sequence (start |

| | | | | | |
|---|---|---|---|---|---|
| NO; | e−value | Subtype | No. | Name | and end position) |

| | | | | | |
|---|---|---|---|---|---|
| 17 | 3.813e−14 | 18.26 | BL01113B | C1q domain | PYGVDLLLNNLGETFDL |
| | | | | proteins | QLGRFNCPVNGTYVFIFH |
| | | | | | M (599-635) |
| 18 | 8.235e−12 | 15.60 | PR00007C | Complement | ETASNHAILQLFQGDQIW |
| | | | | C1Q domain | LRLH (667-689) |
| | | | | signature | |
| 19 | 4.857e−11 | 13.18 | BL01113C | C1q domain | ETASNHAILQLFQGDQIW |
| | | | | proteins | LR (667-687) |
| 20 | 1.250e−10 | 9.64 | PR00007D | Complement | KYSTFSGYLLYQ |
| | | | | C1Q domain | (698-709) |
| | | | | signature | |
| 21 | 2.161e−10 | 7.47 | BL01113D | C1q domain | STFSGYLLYQ |
| | | | | proteins | (700-710) |
| 22 | 7.107e-10 | 14.16 | PR00007B | Complement | FNCPVNGTYVFIFHMLKL |
| | | | | C1Q domain | AV (620-640) |
| | | | | signature | |

The third adiponectin-like polypeptide of SEQ ID NO: 28 is an approximately 744-amino acid protein with a predicted molecular mass of approximately 83-kDa unglycosylated. The initial methionine starts at position 235 of SEQ ID NO: 27 and the putative stop codon begins at positions 2467 of SEQ ID NO: 27. Protein database searches with the BLASTP algorithm (Altschul S. F. et al., J. Mol. Evol. 36:290-300 (1993) and Altshul S. F. et al., J. Mol. Biol. 21:403-10 (1990), herein incorporated by reference) indicate that SEQ ID NO: 28 is homologous to adiponectin. Using the Pfam software program (Sonnhammer et al., Nucleic Acids Res., 26:320-322 (1998) herein incorporated by reference), adiponectin-like polypeptide of SEQ ID NO: 28 revealed its structural homology to C1q, and collagen domains. Further description of the Pfam models can be found at http://pfam.wustl.edu/.

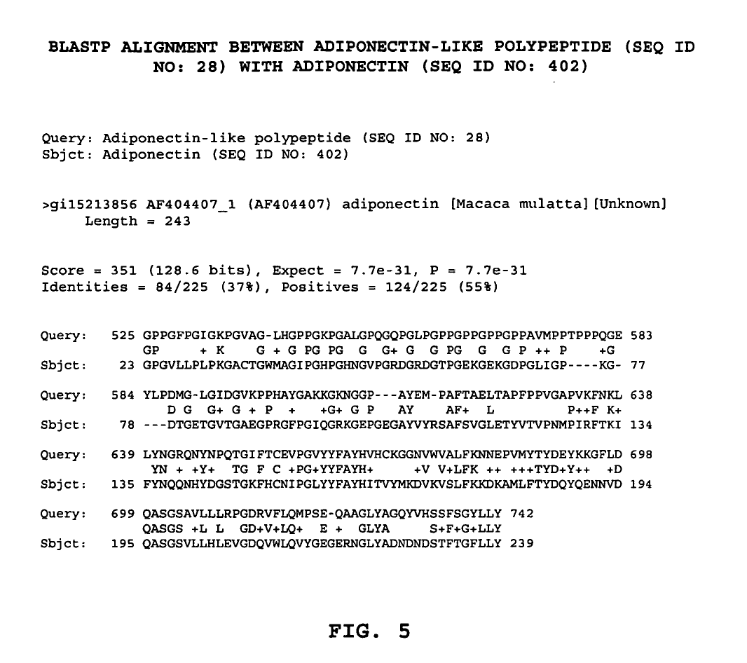

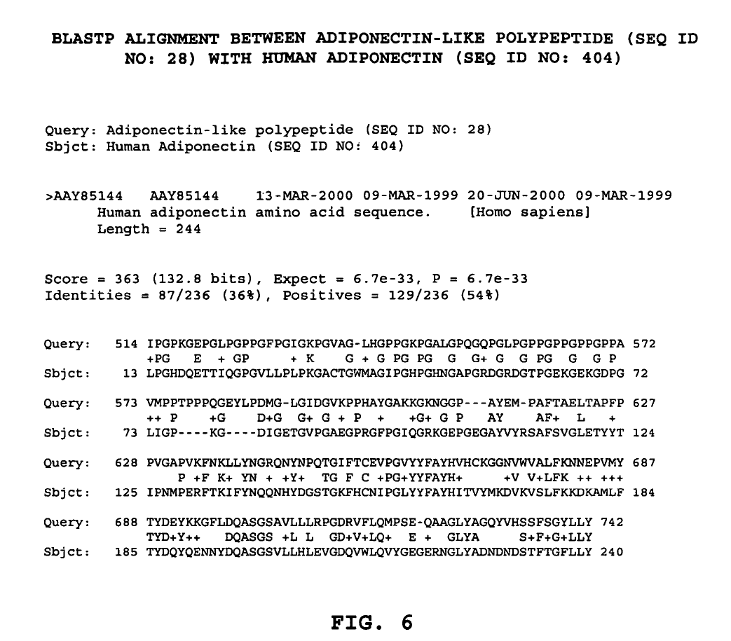

FIG. 5 shows the BLASTP amino acid sequence alignment between adiponectin-like polypeptide SEQ ID NO: 28 and adiponectin SEQ ID NO: 402 (Hotta et al, Diabetes 50:1126-1133 (2001)), indicating that the two sequences share 55% similarity over 225 amino acid residues and 37% identity over the same 225 amino acid residues, wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine. Gaps are presented as dashes.

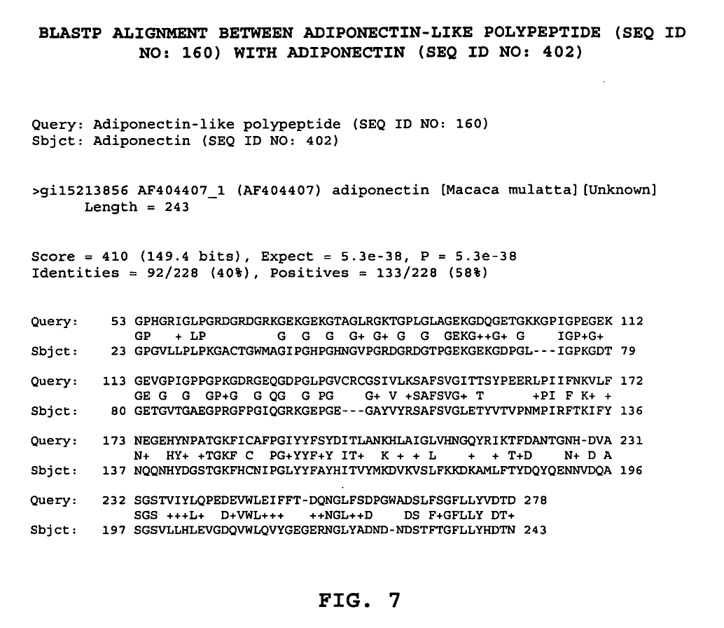

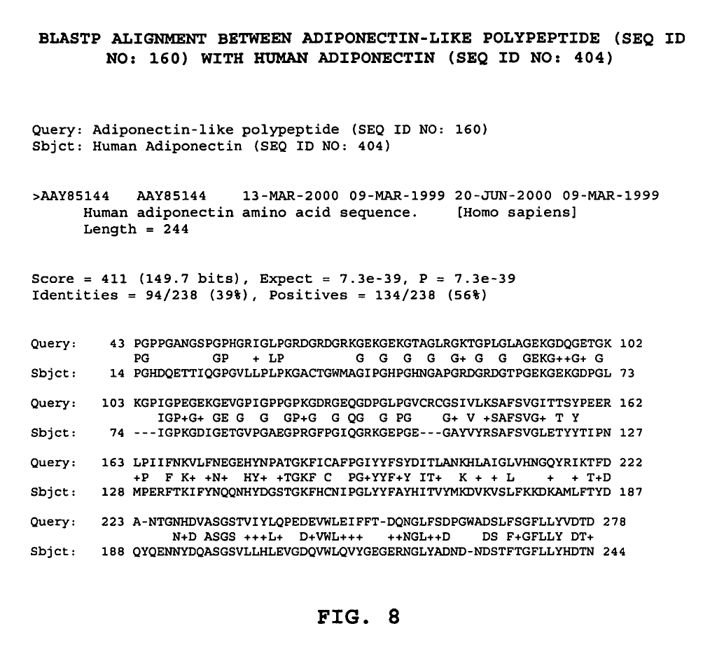

FIG. 6 shows the BLASTP amino acid sequence alignment between adiponectin-like polypeptide SEQ ID NO: 28 and human adiponectin SEQ ID NO: 404 (Patent No. JP3018186-B1), indicating that the two sequences share 54% similarity over 236 amino acid residues and 36% identity over the same 236 amino acid residues, wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine. Gaps are presented as dashes.

Using eMATRIX software package (Stanford University, Stanford, Calif.) (Wu et al., J. Comp. Biol., 6:219-235 (1999), herein incorporated by reference), adiponectin-like polypeptide of SEQ ID NO: 28 was determined to have following eMATRIX domain hits. The results describe: corresponding SEQ ID NO: in sequence listing, e-value, subtype, Accession number, name, position of the domain in the full-length protein, and the amino acid sequence and are shown in Table 4 below, wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine.

TABLE 4

| | |
|---|---|
| SEQ | Amino acid |

| | | |
|---|---|---|
| ID | Accession | sequence (start |

| | | | | | |
|---|---|---|---|---|---|
| NO; | e−value | Subtype | No. | Name | and end position) |

| | | | | | |
|---|---|---|---|---|---|
| 32 | 5.500e-35 | 18.26 | BL01113B | C1q domain | PVKFNKLLYNGRQNY |
| | | | | proteins | NPQTGIFTCEVPGVYY |
| | | | | | FAYHV (632-668) |
| 33 | 8.615e-23 | 14.16 | PR00007B | Complement | FTCEVPGVYYFAYHV |
| | | | | C1q domain | HCKGG |
| | | | | signature | (653-673) |
| 34 | 6.192e-22 | 19.33 | PR00007A | Complement | FPPVGAPVKFNKLLY |
| | | | | C1q domain | NGRQNYNPQTGI |
| | | | | signature | (626-653) |
| 35 | 5.846e−19 | 15.60 | PR00007C | Complement | DQASGSAVLLLRPGD |
| | | | | C1q domain | RVFLQ (698-720) |
| | | | | signature | |
| 36 | 6.700e−17 | 13.18 | BL01113C | C1q domain | DQASGSAVLLLRPGD |
| | | | | proteins | RVFLQ (698-718) |
| 37 | 6.885e−17 | 17.99 | BL01113A | C1q domain | PGPHGLPGIGKPGGPG |
| | | | | proteins | LPGQPGPKGDR |
| | | | | | (199-226) |
| 38 | 9.357e−15 | 20.42 | BL00420A | Speract receptor | GPPGAIGFPGPKGEGG |
| | | | | repeat proteins | IVGPQGPPGPKGE |
| | | | | domain proteins | (402-431) |
| 39 | 4.545e−14 | 17.99 | BL01113A | C1q domain | GPPGIPGIGGPSGPIGP |
| | | | | proteins | PGIPGPKGEP |
| | | | | | (495-522) |
| 40 | 8.636e−14 | 17.99 | BL01113A | C1q domain | GPPGEPGLPGIPGPMG |
| | | | | proteins | PPGAIGFPGPK |
| | | | | | (387-414) |
| 41 | 1.486e−13 | 17.99 | BL01113A | C1q domain | GVPGLLGPKGEPGIPG |
| | | | | proteins | DQGLQGPPGIP |
| | | | | | (474-501) |
| 42 | 1.730e−13 | 17.99 | BL01113A | C1q domain | GKPGMPGMPGKPGA |
| | | | | proteins | MGMPGAKGEIGQK |
| | | | | | (158-185) |
| 43 | 3.647e−13 | 9.64 | PR00007D | Complement | VHSSFSGYLLY |
| | | | | C1q domain | (732-743) |
| | | | | signature | |
| 44 | 4.162e−13 | 17.99 | BL01113A | C1q domain | GGPGLPGQPGPKGDR |
| | | | | proteins | GPKGLPGPQGLR |
| | | | | | (211-238) |
| 45 | 5.408e−13 | 20.42 | BL00420A | Speract receptor | GKPGMPGMPGKPGA |
| | | | | repeat proteins | MGMPGAKGEIGQKG |
| | | | | domain proteins | E (158-187) |
| 46 | 6.838e−13 | 17.99 | BL01113A | C1q domain | GIPGQPGFPGGKGEQ |
| | | | | proteins | GLPGLPGPPGLP |
| | | | | | (322-349) |
| 47 | 6.838e−13 | 17.99 | BL01113A | C1q domain | GAPGIGGPPGEPGLPG |
| | | | | proteins | IPGPMGPPGAI |
| | | | | | (381-408) |
| 48 | 7.081e−13 | 17.99 | BL01113A | C1q domain | GKPGQDGIPGQPGFPG |
| | | | | proteins | GKGEQGLPGLP |
| | | | | | (316-343) |
| 49 | 7.245e−13 | 20.42 | BL00420A | Speract receptor | GFPGKPGFLGEVGPPG |
| | | | | repeat proteins | MRGFPGPIGPKGE |
| | | | | domain proteins | (435-464) |
| 50 | 8.541e−13 | 17.99 | BL01113A | C1q domain | GPPGIPGPKGEPGLPG |
| | | | | proteins | PPGFPGIGKPG |
| | | | | | (510-537) |
| 51 | 9.027e−13 | 17.99 | BL01113A | C1q domain | GMPGAPGVKGPPGM |
| | | | | proteins | HGPPGPVGLPGVG |
| | | | | | (246-273) |
| 52 | 9.027e−13 | 17.99 | BL01113A | C1q domain | GFPGPQGPLGKPGAP |
| | | | | proteins | GEPGPQGPIGVP |
| | | | | | (278-305) |
| 53 | 1.231e−12 | 17.99 | BL01113A | C1q domain | GPPGKPGALGPQGQP |
| | | | | proteins | GLPGPPGPPGPP |
| | | | | | (542-569) |
| 54 | 2.154e−12 | 17.99 | BL01113A | C1q domain | GPSGPIGPPGIPGPKGE |
| | | | | proteins | PGLPGPPGFP |
| | | | | | (504-531) |
| 55 | 2.615e−12 | 17.99 | BL01113A | C1q domain | GLPGIPGPMGPPGAIG |
| | | | | proteins | FPGPKGEGGIV |
| | | | | | (393-420) |
| 56 | 4.231e−12 | 17.99 | BL01113A | C1q domain | GKPGALGPQGQPGLP |
| | | | | proteins | GPPGPPGPPGPP |
| | | | | | (545-572) |
| 57 | 5.154e−12 | 20.42 | BL00420A | Speract receptor | GPPGEPGLPGIPGPMG |
| | | | | repeat proteins | PPGAIGFPGPKGE |
| | | | | domain proteins | (387-416) |
| 58 | 5.327e−12 | 20.42 | BL00420A | Speract receptor | GPIGPKGEHGQKGVP |
| | | | | repeat proteins | GLPGVPGLLGPKGE |
| | | | | domain proteins | (456-485) |
| 59 | 7.462e−12 | 17.99 | BL01113A | C1q domain | PGIGKPGGPGLPGQPG |
| | | | | proteins | PKGDRGPKGLP |
| | | | | | (205-232) |
| 60 | 8.385e−12 | 17.99 | BL01113A | C1q domain | GIGGPSGPIGPPGIPGP |
| | | | | proteins | KGEPGLPGPP |
| | | | | | (501-528) |
| 61 | 8.846e−12 | 17.99 | BL01113A | C1q domain | GPPGMRGFPGPIGPKG |
| | | | | proteins | EHGQKGVPGLP |
| | | | | | (447-474) |
| 62 | 1.000e−11 | 7.47 | BL01113D | C1q domain | SSFSGYLLYP |
| | | | | proteins | (734-744) |
| 63 | 1.818e−11 | 17.99 | BL01113A | C1q domain | GKPGGPGLPGQPGPK |
| | | | | proteins | GDRGPKGLPGPQ |
| | | | | | (208-235) |
| 64 | 4.764e−11 | 20.42 | BL00420A | Speract receptor | GEPGLPGIPGPMGPPG |
| | | | | repeat proteins | AIGFPGPKGEGGI |
| | | | | domain proteins | (390-419) |
| 65 | 5.418e−11 | 20.42 | BL00420A | Speract receptor | PGIGKPGFPGPKGDRG |
| | | | | repeat proteins | MGGVPGALGPRGE |
| | | | | domain proteins | (348-377) |
| 66 | 5.500e−11 | 17.99 | BL01113A | C1q domain | GPQGPPGPKGEPGLQ |
| | | | | proteins | GFPGKPGFLGEV |
| | | | | | (420-447) |
| 67 | 5.705e−11 | 17.99 | BL01113A | C1q domain | PGPQGYPGVGKPGMP |
| | | | | proteins | GMPGKPGAMGMP |
| | | | | | (149-176) |
| 68 | 6.114e−11 | 17.99 | BL01113A | C1q domain | GIPGIGGPSGPIGPPGIP |
| | | | | proteins | GPKGEPGLP |
| | | | | | (498-525) |
| 69 | 6.318e−11 | 17.99 | BL01113A | C1q domain | GPRGEKGPIGAPGIGG |
| | | | | proteins | PPGEPGLPGIP |
| | | | | | (372-399) |
| 70 | 6.891e−11 | 20.42 | BL00420A | Speract receptor | GKPGFLGEVGPPGMR |
| | | | | repeat proteins | GFPGPIGPKGEHGQ |
| | | | | domain proteins | (438-467) |
| 71 | 7.545e−11 | 17.99 | BL01113A | C1q domain | GEPGPQGPIGVPGVQ |
| | | | | proteins | GPPGIPGIGKPG |
| | | | | | (293-320) |
| 72 | 8.773e−11 | 17.99 | BL01113A | C1q domain | GIGGPPGEPGLPGIPGP |
| | | | | proteins | MGPPGAIGFP |
| | | | | | (384-411) |
| 73 | 9.386e−11 | 17.99 | BL01113A | C1q domain | GKPGAPGEPGPQGPIG |
| | | | | proteins | VPGVQGPPGIP |
| | | | | | (287-314) |
| 74 | 9.795e-11 | 17.99 | BL01113A | C1q domain | GLPGQPGPKGDRGPK |
| | | | | proteins | GLPGPQGLRGPK |
| | | | | | (214-241) |
| 75 | 1.000e−10 | 17.99 | BL01113A | C1q domain | GVPGLPGVPGLLGPK |
| | | | | proteins | GEPGIPGDQGLQ |
| | | | | | (468-495) |
| 76 | 1.574e−10 | 17.99 | BL01113A | C1q domain | GKPGFLGEVGPPGMR |
| | | | | proteins | GFPGPIGPKGEH |
| | | | | | (438-465) |
| 77 | 1.766e−10 | 17.99 | BL01113A | C1q domain | GFPGPIGPKGEHGQK |
| | | | | proteins | GVPGLPGVPGLL |
| | | | | | (453-480) |
| 78 | 2.149e−10 | 17.99 | BL01113A | C1q domain | QGPPGIPGIGKPGQDG |
| | | | | proteins | IPGQPGFPGGK |
| | | | | | (307-334) |
| 79 | 2.149e−10 | 17.99 | BL01113A | C1q domain | PGPPGFPGIGKPGVAG |
| | | | | proteins | LHGPPGKPGAL |
| | | | | | (524-551) |
| 80 | 2.532e−10 | 17.99 | BL01113A | C1q domain | GQDGIPGQPGFPGGK |
| | | | | proteins | GEQGLPGLPGPP |
| | | | | | (319-346) |
| 81 | 2.532e−10 | 17.99 | BL01113A | C1q domain | GPIGAPGIGGPPGEPG |
| | | | | proteins | LPGIPGPMGPP |
| | | | | | (378-405) |
| 82 | 2.723e−10 | 17.99 | BL01113A | C1q domain | GPMGPPGAIGFPGPKG |
| | | | | proteins | EGGIVGPQGPP |
| | | | | | (399-426) |
| 83 | 2.918e−10 | 20.42 | BL00420A | Speract receptor | GPIGAPGIGGPPGEPG |
| | | | | repeat proteins | LPGIPGPMGPPGA |
| | | | | domain proteins | (378-407) |
| 84 | 3.489e−10 | 17.99 | BL01113A | C1q domain | GPLGKPGAPGEPGPQ |
| | | | | proteins | GPIGVPGVQGPP |
| | | | | | (284-311) |
| 85 | 3.681e−10 | 17.99 | BL01113A | C1q domain | PGVGKPGMPGMPGKP |
| | | | | proteins | GAMGMPGAKGEI |
| | | | | | (155-182) |
| 86 | 3.681e−10 | 17.99 | BL01113A | C1q domain | GMPGMPGKPGAMGM |
| | | | | proteins | PGAKGEIGQKGEI |
| | | | | | (161-188) |
| 87 | 3.872e−10 | 17.99 | BL01113A | C1q domain | GEPGLQGFPGKPGFL |
| | | | | proteins | GEVGPPGMRGFP |
| | | | | | (429-456) |
| 88 | 4.255e−10 | 17.99 | BL01113A | C1q domain | GQPGLPGPPGPPGPPG |
| | | | | proteins | PPAVMPPTPPP |
| | | | | | (554-581) |
| 89 | 4.447e−10 | 17.99 | BL01113A | C1q domain | GLPGVPGLLGPKGEP |
| | | | | proteins | GIPGDQGLQGPP |
| | | | | (471-498) | |
| 90 | 4.830e−10 | 17.99 | BL01113A | C1q domain | GLLGPKGEPGIPGDQG |
| | | | | proteins | LQGPPGIPGIG |
| | | | | | (477-504) |
| 91 | 5.787e−10 | 17.99 | BL01113A | C1q domain | GFPGGKGEQGLPGLP |
| | | | | proteins | GPPGLPGIGKPG |
| | | | | | (328-355) |
| 92 | 5.787e−10 | 17.99 | BL01113A | C1q domain | GFPGKPGFLGEVGPPG |
| | | | | proteins | MRGFPGPIGPK |
| | | | | | (435-462) |
| 93 | 5.979e−10 | 17.99 | BL01113A | C1q domain | GPQGQPGLPGPPGPPG |
| | | | | proteins | PPGPPAVMPPT |
| | | | | | (551-578) |
| 94 | 6.016e−10 | 20.42 | BL00420A | Speract receptor | GIPGQPGFPGGKGEQ |
| | | | | repeat proteins | GLPGLPGPPGLPGI |
| | | | | domain proteins | (322-351) |
| 95 | 6.170e−10 | 17.99 | BL01113A | C1q domain | PGIGKPGQDGIPGQPG |
| | | | | proteins | FPGGKGEQGLP |
| | | | | | (313-340) |
| 96 | 6.170e−10 | 17.99 | BL01113A | C1q domain | GLHGPPGKPGALGPQ |
| | | | | proteins | GQPGLPGPPGPP |
| | | | | | (539-566) |
| 97 | 6.459e−10 | 20.42 | BL00420A | Speract receptor | QGYPGVGKPGMPGM |
| | | | | repeat proteins | PGKPGAMGMPGAKG |
| | | | | domain proteins | E (152-181) |
| 98 | 6.553e−10 | 17.99 | BL01113A | C1q domain | GQKGVPGLPGVPGLL |
| | | | | proteins | GPKGEPGIPGDQ |
| | | | | | (465-492) |
| 99 | 6.553e−10 | 17.99 | BL01113A | C1q domain | GIPGPKGEPGLPGPPG |
| | | | | proteins | FPGIGKPGVAG |
| | | | | | (513-540) |
| 100 | 6.902e−10 | 20.42 | BL00420A | Speract receptor | GMPGMPGKPGAMGM |
| | | | | repeat proteins | PGAKGEIGQKGEIGP |
| | | | | domain proteins | (161-190) |
| 101 | 6.936e−10 | 17.99 | BL01113A | C1q domain | GALGPQGQPGLPGPP |
| | | | | proteins | GPPGPPGPPAVM |
| | | | | | (548-575) |
| 102 | 7.511e−10 | 17.99 | BL01113A | C1q domain | GVAGLHGPPGKPGAL |
| | | | | proteins | GPQGQPGLPGPP |
| | | | | | (536-563) |
| 103 | 7.702e−10 | 17.99 | BL01113A | C1q domain | PGPPGLPGIGKPGFPG |
| | | | | proteins | PKGDRGMGGVP |
| | | | | | (342-369) |
| 104 | 7.787e−10 | 20.42 | BL00420A | Speract receptor | GPPGKPGALGPQGQP |
| | | | | repeat proteins | GLPGPPGPPGPPGP |
| | | | | domain proteins | (542-571) |
| 105 | 8.277e−10 | 17.99 | BL01113A | C1q domain | GQPGFPGGKGEQGLP |
| | | | | proteins | GLPGPPGLPGIG |
| | | | | | (325-352) |
| 106 | 8.672e−10 | 20.42 | BL00420A | Speract receptor | GKPGFPGPKGDRGMG |
| | | | | repeat proteins | GVPGALGPRGEKGP |
| | | | | domain proteins | (351-380) |
| 107 | 9.071e−10 | 0.00 | PR00049D | Wilm+S Tumor | GPPGPPAVMPPTPPP |
| | | | | protein | (566-581) |
| | | | | signature | |
| 108 | 9.115e−10 | 20.42 | BL00420A | Speract receptor | PGVGKPGMPGMPGKP |
| | | | | repeat proteins | GAMGMPGAKGEIGQ |
| | | | | domain proteins | (155-184) |
| 109 | 9.234e−10 | 17.99 | BL01113A | C1q domain | GPKGEHGQKGVPGLP |
| | | | | proteins | GVPGLLGPKGEP |
| | | | | | (459-486) |
| 110 | 9.426e−10 | 17.99 | BL01113A | C1q domain | GPQGPLGKPGAPGEP |
| | | | | proteins | GPQGPIGVPGVQ |
| | | | | | (281-308) |
| 111 | 9.518e−10 | 19.43 | DM00215 | Proline-rich | LGPQGQPGLPGPPGPP |
| | | | | protein 3 | GPPGPPAVMPPTPPPQ |
| | | | | | G (550-583) |
| 112 | 1.000e−09 | 17.99 | BL01113A | C1q domain | GMPGKPGAMGMPGA |
| | | | | proteins | KGEIGQKGEIGPM |
| | | | | | (164-191) |
| 113 | 1.173e−09 | 17.99 | BL01113A | C1q domain | GVPGALGPRGEKGPI |
| | | | | proteins | GAPGIGGPPGEP |
| | | | | | (366-393) |
| 114 | 1.692e−09 | 17.99 | BL01113A | C1q domain | GQPGPKGDRGPKGLP |
| | | | | proteins | GPQGLRGPKGDK |
| | | | | | (217-244) |
| 115 | 1.692e−09 | 17.99 | BL01113A | C1q domain | GPIGPPGIPGPKGEPGL |
| | | | | proteins | PGPPGFPGIG (507-534) |
| 116 | 1.692e−09 | 17.99 | BL01113A | C1q domain | GKPGVAGLHGPPGKP |
| | | | | proteins | GALGPQGQPGLP |
| | | | | | (533-560) |
| 117 | 1.865e−09 | 17.99 | BL01113A | C1q domain | GEPGLPGIPGPMGPPG |
| | | | | proteins | AIGFPGPKGEG |
| | | | | | (390-417) |
| 118 | 2.212e−09 | 17.99 | BL01113A | C1q domain | PGPVGLPGVGKPGVT |
| | | | | proteins | GFPGPQGPLGKP |
| | | | | | (263-290) |
| 119 | 2.385e−09 | 17.99 | BL01113A | C1q domain | GAPGEPGPQGPIGVPG |
| | | | | proteins | VQGPPGIPGIG |
| | | | | | (290-317) |
| 120 | 2.731e−09 | 17.99 | BL01113A | C1q domain | PGVGKPGVTGFPGPQ |
| | | | | proteins | GPLGKPGAPGEP |
| | | | | | (269-296) |
| 121 | 2.938e−09 | 20.42 | BL00420A | Speract receptor | GIPGDQGLQGPPGIPGI |
| | | | | repeat proteins | GGPSGPIGPPGI |
| | | | | domain proteins | (486-515) |
| 122 | 3.423e−09 | 17.99 | BL01113A | C1q domain | GEGGIVGPQGPPGPK |
| | | | | proteins | GEPGLQGFPGKP |
| | | | | | (414-441) |
| 123 | 3.492e−09 | 20.42 | BL00420A | Speract receptor | GLQGPPGIPGIGGPSG |
| | | | | repeat proteins | PIGPPGIPGPKGE |
| | | | | domain proteins | (492-521) |
| 124 | 3.797e−09 | 13.84 | DM00250B | kw Annexin | GQPGLPGPPGPPGPPG |
| | | | | antigen proline | PPAVMPPT (554-578) |
| | | | | tumor | |
| 125 | 4.288e−09 | 17.99 | BL01113A | C1q domain | GPPGPKGEPGLQGFPG |
| | | | | proteins | KPGFLGEVGPP |
| | | | | | (423-450) |
| 126 | 4.288e−09 | 17.99 | BL01113A | C1q domain | GIPGDQGLQGPPGIPGI |
| | | | | proteins | GGPSGPIGPP |
| | | | | | (486-513) |
| 127 | 4.323e−09 | 20.42 | BL00420A | Speract receptor | GEPGLQGFPGKPGFL |
| | | | | repeat proteins | GEVGPPGMRGFPGP |
| | | | | domain proteins | (429-458) |
| 128 | 5.073e−09 | 4.29 | BL00415N | Synapsins | PPGKPGALGPQGQPG |
| | | | | proteins | LPGPPGPPGPPGPPAV |
| | | | | | MPPTPPPQGEYLP |
| | | | | | (543-587) |
| 129 | 5.401e−09 | 4.29 | BL00415N | Synapsins | MPGAPGVKGPPGMH |
| | | | | proteins | GPPGPVGLPGVGKPG |
| | | | | | VTGFPGPQGPLGKPG |
| | | | | | (247-291) |
| 130 | 5.467e−09 | 4.29 | BL00415N | Synapsins | PQGPLGKPGAPGEPGP |
| | | | | proteins | QGPIGVPGVQGPPGIP |
| | | | | | GIGKPGQDGIPG |
| | | | | | (282-326) |
| 131 | 5.569e−09 | 20.42 | BL00420A | Speract receptor | GPPGIPGIGGPSGPIGP |
| | | | | repeat proteins | PGIPGPKGEPGL |
| | | | | domain proteins | (495-524) |
| 132 | 5.821e−09 | 15.53 | PD01234B | Protein nuclear | PGPPGPPGPPAVMPPT |
| | | | | bromodomain | PP (562-580) |
| | | | | trans. | |
| 133 | 6.019e−09 | 17.99 | BL01113A | C1q domain | GEVGPPGMRGFPGPIG |
| | | | | proteins | PKGEHGQKGVP |
| | | | | | (444-471) |
| 134 | 6.019e−09 | 17.99 | BL01113A | C1q domain | GEHGQKGVPGLPGVP |
| | | | | proteins | GLLGPKGEPGIP |
| | | | | | (462-489) |
| 135 | 6.186e−09 | 0.00 | PR00049D | Wilm's Tumor | GLPGPPGPPGPPGPP |
| | | | | protein | (557-572) |
| | | | | signature | |
| 136 | 6.365e−09 | 17.99 | BL01113A | C1q domain | GLPGPPGPPGPPGPPA |
| | | | | proteins | VMPPTPPPQGE |
| | | | | | (557-584) |
| 137 | 6.365e−09 | 17.99 | BL01113A | C1q domain | GPPGPPGPPGPPAVMP |
| | | | | proteins | PTPPPQGEYLP |
| | | | | | (560-587) |
| 138 | 6.954e−09 | 20.42 | BL00420A | Speract receptor | GGPGLPGQPGPKGDR |
| | | | | repeat proteins | GPKGLPGPQGLRGP |
| | | | | domain proteins | (211-240) |
| 139 | 7.404e−09 | 17.99 | BL01113A | C1q domain | GMPGAKGEIGQKGEI |
| | | | | proteins | GPMGIPGPQGPP |
| | | | | | (173-200) |
| 140 | 7.621e−09 | 4.49 | BL00291A | Prion protein | PGIGKPGGPGLPGQPG |
| | | | | | PKGDRGPKGLPGPQG |
| | | | | | LRGP (205-240) |
| 141 | 7.923e−09 | 17.99 | BL01113A | C1q domain | GKPGVTGFPGPQGPL |
| | | | | proteins | GKPGAPGEPGPQ |
| | | | | | (272-299) |
| 142 | 8.477e−09 | 20.42 | BL00420A | Speract receptor | GPKGEHGQKGVPGLP |
| | | | | repeat proteins | GVPGLLGPKGEPGI |
| | | | | domain | (459-488) |
| | | | | proteins. | |
| 143 | 8.615e−09 | 20.42 | BL00420A | Speract receptor | GQPGFPGGKGEQGLP |
| | | | | repeat proteins | GLPGPPGLPGIGKP |
| | | | | domain proteins | (325-354) |
| 144 | 8.615e−09 | 17.99 | BL01113A | C1q domain | GAIGFPGPKGEGGIVG |
| | | | | proteins | PQGPPGPKGEP |
| | | | | | (405-432) |
| 145 | 8.752e−09 | 4.29 | BL00415N | Synapsins | PKGEPGLPGPPGFPGI |
| | | | | proteins | GKPGVAGLHGPPGKP |
| | | | | | GALGPQGQGLPG |
| | | | | | (517-561) |
| 146 | 8.754e−09 | 20.42 | BL00420A | Speract receptor | GAPGIGGPPGEPGLPG |
| | | | | repeat proteins | IPGPMGPPGAIGF |
| | | | | domain proteins | (381-410) |
| 147 | 9.169e−09 | 20.42 | BL00420A | Speract receptor | GLPGQPGPKGDRGPK |
| | | | | repeat proteins | GLPGPQGLRGPKGD |
| | | | | domain proteins | (214-243) |
| 148 | 9.169e−09 | 20.42 | BL00420A | Speract receptor | GMGGVPGALGPRGE |
| | | | | repeat proteins | KGPIGAPGIGGPPGE |
| | | | | domain proteins | (363-392) |
| 149 | 9.308e−09 | 20.42 | BL00420A | Speract receptor | GPIGPPGIPGPKGEPGL |
| | | | | repeat proteins | PGPPGFPGIGKP |
| | | | | domain proteins | (507-536) |
| 150 | 9.542e−09 | 0.00 | PR00049D | Wilm's Tumor | PGPPGPPAVMPPTPP |
| | | | | protein | (565-580) |
| | | | | signature | |
| 151 | 9.585e−09 | 20.42 | BL00420A | Speract receptor | GKPGVTGFPGPQGPL |
| | | | | repeat proteins | GKPGAPGEPGPQGP |
| | | | | domain proteins | (272-301) |
| 152 | 9.827e−09 | 17.99 | BL01113A | C1q domain | GKPGAMGMPGAKGEI |
| | | | | proteins | GQKGEIGPMGIP |
| | | | | | (167-194) |
| 153 | 1.000e−08 | 20.42 | BL00420A | Speract receptor | GFLGEVGPPGMRGFP |
| | | | | repeat proteins | GPIGPKGEHGQKGV |
| | | | | domain proteins | (441-470) |
| 154 | 1.000e−08 | 17.99 | BL01113A | C1q domain | SLRGEQGPRGEPGPR |
| | | | | proteins | GPPGPPGLPGHG |
| | | | | | (115-142) |
| 155 | 1.000e−08 | 17.99 | BL01113A | C1q domain | GPKGEPGLQGFPGKP |
| | | | | proteins | GFLGEVGPPGMR |
| | | | | | (426-453) |

A predicted approximately twenty seven-residue signal peptide is encoded from approximately residue 1 to residue 27 of SEQ ID NO: 28 (SEQ ID NO: 30). The extracellular portion is useful on its own. This can be confirmed by expression in mammalian cells and sequencing of the cleaved product. The signal peptide region was predicted using the Neural Network SignalP V1.1 program (Nielsen et al, Int. J. Neural Syst. 8:581-599 (1997)). One of skill in the art will recognize that the actual cleavage site may be different than that predicted by the computer program. SEQ ID NO: 31 is the resulting peptide when the signal peptide is removed from SEQ ID NO: 28.

The fourth adiponectin-like polypeptide of SEQ ID NO: 160 is an approximately 289-amino acid protein with a predicted molecular mass of approximately 32-kDa unglycosylated. The initial methionine starts at position 80 of SEQ ID NO: 159 and the putative stop codon begins at positions 947 of SEQ ID NO: 159. Protein database searches with the BLASTP algorithm (Altschul S. F. et al., J. Mol. Evol. 36:290-300 (1993) and Altschul S. F. et al., J. Mol. Biol. 21:403-10 (1990), herein incorporated by reference) indicate that SEQ ID NO: 160 is homologous to adiponectin. Using the Pfam software program (Sonnhammer et al., Nucleic Acids Res., 26:320-322 (1998) herein incorporated by reference), adiponectin-like polypeptide of SEQ ID NO: 160 revealed its structural homology to C1q and collagen domains. Further description of the Pfam models can be found at http://pfam.wustl.edu/.

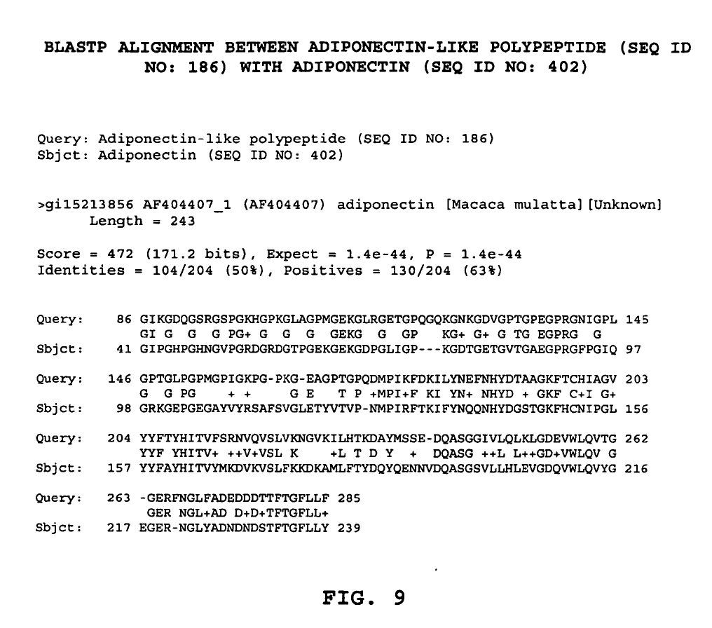

FIG. 7 shows the BLASTP amino acid sequence alignment between adiponectin-like polypeptide SEQ ID NO: 160 and adiponectin SEQ ID NO: 402 (Hotta et al, Diabetes 50:1126-1133 (2001)), indicating that the two sequences share 58% similarity over 228 amino acid residues and 40% identity over the same 228 amino acid residues, wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine. Gaps are presented as dashes.

FIG. 8 shows the BLASTP amino acid sequence alignment between adiponectin-like polypeptide SEQ ID NO: 160 and human adiponectin SEQ ID NO: 404 (Patent No. JP3018186-B1), indicating that the two sequences share 56% similarity over 238 amino acid residues and 39% identity over the same 238 amino acid residues, wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine. Gaps are presented as dashes.

Using eMATRIX software package (Stanford University, Stanford, Calif.) (Wu et al., J. Comp. Biol., 6:219-235 (1999), herein incorporated by reference), adiponectin-like polypeptide of SEQ ID NO: 160 was determined to have following eMATRIX domain hits. The results describe: corresponding SEQ ID NO: in sequence listing, e-value, subtype, Accession number, name, position of the domain in the full-length protein, and the amino acid sequence and are shown in Table 5 below, wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine.

TABLE 5

| | |
|---|---|
| SEQ | Amino acid |

| | | |
|---|---|---|
| ID | Accession | sequence (start |

| | | | | | |
|---|---|---|---|---|---|
| NO; | e−value | Subtype | No. | Name | and end position) |

| | | | | | |
|---|---|---|---|---|---|
| 164 | 1.581e-29 | 18.26 | BL01113B | C1q domain | PIIFNKVLFNEGEHYN |
| | | | | proteins | PATGKFICAFPGIYYFS |
| | | | | | YDI (164-200) |
| 165 | 1.000e−16 | 19.33 | PR00007A | Complement | YPEERLPIIFNKVLFNE |
| | | | | C1q domain | GEHYNPATGK |
| | | | | signature | (158-185) |
| 166 | 3.077e−15 | 13.18 | BL01113C | C1q domain | DVASGSTVIYLQPEDE |
| | | | | proteins | VWLE (229-249) |
| 167 | 8.200e−15 | 15.60 | PR00007C | Complement | DVASGSTVIYLQPEDE |
| | | | | C1q domain | VWLEIF (229-251) |
| | | | | signature | |
| 168 | 5.846e−14 | 14.16 | PR00007B | Complement | FICAFPGYYFSYDITL |
| | | | | C1q domain | ANK (185-205) |
| | | | | signature | |
| 169 | 1.243e−13 | 17.99 | BL01113A | C1q domain | GSPGPHGRIGLPGRDG |
| | | | | proteins | RDGRKGEKGEK |
| | | | | | (50-77) |
| 170 | 6.108e−13 | 17.99 | BL01113A | C1q domain | SIPGLPGPPGPPGANG |
| | | | | proteins | SPGPHGRIGLP (35-62) |
| 171 | 3.077e−12 | 17.99 | BL01113A | C1q domain | GPPGPPGANGSPGPH |
| | | | | proteins | GRIGLPGRDGRD |
| | | | | | (41-68) |
| 172 | 5.154e−12 | 20.42 | BL00420A | Speract receptor | GPPGANGSPGPHGRIG |
| | | | | repeat proteins | LPGRDGRDGRKGE |
| | | | | domain proteins | (44-73) |
| 173 | 1.655e−11 | 20.42 | BL00420A | Speract receptor | GPLGLAGEKGDQGET |
| | | | | repeat proteins | GKKGPIGPEGEKGE |
| | | | | domain proteins | (86-115) |
| 174 | 1.574e−10 | 17.99 | BL01113A | C1q domain | GLPGPPGPPGANGSPG |
| | | | | proteins | PHGRIGLPGRD |
| | | | | | (38-65) |
| 175 | 2.328e−10 | 20.42 | BL00420A | Speract receptor | GKKGPIGPEGEKGEV |
| | | | | repeat proteins | GPIGPPGPKGDRGE |
| | | | | domain proteins | (101-130) |
| 176 | 5.250e−10 | 9.64 | PR00007D | Complement | ADSLFSGFLLY |
| | | | | C1q domain | (264-275) |
| | | | | signature | |
| 177 | 9.617e−10 | 17.99 | BL01113A | C1q domain | GPPGANGSPGPHGRIG |
| | | | | proteins | LPGRDGRDGRK |
| | | | | | (44-71) |
| 178 | 4.185e−09 | 20.42 | BL00420A | Speract receptor | GANGSPGPHGRIGLPG |
| | | | | repeat proteins | RDGRDGRKGEKGE |
| | | | | domain proteins | (47-76) |
| 179 | 7.577e−09 | 17.99 | BL01113A | C1q domain | GLPGRDGRDGRKGEK |
| | | | | proteins | GEKGTAGLRGKT |
| | | | | | (59-86) |
| 180 | 7.577e−09 | 17.99 | BL01113A | C1q domain | GEKGEVGPIGPPGPKG |
| | | | | proteins | DRGEQGDPGL |
| | | | | | (110-137) |
| 181 | 9.031e−09 | 20.42 | BL00420A | Speract receptor | GSPGPHGRIGLPGRDG |
| | | | | repeat proteins | RDGRKGEKGEKGT |
| | | | | domain proteins | (50-79) |

A predicted approximately sixteen-residue signal peptide is encoded from approximately residue 1 to residue 16 of SEQ ID NO: 160 (SEQ ID NO: 162). The extracellular portion is useful on its own. This can be confirmed by expression in mammalian cells and sequencing of the cleaved product. The signal peptide region was predicted using the Neural Network SignalP V 1.1 program (Nielsen et al, Int. J. Neural Syst. 8:581-599 (1997)). One of skill in the art will recognize that the actual cleavage site may be different than that predicted by the computer program. SEQ ID NO: 163 is the resulting peptide when the signal peptide is removed from SEQ ID NO: 160.

The fifth adiponectin-like polypeptide of SEQ ID NO: 186 is an approximately 288-amino acid protein with a predicted molecular mass of approximately 32-kDa unglycosylated. The initial methionine starts at position 18 of SEQ ID NO: 185 and the putative stop codon begins at positions 882 of SEQ ID NO: 185. Protein database searches with the BLASTP algorithm (Altschul S. F. et al., J. Mol. Evol. 36:290-300 (1993) and Altschul S. F. et al., J. Mol. Biol. 21:403-10 (1990), herein incorporated by reference) indicate that SEQ ID NO: 186 is homologous to adiponectin. Using the Pfam software program (Sonnhammer et al., Nucleic Acids Res., 26:320-322 (1998) herein incorporated by reference), adiponectin-like polypeptide of SEQ ID NO: 186 revealed its structural homology to C1q and collagen domains. Further description of the Pfam models can be found at http://pfam.wustl.edu/.

FIG. 9 shows the BLASTP amino acid sequence alignment between adiponectin-like polypeptide SEQ ID NO: 186 and adiponectin SEQ ID NO: 402 (Hotta et al, Diabetes 50:1126-1133 (2001)), indicating that the two sequences share 63% similarity over 204 amino acid residues and 50% identity over the same 204 amino acid residues, wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine. Gaps are presented as dashes.

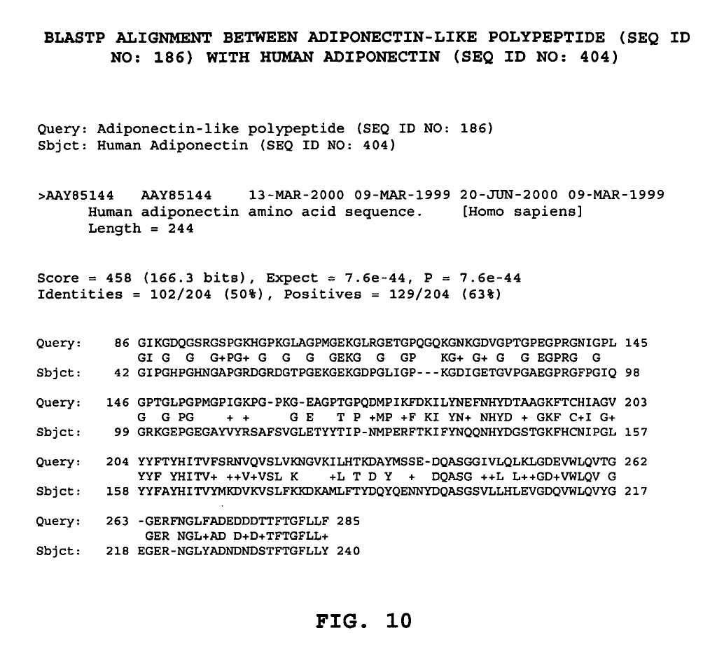

FIG. 10 shows the BLASTP amino acid sequence alignment between adiponectin-like polypeptide SEQ ID NO: 186 and human adiponectin SEQ ID NO: 404 (Patent No. JP3018186-B1), indicating that the two sequences share 63% similarity over 204 amino acid residues and 50% identity over the same 204 amino acid residues, wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine. Gaps are presented as dashes.

Using eMATRIX software package (Stanford University, Stanford, Calif.) (Wu et al., J. Comp. Biol., 6:219-235 (1999), herein incorporated by reference), adiponectin-like polypeptide of SEQ ID NO: 186 was determined to have following eMATRIX domain hits. The results describe: corresponding SEQ ID NO: in sequence listing, e-value, subtype, Accession number, name, position of the domain in the full-length protein, and the amino acid sequence and are shown in Table 6 below A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine.

TABLE 6

| | |
|---|---|
| SEQ | Amino acid |

| | | |
|---|---|---|
| ID | Accession | sequence (start |

| | | | | | |
|---|---|---|---|---|---|
| NO: | e−value | Subtype | No. | Name | and end position) |

| | | | | | |
|---|---|---|---|---|---|
| 190 | 2.750e-26 | 18.26 | BL01113B | C1q domain | PIKFDKILYNEFNHYD |
| | | | | proteins | TAAGKFTCHIAGVYY |
| | | | | | FTYHI (175-211) |
| 191 | 2.000e−16 | 15.60 | PR00007C | Complement | DQASGGIVLQLKLGD |
| | | | | C1q domain | EVWLQVT (240-262) |
| | | | | signature | |
| 192 | 6.143e−16 | 13.18 | BL01113C | C1q domain | DQASGGIVLQLKLGD |
| | | | | proteins | EVWLQ (240-260) |
| 193 | 1.771e−15 | 14.16 | PR00007B | Complement | FTCHIAGVYYFTYHIT |
| | | | | C1q domain | VFSR (196-216) |
| | | | | signature | |
| 194 | 4.064e−13 | 19.33 | PR00007A | Complement | TGPQDMPIKFDKILYN |
| | | | | C1q domain | EFNHYDTAAGK |
| | | | | signature | (169-196) |
| 195 | 5.622e−13 | 17.99 | BL01113A | C1q domain | GIPGNPGHNGLPGRD |
| | | | | proteins | GRDGAKGDKGDA |
| | | | | | (29-56) |
| 196 | 3.077e−12 | 17.99 | BL01113A | C1q domain | GLPGPMGPIGKPGPK |
| | | | | proteins | GEAGPTGPQDMP |
| | | | | | (149-176) |
| 197 | 3.455e−11 | 20.42 | BL00420A | Speract receptor | GRDGAKGDKGDAGE |
| | | | | repeat proteins | PGRPGSPGKDGTSGE |
| | | | | domain proteins | (44-73) |
| 198 | 3.618e−11 | 20.42 | BL00420A | Speract receptor | GIPGNPGHNGLPGRD |
| | | | | repeat proteins | GRDGAKGDKGDAGE |
| | | | | domain proteins | (29-58) |
| 199 | 9.673e−11 | 20.42 | BL00420A | Speract receptor | GDQGSRGSPGKHGPK |
| | | | | repeat proteins | GLAGPMGEKGLRGE |
| | | | | domain proteins | (89-118) |
| 200 | 1.191e−10 | 17.99 | BL01113A | C1q domain | GHPGIPGNPGHNGLP |
| | | | | proteins | GRDGRDGAKGDK |
| | | | | | (26-53) |
| 201 | 1.383e−10 | 17.99 | BL01113A | C1q domain | GLPGRDGRDGAKGD |
| | | | | proteins | KGDAGEPGRPGSP |
| | | | | | (38-65) |
| 202 | 3.489e−10 | 17.99 | BL01113A | C1q domain | GNPGHNGLPGRDGRD |
| | | | | proteins | GAKGDKGDAGEP |
| | | | | | (32-59) |
| 203 | 4.246e−10 | 20.42 | BL00420A | Speract receptor | GHPGIPGNPGHNGLP |
| | | | | repeat proteins | GRDGRDGAKGDKGD |
| | | | | domain proteins | (26-55) |
| 204 | 7.319e−10 | 17.99 | BL01113A | C1q domain | GDKGDAGEPGRPGSP |
| | | | | proteins | GKDGTSGEKGER |
| | | | | | (50-77) |
| 205 | 7.934e−10 | 20.42 | BL00420A | Speract receptor | GAKGDKGDAGEPGRP |
| | | | | repeat proteins | GSPGKDGTSGEKGE |
| | | | | domain proteins | (47-76) |
| 206 | 3.908e−09 | 20.42 | BL00420A | Speract receptor | GDKGDAGEPGRPGSP |
| | | | | repeat proteins | GKDGTSGEKGERGA |
| | | | | domain proteins | (50-79) |
| 207 | 4.323e−09 | 20.42 | BL00420A | Speract receptor | GPEGPRGNIGPLGPTG |
| | | | | repeat proteins | LPGPMGPIGKPGP |
| | | | | domain proteins | (134-163) |
| 208 | 4.349e−09 | 9.64 | PR00007D | Complement | DDTTFTGFLLF |
| | | | | C1q domain | (275-286) |
| | | | | signature | |
| 209 | 6.625e−09 | 7.47 | BL01113D | C1q domain | TTFTGFLLFS |
| | | | | proteins | (277-287) |
| 210 | 6.885e−09 | 17.99 | BL01113A | C1q domain | GAKGDKGDAGEPGRP |
| | | | | proteins | GSPGKDGTSGEK |
| | | | | | (47-74) |
| 211 | 8.096e−09 | 17.99 | BL01113A | C1q domain | GSPGKDGTSGEKGER |
| | | | | proteins | GADGKVEAKGIK |
| | | | | | (62-89) |
| 212 | 8.788e−09 | 17.99 | BL01113A | C1q domain | CRQGHPGIPGNPGHN |
| | | | | proteins | GLPGRDGRDGAK |
| | | | | | (23-50) |
| 213 | 9.585e−09 | 20.42 | BL00420A | Speract receptor | GPRGNIGPLGPTGLPG |
| | | | | repeat proteins | PMGPIGKPGPKGE |
| | | | | domain proteins | (137-166) |

The sixth adiponectin-like polypeptide of SEQ ID NO: 215 is an approximately 300-amino acid protein with a predicted molecular mass of approximately 34-kDa unglycosylated. The initial methionine starts at position 18 of SEQ ID NO: 214 and the putative stop codon begins at positions 918 of SEQ ID NO: 214. Protein database searches with the BLASTP algorithm (Altschul S. F. et al., J. Mol. Evol. 36:290-300 (1993) and Altshul S. F. et al., J. Mol. Biol. 21:403-10 (1990), herein incorporated by reference) indicate that SEQ ID NO: 215 is homologous to adiponectin. Using the Pfam software program (Sonnhammer et al., Nucleic Acids Res., 26:320-322 (1998) herein incorporated by reference), adiponectin-like polypeptide of SEQ ID NO: 215 revealed its structural homology to C1q and collagen domains. Further description of the Pfam models can be found at http://pfam.wustl.edu/.

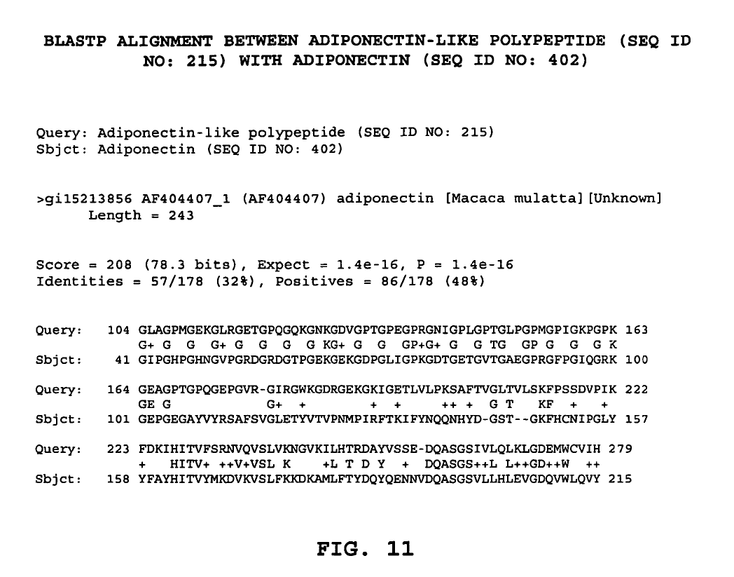

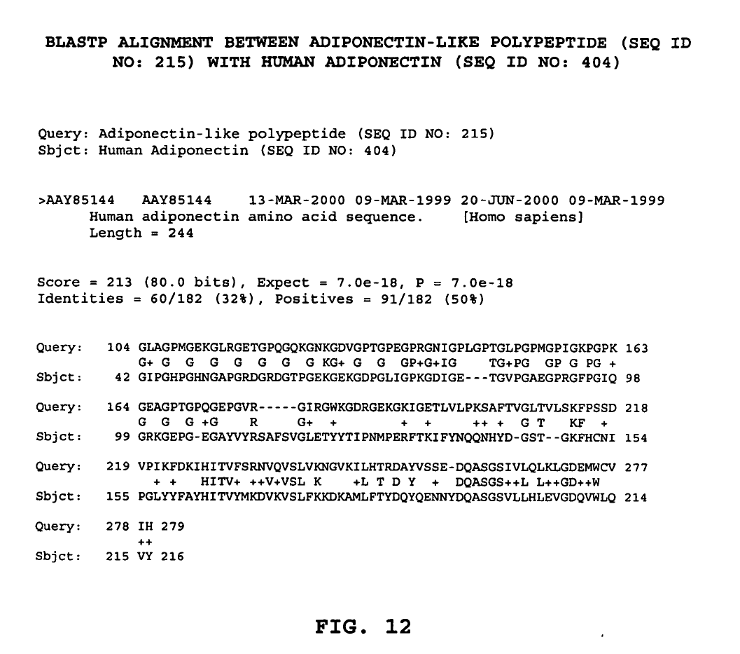

FIG. 11 shows the BLASTP amino acid sequence alignment between adiponectin-like polypeptide SEQ ID NO: 215 and adiponectin SEQ ID NO: 402 (Hotta et al, Diabetes 50:1126-1133 (2001)), indicating that the two sequences share 48% similarity over 178 amino acid residues and 32% identity over the same 178 amino acid residues, wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine. Gaps are presented as dashes.

FIG. 12 shows the BLASTP amino acid sequence alignment between adiponectin-like polypeptide SEQ ID NO: 215 and human adiponectin SEQ ID NO: 404 (Patent No. JP3018186-B1), indicating that the two sequences share 50% similarity over 182 amino acid residues and 32% identity over the same 182 amino acid residues, wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine. Gaps are presented as dashes.

Using eMATRIX software package (Stanford University, Stanford, Calif.) (Wu et al., J. Comp. Biol., 6:219-235 (1999), herein incorporated by reference), adiponectin-like polypeptide of SEQ ID NO: 215 was determined to have following eMATRIX domain hits. The results describe: corresponding SEQ ID NO: in sequence listing, e-value, subtype, Accession number, name, position of the domain in the full-length protein, and the amino acid sequence (both amino- and carboxy-flanking regions have been provided for the ease of viewing) and are shown in Table 7 below wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine.

TABLE 7

| | |
|---|---|
| SEQ | Amino acid |

| | | |
|---|---|---|
| ID | Accession | sequence (start |

| | | | | | |
|---|---|---|---|---|---|
| NO: | e−value | Subtype | No. | Name | and end position) |

| | | | | | |
|---|---|---|---|---|---|
| 218 | 8.909e−14 | 17.99 | BL01113A | C1q domain | GLPGPMGPIGKPGPK |
| | | | | proteins | GEAGPTGPQGEP |
| | | | | | (149-176) |
| 219 | 5.622e−13 | 17.99 | BL01113A | C1q domain | GIPGNPGHNGLPGRD |
| | | | | proteins | GRDGAKGDKGDA |
| | | | | | (29-56) |
| 220 | 3.455e−11 | 20.42 | BL00420A | Speract receptor | GRDGAKGDKGDAGE |
| | | | | repeat proteins | PGRPGSPGKDGTSGE |
| | | | | domain proteins | (44-73) |
| 221 | 3.618e−11 | 20.42 | BL00420A | Speract receptor | GIPGNPGHNGLPGRD |
| | | | | repeat proteins | GRDGAKGDKGDAGE |
| | | | | domain proteins | (29-58) |
| 222 | 9.673e−11 | 20.42 | BL00420A | Speract receptor | GDQGSRGSPGKHGPK |
| | | | | repeat proteins | GLAGPMGEKGLRGE |
| | | | | domain proteins | (89-118) |
| 223 | 1.191e−10 | 17.99 | BL01113A | C1q domain | GHPGIPGNPGHNGLP |
| | | | | proteins | GRDGRDGAKGDK |
| | | | | | (26-53) |
| 224 | 1.383e−10 | 17.99 | BL01113A | C1q domain | GLPGRDGRDGAKGD |
| | | | | proteins | KGDAGEPGRPGSP |
| | | | | | (38-65) |
| 225 | 3.489e−10 | 17.99 | BL01113A | C1q domain | GNPGHNGLPGRDGRD |
| | | | | proteins | GAKGDKGDAGEP |
| | | | | | (32-59) |
| 226 | 4.246e−10 | 20.42 | BL00420A | Speract receptor | GHPGIPGNPGHNGLP |
| | | | | repeat proteins | GRDGRDGAKGDKGD |
| | | | | domain proteins | (26-55) |
| 227 | 7.128e−10 | 17.99 | BL01113A | C1q domain | GKPGPKGEAGPTGPQ |
| | | | | proteins | GEPGVRGIRGWK |
| | | | | | (158-185) |
| 228 | 7.319e−10 | 17.99 | BL01113A | C1q domain | GDKGDAGEPGRPGSP |
| | | | | proteins | GKDGTSGEKGER |
| | | | | | (50-77) |
| 229 | 7.934e−10 | 20.42 | BL00420A | Speract receptor | GAKGDKGDAGEPGRP |
| | | | | repeat proteins | GSPGKDGTSGEKGE |
| | | | | domain proteins | (47-76) |
| 230 | 2.108e−09 | 20.42 | BL00420A | Speract receptor | GPKGEAGPTGPQGEP |
| | | | | repeat proteins | GVRGIRGWKGDRGE |
| | | | | domain proteins | (161-190) |
| 231 | 2.108e−09 | 13.18 | BL01113C | C1q domain | DASGSIVLQLKLGDE |
| | | | | proteins. | MWCV (258-278) |
| 232 | 3.596e−09 | 17.99 | BL01113A | C1q domain | GPIGKPGPKGEAGPTG |
| | | | | proteins | PQGEPGVRGIR |
| | | | | | (155-182) |
| 233 | 3.631e−09 | 15.60 | PR00007C | Complement | DQASGSIVLQLKLGD |
| | | | | C1q domain | EMWCVIH (258-280) |
| | | | | signature | |
| 234 | 3.908e−09 | 20.42 | BL00420A | Speract receptor | GDKGDAGEPGRPGSP |
| | | | | repeat proteins | GKDGTSGEKGERGA |
| | | | | domain proteins | (50-79) |
| 235 | 4.323e−09 | 20.42 | BL00420A | Speract receptor | GPEGPRGNIGPLGPTG |
| | | | | repeat proteins | LPGPMGPIGKPGP |
| | | | | domain proteins | (134-163) |
| 236 | 6.885e−09 | 17.99 | BL01113A | C1q domain | GAKGDKGDAGEPGRP |
| | | | | proteins | GSPGKDGTSGEK |
| | | | | | (47-74) |
| 237 | 8.096e−09 | 17.99 | BL01113A | C1q domain | GSPGKDGTSGEKGER |
| | | | | proteins | GADGKVEAKGIK |
| | | | | | (62-89) |
| 238 | 8.788e−09 | 17.99 | BL01113A | C1q domain | CRQGHPGIPGNPGHN |
| | | | | proteins | GLPGRDGRDGAK |
| | | | | | (23-50) |
| 239 | 9.585e−09 | 20.42 | BL00420A | Speract receptor | GPRGNIGPLGPTGLPG |
| | | | | repeat proteins | PMGPIGKPGPKGE |
| | | | | domain proteins | (137-166) |

The seventh adiponectin-like polypeptide of SEQ ID NO: 241 is an approximately 314-amino acid protein with a predicted molecular mass of approximately 35-kDa unglycosylated. The initial methionine starts at position 25 of SEQ ID NO: 240 and the putative stop codon begins at positions 1024 of SEQ ID NO: 240. Protein database searches with the BLASTP algorithm (Altschul S. F. et al., J. Mol. Evol. 36:290-300 (1993) and Altschul S. F. et al., J Mol. Biol. 21:403-10 (1990), herein incorporated by reference) indicate that SEQ ID NO: 241 is homologous to adiponectin. Using the Pfam software program (Sonnhammer et al., Nucleic Acids Res., 26:320-322 (1998) herein incorporated by reference), adiponectin-like polypeptide of SEQ ID NO: 241 revealed its structural homology to C1q and collagen domains. Further description of the Pfam models can be found at http://pfam.wustl.edu/.

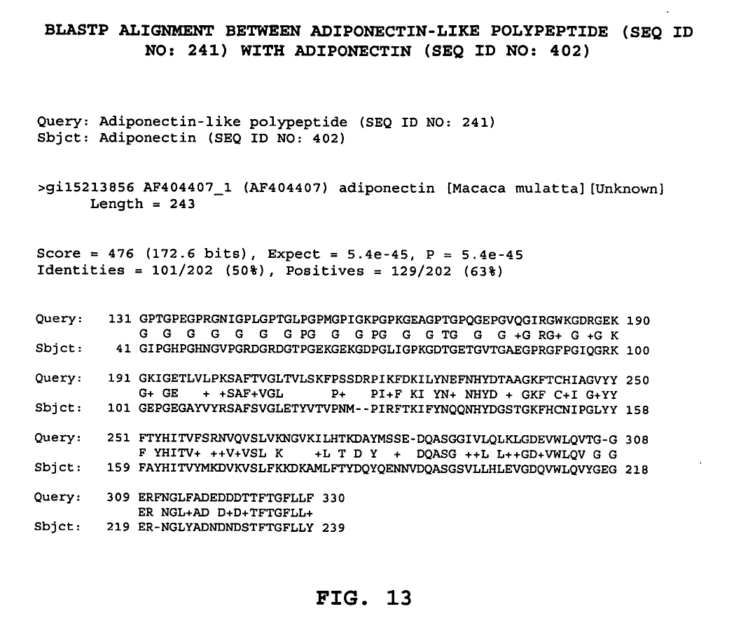

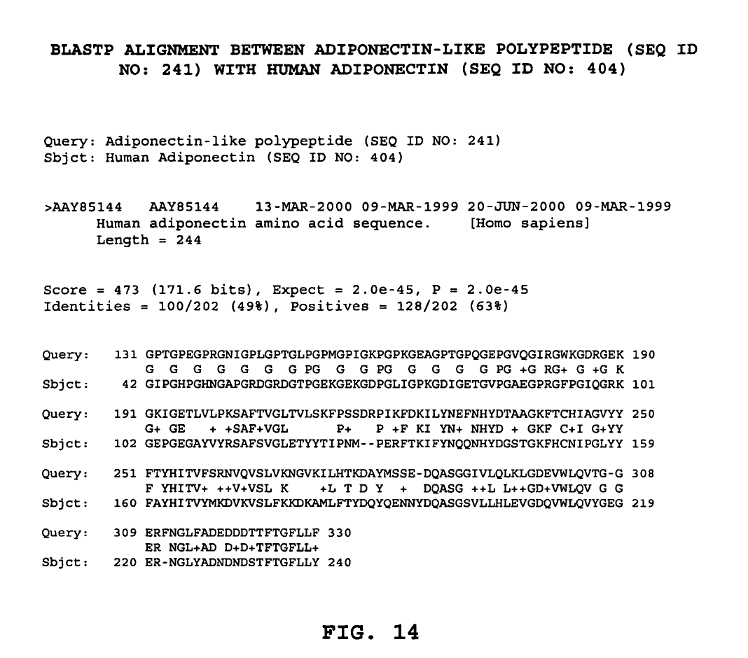

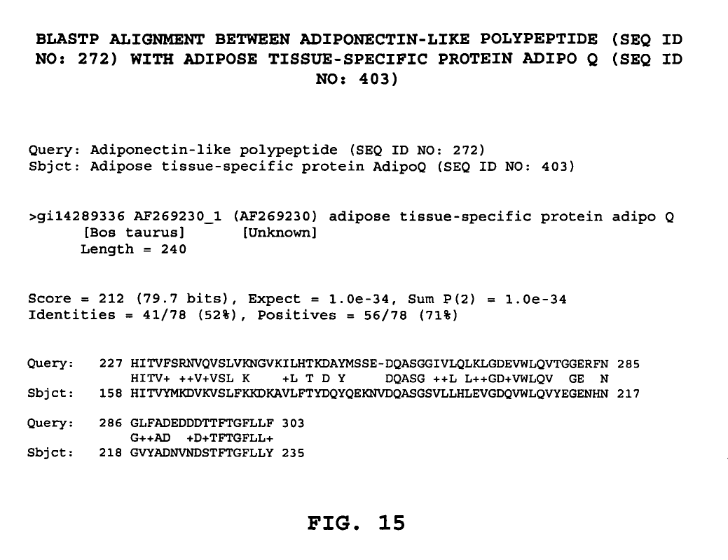

FIG. 13 shows the BLASTP amino acid sequence alignment between adiponectin-like polypeptide SEQ ID NO: 241 and adiponectin SEQ ID NO: 402 (Hotta et al, Diabetes 50:1126-1133 (2001)), indicating that the two sequences share 63% similarity over 202 amino acid residues and 50% identity over the same 202 amino acid residues, wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine. Gaps are presented as dashes.

FIG. 14 shows the BLASTP amino acid sequence alignment between adiponectin-like polypeptide SEQ ID NO: 241 and human adiponectin SEQ ID NO: 404 (Patent No. JP3018186-B1), indicating that the two sequences share 63% similarity over 202 amino acid residues and 49% identity over the same 202 amino acid residues, wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine. Gaps are presented as dashes.

Using eMATRIX software package (Stanford University, Stanford, Calif.) (Wu et al., J. Comp. Biol., 6:219-235 (1999), herein incorporated by reference), adiponectin-like polypeptide of SEQ ID NO: 241 was determined to have following eMATRIXdomain hits. The results describe: corresponding SEQ ID NO: in sequence listing, e-value, subtype, Accession number, name, position of the domain in the full-length protein, and the amino acid sequence and are shown in Table 8 below wherein A=Alanine, C=Cysteine, D=Aspartic E=Glutamic Acid, F=Phenylalanine, G=Glycine, H=Histidine, I=Isoleucine, ne, L=Leucine, M=Methionine, N=Asparagine, P=Proline, Q=Glutamine, nine, S=Serine, T=Threonine, V=Valine, W=Tryptophan, Y=Tyrosine.

TABLE 8

| | | | | | |
|---|---|---|---|---|---|
| SEQ | | | | | |
| ID | | | Accession | | Amino acid sequence |
| NO: | e-value | Subtype | No. | Name | (position) |

| | | | | | |
|---|---|---|---|---|---|
| 244 | 2.750e−26 | 18.26 | BL01113B | C1q domain | PIKFDKILYNEFNHYD |
| | | | | proteins | TAAGKFTCHIAGVYY |
| | | | | | FTYHI (220-256) |
| 245 | 2.000e−16 | 15.60 | PR00007C | Complement | DQASGGIVLQLKLGD |
| | | | | C1q domain | EVWLQVT |
| | | | | signature | (285-307) |
| 246 | 6.143e−16 | 13.18 | BL01113C | C1q domain | DQASGGIVLQLKLGD |
| | | | | proteins | EVWLQ (285-305) |
| 247 | 1.771e−15 | 14.16 | PR00007B | Complement | FTCHIAGVYYFTYHIT |
| | | | | C1q domain | VFSR (241-261) |
| | | | | signature | |
| 248 | 9.143e−15 | 19.33 | PR00007A | Complement | FPSSDRPIKFDKILYNE |
| | | | | C1q domain | FNHYDTAAGK |
| | | | | signature | (214-241) |
| 249 | 8.909e−14 | 17.99 | BL01113A | C1q domain | GLPGPMGPIGKPGPK |
| | | | | proteins | GEAGPTGPQGEP |
| | | | | | (149-176) |
| 250 | 5.622e−13 | 17.99 | BL01113A | C1q domain | GIPGNPGHNGLPGRD |
| | | | | proteins. | GRDGAKGDKGDA |
| | | | | | (29-56) |
| 251 | 3.455e−11 | 20.42 | BL00420A | Speract receptor | GRDGAKGDKGDAGE |
| | | | | repeat proteins | PGRPGSPGKDGTSGE |
| | | | | domain proteins | (44-73) |
| 252 | 3.618e−11 | 20.42 | BL00420A | Speract receptor | GIPGNPGHNGLPGRD |
| | | | | repeat proteins | GRDGAKGDKGDAGE |
| | | | | domain proteins | (29-58) |
| 253 | 9.673e−11 | 20.42 | BL00420A | Speract receptor | GDQGSRGSPGKHGPK |
| | | | | repeat proteins | GLAGPMGEKGLRGE |
| | | | | domain proteins | (89-118) |
| 254 | 1.191e−10 | 17.99 | BL01113A | C1q domain | GHPGIPGNPGHNGLP |
| | | | | proteins | GRDGRDGAKGDK |
| | | | | | (26-53) |
| 255 | 1.383e−10 | 17.99 | BL01113A | C1q domain | GLPGRDGRDGAKGD |
| | | | | proteins | KGDAGEPGRPGSP |
| | | | | | (38-65) |
| 256 | 1.957e−10 | 17.99 | BL01113A | C1q domain | GKPGPKGEAGPTGPQ |
| | | | | proteins | GEPGVQGIRGWK |
| | | | | | (158-185) |
| 257 | 3.489e−10 | 17.99 | BL01113A | C1q domain | GNPGHNGLPGRDGRD |
| | | | | proteins | GAKGDKGDAGEP |
| | | | | | (32-59) |
| 258 | 4.246e−10 | 20.42 | BL00420A | Speract receptor | GHPGIPGNPGHNGLP |
| | | | | repeat proteins | GRDGRDGAKGDKGD |
| | | | | domain proteins | (26-55) |
| 259 | 7.319e−10 | 17.99 | BL01113A | C1q domain | GDKGDAGEPGRPGSP |
| | | | | proteins | GKDGTSGEKGER |
| | | | | | (50-77) |
| 260 | 7.934e−10 | 20.42 | BL00420A | Speract receptor | GAKGDKGDAGEPGRP |
| | | | | repeat proteins | GSPGKDGTSGEKGE |
| | | | | domain proteins | (47-76) |
| 261 | 9.852e−10 | 20.42 | BL00420A | Speract receptor | GPKGEAGPTGPQGEP |
| | | | | repeat proteins | GVQGIRGWKGDRGE |
| | | | | domain proteins | (161-190) |
| 262 | 3.908e−09 | 20.42 | BL00420A | Speract receptor | GDKGDAGEPGRPGSP |
| | | | | repeat proteins | GKDGTSGEKGERGA |
| | | | | domain proteins | (50-79) |
| 263 | 4.323e−09 | 20.42 | BL00420A | Speract receptor | GPEGPRGNIGPLGPTG |
| | | | | repeat proteins | LPGPMGPIGKPGP |
| | | | | domain proteins | (134-163) |
| 264 | 4.349e−09 | 9.64 | PR00007D | Complement | DDTTFTGFLLF |
| | | | | C1q domain | (320-331) |
| | | | | signature | |
| 265 | 4.462e−09 | 17.99 | BL01113A | C1q domain | GPIGKPGPKGEAGPTG |
| | | | | proteins | PQGEPGVQGIR |
| | | | | | (155-182) |
| 266 | 6.625e−09 | 7.47 | BL01113D | C1q domain | TTFTGFLLFS |
| | | | | proteins | (322-332) |
| 267 | 6.885e−09 | 17.99 | BL01113A | C1q domain | GAKGDKGDAGEPGRP |
| | | | | proteins | GSPGKDGTSGEK |
| | | | | | (47-74) |
| 268 | 8.096e−09 | 17.99 | BL01113A | C1q domain | GSPGKDGTSGEKGER |
| | | | | proteins | GADGKVEAKGIK |
| | | | | | (62-89) |
| 269 | 8.788e−09 | 17.99 | BL01113A | C1q domain | CRQGHPGIPGNPGHN |
| | | | | proteins | GLPGRDGRDGAK |
| | | | | | (23-50) |
| 270 | 9.585e−09 | 20.42 | BL00420A | Speract receptor | GPRGNIGPLGPTGLPG |
| | | | | repeat proteins | PMGPIGKPGPKGE |
| | | | | domain proteins | (137-166) |

The eighth adiponectin-like polypeptide of SEQ ID NO: 272 is an approximately 306-amino acid protein with a predicted molecular mass of approximately 34-kDa unglycosylated. The initial methionine starts at position 25 of SEQ ID NO: 271 and the putative stop codon begins at positions 943 of SEQ ID NO: 271. Protein database searches with the BLASTP algorithm (Altschul S. F. et al., J. Mol. Evol. 36:290-300 (1993) and Altschul S. F. et al., J. Mol. Biol. 21:403-10 (1990), herein incorporated by reference) indicate that SEQ ID NO: 272 is homologous to adiponectin. Using the Pfam software program (Sonnhammer et al., Nucleic Acids Res., 26:320-322 (1998) herein incorporated by reference), adiponectin-like polypeptide of SEQ ID NO: 272 revealed its structural homology to C1q and collagen domains. Further description of the Pfam models can be found at http://pfam.wustl.edu/.

FIG. 15 shows the BLASTP amino acid sequence alignment between adiponectin-like polypeptide SEQ ID NO: 272 and adipose tissue-specific protein AdipoQ SEQ ID NO: 403 (Sato et al, J. Biol. Chem. 276:28849-28856 (2001)), indicating that the two sequences share 71% similarity over 78 amino acid residues and 52% identity over the same 78 amino acid residues, wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine. Gaps are presented as dashes.

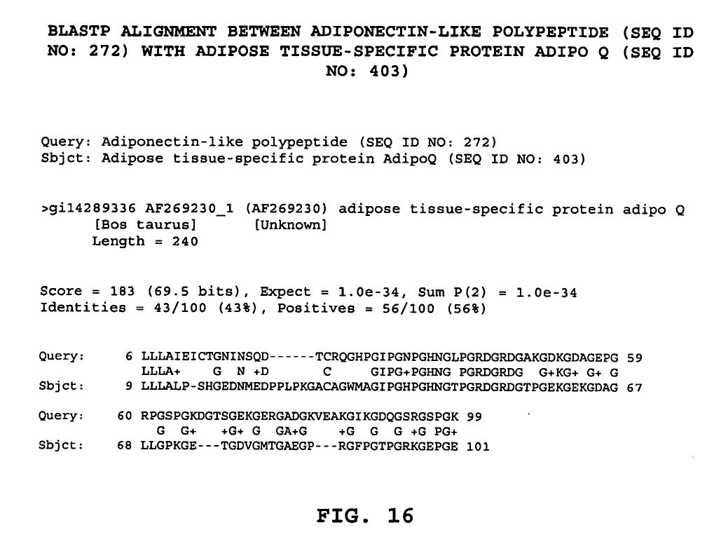

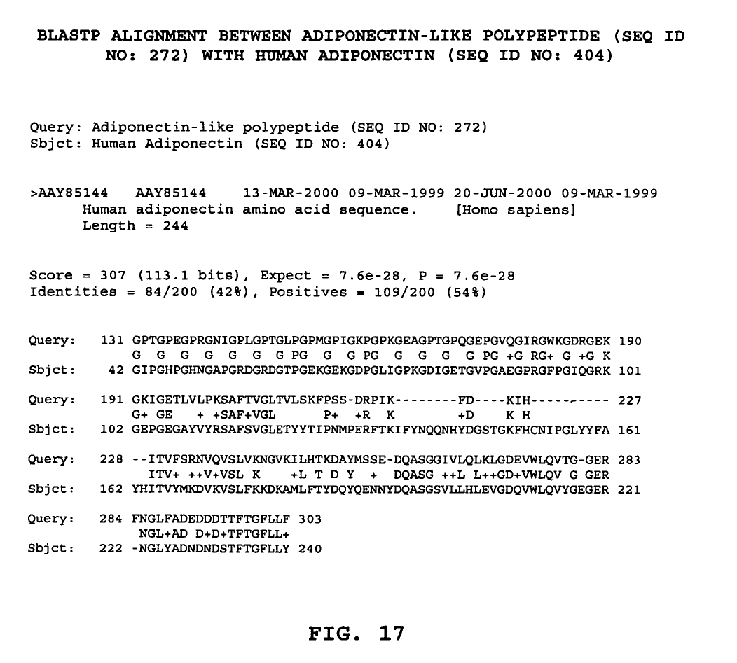

FIG. 16 shows the BLASTP amino acid sequence alignment between adiponectin-like polypeptide SEQ ID NO: 272 and adipose tissue-specific protein AdipoQ SEQ ID NO: 403 (Sato et al, J. Biol. Chem. 276:28849-28856 (2001)), indicating that the two sequences share 56% similarity over 100 amino acid residues and 43% identity over the same 100 amino acid residues, wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine. Gaps are presented as dashes.

FIG. 17 shows the BLASTP amino acid sequence alignment between adiponectin-like polypeptide SEQ ID NO: 272 and human adiponectin SEQ ID NO: 404 (Patent No. JP3018186-B1), indicating that the two sequences share 54% similarity over 200 amino acid residues and 42% identity over the same 200 amino acid residues, wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine. Gaps are presented as dashes.

Using eMATRIX software package (Stanford University, Stanford, Calif.) (Wu et al., J. Comp. Biol., 6:219-235 (1999), herein incorporated by reference), adiponectin-like polypeptide of SEQ ID NO: 272 was determined to have following eMATRIX domain hits. The results describe: corresponding SEQ ID NO: in sequence listing, e-value, subtype, Accession number, name, position of the domain in the full-length protein, and the amino acid sequence and are shown in Table 9 below wherein A=Alanine, C=Cysteine, D=Aspartic Acid E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine.

TABLE 9

| | | | | | |
|---|---|---|---|---|---|
| SEQ | | | | | |
| ID | | | Accession | | Amino acid sequence |
| NO: | e-value | Subtype | No. | Name | (start and end position) |

| | | | | | |
|---|---|---|---|---|---|
| 275 | 2.000e−16 | 15.60 | PR00007C | Complement | DQASGGIVLQLKLGD |
| | | | | C1q domain | EVWLQVT (258-280) |
| | | | | signature | |
| 276 | 6.143e−16 | 13.18 | BL01113C | C1q domam | DQASGGIVLQLKLGD |
| | | | | proteins | EVWLQ (258-278) |
| 277 | 8.909e−14 | 17.99 | BL01113A | C1q domain | GLPGPMGPIGKPGPK |
| | | | | proteins | GEAGPTGPQGEP |
| | | | | | (149-176) |
| 278 | 5.622e−13 | 17.99 | BL01113A | C1q domain | GIPGNPGHNGLPGRD |
| | | | | proteins | GRDGAKGDKGDA |
| | | | | | (29-56) |
| 279 | 3.455e−11 | 20.42 | BL00420A | Speract receptor | GRDGAKGDKGDAGE |
| | | | | repeat proteins | PGRPGSPGKDGTSGE |
| | | | | domain proteins | (44-73) |
| 280 | 3.618e−11 | 20.42 | BL00420A | Speract receptor | GIPGNPGHNGLPGRD |
| | | | | repeat proteins | GRDGAKGDKGDAGE |
| | | | | domain proteins | (29-58) |
| 281 | 9.673e−11 | 20.42 | BL00420A | Speract receptor | GDQGSRGSPGKHGPK |
| | | | | repeat proteins | GLAGPMGEKGLRGE |
| | | | | domain proteins | (89-118) |
| 282 | 1.191e−10 | 17.99 | BL01113A | C1q domain | GHPGIPGNPGHNGLP |
| | | | | proteins | GRDGRDGAKGDK |
| | | | | | (26-53) |
| 283 | 1.383e−10 | 17.99 | BL01113A | C1q domain | GLPGRDGRDGAKGD |
| | | | | proteins | KGDAGEPGRPGSP |
| | | | | | (38-65) |
| 284 | 1.957e−10 | 17.99 | BL01113A | C1q domain | GKPGPKGEAGPTGPQ |
| | | | | proteins | GEPGVQGIRGWK |
| | | | | | (158-185) |
| 285 | 3.489e−10 | 17.99 | BL01113A | C1q domain | GNPGHNGLPGRDGRD |
| | | | | proteins | GAKGDKGDAGEP |
| | | | | | (32-59) |
| 286 | 4.246e−10 | 20.42 | BL00420A | Speract receptor | GHPGIPGNPGHNGLP |
| | | | | repeat proteins | GRDGRDGAKGDKGD |
| | | | | domain proteins | (26-55) |
| 287 | 7.319e−10 | 17.99 | BL01113A | C1q domain | GDKGDAGEPGRPGSP |
| | | | | proteins | GKDGTSGEKGER |
| | | | | | (50-77) |
| 288 | 7.934e−10 | 20.42 | BL00420A | Speract receptor | GAKGDKGDAGEPGRP |
| | | | | repeat proteins | GSPGKDGTSGEKGE |
| | | | | domain proteins | (47-76) |
| 289 | 9.852e−10 | 20.42 | BL00420A | Speract receptor | GPKGEAGPTGPQGEP |
| | | | | repeat proteins | GVQGIRGWKGDRGE |
| | | | | domain proteins | (161-190) |
| 290 | 3.908e−09 | 20.42 | BL00420A | Speract receptor | GDKGDAGEPGRPGSP |
| | | | | repeat proteins | GKDGTSGEKGERGA |
| | | | | domain proteins | (50-79) |
| 291 | 4.323e−09 | 20.42 | BL00420A | Speract receptor | GPEGPRGNIGPLGPTG |
| | | | | repeat proteins | LPGPMGPIGKPGP |
| | | | | domain proteins | (134-163) |
| 292 | 4.349e−09 | 9.64 | PR00007D | Complement | DDTTFTGFLLF |
| | | | | C1q domain | (293-304) |
| | | | | signature | |
| 293 | 4.462e−09 | 17.99 | BL01113A | C1q domain | GPIGKPGPKGEAGPTG |
| | | | | proteins | PQGEPGVQGIR |
| | | | | | (155-182) |
| 294 | 6.625e−09 | 7.47 | BL01113D | C1q domain | TTFTGFLLFS (295-305) |
| | | | | proteins | |
| 295 | 6.885e−09 | 17.99 | BL01113A | C1q domain | GAKGDKGDAGEPGRP |
| | | | | proteins | GSPGKDGTSGEK |
| | | | | | (47-74) |
| 296 | 8.096e−09 | 17.99 | BL01113A | C1q domain | GSPGKDGTSGEKGER |
| | | | | proteins | GADGKVEAKGIK |
| | | | | | (62-89) |
| 297 | 8.788e−09 | 17.99 | BL01113A | C1q domain | CRQGHPGIPGNPGHN |
| | | | | proteins | GLPGRDGRDGAK |
| | | | | | (23-50) |
| 298 | 9.585e−09 | 20.42 | BL00420A | Speract receptor | GPRGNIGPLGPTGLPG |
| | | | | repeat proteins | PMGPIGKPGPKGE |
| | | | | domain proteins | (137-166) |

A predicted approximately nineteen-residue signal peptide is encoded from approximately residue 1 to residue 19 of SEQ ID NO: 186, 215, 241, and 272 (SEQ ID NO: 188). The extracellular portion is useful on its own. This can be confirmed by expression in mammalian cells and sequencing of the cleaved product. The signal peptide region was predicted using the Neural Network SignalP V1.1 program (Nielsen et al, Int. J. Neural Syst. 8:581:599 (1997)). One of skill in the art will recognize that the actual cleavage site may be different than that predicted by the computer program. SEQ ID NO: 189 is the resulting peptide when the signal peptide is removed from SEQ ID NO: 186. SEQ ID NO: 217 is the resulting peptide when the signal peptide is removed from SEQ ID NO: 215. SEQ ID NO: 243 is the resulting peptide when the signal peptide is removed from SEQ ID NO: 241. SEQ ID NO: 274 is the resulting peptide when the signal peptide is removed from SEQ ID NO: 272.

The ninth adiponectin-like polypeptide of SEQ ID NO: 302 is an approximately 338-amino acid protein with a predicted molecular mass of approximately 38-kDa unglycosylated. The initial methionine starts at position 199 of SEQ ID NO: 301 and the putative stop codon begins at positions 1213 of SEQ ID NO: 301. Protein database searches with the BLASTP algorithm (Altschul S. F. et al., J. Mol. Evol. 36:290-300 (1993) and Altschul S. F. et al., J. Mol. Biol. 21:403-10 (1990), herein incorporated by reference) indicate that SEQ ID NO: 301 is homologous to adiponectin. Using the Pfam software program (Sonnhammer et al., Nucleic Acids Res., 26:320-322 (1998) herein incorporated by reference), adiponectin-like polypeptide of SEQ ID NO: 302 revealed its structural homology to C1q and collagen domains. Further description of the Pfam models can be found at http://pfam.wustl.edu/.

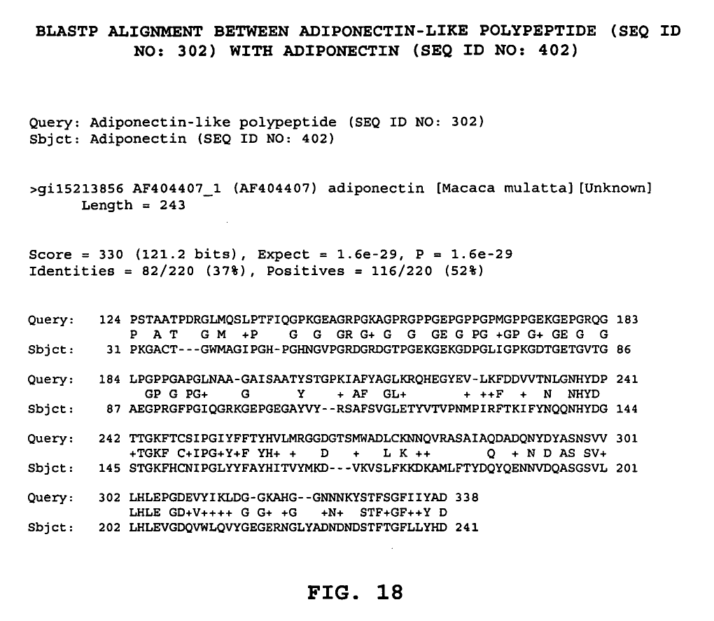

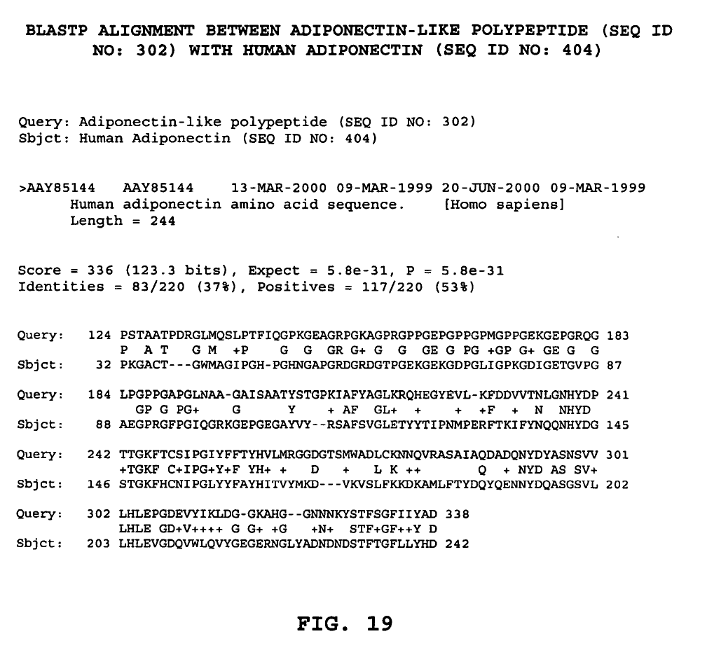

FIG. 18 shows the BLASTP amino acid sequence alignment between adiponectin-like polypeptide SEQ ID NO: 302 and adiponectin SEQ ID NO: 402 (Hotta et al, Diabetes 50:1126-1133 (2001)), indicating that the two sequences share 52% similarity over 220 amino acid residues and 37% identity over the same 220 amino acid residues, wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine. Gaps are presented as dashes.

FIG. 19 shows the BLASTP amino acid sequence alignment between adiponectin-like polypeptide SEQ ID NO: 302 and human adiponectin SEQ ID NO: 404 (Patent No. JP3018186-B1), indicating that the two sequences share 53% similarity over 220 amino acid residues and 37% identity over the same 220 amino acid residues, wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine. Gaps are presented as dashes.

Using eMATRIX software package (Stanford University, Stanford, Calif.) (Wu et al., J. Comp. Biol., 6:219-235 (1999), herein incorporated by reference), adiponectin-like polypeptide of SEQ ID NO: 302 was determined to have following eMATRIX domain hits. The results describe: corresponding SEQ ID NO: in sequence listing, e-value, subtype, Accession number, name, position of the domain in the full-length protein, and the amino acid sequence and shown in Table 10 below wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine.

TABLE 10

| | | | | | |
|---|---|---|---|---|---|
| SEQ | | | | | |
| ID | | | Accession | | Amino acid sequence |
| NO: | e-value | Subtype | No. | Name | (start and end position) |

| | | | | | |
|---|---|---|---|---|---|
| 304 | 3.647e−27 | 18.26 | BL01113B | C1q domain | VLKFDDVVTNLGNHY |
| | | | | proteins | DPTTGKFTCSIPGIYFF |
| | | | | | TYHV (225-261) |
| 305 | 6.657e−15 | 14.16 | PR00007B | Complement | FTCSIPGIYFFTYHVL |
| | | | | C1q domain | MRGG (246-266) |
| | | | | signature | |
| 306 | 2.047e−14 | 15.60 | PR00007C | Complement | DYASNSVVLHLEPGD |
| | | | | C1q domain | EVYIKLD (294-316) |
| | | | | signature | |
| 307 | 1.000e−13 | 17.99 | BL01113A | C1q domain | GEPGPPGPMGPPGEK |
| | | | | proteins | GEPGRQGLPGPP |
| | | | | | (162-189) |
| 308 | 2.532e−13 | 13.18 | BL01113C | C1q domain | DYASNSVVLHLEPGD |
| | | | | proteins | EVYIK (294-314) |
| 309 | 7.081e−13 | 17.99 | BL01113A | C1q domain | GKAGPRGPPGEPGPP |
| | | | | proteins | GPMGPPGEKGEP |
| | | | | | (153-180) |
| 310 | 8.297e−13 | 17.99 | BL01113A | C1q domain | GRPGKAGPRGPPGEP |
| | | | | proteins | GPPGPMGPPGEK |
| | | | | | (150-177) |
| 311 | 3.538e−12 | 17.99 | BL01113A | C1q domain | GPPGEPGPPGPMGPPG |
| | | | | proteins | EKGEPGRQGLP |
| | | | | | (159-186) |
| 312 | 4.808e−12 | 20.42 | BL00420A | Speract receptor | GRPGKAGPRGPPGEP |
| | | | | repeat proteins | GPPGPMGPPGEKGE |
| | | | | domain proteins | (150-179) |
| 313 | 5.385e−12 | 17.99 | BL01113A | C1q domain | GPPGPMGPPGEKGEP |
| | | | | proteins | GRQGLPGPPGAP |
| | | | | | (165-192) |
| 314 | 8.412e−12 | 19.33 | PR00007A | Complement | QHEGYEVLKFDDVVT |
| | | | | C1q domain | NLGNHYDPTTGK |
| | | | | signature | (219-246) |
| 315 | 5.909e−11 | 17.99 | BL01113A | C1q domain | GPMGPPGEKGEPGRQ |
| | | | | proteins | GLPGPPGAPGLN |
| | | | | | (168-195) |
| 316 | 8.773e−11 | 17.99 | BL01113A | C1q domain | GPRGPPGEPGPPGPM |
| | | | | proteins | GPPGEKGEPGRQ |
| | | | | | (156-183) |
| 317 | 8.967e−10 | 20.42 | BL00420A | Speract receptor | GEAGRPGKAGPRGPP |
| | | | | repeat proteins | GEPGPPGPMGPPGE |
| | | | | domain proteins | (147-176) |
| 318 | 7.231e−09 | 20.42 | BL00420A | Speract receptor | GPPGPMGPPGEKGEP |
| | | | | repeat proteins | GRQGLPGPPGAPGL |
| | | | | domain proteins | (165-194) |
| 319 | 7.307e−09 | 4.29 | BL00415N | Synapsins | PRGPPGEPGPPGPMGP |
| | | | | proteins | PGEKGEPGRQGLPGPP |
| | | | | | GAPGLNAAGAIS |
| | | | | | (157-201) |
| 320 | 9.135e-09 | 17.99 | BL01113A | C1q domain | GEAGRPGKAGPRGPP |
| | | | | proteins | GEPGPPGPMGPP |
| | | | | | (147-174) |
| 321 | 9.169e-09 | 20.42 | BL00420A | Speract receptor | GPPGEKGEPGRQGLP |
| | | | | repeat proteins | GPPGAPGLNAAGAI |
| | | | | domain proteins | (171-200) |

The tenth adiponectin-like polypeptide of SEQ ID NO: 323 is an approximately 244-amino acid protein with a predicted molecular mass of approximately 27-kDa unglycosylated. The initial methionine starts at position 161 of SEQ ID NO: 322 and the putative stop codon begins at positions 893 of SEQ ID NO: 322. Protein database searches with the BLASTP algorithm (Altschul S. F. et al., J. Mol. Evol. 36:290-300 (1993) and Altschul S. F. et al., J. Mol. Biol. 21:403-10 (1990), herein incorporated by reference) indicate that SEQ ID NO: 323 is homologous to adiponectin. Using the Pfam software program (Sonnhammer et al., Nucleic Acids Res., 26:320-322 (1998) herein incorporated by reference), adiponectin-like polypeptide of SEQ ID NO: 323 revealed its structural homology to C1q and collagen domains. Further description of the Pfam models can be found at http://pfam.wustl.edu/.

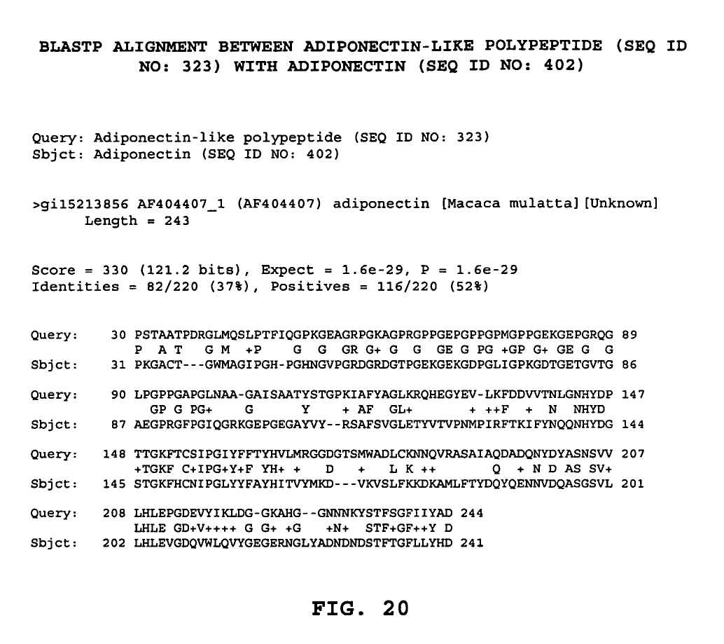

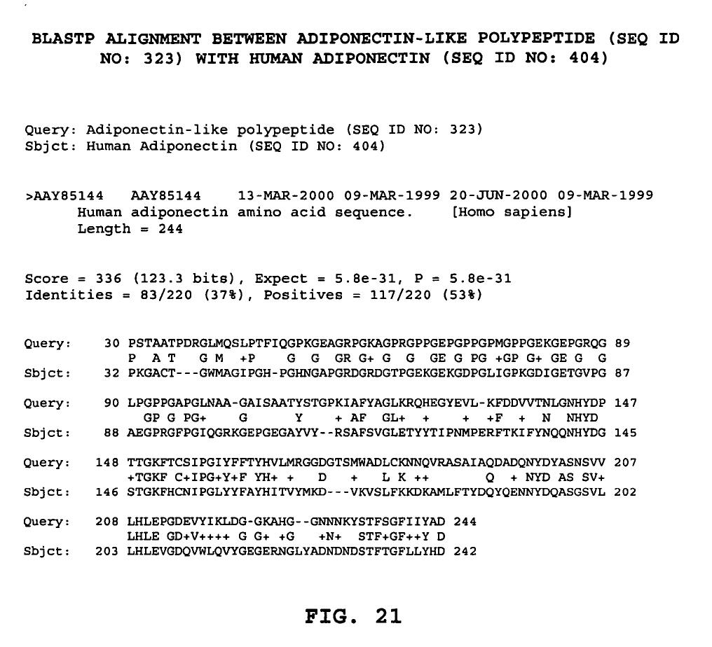

FIG. 20 shows the BLASTP amino acid sequence alignment between adiponectin-like polypeptide SEQ ID NO: 323 and adiponectin SEQ ID NO: 402 (Hotta et al, Diabetes 50:1126-1133 (2001)), indicating that the two sequences share 52% similarity over 220 amino acid residues and 37% identity over the same 220 amino acid residues, wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine. Gaps are presented as dashes.

FIG. 21 shows the BLASTP amino acid sequence alignment between adiponectin-like polypeptide SEQ ID NO: 323 and human adiponectin SEQ ID NO: 404 (Patent No. JP3018186-B1), indicating that the two sequences share 53% similarity over 220 amino acid residues and 37% identity over the same 220 amino acid residues, wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine. Gaps are presented as dashes.

Using eMATRIX software package (Stanford University, Stanford, Calif.) (Wu et al., J. Comp. Biol., 6:219-235 (1999), herein incorporated by reference), adiponectin-like polypeptide of SEQ ID NO: 323 was determined to have following eMATRIX domain hits. The results describe: corresponding SEQ ID NO: in sequence listing, e-value, subtype, Accession number, name, position of the domain in the full-length protein, and the amino acid sequence and are shown in Table 11 below wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine.

TABLE 11

| | | | | | |
|---|---|---|---|---|---|
| SEQ | | | | | |
| ID | | | Accession | | Amino acid sequence |
| NO: | e-value | Subtype | No. | Name | (start and end position) |

| | | | | | |
|---|---|---|---|---|---|
| 327 | 3.647e−27 | 18.26 | BL01113B | C1q domain | VLKFDDVVTNLGNHY |
| | | | | proteins | DPTTGKFTCSIPGIYFF |
| | | | | | TYHV (131-167) |
| 328 | 6.657e−15 | 14.16 | PR00007B | Complement | FTCSIPGIYFFTYHVL |
| | | | | C1q domain | MRGG (152-172) |
| | | | | signature | |
| 329 | 2.047e−14 | 15.60 | PR00007C | Complement | DYASNSVVLHLEPGD |
| | | | | C1q domain | EVYIKLD (200-222) |
| | | | | signature | |
| 330 | 1.000e−13 | 17.99 | BL01113A | C1q domain | GEPGPPGPMGPPGEK |
| | | | | proteins | GEPGRQGLPGPP |
| | | | | | (68-95) |
| 331 | 2.532e−13 | 13.18 | BL01113C | C1q domain | DYASNSVVLHLEPGD |
| | | | | proteins | EVYIK (200-220) |
| 332 | 7.081e−13 | 17.99 | BL01113A | C1q domain | GKAGPRGPPGEPGPP |
| | | | | proteins | GPMGPPGEKGEP |
| | | | | | (59-86) |
| 333 | 8.297e−13 | 17.99 | BL01113A | C1q domain | GRPGKAGPRGPPGEP |
| | | | | proteins | GPPGPMGPPGEK |
| | | | | | (56-83) |
| 334 | 3.538e−12 | 17.99 | BL01113A | C1q domain | GPPGEPGPPGPMGPPG |
| | | | | proteins | EKGEPGRQGL |
| | | | | | (65-92) |
| 335 | 4.808e−12 | 20.42 | BL00420A | Speract receptor | GRPGKAGPRGPPGEP |
| | | | | repeat proteins | GPPGPMGPPGEKGE |
| | | | | domain proteins | (56-85) |
| 336 | 5.385e−12 | 17.99 | BL01113A | C1q domain | GPPGPMGPPGEKGEP |
| | | | | proteins | GRQGLPGPPGAP |
| | | | | | (71-98) |
| 337 | 8.412e−12 | 19.33 | PR00007A | Complement | QHEGYEVLKFDDVVT |
| | | | | C1q domain | NLGNHYDPTTGK |
| | | | | signature | (125-152) |
| 338 | 5.909e−11 | 17.99 | BL01113A | C1q domain | GPMGPPGEKGEPGRQ |
| | | | | proteins | GLPGPPGAPGLN |
| | | | | | (74-101) |
| 339 | 8.773e−11 | 17.99 | BL01113A | C1q domain | GPRGPPGEPGPPGPM |
| | | | | proteins | GPPGEKGEPGRQ |
| | | | | | (62-89) |
| 340 | 8.967e−10 | 20.42 | BL00420A | Speract receptor | GEAGRPGKAGPRGPP |
| | | | | repeat proteins | GEPGPPGPMGPPGE |
| | | | | domain proteins | (53-82) |
| 341 | 7.231e−09 | 20.42 | BL00420A | Speract receptor | GPPGPMGPPGEKGEP |
| | | | | repeat proteins | GRQGLPGPPGAPGL |
| | | | | domain proteins | (71-100) |
| 342 | 7.307e−09 | 4.29 | BL00415N | Synapsins | PRGPPGEPGPPGPMGP |
| | | | | proteins | PGEKGEPGRQGLPGPP |
| | | | | | GAPGLNAAGAIS |
| | | | | | (63-107) |
| 343 | 9.135e−09 | 17.99 | BL01113A | C1q domain | GEAGRPGKAGPRGPP |
| | | | | proteins | GEPGPPGPMGPP |
| | | | | | (53-80) |
| 344 | 9.169e−09 | 20.42 | BL00420A | Speract receptor | GPPGEKGEPGRQGLP |
| | | | | repeat proteins | GPPGAPGLNAAGAI |
| | | | | domain proteins | (77-106) |

A predicted approximately nineteen-residue signal peptide is encoded from approximately residue 1 to residue 19 of SEQ ID NO: 323 (SEQ ID NO: 325). The extracellular portion is useful on its own. This can be confirmed by expression in mammalian cells and sequencing of the cleaved product. The signal peptide region was predicted using the Neural Network SignalP V1.1 program (Nielsen et al, Int. J. Neural Syst. 8:581-599 (1997)). One of skill in the art will recognize that the actual cleavage site may be different than that predicted by the computer program. SEQ ID NO: 326 is the resulting peptide when the signal peptide is removed from SEQ ID NO: 323.

The eleventh adiponectin-like polypeptide of SEQ ID NO: 348 is an approximately 513-amino acid protein with a predicted molecular mass of approximately 57-kDa unglycosylated. The initial methionine starts at position 1 of SEQ ID NO: 347 and the putative stop codon begins at positions 1540 of SEQ ID NO: 347. Protein database searches with the BLASTP algorithm (Altschul S. F. et al., J. Mol. Evol. 36:290-300 (1993) and Altschul S. F. et al., J. Mol. Biol. 21:403-10 (1990), herein incorporated by reference) indicate that SEQ ID NO: 348 is homologous to adiponectin. Using the Pfam software program (Sonnhammer et al., Nucleic Acids Res., 26:320-322 (1998) herein incorporated by reference), adiponectin-like polypeptide of SEQ ID NO: 348 revealed its structural homology to C1q and collagen domains. Further description of the Pfam models can be found at http://pfam.wustl.edu/.

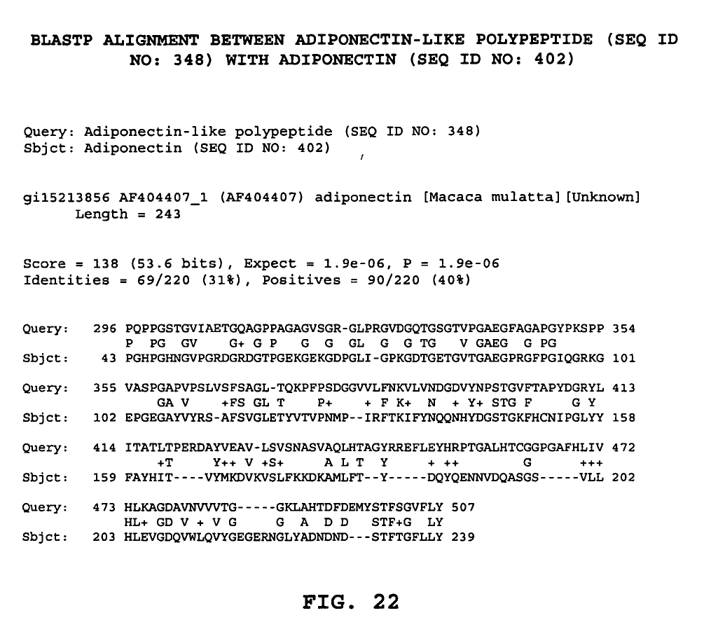

FIG. 22 shows the BLASTP amino acid sequence alignment between adiponectin-like polypeptide SEQ ID NO: 348 and adiponectin SEQ ID NO: 402 (Hotta et al, Diabetes 50:1126-1133 (2001)), indicating that the two sequences share 40% similarity over 220 amino acid residues and 31% identity over the same 220 amino acid residues, wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine. Gaps are presented as dashes.

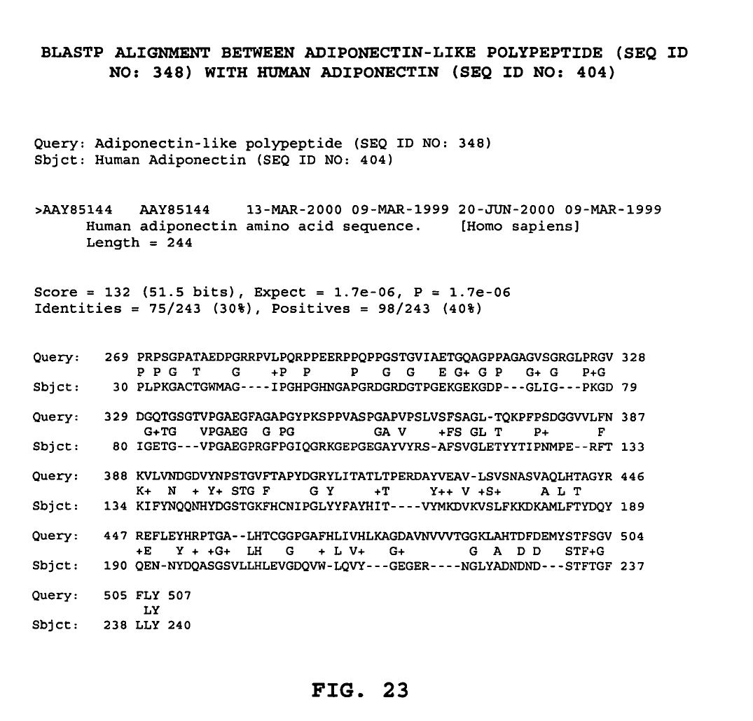

FIG. 23 shows the BLASTP amino acid sequence alignment between adiponectin-like polypeptide SEQ ID NO: 348 and human adiponectin SEQ ID NO: 404 (Patent No. JP3018186-B1), indicating that the two sequences share 40% similarity over 243 amino acid residues and 30% identity over the same 243 amino acid residues, wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine. Gaps are presented as dashes.

Using eMATRIX software package (Stanford University, Stanford, Calif.) (Wu et al., J. Comp. Biol., 6:219-235 (1999), herein incorporated by reference), adiponectin-like polypeptide of SEQ ID NO: 348 was determined to have following eMATRIX domain hits. The results describe: corresponding SEQ ID NO: in sequence listing, e-value, subtype, Accession number, name, position of the domain in the full-length protein, and the amino acid sequence and are shown in Table 12 below wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine.

TABLE 12

| SEQ | | | | | |
|---|---|---|---|---|---|
| ID | | | Accession | | Amino acid sequence |
| NO: | e-value | Subtype | No. | Name | (start and end position) |
| 350 | 5.421e−16 | 18.26 | BL01113B | C1q domain | VVLFNKVLVNDGDVYNP |
| | | | | proteins | STGVFTAPYDGRYLITAT |
| | | | | | L (383-419) |
| 351 | 8.568e−14 | 19.33 | PR00007A | Complement | FPSDGGVVLFNKVLVND |
| | | | | C1q domain | GDVYNPSTGV (377-404) |
| | | | | signature | |

The twelfth adiponectin-like polypeptide of SEQ ID NO: 355 is an approximately 293-amino acid protein with a predicted molecular mass of approximately 33-kDa unglycosylated. The initial methionine starts at position 683 of SEQ ID NO: 354 and the putative stop codon begins at positions 1556 of SEQ ID NO: 354. Protein database searches with the BLASTP algorithm (Altschul S. F. et al., J. Mol. Evol. 36:290-300 (1993) and Altschul S. F. et al., J. Mol. Biol. 21:403-10 (1990), herein incorporated by reference) indicate that SEQ ID NO: 355 is homologous to adiponectin. Using the Pfam software program (Sonnhammer et al., Nucleic Acids Res., 26:320-322 (1998) herein incorporated by reference), adiponectin-like polypeptide of SEQ ID NO: 355 revealed its structural homology to C1q and collagen domains. Further description of the Pfam models can be found at http://pfam.wustl.edu/.

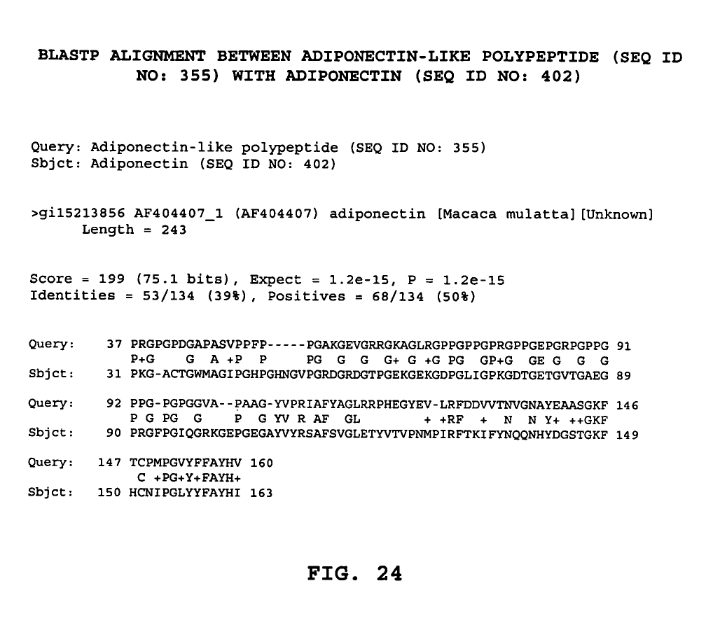

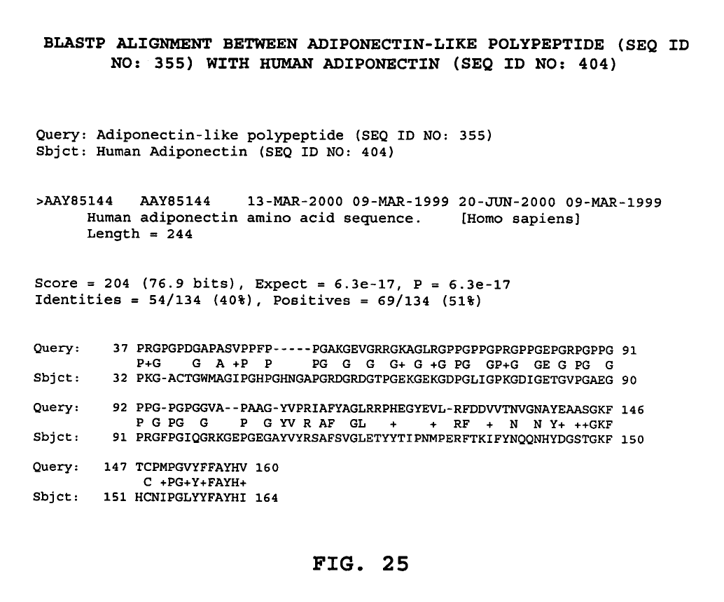

FIG. 24 shows the BLASTP amino acid sequence alignment between adiponectin-like polypeptide SEQ ID NO: 355 and adiponectin SEQ ID NO: 402 (Hotta et al, Diabetes 50:1126-1133 (2001)), indicating that the two sequences share 50% similarity over 134 amino acid residues and 39% identity over the same 134 amino acid residues, wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine. Gaps are presented as dashes.

FIG. 25 shows the BLASTP amino acid sequence alignment between adiponectin-like polypeptide SEQ ID NO: 355 and human adiponectin SEQ ID NO: 404 (Patent No. JP3018186-B1), indicating that the two sequences share 51% similarity over 134 amino acid residues and 40% identity over the same 134 amino acid residues, wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine. Gaps are presented as dashes.

Using eMATRIX software package (Stanford University, Stanford, Calif.) (Wu et al., J. Comp. Biol., 6:219-235 (1999), herein incorporated by reference), adiponectin-like polypeptide of SEQ ID NO: 355 was determined to have following eMATRIX domain hits. The results describe: corresponding SEQ ID NO: in sequence listing, e-value, subtype, Accession number, name, position of the domain in the full-length protein, and the amino acid sequence and are shown in Table 13 below wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine.

TABLE 13

| | | | | | |
|---|---|---|---|---|---|
| SEQ | | | | | |
| ID | | | Accession | | Amino acid sequence |
| NO: | e-value | Subtype | No. | Name | (start and end position) |

| | | | | | |
|---|---|---|---|---|---|
| 359 | 3.786e−23 | 18.26 | BL01113B | C1q domain | VLRFDDVVTNVGNA |
| | | | | proteins | YEAASGKFTCPMPGV |
| | | | | | YFFAYHV (125-161) |
| 360 | 5.114e−15 | 14.16 | PR00007B | Complement | FTCPMPGVYFFAYHV |
| | | | | C1q domain | LMRGG (146-166) |
| | | | | signature | |
| 361 | 7.968e−15 | 17.99 | BL01113A | C1q domain | GPPGPRGPPGEPGRPG |
| | | | | proteins | PPGPPGPGPGG |
| | | | | | (73-100) |
| 362 | 5.091e−14 | 17.99 | BL01113A | C1q domain | GPPGPPGPRGPPGEPG |
| | | | | proteins | RPGPPGPPGPG |
| | | | | | (70-97) |
| 363 | 5.295e−11 | 17.99 | BL01113A | C1q domain | GKAGLRGPPGPPGPR |
| | | | | proteins | GPPGEPGRPGPP |
| | | | | | (64-91) |
| 364 | 8.568e−11 | 17.99 | BL01113A | C1q domain | GPPGEPGRPGPPGPPG |
| | | | | proteins | PGPGGVAPAAG |
| | | | | | (79-106) |
| 365 | 8.691e−11 | 20.42 | BL00420A | Speract receptor | GPPGPRGPPGEPGRPG |
| | | | | repeat proteins | PPGPPGPGPGGVA |
| | | | | domain proteins | (73-102) |
| 366 | 8.977e−11 | 17.99 | BL01113A | C1q domain | GLRGPPGPPGPRGPPG |
| | | | | proteins | EPGRLPGPPGPP (67-94) |
| 367 | 9.673e−11 | 20.42 | BL00420A | Speract receptor | GPPGPPGPRGPPGEPG |
| | | | | repeat proteins | RPGPPGPPGPGPG |
| | | | | domain proteins | (70-99) |
| 368 | 2.180e−10 | 20.42 | BL00420A | Speract receptor | GAKGEVGRRGKAGL |
| | | | | repeat proteins | RGPPGPPGPRGPPGE |
| | | | | domain proteins | (55-84) |
| 369 | 7.052e−10 | 19.33 | PR00007A | Complement | PHEGYEVLRFDDVVT |
| | | | | C1q domain | NVGNAYEAASGK |
| | | | | Signature | (119-146) |
| 370 | 4.351e−09 | 5.36 | PR00524F | Cholecystokinin | GPPGPPGPRGPPGE |
| | | | | type A receptor | (70-84) |
| | | | | signature | |
| 371 | 4.635e−09 | 17.99 | BL01113A | C1q domain | GEPGRPGPPGPPGPGP |
| | | | | proteins | GGVAPAAGYVP |
| | | | | | (82-109) |
| 372 | 6.192e−09 | 17.99 | BL01113A | C1q domain | GPRGPPGEPGRPGPPG |
| | | | | proteins | PPGPGPGGVAP |
| | | | | | (76-103) |
| 373 | 6.595e−09 | 13.84 | DM00250B | Kw Annexin | GEPGRPGPPGPPGPGP |
| | | | | antiben proline | GGVAPAAG (82-106) |
| | | | | tumor | |
| 374 | 7.372e−09 | 4.29 | BL00415N | Synapsins | RRGKAGLRGPPGPPG |
| | | | | proteins | PRGPPGEPGRPGPPGP |
| | | | | | PGPGPGGVAPAAG |
| | | | | | (62-106) |
| 375 | 7.750e−09 | 17.99 | BL01113A | C1q domain | GRRGKAGLRGPPGPP |
| | | | | proteins | GPRGPPGEPGRPGPP |
| | | | | | (61-88) |
| 376 | 8.062e−09 | 20.42 | BL00420A | Speract receptor | FPPGAKGEVGRRGKA |
| | | | | repeat proteins | GLRGPPGPPGPRGP |
| | | | | domain proteins | (52-81) |

The thirteenth adiponectin-like polypeptide of SEQ ID NO: 378 is an approximately 238-amino acid protein with a predicted molecular mass of approximately 27-kDa unglycosylated. The initial methionine starts at position 683 of SEQ ID NO: 377 and the putative stop codon begins at positions 1391 of SEQ ID NO: 377. Protein database searches with the BLASTP algorithm (Altschul S. F. et al., J. Mol. Evol. 36:290-300 (1993) and Altschul S. F. et al., J. Mol. Biol. 21:403-10 (1990), herein incorporated by reference) indicate that SEQ ID NO: 378 is homologous to adiponectin. Using the Pfam software program (Sonnhammer et al., Nucleic Acids Res., 26:320-322 (1998) herein incorporated by reference), adiponectin-like polypeptide of SEQ ID NO: 355 revealed its structural homology to C1q and collagen domains. Further description of the Pfam models can be found at http://pfam.wustl.edu/.

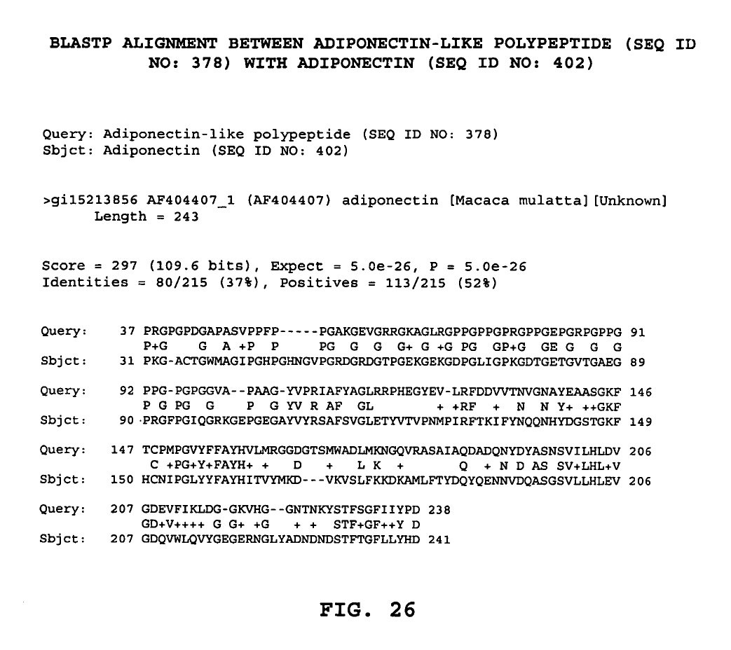

FIG. 26 shows the BLASTP amino acid sequence alignment between adiponectin-like polypeptide SEQ ID NO: 378 and adiponectin SEQ ID NO: 402 (Hotta et al, Diabetes 50:1126-1133 (2001)), indicating that the two sequences share 52% similarity over 215 amino acid residues and 37% identity over the same 215 amino acid residues, wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine. Gaps are presented as dashes.

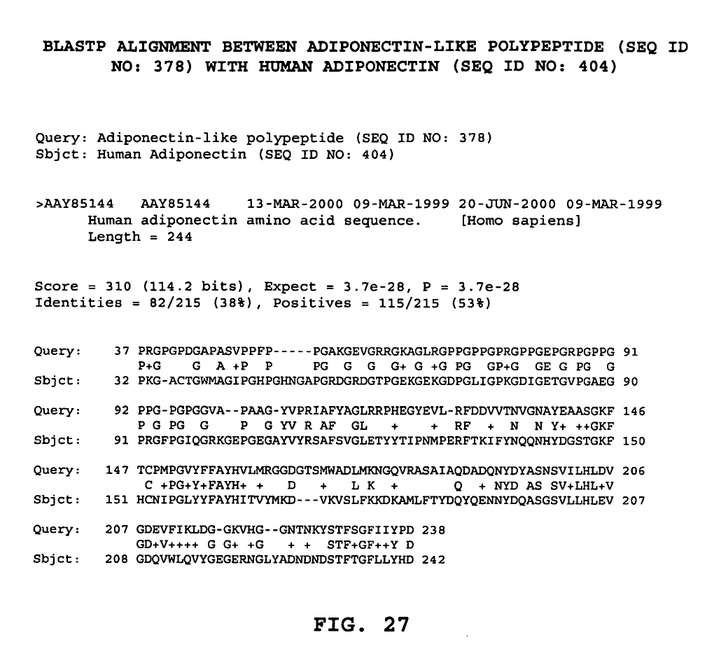

FIG. 27 shows the BLASTP amino acid sequence alignment between adiponectin-like polypeptide SEQ ID NO: 378 and human adiponectin SEQ ID NO: 404 (Patent No. JP3018186-B1), indicating that the two sequences share 53% similarity over 215 amino acid residues and 38% identity over the same 215 amino acid residues, wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine. Gaps are presented as dashes.

Using eMATRIX software package (Stanford University, Stanford, Calif.) (Wu et al., J. Comp. Biol., 6:219-235 (1999), herein incorporated by reference), adiponectin-like polypeptide of SEQ ID NO: 378 was determined to have following eMATRIX domain hits. The results describe: corresponding SEQ ID NO: in sequence listing, e-value, subtype, Accession number, name, position of the domain in the full-length protein, and the amino acid sequence and are shown in Table 14 below wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine.

TABLE 14

| | | | | | |
|---|---|---|---|---|---|
| SEQ | | | | | |
| ID | | | Accession | | Amino acid sequence |
| NO: | e-value | Subtype | No. | Name | (start and end position) |

| | | | | | |
|---|---|---|---|---|---|
| 381 | 3.786e−23 | 18.26 | BL01113B | C1q domain | VLRFDDVVTNVGNA |
| | | | | proteins | YEAASGKFTCPMPGV |
| | | | | | YFFAYHV (125-161) |
| 382 | 5.114e−15 | 14.16 | PR00007B | Complement | FTCPMPGVYFFAYHV |
| | | | | C1q domain | LMRGG (146-166) |
| | | | | signature | |
| 383 | 7.968e−15 | 17.99 | BL01113A | C1q domain | GPPGPRGPPGEPGRPG |
| | | | | proteins | PPGPPGPGPGG |
| | | | | | (73-100) |
| 384 | 5.091e−14 | 17.99 | BL01113A | C1q domain | GPPGPPGPRGPPGEPG |
| | | | | proteins | RPGPPGPPGPG |
| | | | | | (70-97) |
| 385 | 5.875e−13 | 15.60 | PR00007C | Complement | DYASNSVILHLDVGD |
| | | | | C1q domain | EVFIKLD (194-216) |
| | | | | signature | |
| 386 | 4.000e−12 | 13.18 | BL01113C | C1q domain | DYASNSVILHLDVGD |
| | | | | proteins | EVFLK (194-214) |
| 387 | 5.295e−11 | 17.99 | BL01113A | C1q domain | GKAGLRGPPGPPGPR |
| | | | | proteins | GPPGEPGRPGPPGPP |
| | | | | | (64-91) |
| 388 | 8.568e−11 | 17.99 | BL01113A | C1q domain | GPPGEPGRPGPPGPPG |
| | | | | proteins | PGPGGVAPAAG |
| | | | | | (79-106) |
| 389 | 8.691e−11 | 20.42 | BL00420A | Speract receptor | GPPGPRGPPGEPGRPG |
| | | | | repeat proteins | PPGPPGPGPGGVA |
| | | | | domain proteins | (73-102) |
| 390 | 8.977e−11 | 17.99 | BL01113A | C1q domain | GLRGPPGPPGPRGPPG |
| | | | | proteins | EPGRPGPPGPP |
| | | | | | (67-94) |
| 391 | 9.673e−11 | 20.42 | BL00420A | Speract receptor | GPPGPPGPRGPPGEPG |
| | | | | repeat proteins | RPGPPGPPGPGPG |
| | | | | domain proteins | (70-99) |
| 392 | 2.180e−10 | 20.42 | BL00420A | Speract receptor | GAKGEVGRRGKAGL |
| | | | | repeat proteins | RGPPGPPGPRGPPGE |
| | | | | domain proteins | (55-84) |
| 393 | 7.052e−10 | 19.33 | PR00007A | Complement | PHEGYEVLRFDDVVT |
| | | | | C1q domain | NVGNAYEAASGK |
| | | | | signature | (119-146) |
| 394 | 4.351e−09 | 5.36 | PR00524F | Cholecystokinin | GPPGPPGPRGPPGE |
| | | | | type A receptor | (70-84) |
| | | | | signature | |
| 395 | 4.635e−09 | 17.99 | BL01113A | C1q domain | GEPGRPGPPGPPGPGP |
| | | | | proteins | GGVAPAAGYVP |
| | | | | | (82-109) |
| 396 | 6.192e−09 | 17.99 | BL01113A | C1q domain | GPRGPPGEPGRPGPPG |
| | | | | proteins | PPGPGPGGVAP |
| | | | | | (76-103) |
| 397 | 6.595e−09 | 13.84 | DM00250B | kw Annexin | GEPGRPGPPGPPGPGP |
| | | | | antigen proline | GGVAPAAG (82-106) |
| | | | | tumor | |
| 398 | 7.372e−09 | 4.29 | BL00415N | Synapsins | RRGKAGLRGPPGPPG |
| | | | | proteins | PRGPPGEPGRPGPPGP |
| | | | | | PGPGPGGVAPAAG |
| | | | | | (62-106) |
| 399 | 7.750e−09 | 17.99 | BL01113A | C1q domain | GRRGKAGLRGPPGPP |
| | | | | proteins | GPRGPPGEPGRP |
| | | | | | (61-88) |
| 400 | 7.750e−09 | 7.47 | BL01113D | C1q domain | STFSGFIIYP (228-238) |
| | | | | proteins | |
| 401 | 8.062e−09 | 20.42 | BL00420A | Speract receptor | FPPGAKGEVGRRGKA |
| | | | | repeat proteins | GLRGPPGPPGPRGP |
| | | | | domain proteins | (52-81) |

A predicted approximately fifteen-residue signal peptide is encoded from approximately residue 1 to residue 15 of SEQ ID NO: 355, or 378 (SEQ ID NO: 357). The extracellular portion is useful on its own. This can be confirmed by expression in mammalian cells and sequencing of the cleaved product. The signal peptide region was predicted using the Neural Network SignalP V1.1 program (Nielsen et al, Int. J. Neural Syst. 8:581-599 (1997)). One of skill in the art will recognize that the actual cleavage site may be different than that predicted by the computer program. SEQ ID NO: 358 is the resulting peptide when the signal peptide is removed from SEQ ID NO: 355. SEQ ID NO: 380 is the resulting peptide when the signal peptide is removed from SEQ ID NO: 378.

The adiponectin-like polypeptides and polynucleotides of the invention may be used to treat obesity, diabetes, lipoatrophy, coronary artery diseases, atherosclerosis, and other obesity and diabetes-related cardiovascular pathologies. Adiponectin-like polypeptides and polynucleotides of the invention may also be used in treatment of autoimmune diseases and inflammation, to modulate immune responses, and to treat transplant patients.

4.2 Serpin-Like Polypeptides and Polynucleotides

Proteinases play many important physiological functions in the body, including food digestion, remodeling of extracellular matrices, blood coagulation, and immune processes (Salzet et al., Trends Immunol. 20:541-544 (1999), herein incorporated by reference in its entirety). Proteinases have also been implicated in maturation of signaling proteins (e.g. methionine enkaphalin), hormones, and digestive enzymes. Proteinases are classified based on the central amino acid residue in the active site of the proteinase (like serine proteinases, cysteine proteinases, or aspartate proteinases). Proteinases are implicated in many pathologies including emphysema, arthritis, and cardiovascular diseases. Proteinases are regulated by binding of inhibitory proteins in the extracellular environment.

Serpins (serine proteinase inhibitors) are a superfamily of more than 500 proteins, about 350-500 amino acids in size, that fold into a conserved structure and employ a unique suicide substrate-like inhibitory strategy (Silverman et al., J. Biol. Chem. 276:33293-33296 (2001), herein incorporated by reference in its entirety). The serpin superfamily has evolved over 500 million years with representatives found in viruses, plants, protozoa, insects, and higher vertebrates (Schich et al., J. Biol. Chem. 272:1849-1855 (1997), herein incorporated by reference in its entirety). The tertiary structures of serpins demonstrate 3β-sheets, ˜9α-helices, and several loops that are arranged into a metastable conformation (Askew et al., J. Biol. Chem. 276:49320-49330 (2001), herein incorporated by reference in its entirety). The mobile reactive site loop (RSL) is displayed on the surface, and serves as pseudo-substrate to bind to proteinase. Upon binding to proteinase and cleavage of the RSL loop the serpin molecule undergoes a conformational change that traps the proteinase in a covalent acyl-enzyme intermediate. Serpins regulate serine proteinases involved in coagulation, fibrinolysis, inflammation, cell migration, and extracellular matrix remodeling.

A subclass of serpins exhibits strong sequence similarity to chicken ovalbumin. The serpin-like molecule of present invention which has strong homology to SERPINB12, belong to this subclass of serpins. These ov-serpins lack both the N-terminal signal peptides and C-terminal extensions of other serpins. They also exhibit a variable length loop between C and D helices that may harbor functional motifs. The ov-serpins are proposed to be either cytoplasmic or nucleocytoplasmic proteins. However, many of them (maspin, megsin, and SCCAs) may function extracellularly as they are released from cells under certain conditions. The ov-serpins are functional inhibitors of serine or cysteine proteinases. Many of them inhibit more than one class of proteinases. Many of the ov-serpins are present in the same cells that secrete the proteinases and thus may have regulatory functions. They may also help protect the secreting cell from the proteinases.

Thus, the Serpin-like polypeptides and polynucleotides of the invention may be used to treat emphysema, arthritis, blood clotting disorders, and cardiovascular disease. Serpin-like polypeptides and polynucleotides of the invention may also be used in treatment of immune disorders and inflammation, to modulate immune responses, and to treat transplant patients. Serpin-like polypeptides may also be useful as marker in diagnosis and prognosis of certain cancers.

The Serpin-like polypeptide of SEQ ID NO: 408 is an approximately 425-amino acid protein with a predicted molecular mass of approximately 48-kDa unglycosylated. The initial methionine starts at position 78 of SEQ ID NO: 407 and the putative stop codon begins at positions 1353 of SEQ ID NO: 407. Protein database searches with the BLASTP algorithm (Altschul S. F. et al., J. Mol. Evol. 36:290-300 (1993) and Altschul S. F. et al., J. Mol. Biol. 21:403-10 (1990), herein incorporated by reference in their entirety) indicate that SEQ ID NO: 408 is homologous to SERPINB12 and squamous cell carcinoma antigen 2 (SCCA2). Using the Pfam software program (Sonnhammer et al., Nucleic Acids Res. 26:320-322 (1998), herein incorporated by reference in its entirety), Serpin-like polypeptide of SEQ ID NO: 408 revealed its sequence homology to serpins. Further description of the Pfam models can be found at http://pfam.wustl.edu/.

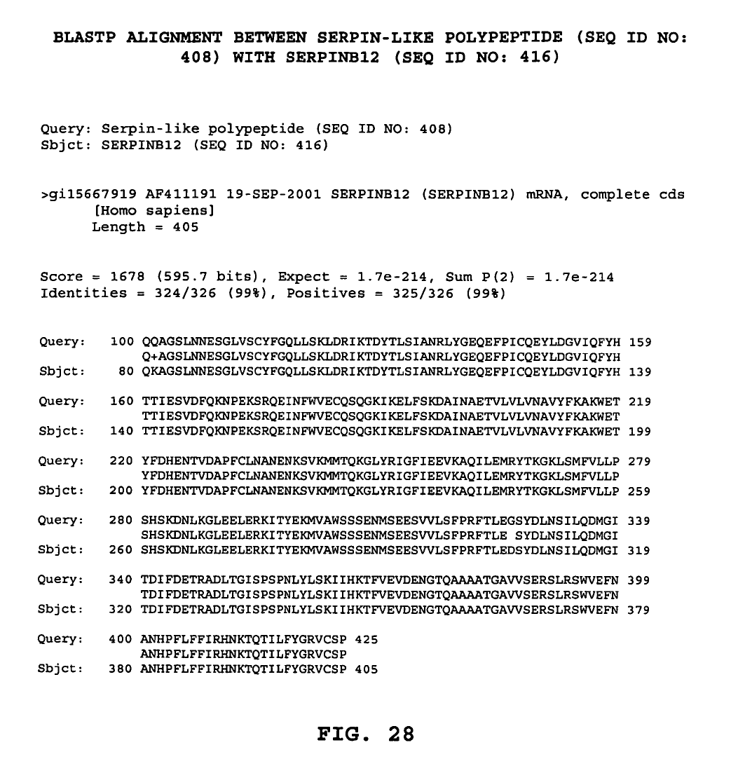

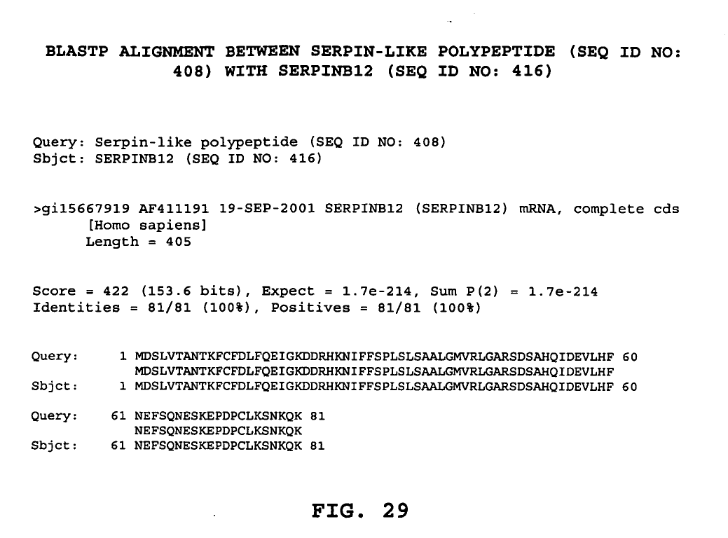

FIG. 28 shows the BLASTP amino acid sequence alignment of the first high scoring pair (HSP) between Serpin-like polypeptide SEQ ID NO: 408 and SERPINB12 SEQ ID NO: 416 (Askew et al., J. Biol. Chem. 276:49320-49330 (2001), herein incorporated by reference in its entirety), indicating that the two sequences share 99% similarity over 326 amino acid residues and 99% identity over the same 326 amino acid residues, wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine. Gaps are presented as dashes.

FIG. 29 shows the BLASTP amino acid sequence alignment of the second high scoring pair (HSP) between Serpin-like polypeptide SEQ ID NO: 408 and SERPINB12 SEQ ID NO: 416 (Askew et al., J. Biol. Chem. 276:49320-49330 (2001), herein incorporated by reference in its entirety), indicating that the two sequences share 100% similarity over 81 amino acid residues and 100% identity over the same 81 amino acid residues, wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine. Gaps are presented as dashes.

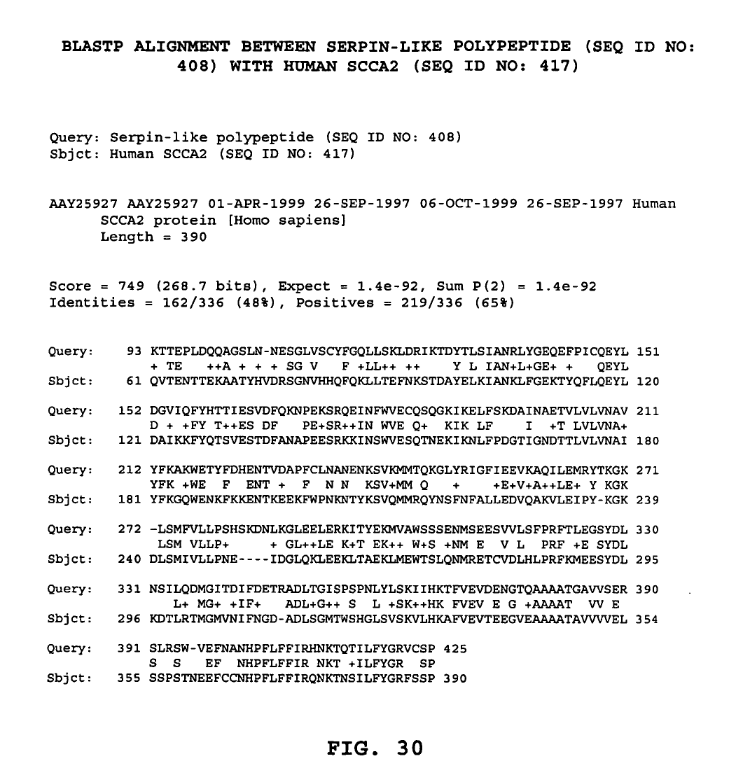

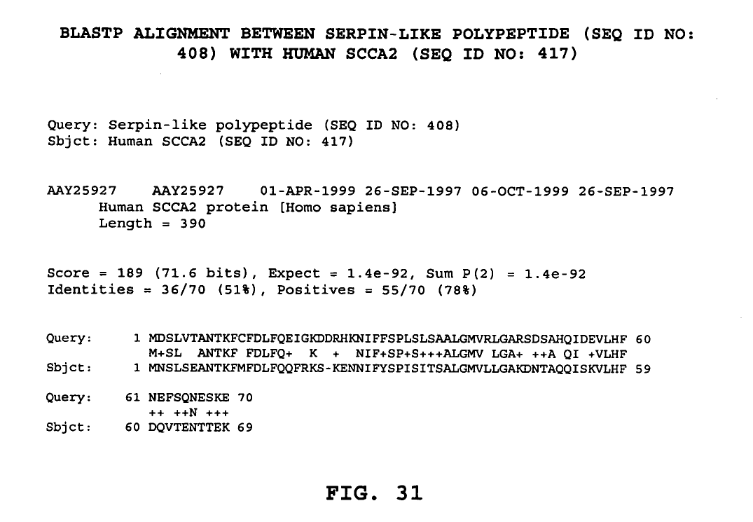

FIG. 30 shows the BLASTP amino acid sequence alignment of the first high scoring pair (HSP) between Serpin-like polypeptide SEQ ID NO: 408 and human SCCA2 protein SEQ ID NO: 417 (Patent No. DE19742725-A1, herein incorporated by reference in its entirety), indicating that the two sequences share 65% similarity over 336 amino acid residues and 48% identity over the same 336 amino acid residues, wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine. Gaps are presented as dashes.

FIG. 31 shows the BLASTP amino acid sequence alignment of the second high scoring pair (HSP) between Serpin-like polypeptide SEQ ID NO: 408 and human SCCA2 protein SEQ ID NO: 417 (Patent No. DE19742725-A1, herein incorporated by reference in its entirety), indicating that the two sequences share 78% similarity over 70 amino acid residues and 51% identity over the same 70 amino acid residues, wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine. Gaps are presented as dashes.

Using eMATRIX software package (Stanford University, Stanford, Calif.) (Wu et al., J. Comp. Biol., 6:219-235 (1999), herein incorporated by reference in its entirety), Serpin-like polypeptide of SEQ ID NO: 408 was determined to have following eMATRIX domain hits. The results describe: corresponding SEQ ID NO: in sequence listing, e-value, subtype, Accession number, name, position of the domain in the full-length protein, and the amino acid sequence and are shown in Table 15 below wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine.

TABLE 15

| | | | | | Amino |
|---|---|---|---|---|---|
| SEQ | | | | | Acid Sequence |
| ID | | sub- | Accession | | (start and |
| NO: | e-value | type | No. | Name | end position) |
| 410 | 7.600e−25 | 28.56 | BL00284C | Serpins | TVLVLVNAVYFKA |
| | | | | proteins | KWETYFDHENTVD |
| | | | | | APFCLNANENKSV |
| | | | | | KMM (203-245) |
| 411 | 4.375e−23 | 19.15 | BL00284E | Serpins | NHPFLFFIRHNKT |
| | | | | proteins | QTILFYGRVCSP |
| | | | | | (401-426) |
| 412 | 5.286e−21 | 16.34 | BL00284D | Serpins | LSFPRFTLEGSYD |
| | | | | proteins | LNSILQDMGITDI |
| | | | | | F (317-344) |
| 413 | 6.192e−17 | 15.64 | BL00284A | Serpins | NIFFSPLSLSAAL |
| | | | | proteins | GMVRLGARSDS |
| | | | | | (27-51) |
| 414 | 4.414e−13 | 17.99 | BL00284B | Serpins | SRQEINFWVECQS |
| | | | | proteins | QGKIKELF |
| | | | | | (174-195) |

Serpins undergo a conformational change upon binding of the proteinase substrate thereby trapping the proteinase in a covalent acyl-enzyme intermediate (Huntington et al., Nature 407:923-926 (2000), herein incorporated by reference). Serpins utilize this mechanism to regulate proteinase cascades involved in blood clotting, fibrinolysis, complement activation, cell motility, inflammation, and cell death (Silverman et al., J. Biol. Chem. 276:33293-33296 (2001); Carrell et al., Mol. Biol. Med. 6:35-42 (1989); Potempa et al., J. Biol. Chem. 269:15957-15960 (1994), all of which are herein incorporated by reference). Members of the ov-serpin subfamily inhibit various serine or cysteine proteinases and are involved in inhibition of cell migration, protection against apoptosis, and neutralization of endogenous granule proteinases that leak into the cytosol (Silverman et al., J. Biol. Chem. 276:33293-33296 (2001); Bird, Immunol. Cell. Biol. 77:47-57 (1999), both of which are herein incorporated by reference). Specifically, SERPINB12 is a potent inhibitor of trypsin-like serine proteinases, including trypsin and plasmin (Askew et al., J. Biol. Chem. 276:49320-49330 (2001), herein incorporated by reference).

The polypeptides of the invention are expected to have similar functions as serpins, specifically the ov-serpins such as SERPINB12, acting as an inhibitor of serine and cysteine proteinases. The polypeptides, polynucleotides, antibodies, and other compositions of the invention are expected to be useful in treating the following disorders: emphysema, arthritis, blood clotting disorders and cardiovascular diseases. Serpin-like polypeptides and polynucleotides of the invention may also be used in the treatment of immune disorders and inflammation, to modulate immune responses, and to treat transplant patients. Serpin-like polypeptides may also be useful as markers in diagnosis and prognosis of certain cancers.

4.3 Nogo-Receptor-Like (NgRHy) Polypeptides and Polynucleotides

The establishment of neural connections during development is a highly dynamic process. A key aspect of this process is the regulation of axon growth, which is mediated by a variety of chemotropic factors (Skaper, et al., Prog. Neurobiol. 56:593-608 (2001), herein incorporated by reference). Chemotropism, which determines the direction of axonal growth, results from the concerted action of chemoattractant and chemorepellent cues (Yu and Bargmann, Nat. Neurosci. 4(Suppl.):1169-1176 (2001), herein incorporated by reference). Growth cones, the leading edge of the axons, encounter and detect these guiding cues along their trajectories in the form of gradients of diffusible factors, necessary for long-range guidance (Zheng and Kuffler, J. Neurobiol. 42:212-219 (2000), herein incorporated by reference), extracellular matrix-associated molecules, required for both short- and long-range regulation (Hynds and Snow, Exp. Neurol. 160:244-255 (1999), herein incorporated by reference; Skaper et al., supra), and membrane-bound molecules, necessary for short-range regulation (He and Meini, Mol. Cell. Neurosci. 19:18-31 (2002), herein incorporated by reference). It is believed that the inability of mature neurons to regenerate appropriate connections following injury or trauma is in part mediated by chemorepellent molecules present along axonal tracts (Fawcett, Cell Tissue Res. 290:371-377 (1997), herein incorporated by reference).

Results from studies demonstrating that neurons in the adult central nervous system (CNS) have regenerative potential support this hypothesis. For example, it is known that severed fibers of the optic nerve and of the spinal cord are unable to regenerate across the site of lesion (reviewed in Tessler-Lavigne and Goodman, Science 287:813-814 (2000), herein incorporated by reference). In contrast, injuries do not prevent motor and sensory neurons projections to peripheral targets to regenerate. Experiments by David and Aguayo (Science 214:931-933 (1981), herein incorporated by reference) indicate that if the two extremities of severed optic nerves are “bridged” surgically with a graft obtained from peripheral nerves, retinal projection re-growth along the grafted fibers extends well beyond the injured site. These and other experiments led to the hypothesis that the myelin sheath surrounding CNS axons contains inhibitory cues that are absent in myelin of axons in the peripheral nervous system (PNS) (Schwab and Caroni, J. Neurosci. 8: 2381-2393 (1988), herein incorporated by reference).

The search for inhibitory cues present in CNS myelin preparation, led to the identification of an inhibitory activity found only in CNS myelin (GrandPré and Strittmatter, Neuroscientist 7:377-386 (2001), herein incorporated by reference). Protein purification combined with inhibitory activity in vitro assays identified myelin protein fractions of approximately 35 and 250 kD, known as neurite growth inhibitors NI-35 and NI-250. NI-250 is also known as Nogo (Chen et al Nature 403:434-39 (2000); GrandPré et al, Nature 403: 439-444 (2000); Prinjha et al., Nature 403: 383-84 (2000), all of which are herein incorporated by reference).

There are three isoforms of the Nogo protein, Nogo-A, -B, and -C, which result from alternative splicing or promoter usage. Nogo-A is the full-length protein of 1192 amino acids and is expressed primarily in the brain and optic nerve. Nogo-B, 373 amino acids, may correspond to the NI-35 fraction of myelin preparation and is located in small amounts in the optic nerve. Nogo-C, 199 amino acids long, is found primarily in the brain. Nogo-A and -B share the same common N-terminus of 172 amino acids, while all three Nogo isoforms share a common C-terminal region which shows approximately 70% similarity to the C-terminus of the reticulon (Rtn) family of proteins (GrandPré et al, supra). The C-termini contain two hydrophobic transmembrane domains separated by a 66 amino acid hydrophilic loop that protrudes from the cell surface.

Mapping Nogo neuronal growth inhibitory domains demonstrates that two distinct sites play a role in preventing neurite outgrowth. The Nogo-A protein was shown to inhibit axonal growth in dorsal root ganglion (DRG) explants in vitro. Fine mapping of Nogo-A by Chen et al, (supra) demonstrates that the amino terminal portion, known as Amino-Nogo, inhibits neurite outgrowth in culture. The 66 amino acid linker of Nogo-C has inhibitory properties as well, inhibiting growth cone formation and inducing growth cone collapse in chick DRG neurons in vitro (GrandPré et al supra). Further mapping of Nogo-66 revealed that residues 33-55 of the extracellular sequence are responsible for the growth cone inhibition (GrandPré et al supra).

The receptor for the Nogo-66 peptide was identified by Fournier et al. by using a Nogo-66-alkaline phosphatase fusion protein (Nogo-AP) which was shown to bind with high affinity to chick DRG axons (Fournier, et al., supra). The Nogo-66 receptor (NgR) is 473 amino acids, contains a signal sequence followed by eight leucine rich repeat (LRR) domains, an LRR flanking carboxy-terminal (LRRCT) domain that is cysteine-rich, a unique region, and a C-terminal glycophosphtidyl inositol (GPI) anchoring sequence (Fournier, et al., supra). The NgR mRNA is primarily expressed in the brain. Cleavage of NgR from the axonal cell surface renders neurons insensitive to Nogo-66. Furthermore, neurons that do not express NgR are insensitive to Nogo-66-induced growth cone collapse. However, expression of recombinant NgR in these cells renders axonal growth cones sensitive to Nogo-66-induced collapse, indicating that NgR facilitates Nogo activity in neurons (Fournier, et al., supra).

Administration of antibodies generated against the NI-250 myelin fraction (IN-1; Caroni and Schwab, Neuron 1:85-96 (1988), herein incorporated by reference) neutralizes the effects of NI-35/250 in culture and permits axon fibers extension in contrast to untreated cells. IN-1 antibodies also improve the motor capabilities of adult rats after spinal cord injury (Bregman et al., Nature 378:498-501 (1995); Merkler et al., J. Neuroscience 27: 3665-73 (2001), all of which are herein incorporated by reference). These results indicate that Nogo is a major factor in inhibiting CNS axonal regeneration and that blocking Nogo activity can be an effective measure in restoring axonal function after spinal cord trauma.

Thus, there exists a need in the art to identify materials and methods to modulate growth cone collapse and axonal regeneration. Identification and development of such agents provides therapeutic compositions and methods of treatment for neurological conditions such as spinal cord injury, cranial or cerebral trauma, stroke, and demyelinating diseases.

The NgRHy polypeptide of SEQ ID NO: 420 is an approximately 420 amino acid transmembrane protein with a predicted molecular mass of approximately 46 kDa unglycosylated. Protein database searches with the BLASTP algorithm (Altschul S. F. et al., J. Mol. Evol. 36:290-300 (1993) and Altschul S. F. et al., J. Mol. Biol. 21:403-10 (1990), herein incorporated by reference) indicate that SEQ ID NO: 420 is homologous to human NgR.

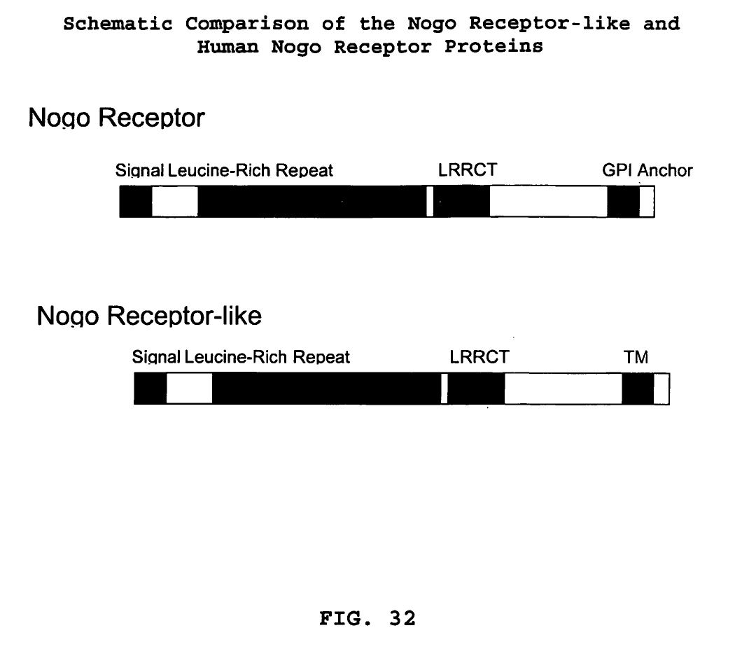

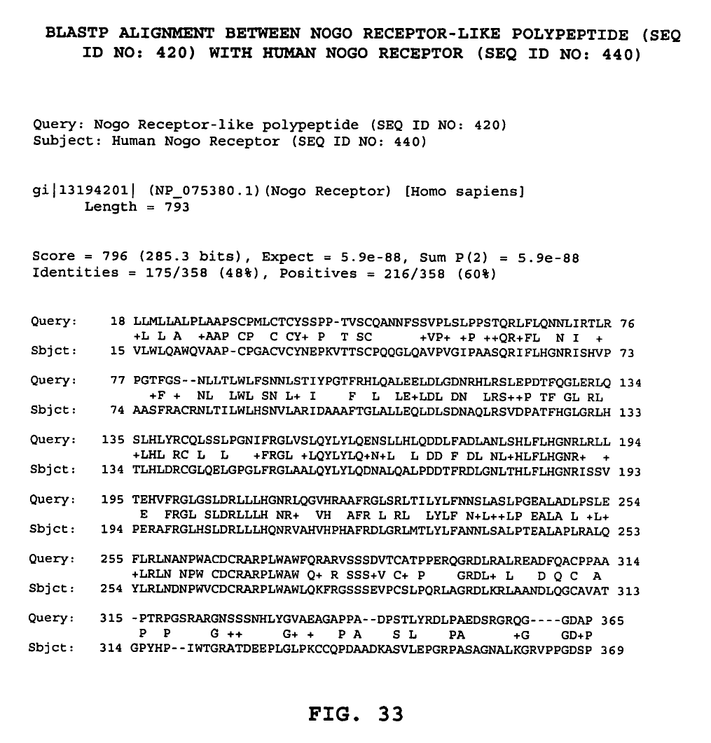

FIG. 32 shows a schematic diagram illustrating the major structural features of the Nogo-receptor, NgR, and the Nogo-receptor homolog, NgRHy.

FIG. 33 shows the BLASTP amino acid sequence alignment between the protein encoded by SEQ ID NO: 419 (i.e. SEQ ID NO: 420), NgRHy, and the human NgR (SEQ ID NO: 440), indicating that the two sequences share 48% identity over 358 amino acids of SEQ ID NO: 420 and 60% similarity over the same 358 amino acids of SEQ ID NO: 420, wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine. Gaps are presented as dashes.

A predicted approximately 16 residue signal peptide is encoded from approximately residue 1 through residue 30 of SEQ ID NO: 420 (SEQ ID NO: 422). The extracellular portion (SEQ ID NO: 439) is useful on its own. This can be confirmed by expression in mammalian cells and sequencing of the cleaved product. The signal peptide region was predicted using the Neural Network SignalP V1.1 program (from Center for Biological Sequence Analysis, The Technical University of Denmark). One of skill in the art will recognize that the actual cleavage site may be different than that predicted by the computer program.

Using eMATRIX software package (Stanford University, Stanford, Calif.) (Wu et al., J. Comp. Biol. 6:219-235 (1999), herein incorporated by reference), NgRHy is expected to have five leucine-rich repeat (LRR) domains at residues 130-144 of SEQ ID NO: 420 (SEQ ID NO: 423), residues 154-168 of SEQ ID NO: 420 (SEQ ID NO: 424), residues 157-171 of SEQ ID NO: 420 (SEQ ID NO: 425), residues 178-192 of SEQ ID NO: 420 (SEQ ID NO: 426), and residues 250-264 of SEQ ID NO: 420 (SEQ ID NO: 427) domain as shown Table 16, wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine:

TABLE 16

| | | | Amino |
|---|---|---|---|
| | | | acid sequence |
| SEQ | | Signature | (start and |
| ID NO: | p-value | Identification No. | end position) |
| 423 | 5.345e−08 | PR00019A | LERLQSLHLYRCQLS |
| | | | (130-144) |
| 424 | 8.448e−08 | PR00019B | LVSLQYLYLQENSLH |
| | | | (154-168) |
| 425 | 4.545e−08 | PR00019A | LQYLYLQENSLLHLQ |
| | | | (157-171) |
| 426 | 2.552e−08 | PR00019B | LANLSHLFLHGNRLR |
| | | | (178-192) |
| 427 | 8.448e−08 | PR00019B | LPSLEFLRLNANPWA |
| | | | (250-264) |

Using hmmpfam software (Washington University School of Medicine, St. Louis, Mo.), NgRHy was determined to have eight leucine-rich repeat (LRR) domain and a leucine-rich region-associated C-terminal (LRRCT) domain as shown in Table 17, wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine:

TABLE 17

| | | | | |
|---|---|---|---|---|
| SEQ | | | | Amino acid sequence |
| ID | | | | (start and |
| NO: | Domain | Score | e-value | end position) |

| | | | | |
|---|---|---|---|---|
| 428 | Leucine-rich | 2.7 | 2.4e+02 | STQRLFQNNLIRTLRPGTF |
| | repeat | | | GS (42-63) |
| 429 | Leucine-rich | 20.0 | 0.057 | NLLTLWLFSNNLSTIYPG |
| | repeat | | | TFRHLQ (64-87) |
| 430 | Leucine-rich | 22.6 | 0.0095 | ALEELDLGDNRHLRSLEP |
| | repeat | | | DTFQGLE (88-112) |
| 431 | Leucine-rich | 23.9 | 0.0037 | RLQSLHLYRCQLSSLPGN |
| | repeat | | | IFRGLV (113-136) |
| 432 | Leucine-rich | 18.3 | 0.18 | SLQYLYLQENSLLHLQD |
| | repeat | | | DLFADLA (137-160) |
| 433 | Leucine-rich | 15.9 | 0.97 | NLSHLFLHFNRLRLLTEH |
| | repeat | | | VFRGLG (161-184) |
| 434 | Leucine-rich | 16.5 | 0.62 | SLDFLLLHGNRLQGVHR |
| | repeat | | | AAFRGLS (185-208) |
| 435 | Leucine-rich | 23.1 | 0.0066 | RLTILYLFNNSLASLPGEA |
| | repeat | | | LADLP (209-232) |
| 436 | Leucine-rich | 38.9 | 1.2e−07 | NPWACDCRARPLWAWF |
| | repeat- | | | QRARVSSSDVTCATPPER |
| | associated | | | QGRDLRALREADFQACP |
| | C-terminal | | | (242-292) |
| | domain | | | |

Using the Kyte-Doolittle hydrophobicity prediction algorithm (J. Mol. Biol., 157:105-131 (1982), incorporated herein by reference), NgRHy is predicted to have a transmembrane domain at residues 382-396 (SEQ ID NO: 437):
LSAGLPSPLLCLLLL
wherein A-Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine. Removal of the transmembrane domain renders a soluble fragment that can be used to inhibit NgRHy and/or NgR activity and is designated as SEQ ID NO: 438.

In particular, the NgRHy polypeptides and polynucleotides of the invention may be used in the treatment of spinal cord injury, cranial or cerebral trauma, stroke, and demyelinating diseases.

The activity of an NgRHy polypeptide of the invention may manifest as modulating neural growth activity, such as stimulation of neurite outgrowth, stimulation of neural cell proliferation, regeneration of nerve and brain tissue, a soluble form of NgRHy can act as a competitive inhibitor to block NgRHy thereby stimulating axonal growth, alternatively, NgRHy can act as a decoy receptor to modulate, i.e. stimulate or inhibit, axonal growth. The mechanism underlying the particular condition or pathology will dictate whether NgRHy polypeptides, binding partners thereof, or inhibitors thereof would be beneficial to the subject in need of treatment.

The present invention provides methods for modifying, such as inducing or inhibiting, proliferation of neural cells and for regeneration of nerve and brain tissue, which comprise administering a composition of NgRHy polypeptides, disclosed in the present invention. Such proteins of the present invention may be used to treat central and peripheral nervous system disorders, neuropathies, and lesions, as well as mechanical and traumatic disorders, which involve degeneration, death or trauma to neural cells or nerve tissue. More specifically, a protein may be used in the treatment of diseases of the peripheral nervous system, such as peripheral nerve injuries, peripheral neuropathy, and localized neuropathies, and central nervous system diseases, such as Alzheimer's disease, Parkinson's disease, Huntington's disease, amyotrophic lateral sclerosis, and Shy-Drager syndrome. Further conditions which may be treated in accordance with the present invention include mechanical and traumatic disorders, such as spinal cord injuries, head trauma, and cerebrovascular diseases including stroke. Peripheral neuropathies resulting from chemotherapy or other medical therapies may also be treatable using a protein of the invention.

NgRHy polypeptides are used to produce antibodies that will bind to NgRHy and/or NgR, thereby inhibiting NgRHy and/or NgR activity. Inhibition of either receptor will block Nogo-induced neurite growth inhibition and can be an effective therapeutic to restore axonal function after injury or disease.

The soluble ectodomain of NgRHy is used as a competitive inhibitor to bind to and/or block the activity of NgRHy or NgR thereby rendering cells insenstitive to Nogo protein inhibition of axonal growth.

NgRHy inhibits Nogo-dependent signaling by acting as a decoy receptor. Binding of Nogo proteins and/or other ligands for NgR and NgRHy to ectopically expressed NgRHy can result in decreased binding of said ligands to NgR thereby reducing the effect of Nogo signaling on axonal growth.

Antibodies raised agains the NgRHy polypeptide or fragment thereof, can be used as a therapeutic for treatment of neurological conditions such as spinal cord injury, cranial or cerebral trauma, stroke, and demyelinating diseases. Anti-NgRHy antibodies can inhibit the activity of either NgRHy or NgR by blocking access, either by sterically inhibiting binding of the ligand or by changing the conformation of the receptor such that ligand binding does not occur or that the receptor is unable to activate downstream signaling molecules even if the ligand is bound.

4.4 Scavenger Receptor-Like Polypeptide

Macrophages actively uptake a wide range of molecules including proteins, bacteria and viral particles, apoptotic cells and red blood cells, and low density lipoproteins (LDLs) (Yamada et al., Cell Molec Life Sc 54:628-640 (1998) herein incorporated by reference). The scavenger receptors were first reported as receptors for oxidized and acetyl-LDLs. From cross-competition experiments it has become clear that macrophages and other cells express several classes of scavenger receptors. These receptors include type I and type II class A receptors, CD36 and SR-B1 class B receptors and CD68 and LOX-1 class C receptors that are distinct from the receptors for plasma LDLs. Atherosclerosis begins when lipoproteins accumulate in the arterial intima and become chemically modified thus initiating local vessel wall inflammation. This brings in monocytes-derived macrophages which avidly take up the modified lipids, becoming fat-laden “foam” cells which reside in the vessel wall and exacerbate the local inflammation.

Class A type I and II macrophage scavenger receptors are trimeric proteins of about 220-250 kDa with an amino-terminal collagenous domain that is essential for ligand binding. Type I receptors have a scavenger receptor Cysteine-rich domain (SRCR) while type II receptors do not. Receptors containing the SRCR domain bind immunoglobulin domain containing proteins and may serve as adhesion receptors. The collagen domains of these receptors have Gly-X-Y repeats and form a triple helical structure. The modified LDL binding site resides at the carboxy terminus of the collagen domain in a stretch of basic amino acid residues. The cytoplasmic domain is essential for cell surface expression and receptor endocytosis.

Type I and II receptors are expressed on all tissue macrophages. They are also expressed in brain in the perivascular macrophages called MATO cells, and endothelial cells of the liver, the adrenal gland and lymph nodes. Cytokines and other growth factors are known to modulate scavenger receptor expression. Type I and type II receptors bind and endocytose multiple ligands including acetyl-LDL, advanced glycation end products (AGE), and apoptotic cells. They also bind bacterial endotoxins, gram-positive bacteria and recognize lipoteichoic acid. The binding of endotoxins does not lead to endotoxin signaling and thus may be a way of getting rid of excess endotoxins. They also recognize Listeria and herpes simplex virus. Type I and type II scavenger receptors also mediate cell adhesion and may assist in developing robust immune response. In the brain, accumulation of the scavenged materials results in the formation of foam cells similar to that found with atherosclerosis and contributes to narrowing of the lumen of the arterioles in the cortex.

Thus, the scavenger receptor-like polypeptides and polynucleotides of the invention may be used in the treatment of atherosclerosis, disorders caused by the accumulation of denatured materials and cellular debris, bacterial and viral infections, inflammation, strengthening of immune response, and Alzheimer's disease.

The scavenger receptor-like polypeptide of SEQ ID NO: 444 is an approximately 495-amino acid protein with a predicted molecular mass of approximately 54 kDa unglycosylated.

Protein database searches with the BLASTX algorithm (Altschul S. F. et al., J. Mol. Evol. 36:290-300 (1993) and Altschul S. F. et al., J. Mol. Biol. 21:403-10 (1990), herein incorporated by reference) indicate that SEQ ID NO: 444 is homologous to macrophage scavenger receptors.

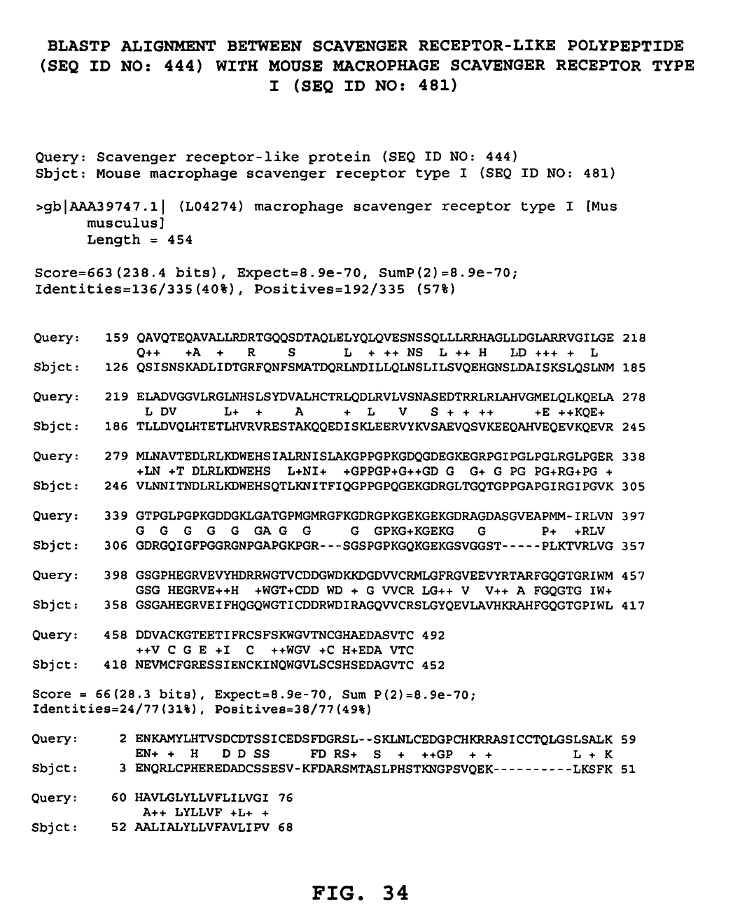

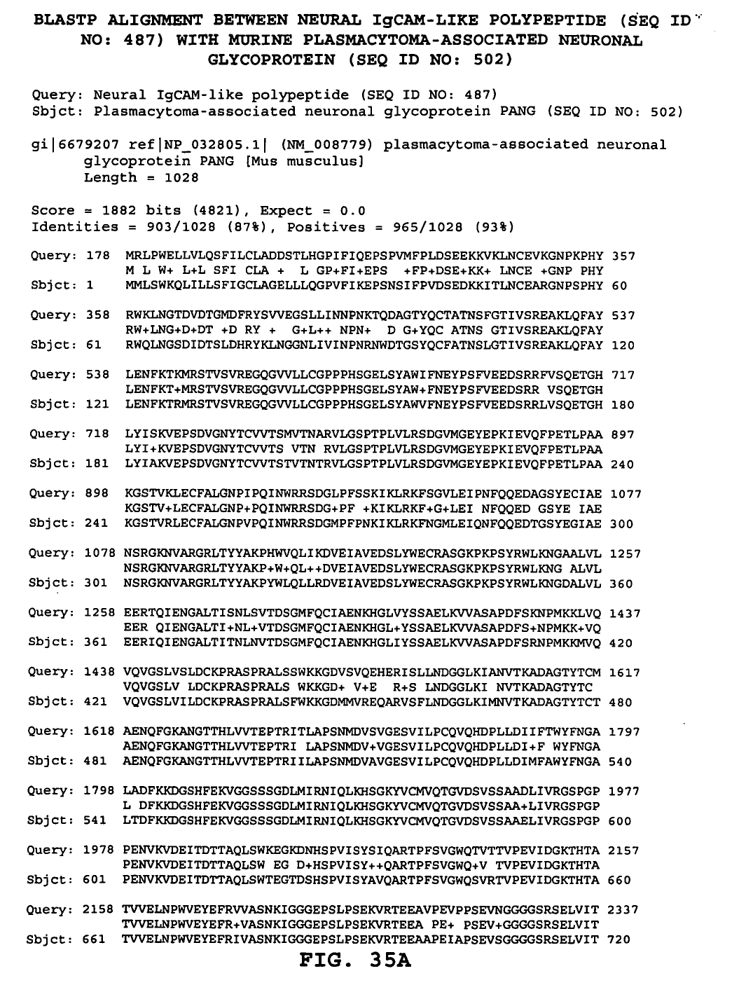

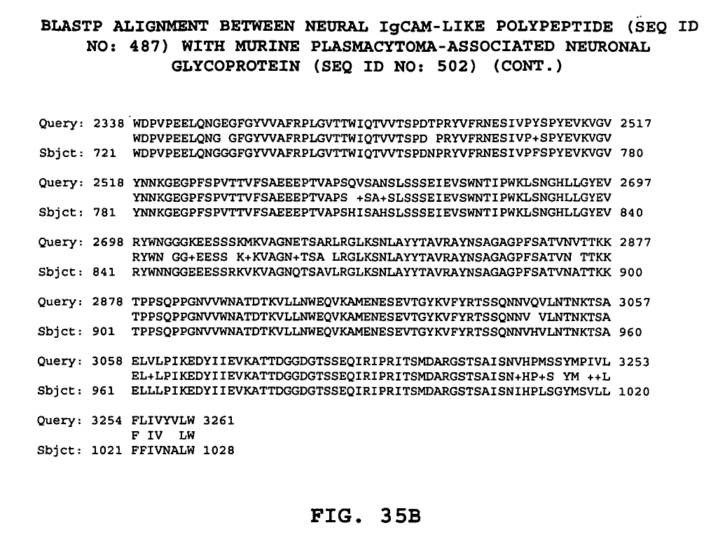

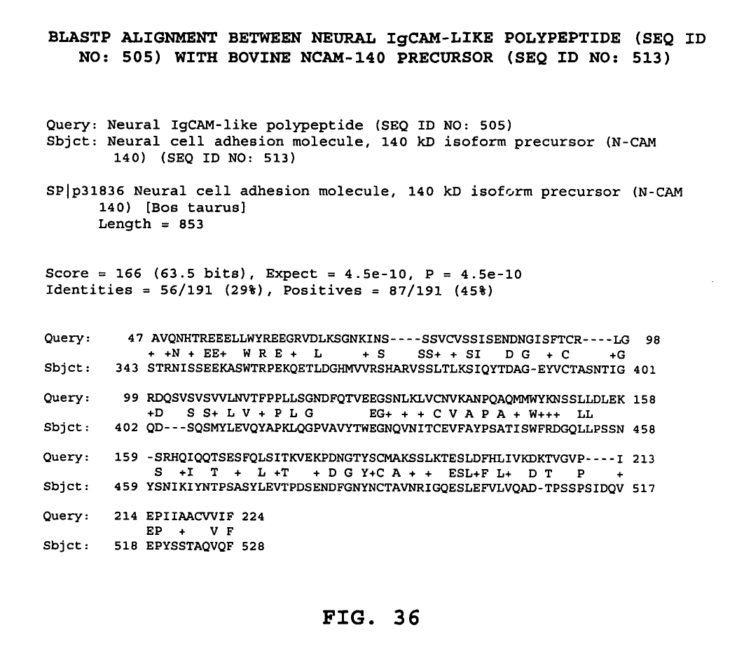

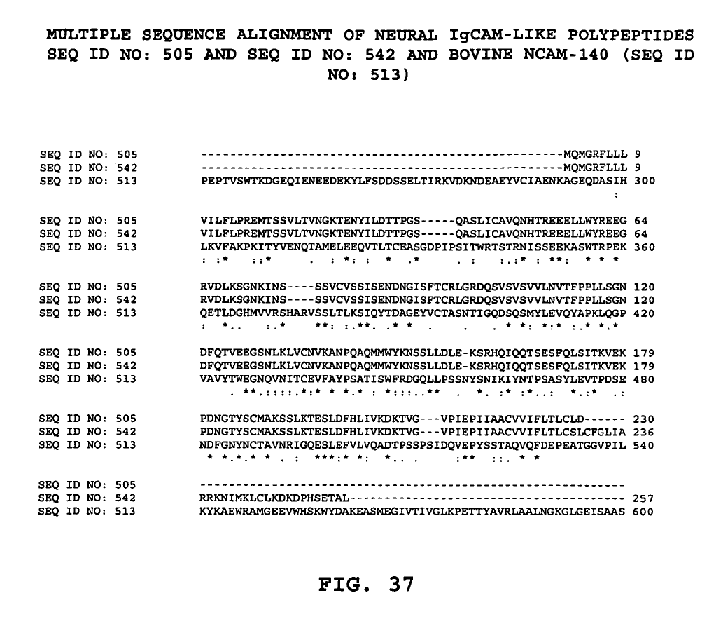

FIG. 34 shows the BLASTX amino acid sequence alignment between the protein encoded by SEQ ID NO: 443 (i.e. SEQ ID NO: 444) scavenger receptor-like polypeptide and mouse macrophage scavenger receptor type I (SEQ ID NO: 481), indicating that the two sequences share 57% similarity over a 335 amino acid residue region of SEQ ID NO: 444 and 40% identity over the same 335 amino acid residues of SEQ ID NO: 444. The results also indicate that the two sequences share 49% similarity over a distinct 77 amino acid residue region of SEQ ID NO: 444 and 31% identity over the same 77 amino acid residues of SEQ ID NO: 444, wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine. Gaps are presented as dashes.

Using the Pfam software program (Sonnhammer et al., Nucleic Acids Res. 26:320-322 (1998) herein incorporated by reference) SEQ ID NO: 444 was examined for domains with homology to certain peptide domains. Table 18 shows the SEQ ID NO: of the Pfam domain within SEQ ID NO: 444, the name of the Pfam model found, the description, the e-value, Pfam score, number of repeats, and position of the domain within SEQ ID NO: 444 for the identified model within the sequence as follows wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine:

TABLE 18

| SEQ | | | | | | |
|---|---|---|---|---|---|---|
| ID | | | | | Re- | |
| NO: | Model | Description | e-value | Score | peats | Position |
| 482 | SRCR | Scavenger | 2e−33 | 172.0 | 1 | 396-493 |
| | | receptor | | | | |
| | | cysteine-rich | | | | |
| | | domain | | | | |
| 483 | Collagen | Collagen triple | 9.1e−13 | 55.8 | 1 | 315-374 |
| | | helix repeat | | | | |

Further description of the Pfam models can be found at http://pfam.wustl.edu/.

A predicted approximately twenty-one residue transmembrane domain is encoded from approximately residue 61 through residue 81 of SEQ ID NO: 444 (SEQ ID NO: 484). The protein (SEQ ID NO: 444) lacking its transmembrane portion may be useful on its own. This can be confirmed by expression in mammalian cells. Presence of the transmembrane region was detected using the TMpred program (Hofmann and Stoffel, Biol. Chem. 374:166 (1993), herein incorporated by reference). One of skill in the art will recognize that the actual transmembrane region may be different than that predicted by the computer program.

Using eMATRIX software package (Stanford University, Stanford, Calif.) (Wu et al., J. Comp. Biol. 6:219-235 (1999), herein incorporated by reference), scavenger receptor-like polypeptide (SEQ ID NO: 444) is expected to have fourteen C1q domain proteins signatures as shown in Table 18. Using eMATRIX software package (Stanford University, Stanford, Calif.) (Wu et al., J. Comp. Biol. 6:219-235 (1999), herein incorporated by reference), scavenger receptor-like polypeptide (SEQ ID NO: 444) is also expected to have sixteen Speract receptor repeat proteins domain proteins signatures as shown in Table 19. Using eMATRIX software package (Stanford University, Stanford, Calif.) (Wu et al., J. Comp. Biol. 6:219-235 (1999), herein incorporated by reference), scavenger receptor-like polypeptide (SEQ ID NO: 444) is also expected to have five Speract receptor signatures as shown in Table 19. The domains corresponding to SEQ ID NO: 445-479 are as follows:

TABLE 19

| SEQ | | | | |
|---|---|---|---|---|
| ID | | Database | | |
| NO | p-Value | Entry ID | Description | Position* |
| 447 | 3.189e−13 | BL01113 | C1q domain proteins | 324-350 |
| 451 | 5.295e−11 | BL01113 | C1q domain proteins | 306-332 |
| 453 | 1.383e−10 | BL01113 | C1q domain proteins | 333-359 |
| 456 | 2.149e−10 | BL01113 | C1q domain proteins | 318-344 |
| 457 | 2.915e−10 | BL01113 | C1q domain proteins | 321-347 |
| 458 | 7.128e−10 | BL01113 | C1q domain proteins | 327-353 |
| 460 | 1.692e−09 | BL01113 | C1q domain proteins | 342-368 |
| 463 | 4.115e−09 | BL01113 | C1q domain proteins | 312-338 |
| 464 | 5.673e−09 | BL01113 | C1q domain proteins | 315-341 |
| 470 | 7.517e−08 | BL01113 | C1q domain proteins | 330-356 |
| 471 | 1.000e−07 | BL01113 | C1q domain proteins | 309-335 |
| 472 | 1.415e−07 | BL01113 | C1q domain proteins | 354-380 |
| 474 | 3.077e−07 | BL01113 | C1q domain proteins | 339-365 |
| 479 | 6.123e−07 | BL01113 | C1q domain proteins | 303-329 |
| 445 | 8.333e−39 | BL00420 | Speract receptor repeat proteins domain proteins | 397-451 |
| 448 | 9.100e−13 | BL00420 | Speract receptor repeat proteins domain proteins | 482-492 |
| 450 | 9.135e−12 | BL00420 | Speract receptor repeat proteins domain proteins | 309-337 |
| 452 | 7.382e−11 | BL00420 | Speract receptor repeat proteins domain proteins | 324-352 |
| 455 | 1.885e−10 | BL00420 | Speract receptor repeat proteins domain proteins | 348-376 |
| 459 | 7.639e−10 | BL00420 | Speract receptor repeat proteins domain proteins | 306-334 |
| 461 | 2.246e−09 | BL00420 | Speract receptor repeat proteins domain proteins | 321-349 |
| 465 | 4.423e−08 | BL00420 | Speract receptor repeat proteins domain proteins | 336-364 |
| 467 | 5.183e−08 | BL00420 | Speract receptor repeat proteins domain proteins | 312-340 |
| 468 | 5.310e−08 | BL00420 | Speract receptor repeat proteins domain proteins | 339-367 |
| 469 | 7.338e−08 | BL00420 | Speract receptor repeat proteins domain proteins | 327-355 |
| 473 | 3.077e−07 | BL00420 | Speract receptor repeat proteins domain proteins | 315-343 |
| 475 | 4.462e−07 | BL00420 | Speract receptor repeat proteins domain proteins | 351-379 |
| 476 | 5.615e−07 | BL00420 | Speract receptor repeat proteins domain proteins | 333-361 |
| 477 | 5.962e−07 | BL00420 | Speract receptor repeat proteins domain proteins | 342-370 |
| 478 | 5.962e−07 | BL00420 | Speract receptor repeat proteins domain proteins | 345-373 |
| 446 | 8.054e−16 | PR00258 | SPERACT RECEPTOR SIGNATURE | 393-409 |
| 449 | 1.509e−12 | PR00258 | SPERACT RECEPTOR SIGNATURE | 412-423 |
| 454 | 1.833e−10 | PR00258 | SPERACT RECEPTOR SIGNATURE | 481-493 |
| 462 | 3.667e−09 | PR00258 | SPERACT RECEPTOR SIGNATURE | 427-437 |
| 466 | 4.971e−08 | PR00258 | SPERACT RECEPTOR SIGNATURE | 458-472 |

*Position of signature in amino acid sequence (i.e. SEQ ID NO: 444)

In particular, the scavenger receptor-like polypeptides and polynucleotides of the invention may be used in the treatment of atherosclerosis, disorders caused by the accumulation of denatured materials and cellular debris, bacterial and viral infections, inflammation, strengthening of the immune response, and Alzheimer's disease.

4.5 Neural Immunoglobulin Cell Adhesion Molecule-Like (Neural IgCAM) Polypeptides

The establishment of neural connections during development is a highly dynamic process. A key aspect of this process is the regulation of axon growth, which is mediated by a variety of chemotropic factors (Skaper, et al., Prog. Neurobiol. 56:593-608 (2001), incorporated herein by reference). Chemotropism, which determines the direction of axonal growth, results from the concerted action of chemoattractant and chemorepellent cues (Yu and Bargmann, Nat. Neurosci. 4 (Suppl.): 1169-1176 (2001), incorporated herein by reference). Growth cones, the leading edge of the axons, encounter and detect these guiding cues along their trajectories in the form of gradients of diffusible factors, necessary for long-range guidance (Zheng and Kuffler, J. Neurobiol. 42:212-219 (2000), incorporated herein by reference), extracellular matrix-associated molecules, required for both short- and long-range regulation (Hynds and Snow, Exp. Neurol. 160:244-255 (1999), incorporated herein by reference; Skaper et al., 2001. supra), and membrane-bound molecules, necessary for short-range regulation (He and Meini, Mol. Cell. Neurosci. 19:18-31 (2002), incorporated herein by reference). It is believed that the inability of mature neurons to regenerate appropriate connections following injury or trauma is in part mediated by chemorepellent molecules present along axonal tracts (Fawcett, Cell Tissue Res. 290:371-377 (1997), incorporated herein by reference). During neural development, both membrane-bound and soluble proteins regulate axonal growth towards their targets. Integrins, cadherins and neural cell adhesion molecules (NCAMs) generally promote neurite outgrowth. Immunoglobulin superfamily members like L1 and NCAM are widely expressed and promote outgrowth of most neurons (Gil et al., J. Neurosci. 18:9312-9325 (1998), incorporated herein by reference).

Signals generated following neural IgCAM binding lead to alterations in cellular signaling and morphology affecting cell migration, proliferation, and differentiation. Subfamilies of neural IgCAMs are categorized according to the number of immunoglobulin (Ig) domains and fibronectin repeats, as well as the mode of attachment to the cell surface (either a transmembrane domain or a glycophosphatidyl inositol linkage), and the presence of a catalytic cytoplasmic domain (reviewed in Crossin and Krushel, Dev. Dyn. 218:260-279 (2000), herein incorporated by reference). A number of studies have correlated NCAM expression with the establishment of learning and memory (reviewed in Rose, Trends Neurosci. 18:502-506 (1995), herein incorporated by reference) as well as in schizophrenia (Poltorak et al., Brain Res. 751:152-154 (1997), herein incorporated by reference). Specific tyrosine kinases have been implicated in the effects of neural IgCAMs in neurite outgrowth (reviewed in Doherty and Walsh, Curr. Opin. Neurobiol. 4:49-55 (1994), herein incorporated by reference). Specifically, the fibroblast growth factor (FGF) receptor has been shown to be stimulated by interactions with neural IgCAMs via a “CAM homology domain” in the FGF receptor (Williams et al., Neuron 13:583-594 (1994); Williams et al., J. Cell Sci. 108:3523-3530 (1995), herein incorporated by reference). Additionally, nonreceptor tyrosine kinases, such as ERK1 and ERK2 have been implicated in signaling pathways associated with neural IgCAM in neurite outgrowth (Schmid et al., J. Neurobiol. 38:542-558 (1999), herein incorporated by reference).

Five exemplary neural IgCAM sequences of the invention are described below: amino acid sequence SEQ ID NO: 487 (and encoding nucleotide sequence SEQ ID NO: 486), amino acid SEQ ID NO: 505 (and encoding nucleotide sequence SEQ ID NO: 504), amino acid sequence SEQ ID NO: 516 (and encoding nucleotide sequence SEQ ID NO: 515), amino acid sequence SEQ ID NO: 528 (and encoding nucleotide sequence SEQ ID NO: 527), and amino acid sequence SEQ ID NO: 542 (and encoding nucleotide sequence SEQ ID NO: 541).

The first neural IgCAM-like polypeptide of SEQ ID NO: 487 is an approximately 1029-amino acid protein with a predicted molecular mass of approximately 1113-kDa unglycosylated. The initial methionine starts at position 178 of SEQ ID NO: 486 and the putative stop codon begins at position 3262 of SEQ ID NO: 486. A signal peptide of 18 residues is predicted from approximately residue 1 to residue 18 of SEQ ID NO: 487 (i.e. SEQ ID NO: 489). The extracellular portion is useful on its own. This can be confirmed by expression in mammalian cells and sequencing of the cleaved product. The signal peptide region was predicted using the Neural Network SignalP V1.1 program (Nielsen et al, Int. J. Neural Syst. 8:581-599 (1997)). One of skill in the art will recognize that the actual cleavage site may be different than that predicted by the computer program.

Using the TMpred program (Hofmann and Stoffel, Biol. Chem. 374:166 (1993), herein incorporated by reference), SEQ ID NO: 487 is predicted to have a transmembrane domain at approximately residue 904 to residue 920. Removal of the transmembrane domain renders soluble fragments that can be used to inhibit receptor activity. An exemplary extracellular domain spans approximately residue 19 to residue 903 of SEQ ID NO: 487 (i.e. SEQ ID NO: 501).

Protein database searches with the BLASTP algorithm (Altschul S. F. et al., J. Mol. Evol. 36:290-300 (1993) and Altschul S. F. et al., J. Mol. Biol. 21:403-10 (1990), herein incorporated by reference) indicate that SEQ ID NO: 487 is homologous to murine PANG, a neuronal CAM (SEQ ID NO: 502).

FIG. 35 shows the BLASTP amino acid sequence alignment between the protein derived from SEQ ID NO: 486 (i.e. SEQ ID NO: 487) and murine PANG amino acids 1-1028 of SEQ ID NO: 502, indicating that the two sequences share 93% similarity over 1028 amino acid residues of SEQ ID NO: 487 and 87% identity over the same 1028 amino acid residues of SEQ ID NO: 487, wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine. Gaps are represented as dashes.

Using the Pfam software program (Sonnhammer et al., Nucleic Acids Res., 26:320-322 (1998) herein incorporated by reference), neural IgCAM-like polypeptide of SEQ ID NO: 487 is predicted to contain five immunoglobulin (Ig) domains and four fibronectin type III (FN3) domains as shown in Table 20, wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine. Further description of the Pfam models can be found at http://pfam.wustl.edu/.

TABLE 20

| | | | | Amino |
|---|---|---|---|---|
| SEQ | | | | acid sequence encoded |
| ID | | | | (start and end amino |
| NO: | Domain | Score | e-value | acid position |
| 491 | Ig domain | 29.4 | 1.4e−07 | EKKVKLNCEVKGNPkPhYRW |
| | | | | KLNGTDVDTGMDFRYSVVEG |
| | | | | SLLINNPNKTQDAGTYQCTA |
| | | | | (43-102) |
| 492 | Ig domain | 23.8 | 8.2e−06 | GQGVVLLCGPPPHSGELSYA |
| | | | | WIFNEYPSFVEEDSRRFVSQE |
| | | | | TGHLYISKVEPSDVGNYTCVV |
| | | | | (137-198) |
| 493 | Ig domain | 38.4 | 2.3e−10 | GSTVKLECFALGNPIPQINWR |
| | | | | RSDGLPFSSKIKLRKFSGVLE |
| | | | | IPNFQQEDAGSYECIA |
| | | | | (242-299) |
| 494 | Ig domain | 32.5 | 1.6e−08 | GSLVSLDCKPRASPRALSSWK |
| | | | | KGDVSVQEHERISLLNDGGLK |
| | | | | IANVTKADAGTYTCMA |
| | | | | (424-481) |
| 495 | Ig domain | 26.2 | 1.4e−06 | ESVILPCQVQHDPLLDIIFTW |
| | | | | YFNGALADFKKDGSHFEKVGG |
| | | | | SSSGDLMLRNIQLKHSGKYVC |
| | | | | MV (514-579) |
| 496 | FN3 domain | 83.0 | 6.0e−21 | PGPPENVKVDEITDTTAQLSW |
| | | | | KEGKDNHSPVISYSIQARTPF |
| | | | | SVGWQTVTTVPEVIDGKTHTA |
| | | | | TVVELNPWVEYEFRVVASNKI |
| | | | | GGGEPS (598-687) |
| 497 | FN3 domain | 30.7 | 3.4e−05 | PEVPPSEVNGGGGSRSELVIT |
| | | | | WDPVPEELQNGEGFGYVVAF |
| | | | | RPLGVTTWIQTVVTSPDTPRY |
| | | | | VFRNESIYPYSPYEVKVGVYN |
| | | | | NKGEGPFS (700-790) |
| 498 | FN3 domain | 61.9 | 1.4e−14 | PTVAPSQVSANSLSSSEIEVS |
| | | | | WNTIPWKLSNGHLLGYEVRYW |
| | | | | NGGGPTVAPSQVSANSLSSSE |
| | | | | IEVSWNTIPWKLSNGHLLGYE |
| | | | | VRYWNGGG (802-891) |
| 499 | FN3 domain | 36.7 | 5.2e−17 | PSQPPGNVVWNATDTKVLLN |
| | | | | WEQVKAMENESEVTGYKVFY |
| | | | | RTSSQNNVQVLNTNKTSAELV |
| | | | | LPIKEDYIIEVKATTDGGDGT |
| | | | | SS (903-986) |

The second neural IgCAM-like polypeptide of SEQ ID NO: 505 is an approximately 231-amino acid protein with a predicted molecular mass of approximately 25-kDa unglycosylated. The initial methionine starts at position 17 of SEQ ID NO: 504 and the putative stop codon begins at position 707 of SEQ ID NO: 504. A signal peptide of 20 residues is predicted from approximately residue 1 to residue 20 of SEQ ID NO: 505 (i.e. SEQ ID NO: 507). The extracellular portion is useful on its own. This can be confirmed by expression in mammalian cells and sequencing of the cleaved product. The signal peptide region was predicted using the Neural Network SignalP V1.1 program (Nielsen et al, Int. J. Neural Syst. 8:581-599 (1997)). One of skill in the art will recognize that the actual cleavage site may be different than that predicted by the computer program.

Using the TMpred program (Hofmann and Stoffel, Biol. Chem. 374:166 (1993), herein incorporated by reference), SEQ ID NO: 505 is predicted to have a transmembrane domain at approximately residue 213 to residue 230. Removal of the transmembrane domain renders soluble fragments that can be used to inhibit receptor activity. An exemplary extracellular domain spans approximately residue 21 to residue 212 of SEQ ID NO: 505 (i.e. SEQ ID NO: 512).

Protein database searches with the BLASTP algorithm (Altschul S. F. et al., J. Mol. Evol. 36:290-300 (1993) and Altschul S. F. et al., J. Mol. Biol. 21:403-10 (1990), herein incorporated by reference) indicate that SEQ ID NO: 505 is homologous to bovine NCAM-140 precursor (SEQ ID NO: 513).

FIG. 36 shows the BLASTP amino acid sequence alignment between the protein derived from SEQ ID NO: 504 (i.e. SEQ ID NO: 505) and bovine NCAM-140 precursor amino acids 343-528 of SEQ ID NO: 513, indicating that the two sequences share 45% similarity over 191 amino acid residues of SEQ ID NO: 505 and 29% identity over the same 191 amino acid residues of SEQ ID NO: 505, wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine. Gaps are represented as dashes.

Using the Pfam software program (Sonnhammer et al., Nucleic Acids Res., 26:320-322 (1998) herein incorporated by reference), neural IgCAM-like polypeptide of SEQ ID NO: 505 is predicted to contain two immunoglobulin (Ig) domains as shown in Table 21, wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine. Further description of the Pfam models can be found at http://pfam.wustl.edu/.

TABLE 21

| | | | | Amino |
|---|---|---|---|---|
| SEQ | | | | acid sequence encoded |
| ID | | | | (start and end amino |
| NO: | Domain | Score | e-value | acid position |
| 509 | Ig domain | 13.1 | 0.017 | GSQASLICAVQNHTREEELLW |
| | | | | YREEGRVDLKSGNKINSSSVC |
| | | | | VSSISENDNGISFTCRL |
| | | | | (39-97) |
| 510 | Ig domain | 43.1 | 7.3e−12 | GSNLKLVCNVKANPQAQMM |
| | | | | WYKNSSLLDLEKSRHQIQQTS |
| | | | | ESFQLSITKVEKPDNGTYSCM |
| | | | | A (128-189) |

The third neural IgCAM-like polypeptide, SEQ ID NO: 541, is a variant of SEQ ID NO: 504. SEQ ID NO: 541 contains a 10 bp insertion between nucleotides 701 and 702 of SEQ ID NO: 504. The neural IgCAM-like polypeptide of SEQ ID NO: 541 (i.e. SEQ ID NO: 542) is an approximately 256 amino acid protein with a prediceted molecular mass of approximately 28 kDa unglycosylated. The initial methionine starts at position 17 of SEQ ID NO: 541 and the putative stop codon begins at position 788 of SEQ ID NO: 541. A signal peptide of 20 residues is predicted from approximately residue 1 to residue 20 of SEQ ID NO: 542 (i.e. SEQ ID NO: 507). The extracellular portion is useful on its own. This can be confirmed by expression in mammalian cells and sequencing of the cleaved product. The signal peptide region was predicted using the Neural Network SignalP V1.1 program (Nielsen et al, Int. J. Neural Syst. 8:581-599 (1997)). One of skill in the art will recognize that the actual cleavage site may be different than that predicted by the computer program.

Using the TMpred program (Hofmann and Stoffel, Biol. Chem. 374:166 (1993), herein incorporated by reference), SEQ ID NO: 542 is predicted to have a transmembrane domain at approximately residue 217 to residue 236 (i.e. SEQ ID NO: 545). Removal of the transmembrane domain renders soluble fragments that can be used to inhibit receptor activity. An exemplary extracellular domain spans approximately 21 to residue 216 of SEQ ID NO: 542 (i.e. SEQ ID NO: 546).

Protein database searches with the BLASTP algorithm (Altschul S. F. et al., J. Mol. Evol. 36:290-300 (1993) and Altschul S. F. et al., J. Mol. Biol. 21:403-10 (1990), herein incorporated by reference) indicate that SEQ ID NO: 542 is homologous to bovine NCAM-140 precursor (SEQ ID NO: 513).

FIG. 37 shows a multiple amino acid sequence alignment between neural IgCAM-like polypeptide SEQ ID NO: 505, neural IgCAM-like polypeptide SEQ ID NO: 542 and bovine NCAM-140 precursor (SEQ ID NO: 513), wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine. Gaps are represented as dashes (-), asterisks (*) represent identical amino acids, colons (:) represent conservative substitutions, and periods (.) represent semi-conservative substitutions.

Using the Pfam software program (Sonnhammer et al., Nucleic Acids Res., 26:320-322 (1998) herein incorporated by reference), neural IgCAM-like polypeptide of SEQ ID NO: 542 is predicted to contain two immunoglobulin (Ig) domains as shown in Table 22, wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine. Further description of the Pfam models can be found at http://pfam.wustl.edu/.

TABLE 22

| | | | | Amino |
|---|---|---|---|---|
| SEQ | | | | acid sequence encoded |
| ID | | | | (start and end amino |
| NO: | Domain | Score | e-value | acid position |
| 509 | Ig domain | 15.6 | 0.012 | GSQASLICAVQNHTREEELLW |
| | | | | YREEGRVDLKSGNKINSSSVC |
| | | | | VSSISENDNGISFTCRL |
| | | | | (39-97) |
| 510 | Ig domain | 44.6 | 1.1e−10 | GSNLKLVCNVKANPQAQMM |
| | | | | WYKNSSLLDLEKSRHQIQQTS |
| | | | | ESFQLSITKVEKPDNGTYSCM |
| | | | | A (128-189) |

The fourth neural IgCAM-like polypeptide of SEQ ID NO: 516 is an approximately 674-amino acid protein with a predicted molecular mass of approximately 74-kDa unglycosylated. The initial methionine starts at position 1 of SEQ ID NO: 516 and the putative stop codon begins at position 2000 of SEQ ID NO: 515. A signal peptide of 32 residues is predicted from approximately residue 1 to residue 32 of SEQ ID NO: 516 (i.e. SEQ ID NO: 518). The extracellular portion is useful on its own. This can be confirmed by expression in mammalian cells and sequencing of the cleaved product. The signal peptide region was predicted using the Neural Network SignalP V1.1 program (Nielsen et al, Int. J. Neural Syst. 8:581-599 (1997)). One of skill in the art will recognize that the actual cleavage site may be different than that predicted by the computer program.

Protein database searches with the BLASTP algorithm (Altschul S. F. et al., J. Mol. Evol. 36:290-300 (1993) and Altschul S. F. et al., J. Mol. Biol. 21:403-10 (1990), herein incorporated by reference) indicate that SEQ ID NO: 516 is homologous to murine CAM, DDM36 (SEQ ID NO: 525).

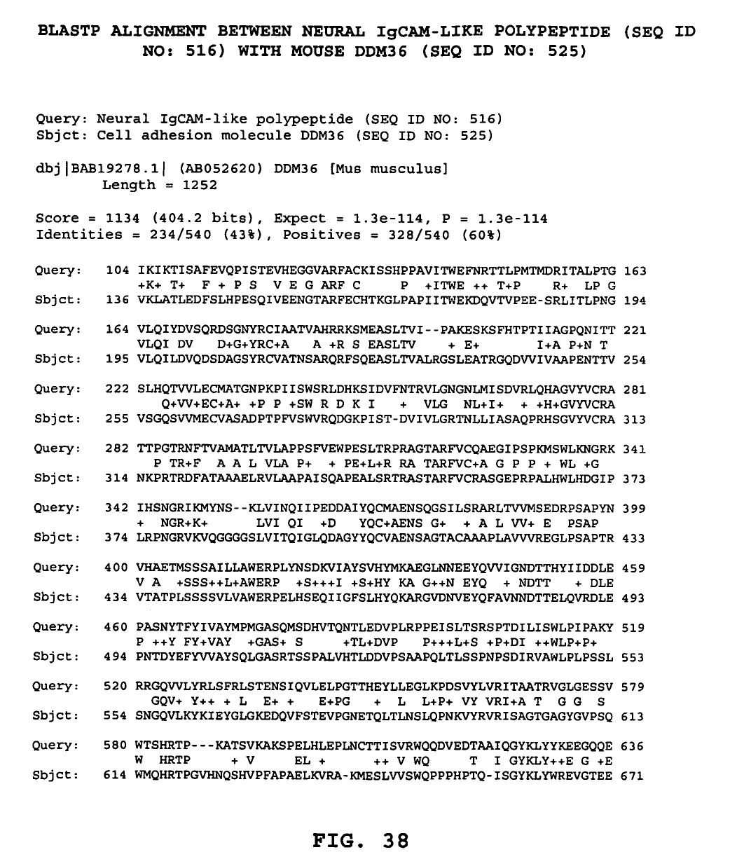

FIG. 38 shows the BLASTP amino acid sequence alignment between the protein derived from SEQ ID NO: 515 (i.e. SEQ ID NO: 514) and murine DDM36 amino acids 136-671 of SEQ ID NO: 525, indicating that the two sequences share 60% similarity over 540 amino acid residues of SEQ ID NO: 516 and 43% identity over the same 540 amino acid residues of SEQ ID NO: 516, wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine. Gaps are represented as dashes.

Using the Pfam software program (Sonnhammer et al., Nucleic Acids Res., 26:320-322 (1998) herein incorporated by reference), neural IgCAM-like polypeptide of SEQ ID NO: 516 is predicted to contain three immunoglobulin (Ig) and two fibronectin type III (FN3) domains as shown in Table 23, wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine. Further description of the Pfam models can be found at http://pfam.wustl.edu/.

TABLE 23

| | | | | Amino |
|---|---|---|---|---|
| SEQ | | | | acid sequence encoded |
| ID | | | | (start and end amino |
| NO: | Domain | Score | e-value | acid position |
| 520 | Ig domain | 28.2 | 3.6e−07 | GGVARFACKISSHPPAVITWE |
| | | | | FNRTTLPMTMDRITALPTGVL |
| | | | | QIYDVSQRDSGNYRCIA |
| | | | | (124-182) |
| 521 | Ig domain | 25.4 | 2.5e−06 | HQTVVLECMATGNPKPIISWS |
| | | | | RLDHKSIDVFNTRVLGNGNL |
| | | | | MISDVRLQHAGVYVCRA |
| | | | | (224-281) |
| 522 | Ig domain | 31.4 | 3.6e−08 | AGTARFVCQAEGIPSPKMSWL |
| | | | | KNGRKIHSNGRIKMYNSKLVI |
| | | | | NQIIPEDDAIYQCMA |
| | | | | (316-372) |
| 523 | FN3 domain | 60.2 | 4.3e−14 | PSAPYNVHAETMSSSAILLAW |
| | | | | ERPLYNSDKVIAYSVHYMKA |
| | | | | EGLNNEEYQVVIGNDTTHYII |
| | | | | DDLEPASNYTFYIVAYMPMG |
| | | | | ASQMS (394-480) |
| 524 | FN3 domain | 62.4 | 9.5e−15 | PLRPPEISLTSRSPTDILISW |
| | | | | LPIPAKYRRGQVVLYRLSFRL |
| | | | | STENSIQVLELPGTTHEYLLE |
| | | | | GLKYPDSVYLVRITAATRVGL |
| | | | | GESS (492-578) |

The fifth neural IgCAM-like polypeptide of SEQ ID NO: 528 is an approximately 1045-amino acid protein with a predicted molecular mass of approximately 115-kDa unglycosylated. The initial methionine starts at position 117 of SEQ ID NO: 527 and the putative stop codon begins at position 3249 of SEQ ID NO: 527. A signal peptide of 18 residues is predicted from approximately residue 1 to residue 18 of SEQ ID NO: 528 (i.e. SEQ ID NO: 530). The extracellular portion is useful on its own. This can be confirmed by expression in mammalian cells and sequencing of the cleaved product. The signal peptide region was predicted using the Neural Network SignalP V1.1 program (Nielsen et al, Int. J. Neural Syst. 8:581-599 (1997)). One of skill in the art will recognize that the actual cleavage site may be different than that predicted by the computer program.

Using the TMpred program (Hofmann and Stoffel, Biol. Chem. 374:166 (1993), herein incorporated by reference), SEQ ID NO: 528 is predicted to have a transmembrane domain at approximately residue 1023 to residue 1040. Removal of the transmembrane domain renders soluble fragments that can be used to inhibit receptor activity. An exemplary extracellular domain spans approximately residue 19 to residue 1022 of SEQ ID NO: 528 (i.e. SEQ ID NO: 539).

Protein database searches with the BLASTP algorithm (Altschul S. F. et al., J. Mol. Evol. 36:290-300 (1993) and Altschul S. F. et al., J. Mol. Biol. 21:403-10 (1990), herein incorporated by reference) indicate that SEQ ID NO: 528 is homologous to a rat CAM, BIG-2 precursor (SEQ ID NO: 540).

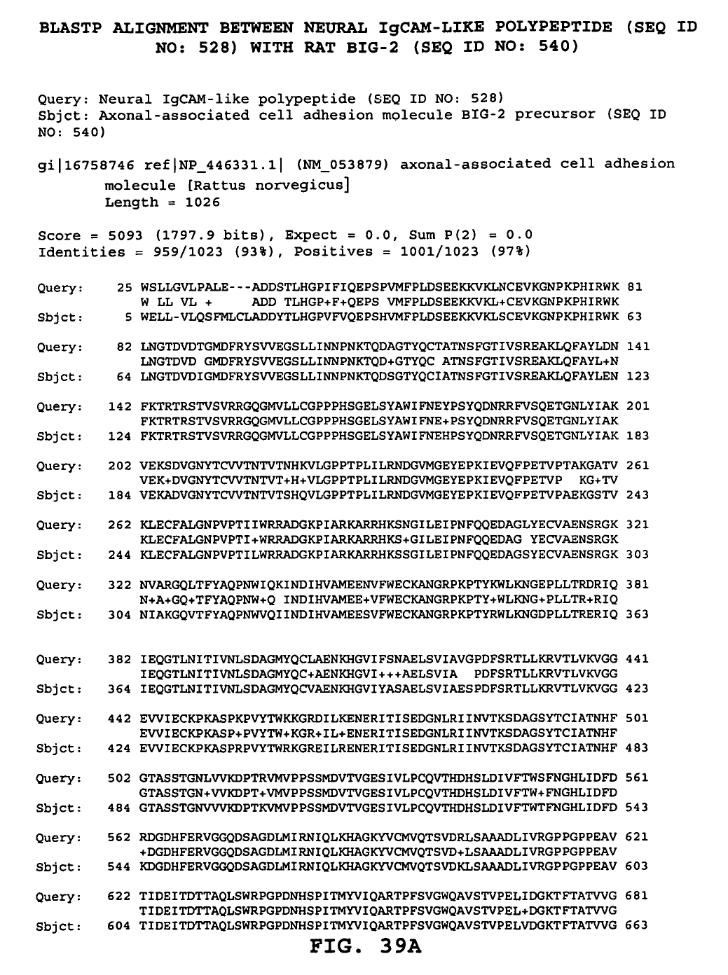

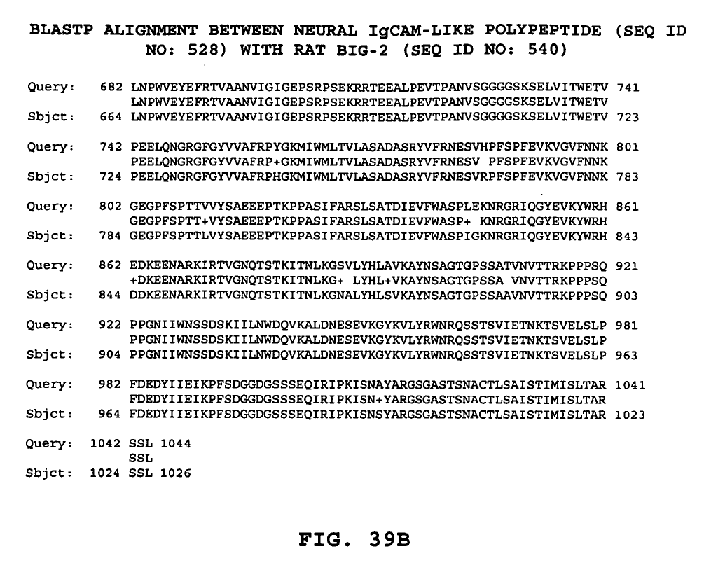

FIG. 39 (A, B) shows the BLASTP amino acid sequence alignment between the protein derived from SEQ ID NO: 527 (i.e. SEQ ID NO: 528) and rat BIG-2 precursor amino acids 5-1026 of SEQ ID NO: 540, indicating that the two sequences share 97% similarity over 1023 amino acid residues of SEQ ID NO: 528 and 93% identity over the same 1023 amino acid residues of SEQ ID NO: 528, wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine. Gaps are represented as dashes.

Using the Pfam software program (Sonnhammer et al., Nucleic Acids Res., 26:320-322 (1998) herein incorporated by reference), neural IgCAM-like polypeptide of SEQ ID NO: 528 is predicted to contain four immunoglobulin (Ig) and two fibronectin type III (FN3) domains as shown in Table 24, wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine. Further description of the Pfam models can be found at http://pfam.wustl.edu/.

TABLE 24

| | | | | Amino |
|---|---|---|---|---|
| SEQ | | | | acid sequence encoded |
| ID | | | | (start and end amino |
| NO: | Domain | Score | e-value | acid position |
| 532 | Ig domain | 30.1 | 8.7e−08 | EKKVKLNCEVKGNPKPHIRW |
| | | | | KLNGTDVDTGMDFRYSVVEG |
| | | | | SLLINNPNKTQDAGTYQCTA |
| | | | | (61-120) |
| 533 | Ig domain | 36.5 | 9.1e−10 | GATVKLECFALGNPVPTIIWR |
| | | | | RADGKPIARKARRHKSNGILE |
| | | | | IPNFQQEDAGLYECVA |
| | | | | (258-315) |
| 534 | Ig domain | 36.0 | 1.3e−09 | GGEVVLECKPKASPKPVYTWK |
| | | | | KGRDILKENERITISEDGNLR |
| | | | | IINVTKSDAGSYTCIA |
| | | | | (440-497) |
| 535 | Ig domain | 26.5 | 1.1e−06 | GESIVLPCQVTHDHSLDIVFT |
| | | | | WSFNGHLIDFDRDGDHEERVG |
| | | | | GQDSAGDLMIRNIQLKHAGK |
| | | | | YVCMV (530-596) |
| 536 | FN3 domain | 73.2 | 5.4e−18 | PGPPEAVTIDEITDTTAQLSW |
| | | | | RPGPDNHSPITMYVIQARTPF |
| | | | | SVGWQAVSTVPELIDGKTFTA |
| | | | | TVVGLNPWVEYEFRTVAANVI |
| | | | | GIGEPS (615-704) |
| 537 | FN3 domain | 51.6 | 1.8e−11 | PTKPPASIFARSLSATDIEVF |
| | | | | WASPLEKNRGRIQGYEVKYWR |
| | | | | HEDKEENARKIRTVGNQTSTK |
| | | | | ITNLKGSVLYHLAVKAYNSAG |
| | | | | TGPSS (819-907) |

Neural IgCAMs, such as BIG-2, PANG, and NCAM-140 mediate the formation, maintenance, and plasticity of functional neuronal networks (Yoshihara, et al., J. Neurobiol., 28:51-69 (1995), herein incorporated by reference). These neural IgCAMs facilitate neurite extension promoting axon growth and guidance (Connelly, et al., Proc. Natl. Acad. Sci. USA, 91:1337-1341 (1994), herein incorporated by reference). Neural IgCAMs mediate interactions with the extracellular environment by binding to extracellular matrix proteins, such as NCAM-140 binding to heparan sulfate proteoglycans (Prag, et al., J. Cell. Sci., 115:283-292 (2002), herein incorporated by reference). Neural IgCAMs are found predominantly on neural cells, but are also found on muscle cells, NK cells, T cells, and transiently expressed on a variety of cells during embryogenesis. PANG is a neural glycoprotein that is found primarily in neuronal cells, but is also ectopically expressed on plasmacytoma cells indicating that it may play a role in tumor metastasis as well as in axon guidance (Connelly, et al., 2001. supra).

The polypeptides of the invention are expected to have similar activities as those listed above, and therefore would be involved in neural development, specifically neurite outgrowth, neural cell proliferation, as well as in learning, behavior, and memory.

The polypeptides, polynucleotides, antibodies and other compositions of the invention are expected to provide potential treatments for disorders involving, but not limited to cognition, memory and learning, mood, dementia (including without limitation Alzheimer's disease, dementia associated with Parkinson's disease, multi-infarct dementia and others), depression, anxiety (including without limitation manic-depressive illness, obsessive-compulsive disorders, generalized anxiety and others), different forms of epilepsy, schizophrenia and schizophrenaform disorders (including without limitation schizoaffecto disorder), cerebral palsy and hypertension (see, e.g. U.S. Pat. No. 5,861,283, incorporated herein by reference). The polypeptides, polynucleotides, antibodies and other compositions of the invention may provide therapeutic compositions and methods of treatment for neurological conditions such as spinal cord injury, cranial or cerebral trauma, stroke, demyelinating diseases, and other neurodegenerative disorders including amyotrophic lateral sclerosis, progressive spinal muscular atrophy, progressive bulbar paralysis of childhood (Fazio-Londe syndrome), poliomyelitis and post polio syndrome, and hereditary motor sensory neuropathy (Charcot-Marie-Tooth Disease).

4.6 Growth Hormone-Like Polypeptides and Polynucleotides

Human growth hormone (hGH), also known as somatotropin, is a member of a family of homologous hormones that include placental lactogens, prolactins, and other genetic and species variants of growth hormone (Nichol et al., Endocrine Reviews, 7:169 (1986), incorporated herein by reference). The hGH gene cluster is located on chromosome 17 and consists of five highly conserved genes, hGH-N, hGH-V, hCS-L, hCS-A, and hCS-B. Human growth hormone-N is a 22,000-dalton hormone expressed in the somatotrope and lactosomatotrope cells of the anterior pituitary. Human growth hormone-N exhibits a multitude of biological effects, including linear growth (somatogenesis), lactation, activation of macrophages, and insulin-like and diabetogenic effects, among others (Chawla, Annu. Rev. Med., 34:519 (1983), incorporated herein by reference; Edwards et al., Science, 39:769 (1988), incorporated herein by reference; Isaksson et al., Annu. Rev. Physiol., 47:483 (1985), incorporated herein by reference; Thomer and Vance, J. Clin. Invest., 82:745 (1988), incorporated herein by reference; Hughes and Friesen, Annu. Rev. Physiol., 47:469 (1985), incorporated herein by reference). It promotes growth in the size of the limbs and internal organs. Hypersecretion of hGH causes giantism or acromegaly while its deficiency in children promotes dwarfism.

The remaining four genes of the growth hormone family, hGH-V, hCS-L, hCS-A, and hCS-B, are expressed in the syncytiotrophoblastic layer of the mid- to late gestational placenta (Su et al., J. Biol. Chem., 275;11 (2000), incorporated herein by reference). The hGH-V gene, also known as growth hormone-2, is a natural analog of hGH-N and is also potent somatogen. Like hGH-N, it binds growth hormone binding protein, increases glucose oxidation, induces refractoriness to insulin-like stimulation and lipolysis in the presence of glucocorticoids.

The biological effects of hGH derive from the interaction between hGH and specific cellular receptors. These interactions activate signaling pathways which contribute to growth hormone-induced changes in enzymatic activity, transport function, and gene expression that ultimately culminate in changes in growth and metabolism (Carter-Su et al., Annu. Rev. Physiol., 5:187 (1996), incorporated herein by reference).

Two exemplary growth hormone-like sequences of the invention are disclosed below: amino acid sequence SEQ ID NO: 548 (and encoding nucleotide sequence SEQ ID NO: 549) and amino acid sequence SEQ ID NO: 557 (and encoding nucleotide sequence SEQ ID NO: 556). The growth hormone-like polypeptide of SEQ ID NO: 548 is an approximately 173-amino acid protein with a predicted molecular mass of approximately 19 kDa unglycosylated. The initial methionine starts at position 58 of SEQ ID NO: 547 and the putative stop codon begins at position 577 of SEQ ID NO: 547. A signal peptide of twenty-six residues is predicted from approximately residue 1 to residue 26 of SEQ ID NO: 548. The extracellular portion is useful on its own. This can be confirmed by expression in mammalian cells and sequencing of the cleaved product. The signal peptide region was predicted using the Neural Network SignalP V1.1 program (Nielsen et al, Int. J. Neural Syst. 8:581-599 (1997)). One of skill in the art will recognize that the actual cleavage site may be different than that predicted by the computer program.

Protein database searches with the BLASTP algorithm (Altschul S. F. et al., J. Mol. Evol. 36:290-300 (1993) and Altschul S. F. et al., J. Mol. Biol. 21:403-10 (1990), herein incorporated by reference) indicate that SEQ ID NO: 548 is homologous to somatotropin/prolactin hormones.

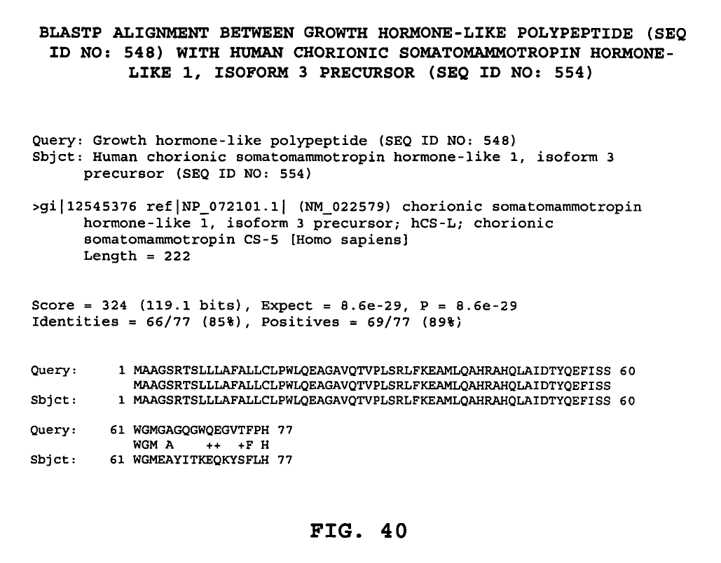

FIG. 40 shows the BLASTP amino acid sequence alignment between growth hormone-like polypeptide SEQ ID NO: 548 and human chorionic somatomammotropin hormone-like 1, isoform 3 precursor (SEQ ID NO: 554), indicating that the two sequences share 89% similarity over 77 amino acid residues and 85% identity over the same 77 amino acid residues, wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine. Gaps are presented as dashes.

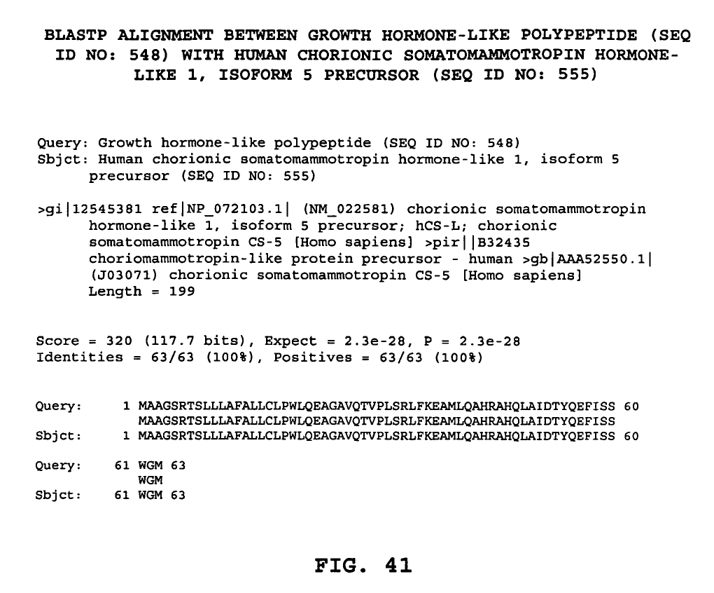

FIG. 41 shows the BLASTP amino acid sequence alignment between growth hormone-like polypeptide SEQ ID NO: 548 and human chorionic somatomammotropin hormone-like 1, isoform 5 percursor (SEQ ID NO: 555), indicating that the two sequences share 100% identity over 63 amino acid residues, wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine. Gaps are presented as dashes.

Using the Pfam software program (Sonnhammer et al., Nucleic Acids Res., 26:320-322 (1998) herein incorporated by reference) SEQ ID NO: 548 was examined for domains with homology to known conserved peptide domains. Table 25 shows the SEQ ID NO: of the Pfam domain, the name of the Pfam model found, the description, the e-value, Pfam score, number of repeats, and position of the domain within SEQ ID NO: 548 for the identified model within the sequence as follows:

TABLE 25

| SEQ | | | | | | |
|---|---|---|---|---|---|---|
| ID | | | | | Re- | |
| NO: | Model | Description | E-value | Score | peats | Position |
| 550 | hormone | Somatotropin | 1.6e−17 | 48.2 | 1 | 9-57 |
| | | hormone family | | | | |

Using the eMATRIX software package (Stanford University, Stanford, Calif.) (Wu et al., J. Comp. Biol., 6:219-235 (1999), herein incorporated by reference), the growth hormone-like polypeptide of SEQ ID NO: 548 was determined to have following the eMATRIX domain hits. The results in Table 26 describe: SEQ ID NO of the eMATRIX domain, the corresponding p-value, subtype, Signature ID number, domain name, the amino acid sequence of the eMATRIX domain and the corresponding position of the amino acids within SEQ ID NO: 548, wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine

TABLE 26

| | | | | Amino |
|---|---|---|---|---|
| | | | | acid sequence |
| SEQ | | | | encoded (start |
| ID | | Signature | | and end amino |
| NO | p-value | ID NO | Name | acid position) |
| 551 | 8.347e−11 | BL00266A | Somatotropin, | LFKEAMLQAHRAHQ |
| | | | prolactin and | LAIDTYQEFISSW |
| | | | related | (35-61) |
| | | | hormones | |
| | | | proteins | |

The second growth hormone-like polypeptide of SEQ ID NO: 557 is an approximately 256-amino acid protein with a predicted molecular mass of approximately 28 kDa unglycosylated. The initial methionine starts at position 58 of SEQ ID NO: 556 and the putative stop codon begins at position 826 of SEQ ID NO: 556. A signal peptide of twenty-six residues is predicted from approximately residue 1 to residue 26 of SEQ ID NO: 557. The extracellular portion is useful on its own. This can be confirmed by expression in mammalian cells and sequencing of the cleaved product. The signal peptide region was predicted using the Neural Network SignalP V1.1 program (Nielsen et al, Int. J. Neural Syst. 8:581-599 (1997)). One of skill in the art will recognize that the actual cleavage site may be different than that predicted by the computer program.

Protein database searches with the BLASTP algorithm (Altschul S. F. et al., J. Mol. Evol. 36:290-300 (1993) and Altschul S. F. et al., J. Mol. Biol. 21:403-10 (1990), herein incorporated by reference) indicate that SEQ ID NO: 557 is homologous to somatotropin/prolactin hormones.

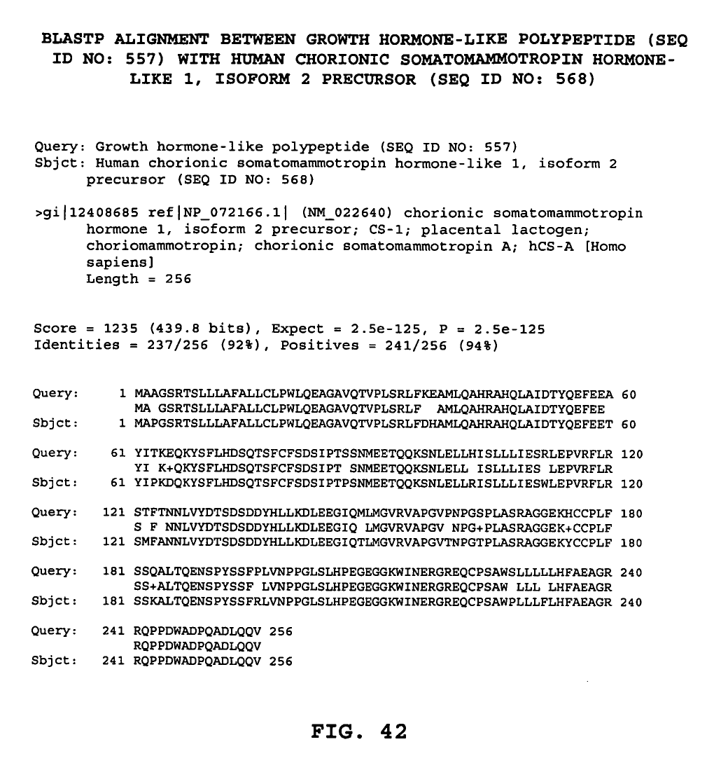

FIG. 42 shows the BLASTP amino acid sequence alignment between growth hormone-like polypeptide (SEQ ID NO: 557) and human chorionic somatomammotropin hormone 1, isoform 2 precursor (SEQ ID NO: 568), indicating that the two sequences share 94% similarity over 256 amino acid residues and 92% identity over the same 256 amino acid residues, wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine. Gaps are presented as dashes.

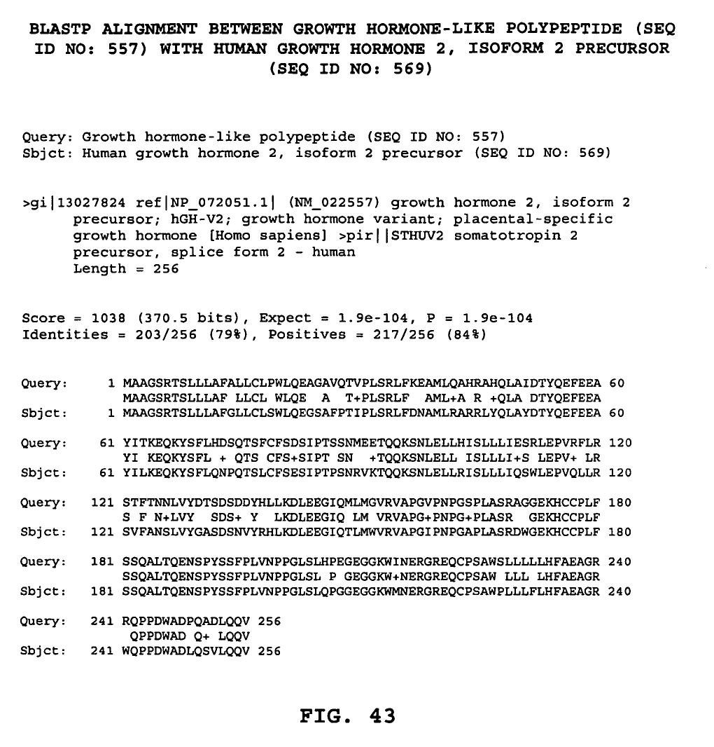

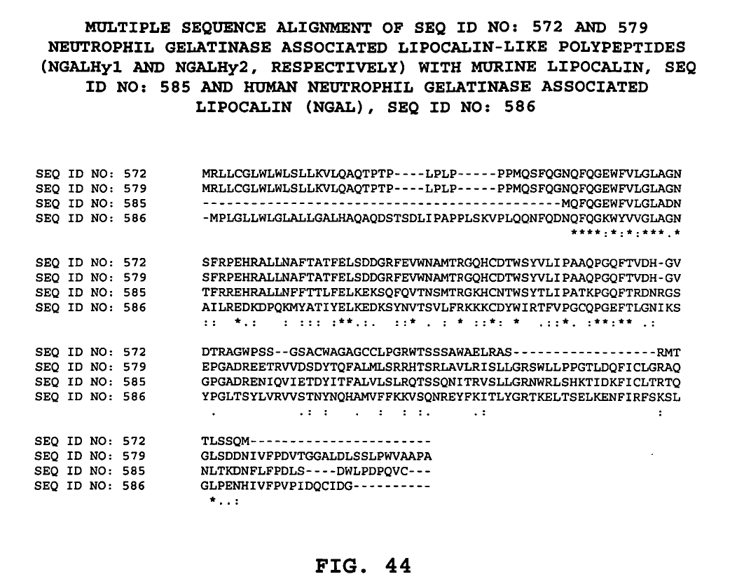

FIG. 43 shows the BLASTP amino acid sequence alignment between growth hormone-like polypeptide (SEQ ID NO: 557) and human growth hormone 2, isoform 2 precursor (SEQ ID NO: 569), indicating that the two sequences share 84% similarity over 256 amino acid residues and 79% identity over the same 256 amino acid residues, wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine. Gaps are presented as dashes.

Using the Pfam software program (Sonnhammer et al., Nucleic Acids Res., 26:320-322 (1998) herein incorporated by reference) SEQ ID NO: 557 was examined for domains with homology to known conserved peptide domains. Table 27 shows the SEQ ID NO: of the Pfam domain, the name of the Pfam model found, the description, the e-value, Pfam score, number of repeats, and position of the domain within SEQ ID NO: 558 for the identified model within the sequence as follows:

TABLE 27

| SEQ | | | | | | |
|---|---|---|---|---|---|---|
| ID | | | | | Re- | |
| NO: | Model | Description | E-value | Score | peats | Position |
| 551 | hormone | Somatotropin | 1.6e−57 | 156.1 | 1 | 9-151 |
| | | hormone family | | | | |

Using the eMATRIX software package (Stanford University, Stanford, Calif.) (Wu et al., J. Comp. Biol., 6:219-235 (1999), herein incorporated by reference), the growth hormone-like polypeptide of SEQ ID NO: 557 was determined to have following the eMATRIX domain hits. The results in Table 28 describe: SEQ ID NO of the eMATRIX domain, the corresponding p-value, subtype, Signature ID number, domain name, the amino acid sequence of the eMATRIX domain and the corresponding position of the amino acids within SEQ ID NO: 557, wherein A=Alanine, C=Cysteine, D=Aspartic Acid, E-Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine:

TABLE 28

| SEQ | | | | Amino acid sequence |
|---|---|---|---|---|
| ID | | Signature | | encoded (start and end |
| NO | p-value ID NO | Domain Name | amino acid position) | |
| 560 | 8.714e−21 | BL00266B | Somatotropin, prolactin | CFSDSIPTSSNMEETQ |
| | | | and related hormones | QKSNLELLHISLLLIES |
| | | | proteins | RLEPV (79-116) |
| 561 | 1.923e−14 | BL00266A | Somatotropin, prolactin | LFKEAMLQAHRAHQL |
| | | | and related hormones | AIDTYQEFEEAY |
| | | | proteins | (35-61) |
| 562 | 2.862e−11 | PR00836A | SOMATOTROPIN | CFSDSIPTSSNMEE |
| | | | HORMONE FAMILY | (79-92) |
| | | | SIGNATURE | |
| 563 | 4.000e−11 | BL00266D | Somatotropin, prolactin | PGLSLHPEGEGGKWI |
| | | | and related hormones | NERGREQCP (201-224) |
| | | | proteins | |
| 564 | 7.000e−11 | PR00836B | SOMATOTROPIN | LLHISLLLIESRLEPVR |
| | | | HORMONE FAMILY | FL (101-119) |
| | | | SIGNATURE | |
| 565 | 3.700e−10 | BL00266C | Somatotropin, prolactin | DDYHLLKDLEEGIQM |
| | | | and related hormones | LM (135-151) |
| | | | proteins | |

The growth hormone-like polypeptides, polynucleotides, antibodies and other compositions of the invention are expected to be useful in treating disorders where the growth of limbs and internal organs are effected, such as dwarfism, giantism, and acromegaly. Growth hormone-like polypeptides, polynucleotides, antibodies and other compositions of the invention may be used to treat metabolic disorders, including diabetes and obesity. Growth hormone-like polypeptides, polynucleotides, antibodies and other compositions of the invention may be used to treat inflammation, autoimmune diseases, and to modulate immune response.

4.7 Neutrophil Gelatinase-Associated Lipocalin-Like (NGALHy) Polypeptides and Polynucleotides

Lipocalins are a diverse family of proteins that are typically small (160-180 residues in length), extracellular proteins that bind small lipophilic molecules (such as retinol), cell surface receptors, and form covalent and non-covalent complexes with other soluble macromolecules (reviewed in Flower et al., Biochim. Biophys. Acta 1482:9-24 (2000), herein incorporated by reference). Proteins in the lipocalin family share a characteristic conserved lipocalin sequence motif as well as a common three-dimensional structure forming a β-barrel. Lipocalins have been shown to be overexpressed in a variety of diseases including cancer and inflammatory diseases.

Neutrophil gelatinase associated lipocalin (NGAL), a constituent of neutrophils granules, is a member of the lipocalin family. NGAL is highly induced in epithelial cells in both inflammatory and neoplastic colorectal disease (Goetz et al., Biochemistry 39:1935-1941 (2000), herein incorporated by reference). NGAL is proposed to mediate inflammatory responses by sequestering neutrophils chemoattractants, particularly N-formylated tripeptides as well as leukotriene B4 and platelet activating factor. Lipocalins are mainly extracellular carriers of lipophilic molecules, although exceptions with properties like prostaglandin synthesis and protease inhibition are observed for specific lipocalins. Study of lipocalins in cancer has so far been focused on the variations in concentration and the modification of their expression in distinct cancer forms. In addition, lipocalins have been assigned a role in cell regulation. Lipocalins have also been used extensively as biochemical markers of disease (see Xu and Venge, Biochim. Biophys. Acta 1482:298-307 (2000), herein incorporated by reference). The clinical indications relate to almost any field of medicine, such as inflammatory disease, cancer, lipid disorders, liver and kidney function.

Two exemplary NGAL-like sequences of the invention (NGALHy1 and NGALHy2) are described below: amino acid sequence SEQ ID NO: 572 (and encoding nucleotide sequence SEQ ID NO: 571), and amino acid SEQ ID NO: 579 (and encoding nucleotide sequence SEQ ID NO: 578).

The NGALHy1 polypeptide of SEQ ID NO: 572 is an approximately 157-amino acid protein with a predicted molecular mass of approximately 17-kDa unglycosylated. The initial methionine starts at position 192 of SEQ ID NO: 571 and the putative stop codon begins at position 660 of SEQ ID NO: 571. A signal peptide of 19 residues is predicted from approximately residue 1 to residue 19 of SEQ ID NO: 572. The extracellular portion is useful on its own. This can be confirmed by expression in mammalian cells and sequencing of the cleaved product. The signal peptide region was predicted using the Neural Network SignalP V1.1 program (Nielsen et al, Int. J. Neural Syst. 8:581-599 (1997)). One of skill in the art will recognize that the actual cleavage site may be different than that predicted by the computer program.

Protein database searches with the BLASTP algorithm (Altschul S. F. et al, J. Mol. Evol. 36:290-300 (1993) and Altschul S. F. et al., J. Mol. Biol. 21:403-10 (1990), herein incorporated by reference) indicate that SEQ ID NO: 572 is homologous to mouse lipocalin (SEQ ID NO: 585) and human NGAL precursor (SEQ ID NO: 586).

FIG. 44 shows a multiple sequence alignment of SEQ ID NO: 572 with other homologous sequences (SEQ ID NO: 585 and 586) showing conserved regions, wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine and asterisks (*) indicate identical residues, colons (:) indicate conserved substitutions, and periods (.) indicate distant substitutions.

Using eMATRIX software package (Stanford University, Stanford, Calif.) (Wu et al., J. Comp. Biol., 6:219-235 (1999), herein incorporated by reference), NGALHy1 polypeptide of SEQ ID NO: 572 was determined to have following the eMATRIX domain hits. The results describe: corresponding SEQ ID NO: in sequence listing, e-value, subtype, Accession number, domain name, amino acids of the full length protein of SEQ ID NO: 572 that correspond to the eMATRIX domain and are displayed in Table 29, wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine:

TABLE 29

| SEQ | | | | | |
|---|---|---|---|---|---|
| ID | | | Accession | Domain | Amino acid sequence |
| NO: | e-value | Subtype | No. | Name | (start and end position) |
| 576 | 5.500e−08 | 13.78 | PR00179A | Lipocalin | NQFQGEWFVLGLAGN |
| | | | | signature | (37-50) |

The NGALHy2 polypeptide of SEQ ID NO: 579 is an approximately 200-amino acid protein with a predicted molecular mass of approximately 22-kDa unglycosylated. The initial methionine starts at position 128 of SEQ ID NO: 578 and the putative stop codon begins at position 725 of SEQ ID NO: 578. A signal peptide of 19 residues is predicted from approximately residue 1 to residue 19 of SEQ ID NO: 579. The extracellular portion is useful on its own. This can be confirmed by expression in mammalian cells and sequencing of the cleaved product. The signal peptide region was predicted using the Neural Network SignalP V1.1 program (Nielsen et al, Int. J. Neural Syst. 8:581-599 (1997)). One of skill in the art will recognize that the actual cleavage site may be different than that predicted by the computer program.

Protein database searches with the BLASTP algorithm (Altschul S. F. et al, J. Mol. Evol. 36:290-300 (1993) and Altschul S. F. et al., J. Mol. Biol. 21:403-10 (1990), herein incorporated by reference) indicate that SEQ ID NO: 579 is homologous to mouse lipocalin (SEQ ID NO: 585) and human NGAL precursor (SEQ ID NO: 586).

FIG. 44 shows a multiple sequence alignment of SEQ ID NO: 579 width other homologous sequences (SEQ ID NO: 585 and 586) showing conserved regions, wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine. Gaps are presented as dashes and asterisks (*) represent identical residues, colons (:) represent conservative substitutions, periods (.) represent semi-conservative substitutions.

Using eMATRIX software package (Stanford University, Stanford, Calif.) (Wu et al., J. Comp. Biol., 6:219-235 (1999), herein incorporated by reference), NGALHy2 polypeptide of SEQ ID NO: 579 was determined to have following the eMATRIX domain hits. The results describe: corresponding SEQ ID NO: in sequence listing, e-value, subtype, Accession number, domain name, amino acids of the full length protein of SEQ ID NO: 579 that correspond to the eMATRIX domain and are shown in Table 30 below, wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine.

TABLE 30

| | | | | | |
|---|---|---|---|---|---|
| SEQ | | | | | |
| ID | | | Accession | Domain | Amino acid sequence |
| NO: | e-value | Subtype | No. | Name | (start and end position) |

| | | | | | |
|---|---|---|---|---|---|
| 583 | 5.500e−08 | 13.78 | PR00179A | Lipocalin | NQFQGEWFVLGLAG |
| | | | | signature | (37-50) |
| 584 | 7.214e−09 | 9.56 | PR00179B | Lipocalin | VDSDYTQFALMLS |
| | | | | signature | (121-134) |

NGAL forms a heterodimeric complex with matrix metalloproteinase 9 (MMP9) which protects MMP9 from degradation and allows MMP9 to degrade the extracellular matrix thereby enhancing tumor cell metastasis (Yan et al., J. Biol. Chem. 276:37258-37265 (2001) herein incorporated by reference). The MMP9/NGAL complex is induced in several cancers and is used as a marker for metastatic cancer. NGAL also modulates the immune response during the acute phase response during inflammation to enhance non-specific host defenses by binding to and neutralizing pro-infectious bacterial products, such as the chemoattractant N-formyl-Met-Leu-Phe (Goetz et al., 2000. supra; Logdberg and Wester, Biochim. Biophys. Acta, 1482:284-297 (2000), herein incorporated by reference). Circulating NGAL levels are used as a marker for inflammatory conditions, such as cystic fibrosis and acute peritonitis, and are capable of distinguishing between bacterial and viral acute infections. NGAL and lipocalins in general, also play a role in cell regulation, cell differentiation, and cell proliferation.

The polypeptides of the invention are expected to have similar functions as NGAL as a marker for diseases including cancer and inflammatory diseases, interacting with matrix metalloproteases to modulate cell proliferation, modulation of inflammation by enhancing non-specific host defenses, via activities such as binding to bacterial pro-inflammatory proteins.

The polypeptides, polynucleotides, antibodies and other compositions of the invention are expected to be useful in treating the following disorders: inflammatory diseases, including bacterial and viral infections, acute peritonitis, cystic fibrosis, asthma, chronic obstructive pulmonary disease, pulmonary emphysema, Sjogren's syndrome, rheumatoid arthritis; neoplastic colorectal disease, colitis, and other disorders in which the barrier of the colorectal mucosa is disrupted; wound healing; cancer, including breast, colorectal, pancreatic, prostate, bladder, renal cancers, colorectal and hepatic tumors, adenocarcinomas, including lung, colon, pancreas; lipid disorders, and modulating liver and kidney function.

4.8 Mucolipin-Like Polypeptides and Polynucleotides

Mucolipidosis IV (MLIV) is an autosomal recessive neurodegenerative lysosomal storage disorder characterized clinically by psychomotor retardation and ophthalmologic abnormalities including corneal opacitiy, retinal degeneration, and strabismus. Maximal development of the patient is between 12 and 15 months and age of the patients with this disease ranges from 1 to 40 years. Life expectancy of the patients is not known. Over 80% of the patients diagnosed with MLIV showing severe or mild symptoms are the Ashknazi Jews. The patients excrete chondroitin sulphate in their urine. The disease is characterized by massive engorgement of superficial and intermediate epithelial cells of both the cornea and conjunctiva with fine granular material consistent with mucopolysaccharide and concentric lamellar bodies. The storage materials have been identified as sphingolipids, phospholipids and acid mucopolysaccharides. In this disease, excessive storage of these materials is also observed in macrophages, plasma cells, ciliary epithelial cells, Schwann cells, retinal ganglion cells and vascular endothelial cells.

Unlike other lysosomal storage disorders, MLIV is not associated with a lack of lysosomal hydrolases. Instead the MLIV cells display abnormal endocytosis of lipids and accumulate large vesicles indicating that a defect in endocytosis may underlie the disease as shown by Chen, et al. (Chen, et al, Proc. Natl. Acad. Sci. USA. 98:6373-6378 (1998), herein incorporated by reference). Bassi, et al (Bassi, et al, Human Genet. 67:1110-1120, (2000)) also suggested that mucolipin 1 plays an important role in endocytosis, a fact that has been borne out by the studies of Fares and Greenwald using C. elegans as an animal model (Fares and Greenwald. Nature Genet. 28:64-68, (2001), herein incorporated by reference). They showed that a loss-of-function mutation in the C. elegans mucolipin 1 homolog, Cup-5 results in increased rate of uptake of fluid-phase markers, decreased degradation of the endocytosed protein and and accumulation of large vacuoles. Overexpression of cup-5 causes the opposite phenotype and rescue with human mucolipin 1 results in normalizing the endocytosis. Cup-5 is also essential for the viability and regulates the lysosomes in multiple cell types in C. elegans (Hersh et al. Proc Natl Acad Sci USA. 99:4355-4360, (2002), herein incorporated by reference).

The metabolic defect causing this accumulation has recently been identified as dysfunctional endocytosis and the gene responsible had been named mucolipin 1 (Bargal, et al., Nature Genet. 26:20-123, (2000), Bassi, et al., Human Genet. 67:1110-1120, (2000), Sun, et al Hum. Molec. Genet. 9:2471-2478, (2000), all of which are herein incorporated by reference) and it is a transcript of the gene MCOLN1 shown to be located on chromosome 19p13.3-p13.2 (Slaugenhaupt et al., Am. J. Hum. Genet. 65:773-778, (1999), herein incorporated by reference).

The MLIV gene consists of 14 exons spanning approximately 14 kb of genomic DNA and encoding a protein of 580 amino acid in length (Bargal, et al. Nature Genet. 26:120-123, (2000), herein incorporated by reference). The mucolipin protein appears to contain one transmembrane helix in the N-terminal region and at least 5 transmembrane domains ion the C-terminal half of the protein. This protein localizes on the plasma membrane and in the C-terminal region shows homology to polycistin-2, the product of the polycystic kidnay disease (PKD2) gene (Bassi, et al., Human Genet. 67:1110-1120, (2000), herein incorporated by reference). The gene also belongs to a family of transient receptor potential calcium ion channels (Sun, et al., Hum. Molec. Genet. 9:2471-2478, (2000), herein incorporated by reference) and may play a role in calcium ion transport.

Since the discovery of mucolipin 1 (also known as mucolipidin), at least two other human, three mouse proteins and a C. elegans cup-5 protein homologous to the mucolipin 1 have been identified creating a novel family of mucolipins. Since, studies on mucolipin 1 and cup-5 have shown the impact these proteins can have on cell viability, normal cellular transport, lysosomal storage and resulting in mental retardation, ophthalmic abnormalities such as corneal opacity, retinal degeneration and strabismus, there clearly exists a need for identifying further members of this family of proteins. Identification of such proteins and their methods of use to modulate cellular lysosomal transport provide therapeutic compositions and methods of treatments for the above-mentioned conditions.

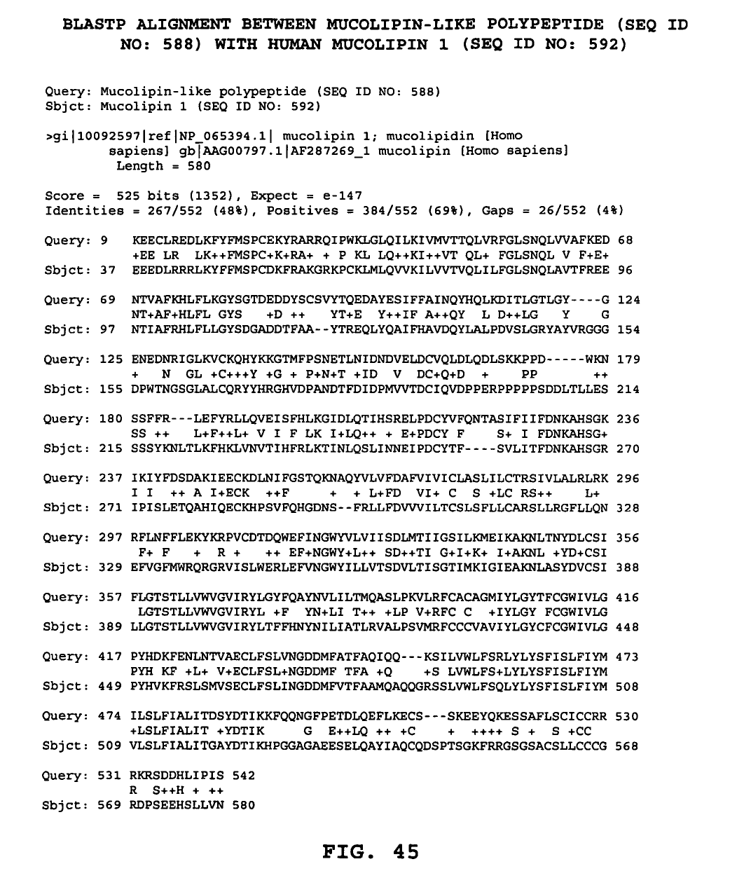

The mucolipin-like polypeptide of SEQ ID NO: 588 is an approximately 542-amino acid protein with a predicted molecular mass of approximately 59.6-kDa unglycosylated. The initial methionine starts at position 1 of SEQ ID NO: 587 and the putative stop codon begins at position 1629 of SEQ ID NO: 587: FIG. 45 shows the alignment between the protein in SEQ ID NO: 588 encoded by SEQ ID NO: 589 and human mucolipin 1 (SEQ ID NO: 592), indicating the two sequences share 48% identity over 542 amino acids wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine. Gaps are presented as dashes.

The mucolipin-like polypetide is not predicted to have a secretion signal peptide. The absence of signal peptide region was predicted using the Neural Network SignalP V1.1 program (Nielsen et al, Int. J. Neural Syst. 8:581-599 (1997), herein incorporated by reference). Using the TMpred program, the transmembrane regions of the polypeptide were determined. The TMpred program makes a prediction of membrane-spanning regions and their orientation. The algorithm is based on the statistical analysis of TMbase, a database of naturally occuring transmembrane proteins. The prediction is made using a combination of several weight-matrices for scoring. (K. Hofmann & W. Stoffel (1993) TMbase—A database of membrane spanning proteins segments. Biol. Chem. Hoppe-Seyler 374,166, herein incorporated by reference). One transmembrane region is predicted be present at the N-terminal end of the protein from 35 amino acid to 65 amino acid of SEQ ID NO: 588. Five additional transmembrane regions have been predicted by the Tmpred program from amino acid 266 to 282, 324 to 339, amino acid 353 to amino acid 370, 400 to amino acid 416, and amino acid 466 to amino acid 483 at the C-terminus of SEQ ID NO: 588.

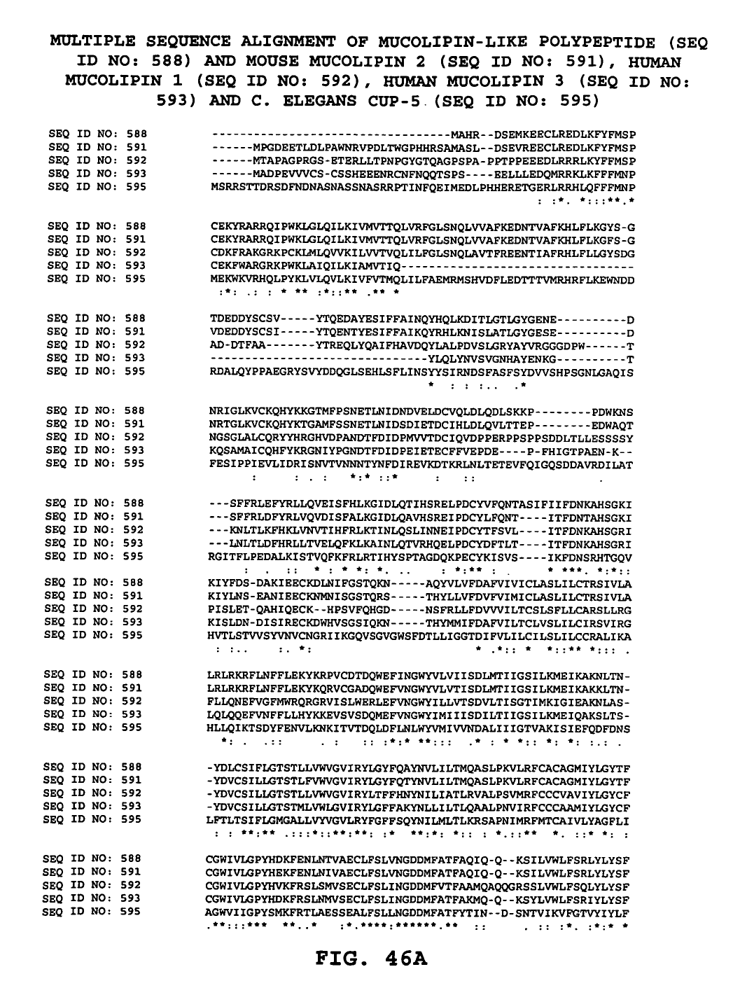

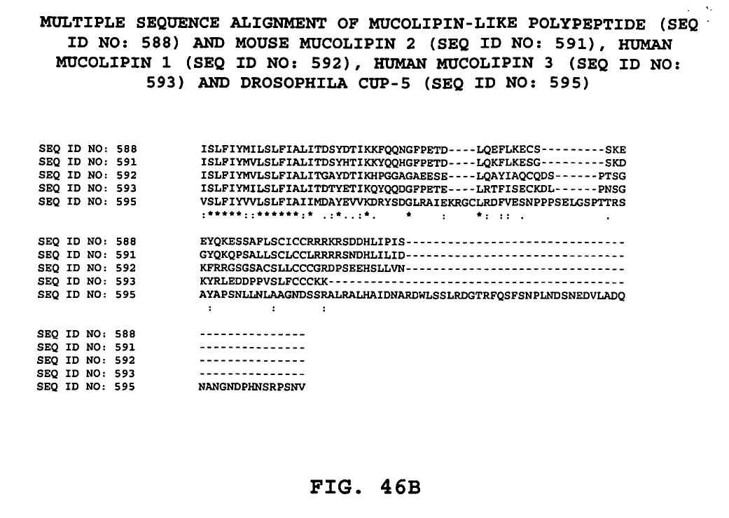

Protein database searches with the BLASTP algorithm (Altschul, et al., J. Mol. Evol. 36:290-300 (1993); Altschul et al, J. Mol. Biol. 21:403-10 (1990), herein incorporated by reference) indicate that SEQ ID NO: 588 is best homologous to mouse mucolipin 2. A multiple sequence alignment of SEQ ID NO: 588 with other homologous sequences showing conserved regions is shown in FIG. 46.

FIG. 46 shows a multiple sequence alignment between mucolipin-like polypeptide (SEQ ID NO: 588) and other members of the family: mouse mucolipin 2 (SEQ ID NO: 591), human mucolipin 1 (SEQ ID NO: 592), human mucolipin 3 (SEQ ID NO: 593), and Caenorhabditis elegans CUP-5 (SEQ ID NO: 595). Asterisks (*) indicate that the amino acid at that position is identical between the different polypetides, colons (:) indicate the amino acids at that postion are conservative replacements and periods (.) indicate the conserved presence of charged amino acids, wherin A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine. Gaps are presented as dashes.

Using the Pfam software program (Sonnhammer et al., Nucleic Acids Res., 26:320-322 (1998) herein incorporated by reference), mucolipin-like polypeptide of SEQ ID NO: 588 revealed its sequence homology to calcium ion transport pfam domain. Further description of the Pfam models can be found at http://pfam.wustl.edu/. Pfam domains hits are as follows: calcium ion_transport protein, score=22.4, e-value=0.0001, and amino acids of the full length protein of SEQ ID NO: 588 that correspond to the Pfam domain stretching from amino acid 322 to amino acid 482 and nucleotides of the open reading frame of SEQ ID NO: 590 that correspond to the domain.

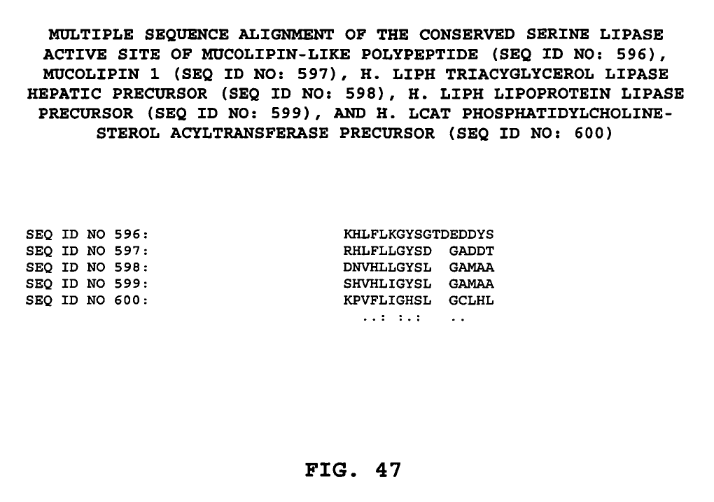

Mucolipin-like polypeptide contains a conserved serine lipase site spanning amino acid residues 74 to 90 of SEQ ID NO: 588 that is found in mucolipin 1 and other lipolytic enzymes. FIG. 47 shows an alignment of the conserved serine lipase active site between mucolipin-like polypeptide (SEQ ID NO: 596) and mucolipin 1 (SEQ ID NO: 597) as well as other lipolytic enzymes: H. liph. triacylglycerol lipase hepatic precursor (SEQ ID NO: 598), H. liph. lipoprotein lipase precursor (SEQ ID NO: 599) and H. lcat. phosphatidylcholine-sterol acyltransferase precursor (SEQ ID NO: 600).

Homologous family members SEQ ID NO: 592 and 595 have the following activities: endocytosis, calcium ion transport, apoptosis induction and lipolysis through a conserved serine lipase domain. The polypeptides of the invention are expected to have the following activities: based on homology and analysis of predicted pfam domains, the mucolipin-like polypeptide is expected to function as not only a calcium ion transport molecule but also as a serine lipase and play a role in apoptosis induction, endocytosis and lipid metabolism. The polypeptides, polynucleotides, antibodies and other compositions of the invention are expected to be useful in treating the following disorders: cholesterol storage diseases such as MLIV, cardiovascular, ophthalmic and neurologic diseases as well as diseases associated with apoptosis such as follicular lymphoma, autoimmune diseases and retinal degeneration.

4.9 Peroxidasin-Like Polypeptides and Polynucleotides

Peroxidasin was first identified and characterized in Drosophila as a novel enzyme-matrix protein based on its hybrid structure which combines an enzymatically active peroxidase motif with domains that usually occur as parts of interacting extracellular proteins (e.g. cell adhesion molecules) (Nelson et al, The EMBO Journal; 13:3438-3447(1994), incorporated herein by reference). Peroxidasin is a 1535 amino acid protein, wherein the amino acid sequence of the peroxidase domain is quite similar to the vertebrate peroxidases myeloperoxidase (MPO), eosinophil peroxidase (EPO), lactoperoxidase (LPO), and thyroid peroxidase (TPO). MPO, EPO, and LPO play key roles in human oxidative defense (Everse et al, Peroxidases in Chemistry and Biology; (1990), incorporated herein by reference). Since the expression of peroxidasin is accompanied by phagocytosis in the Drosophila embryo, peroxidasin may also function in phagocytosis. In addition to its peroxidase domain, peroxidasin possesses six leucine rich repeats (LRR) and four immunoglobulin (Ig) repeats. LRR's and Ig loops are involved in protein-protein interactions and indicate a role for peroxidasin in extracellular matrix consolidation and cell adhesion (Nelson et al, The EMBO Journal; 13:3438-3447(1994), incorporated herein by reference).

Overexpression of p53, a tumor suppressor protein whose inactivation has been observed in a large number of human cancers, leads to either programmed cell death (apoptosis) or growth arrest. A human homologue of Drosophila peroxidasin was shown to be differentially expressed in a human colon cancer cell line undergoing p53-dependent apoptosis (Horikoshi et al, Biochem. Biophys. Res. Commun.; 261:864-869(1999), incorporated herein by reference).

Recently, a novel melanoma gene (MG50) was identified which shows significant similarity to peroxidasin (Mitchell et al, Cancer Research; 60:6448-6456(2000), incorporated herein by reference). There is evidence that suggests MG50 is relatively restricted to tumors such as melanoma, breast cancer, ovarian cancer, and glioblastoma. In contrast, MG50 appears to be absent from archived specimens of normal tissues, with the exception of skin (Mitchell et al, Cancer Research; 60:6448-6456(2000), incorporated herein by reference). Since MG50 seems to be relatively tumor associated, it was hypothesized that MG50 could be a potentially useful immunogen and target for immunotherapy.

There exists a need for identifying further members of this family of proteins.

Six exemplary peroxidasin-like sequences of the invention are disclosed below: amino acid sequence SEQ ID NO: 602 (and encoding nucleotide sequence SEQ ID NO: 601), amino acid sequence SEQ ID NO: 618 (and encoding nucleotide sequence SEQ ID NO: 617), amino acid sequence SEQ ID NO: 622 (and encoding nucleotide sequence SEQ ID NO: 621), amino acid sequence SEQ ID NO: 626 (and encoding nucleotide sequence SEQ ID NO: 625), amino acid sequence SEQ ID NO: 607 (and encoding nucleotide sequence SEQ ID NO: 606), amino acid sequence SEQ ID NO: 612 (and encoding nucleotide sequence SEQ ID NO: 611).

The peroxidasin-like polypeptide of SEQ ID NO: 603 is an approximately 1507-amino acid protein with a predicted molecular mass of approximately 166 kDa unglycosylated. The initial methionine starts at position 261 of SEQ ID NO: 601 and the putative stop codon begins at position 4782 of SEQ ID NO: 601. A signal peptide of twenty three residues (SEQ ID NO: 604) is predicted from approximately residue 1 to residue 23 of SEQ ID NO: 602. The extracellular portion is useful on its own. This can be confirmed by expression in mammalian cells and sequencing of the cleaved product. The signal peptide region was predicted using the Neural Network SignalP V1.1 program (Nielsen et al, Int. J. Neural Syst. 8:581-599 (1997)). One of skill in the art will recognize that the actual cleavage site may be different than that predicted by the computer program.

Using the TMpred program (Hofmann and Stoffel, Biol. Chem. 374:166 (1993), herein incorporated by reference), SEQ ID NO: 602 is predicted to have transmembrane domains at approximately residue 5 to residue 26, residue 505 to residue 518, residue 593 to residue 608, and residue 1086 to residue 1104. Removal of one or more transmembrane domains renders fragments that can be useful on their own. One example is a fragment from residue 24 to residue 504 of SEQ ID NO: 602. One of skill in the art will recognize that the actual transmembrane domains may be different than that predicted by the computer program.

Protein database searches with the BLASTP algorithm (Altschul S. F. et al., J. Mol. Evol. 36:290-300 (1993) and Altschul S. F. et al., J. Mol. Biol. 21:403-10 (1990), herein incorporated by reference) indicate that SEQ ID NO: 602 is homologous to peroxidasin-like proteins.

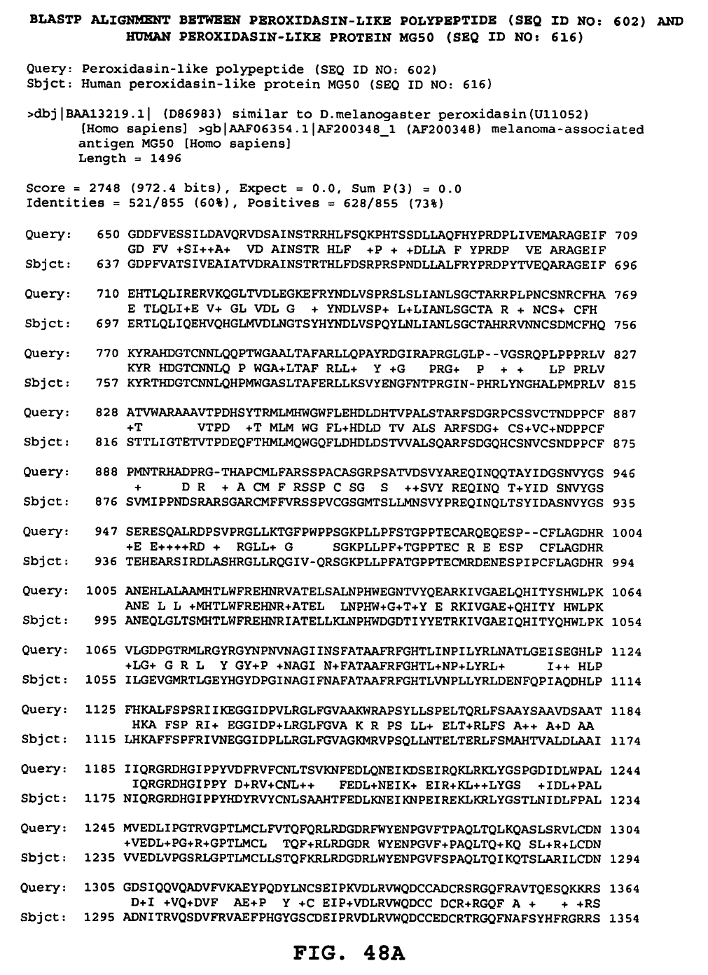

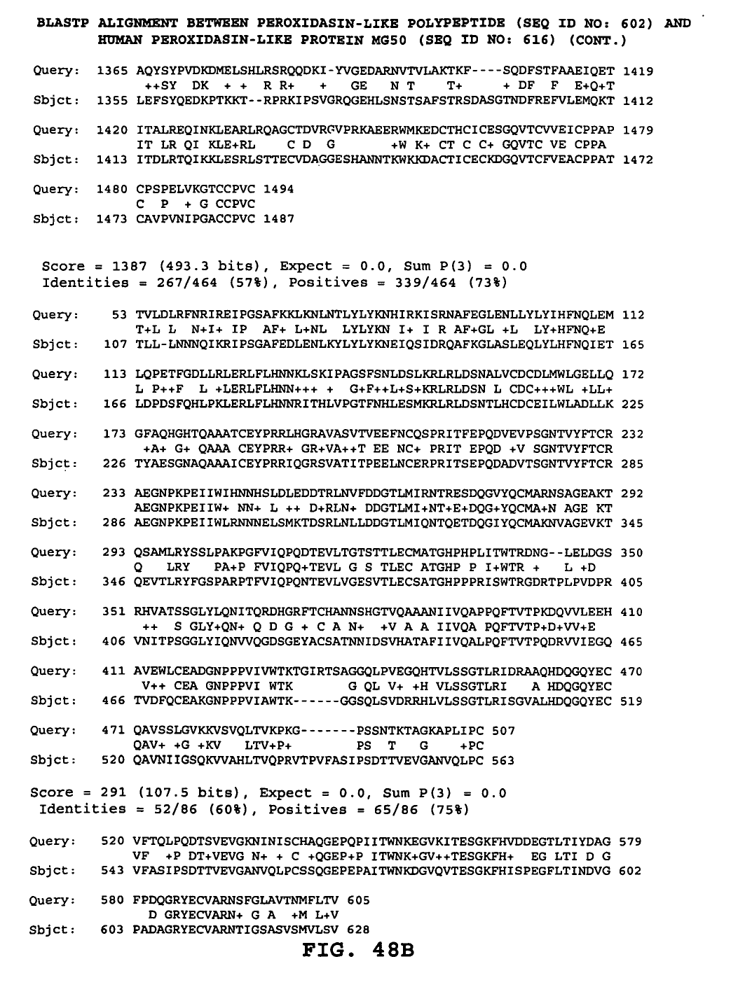

FIG. 48 shows the BLASTP amino acid sequence alignment between peroxidasin-like polypeptide SEQ ID NO: 602 and human peroxidasin-like protein (also known as melanoma-associated antigen, MG50) (SEQ ID NO: 616), indicating that the two sequences share: 73% similarity and 60% identity over 855 amino acid residues, 73% similarity and 57% identity over a distinct 464 amino acid residues, and 75% similarity and 60% identity over a distinct 86 amino acid residues, wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine. Gaps are presented as dashes.

Using the Pfam software program (Sonnhammer et al., Nucleic Acids Res., 26:320-322 (1998) herein incorporated by reference) SEQ ID NO: 602 was examined for domains with homology to known conserved peptide domains. Table 31 shows the name of the Pfam model found, the description, the e-value, Pfam score, number of repeats, and position of the domain(s) within SEQ ID NO: 602 for the identified model within the sequence as follows:

TABLE 31

| | | | | Re- | |
|---|---|---|---|---|---|
| Model | Description | E-value | Score | peats | Position |
| perox- | Peroxidase | 1.1e−40 | 148.6 | 1 | 770-1208 |
| idase | | | | | |
| Ig | Immunoglobulin | 4.1e−35 | 118.2 | 4 | 224-283 |
| | domain | | | | 320-376 |
| | | | | | 416-472 |
| | | | | | 533-590 |
| LRR | Leucine Rich Repeat | 1.4e−19 | 78.5 | 5 | 51-74 |
| | | | | | 75-98 |
| | | | | | 99-122 |
| | | | | | 123-146 |
| | | | | | 147-171 |
| LRRCT | Leucine rich repeat | 9.1e−11 | 49.2 | 1 | 156-208 |
| | C-terminal domain | | | | |
| vwc | von Willebrand factor | 7e−08 | 39.6 | 1 | 1439-1494 |
| | type C domain | | | | |
| TILa | TILa domain | 0.023 | 12.0 | 1 | 1438-1491 |

Using the eMATRIX software package (Stanford University, Stanford, Calif.) (Wu et al., J. Comp. Biol., 6:219-235 (1999), herein incorporated by reference), the peroxidasin-like polypeptide of SEQ ID NO: 602 was determined to have following the eMATRIX domain hits. The results in Table 32 describe: the eMATRIX domain name, the corresponding p-value, Signature ID number, and the corresponding position of the domain within SEQ ID NO: 603:

TABLE 32

| | | Signature | |
|---|---|---|---|
| Name | p-value | ID NO | Position |
| ANIMAL HAEM | 3.118e−22 | PR00457E | 1041-1067 |
| PEROXIDASE | | | |
| SIGNATURE | | | |
| ANIMAL HAEM | 4.194e−21 | PR00457D | 1016-1036 |
| PEROXIDASE | | | |
| SIGNATURE | | | |
| ANIMAL HAEM | 1.675e−13 | PR00457C | 998-1016 |
| PEROXIDASE | | | |
| SIGNATURE | | | |
| ANIMAL HAEM | 5.680e−13 | PR00457H | 1292-1306 |
| PEROXIDASE | | | |
| SIGNATURE | | | |
| ANIMAL HAEM | 4.750e−12 | PR00457F | 1094-1104 |
| PEROXIDASE | | | |
| SIGNATURE | | | |
| ANIMAL HAEM | 8.615e−12 | PR00457G | 1221-1241 |
| PEROXIDASE | | | |
| SIGNATURE | | | |
| VWFC domain | 3.250e−10 | BL01208B | 1480-1494 |
| proteins | | | |
| ANIMAL HAEM | 3.411e−10 | PR00457B | 846-861 |
| PEROXIDASE | | | |
| SIGNATURE | | | |
| Receptor tyrosine | 1.000e−09 | BL00240B | 325-348 |
| kinase class III | | | |
| proteins | | | |
| RECEPTOR FC | 4.581e−09 | PD01270A | 304-343 |
| IMMUNOGLOBULIN | | | |
| AFFIN. | | | |
| LEUCINE-RICH | 7.480e−09 | PR00019B | 73-86 |
| REPEAT | | | |
| SIGNATURE | | | |

A first variant of SEQ ID NO: 602 is SEQ ID NO: 618. The variant is an approximately 1538 amino acid protein with a predicted molecular mass of approximately 169 kDa unglycosylated. The initial methionine starts at position 12 of SEQ ID NO: 617, and the putative stop codon begins at position 4626 of SEQ ID NO: 617. A signal peptide of 54 residues is predicted from approximately residue 1 to residue 54 of SEQ ID NO: 618. The extracellular portion is useful on its own. This can be confirmed by expression in mammalian cells and sequencing of the cleaved product. SEQ ID NO: 618 differs from SEQ ID NO: 602 at the N-terminus where it contains an additional 31 amino acids. The remainder of SEQ ID NO: 618 is identical to SEQ ID NO: 602. Therefore, SEQ ID NO: 618 comprises SEQ ID NO: 602. The signal peptide region was predicted using the Neural Network SignalP V1.1 program (Nielsen et al, Int. J. Neural Syst. 8:581-599 (1997)). One of skill in the art will recognize that the actual cleavage site may be different than that predicted by the computer program.

Using the TMpred program (Hofmann and Stoffel, Biol. Chem. 374:166 (1993), herein incorporated by reference), SEQ ID NO: 618 is predicted to have a transmembrane domain at approximately residue 525 to residue 550. Removal of the transmembrane domain renders fragments that can be useful on their own. One of skill in the art will recognize that the actual transmembrane domain may be different than that predicted by the computer program.

Using the Pfam software program (Sonnhammer et al., Nucleic Acids Res., 26:320-322 (1998) herein incorporated by reference) SEQ ID NO: 618 was examined for domains with homology to known conserved peptide domains. Table 33 shows the name of the Pfam model found, the description, the e-value, Pfam score, number of repeats, and position of the domain(s) within SEQ ID NO: 618 for the identified model within the sequence as follows:

TABLE 33

| | | | | Re- | |
|---|---|---|---|---|---|
| Model | Description | E-value | Score | peats | Position |
| An_per- | Animal haem | 1e−192 | 653.6 | 1 | 801-1340 |
| oxidase | peroxidase | | | | |
| ig | Immunoglobulin | 1.4e−32 | 121.6 | 4 | 255-314: |
| | domain | | | | 351-407: |
| | | | | | 447-503: |
| | | | | | 564-621 |
| LRR | Leucine Rich | 3.3e−16 | 63.7 | 5 | 82-105: |
| | Repeat | | | | 106-129: |
| | | | | | 130-153: |
| | | | | | 154-177: |
| | | | | | 178-189 |
| LRRCT | Leucine rich | 1.2e−14 | 47.5 | 1 | 187-239 |
| | repeat C- | | | | |
| | terminal domain | | | | |
| vwc | von Willebrand | 1.2e−09 | 38.0 | 1 | 1470-1525 |
| | factor type C | | | | |
| | domain | | | | |
| TILa | TILa domain | 0.0017 | 16.9 | 1 | 1469-1508 |
| LRRNT | Leucine rich | 0.025 | 14.9 | 1 | 54-80 |
| | repeat N- | | | | |
| | terminal domain | | | | |

Using the eMATRIX software package (Stanford University, Stanford, Calif.) (Wu et al., J. Comp. Biol., 6:219-235 (1999), herein incorporated by reference), the peroxidasin-like polypeptide of SEQ ID NO: 618 was determined to have following the eMATRIX domain hits. The results in Table 34 describe: the eMATRIX domain name, the corresponding p-value, Signature ID number, and the corresponding position of the domain within SEQ ID NO: 618:

TABLE 34

| | | Signature | |
|---|---|---|---|
| Name | p-value | ID NO | Position |
| ANIMAL HAEM | 8.45e−24 | PR00457E | 1072-1098 |
| PEROXIDASE | | | |
| SIGNATURE V | | | |
| ANIMAL HAEM | 1.53e−20 | PR00457D | 1047-1067 |
| PEROXIDASE | | | |
| SIGNATURE IV | | | |
| ANIMAL HAEM | 9.42e−15 | PR00457C | 1029-1047 |
| PEROXIDASE | | | |
| SIGNATURE III | | | |
| ANIMAL HAEM | 4.48e−14 | PR00457G | 1252-1272 |
| PEROXIDASE | | | |
| SIGNATURE VII | | | |
| ANIMAL HAEM | 5.85e−13 | PR00457H | 1323-1337 |
| PEROXIDASE | | | |
| SIGNATURE VIII | | | |
| ANIMAL HAEM | 6.32e−12 | PR00457F | 1125-1135 |
| PEROXIDASE | | | |
| SIGNATURE VI | | | |
| LEUCINE RICH | 1.00e−10 | IPB000483 | 187-201 |
| REPEAT C- | | | |
| TERMINAL DOMAIN | | | |
| ANIMAL HAEM | 2.29e−10 | PR00457B | 877-892 |
| PEROXIDASE | | | |
| SIGNATURE II | | | |
| IMMUNOGLOBULIN | 2.80e−10 | IPB003006B | 383-420 |
| AND MAJOR HISTO- | | | |
| COMPATIBILITY | | | |
| COMPLEX DOMAIN | | | |
| IMMUNOGLOBULIN | 8.92e−10 | IPB003006B | 479-516 |
| AND MAJOR HISTO- | | | |
| COMPATIBILITY | | | |
| COMPLEX DOMAIN | | | |
| IMMUNOGLOBULIN | 9.28e−10 | IPB003006B | 290-327 |
| AND MAJOR HISTO- | | | |
| COMPATIBILITY | | | |
| COMPLEX DOMAIN | | | |

A second variant of SEQ ID NO: 602 is SEQ ID NO: 622. The splice site occurs after nucleotide 329 of SEQ ID NO: 601. The variant is an approximately 1400 amino acid protein with a predicted molecular mass of approximately 154 kDa unglycosylated. The initial methionine starts at position 103 of SEQ ID NO: 621, and the putative stop codon begins at position 4303 of SEQ ID NO: 621. A signal peptide of 23 residues is predicted from approximately residue 1 to residue 23 of SEQ ID NO: 622. The extracellular portion is useful on its own. This can be confirmed by expression in mammalian cells and sequencing of the cleaved product. The signal peptide region was predicted using the Neural Network SignalP V1.1 program (Nielsen et al, Int. J. Neural Syst. 8:581-599 (1997)). One of skill in the art will recognize that the actual cleavage site may be different than that predicted by the computer program.

Using the Pfam software program (Sonnhammer et al., Nucleic Acids Res., 26:320-322 (1998) herein incorporated by reference) SEQ ID NO: 622 was examined for domains with homology to known conserved peptide domains. Table 35 shows the name of the Pfam model found, the description, the e-value, Pfam score, number of repeats, and position of the domain(s) within SEQ ID NO: 622 for the identified model within the sequence as follows:

TABLE 35

| | | | | Re- | |
|---|---|---|---|---|---|
| Model | Description | E-value | Score | peats | Position |
| An_per- | Animal haem | 1e−192 | 653.6 | 1 | 663-1202 |
| oxidase | peroxidase | | | | |
| Ig | Immunoglobulin | 7.8e−25 | 95.7 | 4 | 201-260 |
| | domain | | | | 297-353 |
| | | | | | 393-449 |
| | | | | | 514-532 |
| LRR | Leucine Rich | 2.7e−14 | 57.0 | 4 | 51-74 |
| | Repeat | | | | 75-98 |
| | | | | | 99-122 |
| | | | | | 123-146 |
| Vwc | von Willebrand | 1.2e−09 | 38.0 | 1 | 1332-1387 |
| | factor type C | | | | |
| | domain | | | | |
| TILa | TILa domain | 0.0017 | 16.9 | 1 | 1331-1370 |
| LRRNT | Leucine rich | 0.025 | 14.9 | 1 | 23-49 |
| | repeat N- | | | | |
| | terminal domain | | | | |

Using the eMATRIX software package (Stanford University, Stanford, Calif.) (Wu et al., J. Comp. Biol., 6:219-235 (1999), herein incorporated by reference), the peroxidasin-like polypeptide of SEQ ID NO: 622 was determined to have following the eMATRIX domain hits. The results in Table 36 describe: the eMATRIX domain name, the corresponding p-value, Signature ID number, and the corresponding position of the domain within SEQ ID NO: 622:

TABLE 36

| | | Signature | |
|---|---|---|---|
| Name | p-value | ID NO | Position |
| ANIMAL HAEM | 8.45e−24 | PR00457E | 934-960 |
| PEROXIDASE | | | |
| SIGNATURE V | | | |
| ANIMAL HAEM | 1.53e−20 | PR00457D | 909-929 |
| PEROXIDASE | | | |
| SIGNATURE IV | | | |
| ANIMAL HAEM | 9.42e−15 | PR00457C | 891-909 |
| PEROXIDASE | | | |
| SIGNATURE III | | | |
| ANIMAL HAEM | 4.48e−14 | PR00457G | 1114-1134 |
| PEROXIDASE | | | |
| SIGNATURE VII | | | |
| ANIMAL HAEM | 5.85e−13 | PR00457H | 1185-1199 |
| PEROXIDASE | | | |
| SIGNATURE VIII | | | |
| ANIMAL HAEM | 6.32e−12 | PR00457F | 987-997 |
| PEROXIDASE | | | |
| SIGNATURE VI | | | |
| ANIMAL HAEM | 2.29e−10 | PR00457B | 739-754 |
| PEROXIDASE | | | |
| SIGNATURE II | | | |
| IMMUNOGLOBULIN | 2.80e−10 | IPB003006B | 329-366 |
| AND MAJOR | | | |
| HISTOCOMPATIBILITY | | | |
| COMPLEX DOMAIN | | | |
| IMMUNOGLOBULIN | 8.92e−10 | IPB003006B | 425-462 |
| AND MAJOR | | | |
| HISTOCOMPATIBILITY | | | |
| COMPLEX DOMAIN | | | |
| IMMUNOGLOBULIN | 9.28e−10 | IPB003006B | 236-273 |
| AND MAJOR | | | |
| HISTOCOMPATIBILITY | | | |
| COMPLEX DOMAIN | | | |
| LEUCINE-RICH | 6.73e−09 | PR00019B | 73-86 |
| REPEAT | | | |
| SIGNATURE II | | | |

A third variant of SEQ ID NO: 602 is SEQ ID NO: 626. The splice site occurs after nucleotide 329 of SEQ ID NO: 601. The variant is an approximately 1439 amino acid protein with a predicted molecular mass of approximately 158 kDa unglycosylated. The initial methionine starts at position 261 of SEQ ID NO: 625, and the putative stop codon begins at position 4578 of SEQ ID NO: 625. A signal peptide of 23 residues is predicted from approximately residue 1 to residue 23 of SEQ ID NO: 626. The extracellular portion is useful on its own. This can be confirmed by expression in mammalian cells and sequencing of the cleaved product. The signal peptide region was predicted using the Neural Network SignalP V1.1 program (Nielsen et al, Int. J. Neural Syst. 8:581-599 (1997)). One of skill in the art will recognize that the actual cleavage site may be different than that predicted by the computer program.

Using the Pfam software program (Sonnhammer et al., Nucleic Acids Res., 26:320-322 (1998) herein incorporated by reference) SEQ ID NO: 626 was examined for domains with homology to known conserved peptide domains. Table 37 shows the name of the Pfam model found, the description, the e-value, Pfam score, number of repeats, and position of the domain(s) within SEQ ID NO: 626 for the identified model within the sequence as follows:

TABLE 37

| | | | | Re- | |
|---|---|---|---|---|---|
| Model | Description | E-value | Score | peats | Position |
| An_per- | Animal haem | 9.1e−194 | 657.1 | 1 | 702-1241 |
| oxidase | peroxidase | | | | |
| ig | Immunoglobulin | 6.2e−34 | 126.2 | 4 | 224-283 |
| | domain | | | | 320-376 |
| | | | | | 416-466 |
| | | | | | 501-558 |
| LRR | Leucine Rich | 3.3e−16 | 63.7 | 5 | 51-74 |
| | Repeat | | | | 75-98 |
| | | | | | 99-122 |
| | | | | | 123-146 |
| | | | | | 147-158 |
| LRRCT | Leucine rich | 1.2e−14 | 47.5 | 1 | 156-208 |
| | repeat C- | | | | |
| | terminal domain | | | | |
| vwc | von Willebrand | 1.2e−09 | 38.0 | 1 | 1371-1426 |
| | factor type C | | | | |
| | domain | | | | |
| TILa | TILa domain | 0.0017 | 16.9 | 1 | 1370-1409 |
| LRRNT | Leucine rich | 0.025 | 14.9 | 1 | 23-49 |
| | repeat N- | | | | |
| | terminal domain | | | | |

Using the eMATRIX software package (Stanford University, Stanford, Calif.) (Wu et al., J. Comp. Biol., 6:219-235 (1999), herein incorporated by reference), the peroxidasin-like polypeptide of SEQ ID NO: 626 was determined to have following the eMATRIX domain hits. The results in Table 38 describe: the eMATRIX domain name, the corresponding p-value, Signature ID number, and the corresponding position of the domain within SEQ ID NO: 626:

TABLE 38

| | | Signature | |
|---|---|---|---|
| Name | p-value | ID NO | Position |
| ANIMAL HAEM | 8.45e−24 | PR00457E | 973-999 |
| PEROXIDASE | | | |
| SIGNATURE V | | | |
| ANIMAL HAEM | 1.53e−20 | PR00457D | 948-968 |
| PEROXIDASE | | | |
| SIGNATURE IV | | | |
| ANIMAL HAEM | 9.42e−15 | PR00457C | 930-948 |
| PEROXIDASE | | | |
| SIGNATURE III | | | |
| ANIMAL HAEM | 4.48e−14 | PR00457G | 1153-1173 |
| PEROXIDASE | | | |
| SIGNATURE VII | | | |
| ANIMAL HAEM | 5.85e−13 | PR00457H | 1224-1238 |
| PEROXIDASE | | | |
| SIGNATURE VIII | | | |
| ANIMAL HAEM | 6.32e−12 | PR00457F | 1026-1036 |
| PEROXIDASE | | | |
| SIGNATURE VI | | | |
| LEUCINE RICH | 1.00e−10 | IPB000483 | 156-170 |
| REPEAT C- | | | |
| TERMINAL DOMAIN | | | |
| ANIMAL HAEM | 2.29e−10 | PR00457B | 778-793 |
| PEROXIDASE | | | |
| SIGNATURE II | | | |
| IMMUNOGLOBULIN | 2.80e−10 | IPB003006B | 352-389 |
| AND MAJOR | | | |
| HISTOCOMPATIBILITY | | | |
| COMPLEX DOMAIN | | | |
| IMMUNOGLOBULIN | 8.92e−10 | IPB003006B | 442-479 |
| AND MAJOR | | | |
| HISTOCOMPATIBILITY | | | |
| COMPLEX DOMAIN | | | |
| IMMUNOGLOBULIN | 9.28e−10 | IPB003006B | 259-296 |
| AND MAJOR | | | |
| HISTOCOMPATIBILITY | | | |
| COMPLEX DOMAIN | | | |

Protein database searches with the BLASTP algorithm (Altschul S. F. et al., J. Mol. Evol. 36:290-300 (1993) and Altschul S. F. et al., J. Mol. Biol. 21:403-10 (1990), herein incorporated by reference) indicate that the variant sequences SEQ ID NO: 619, 623 and 627 are homologous to the human peroxidasin-like protein (accession number BAA13219.1) that is also known as the melanoma-associated antigen MG50 (Accession number AF200349_{—}1) (SEQ ID NO: 617).

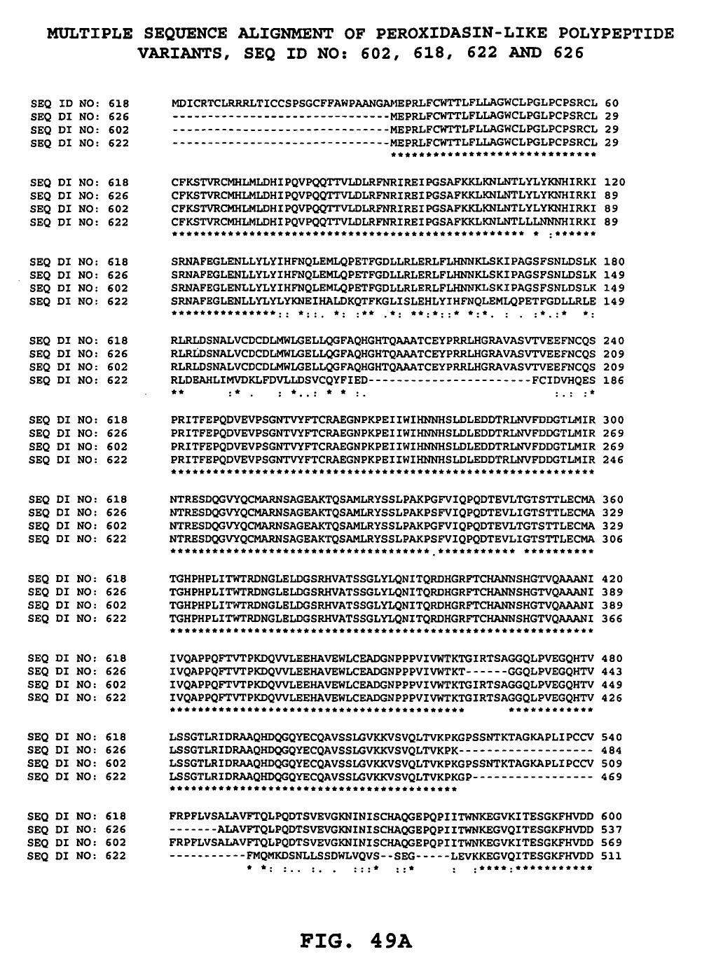

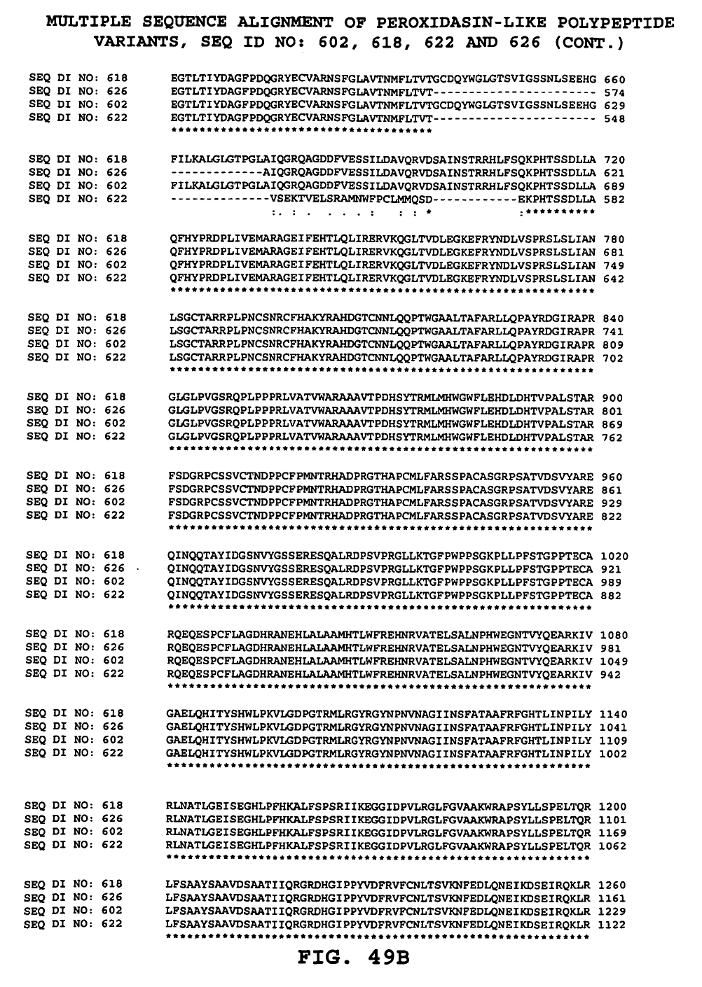

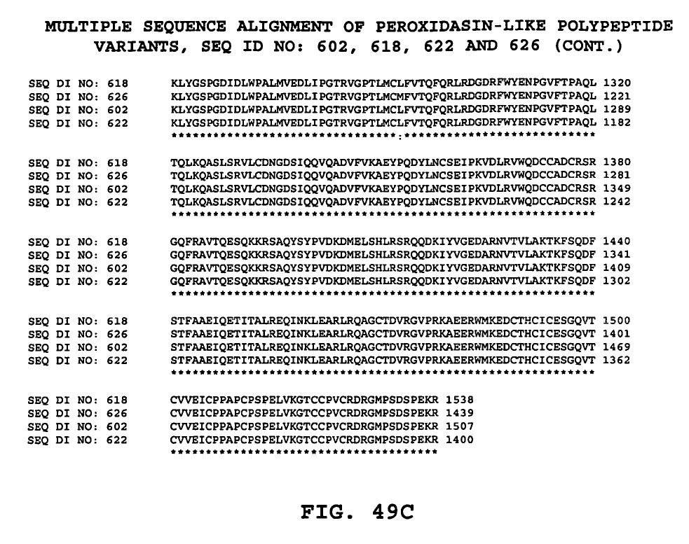

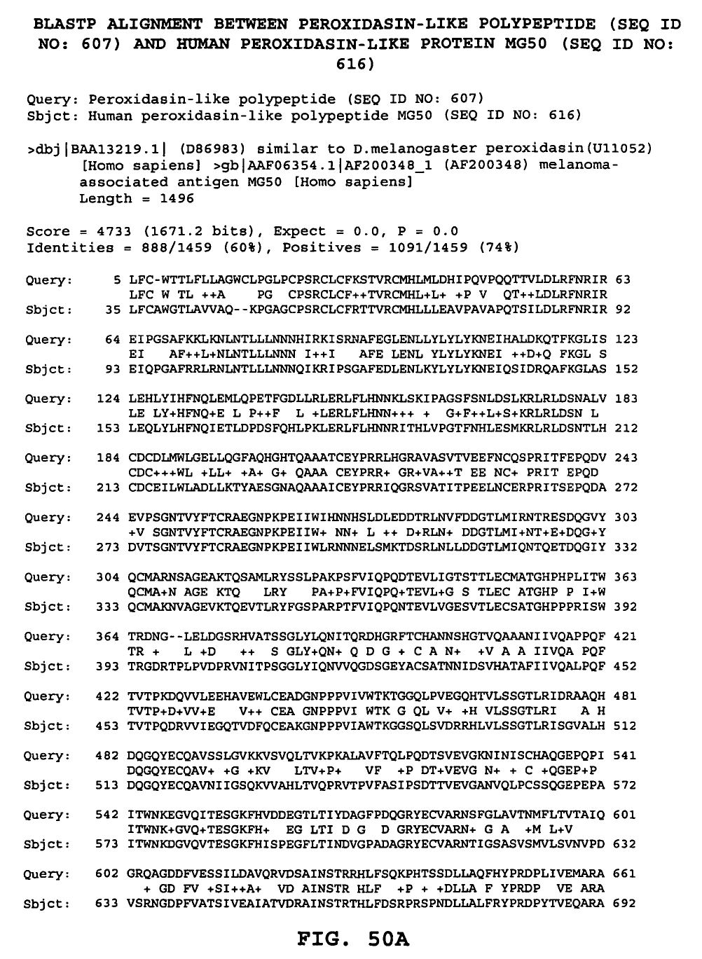

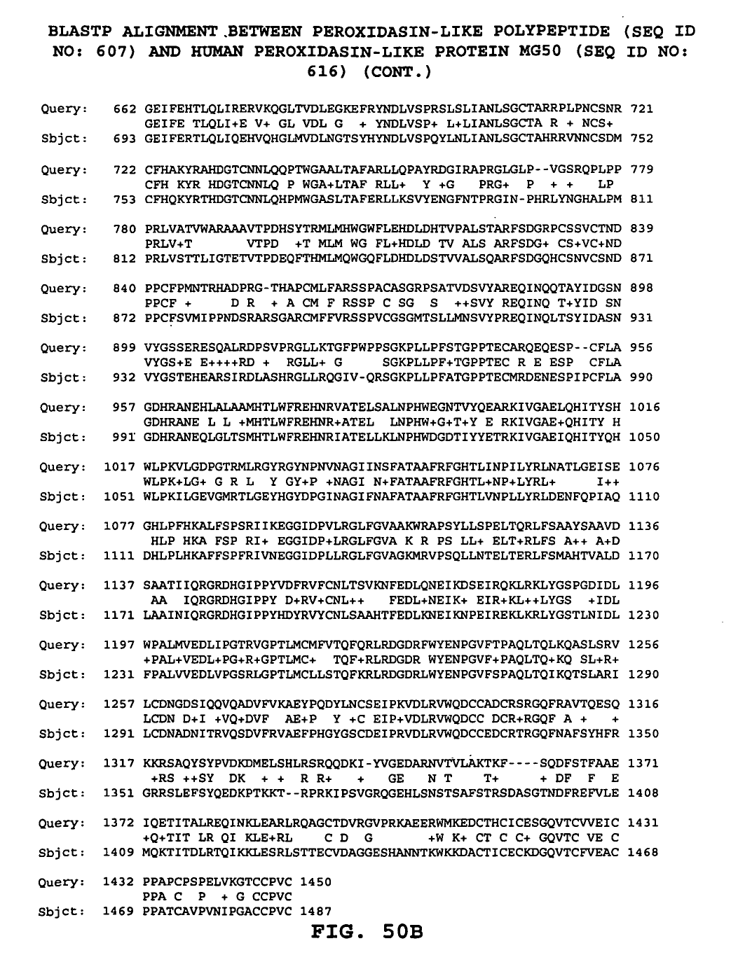

FIG. 49 shows a multiple sequence alignment between the three variants of peroxidase-like polypeptide SEQ ID NO: 602, namely SEQ ID NO: 618, 624, and 626, wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine. Gaps are presented as dashes, asterisks (*) represent identical residues, colons (:) represent conservative substitutions, and periods (.) represent semi-conservative substitutions.

The peroxidasin-like polypeptide of SEQ ID NO: 607 is an approximately 1463-amino acid protein with a predicted molecular mass of approximately 161 kDa unglycosylated. The initial methionine starts at position 145 of SEQ ID NO: 606 and the putative stop codon begins at position 4534 of SEQ ID NO: 606. A signal peptide of twenty three residues (SEQ ID NO: 609) is predicted from approximately residue 1 to residue 23 of SEQ ID NO: 607. The extracellular portion is useful on its own. This can be confirmed by expression in mammalian cells and sequencing of the cleaved product. The signal peptide region was predicted using the Neural Network SignalP V1.1 program (Nielsen et al, Int. J. Neural Syst. 8:581-599 (1997)). One of skill in the art will recognize that the actual cleavage site may be different than that predicted by the computer program.

Using the TMpred program (Hofmann and Stoffel, Biol. Chem. 374:166 (1993), herein incorporated by reference), SEQ ID NO: 607 is predicted to have transmembrane domains at approximately residue 6 to residue 20, residue 585 to residue 600, and residue 1042 to residue 1060. Removal of one or more transmembrane domains renders fragments that can be useful on their own. One example is a fragment from residue 24 to residue 584 of SEQ ID NO: 607. One of skill in the art will recognize that the actual transmembrane domains may be different than that predicted by the computer program.

Protein database searches with the BLASTP algorithm (Altschul S. F. et al., J. Mol. Evol. 36:290-300 (1993) and Altschul S. F. et al., J. Mol. Biol. 21:403-10 (1990), herein incorporated by reference) indicate that SEQ ID NO: 608 is homologous to peroxidasin-like proteins.

FIG. 50 shows the BLASTP amino acid sequence alignment between peroxidasin-like polypeptide SEQ ID NO: 607 and human peroxidasin-like protein (also known as melanoma-associated antigen, MG50) (SEQ ID NO: 616), indicating that the two sequences share 74% similarity and 60% identity over 1459 amino acid residues, wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine. Gaps are presented as dashes.

Using the Pfam software program (Sonnhammer et al., Nucleic Acids Res., 26:320-322 (1998) herein incorporated by reference) SEQ ID NO: 607 was examined for domains with homology to known conserved peptide domains. Table 39 shows the name of the Pfam model found, the description, the e-value, Pfam score, number of repeats, and position of the domain(s) within SEQ ID NO: 607 for the identified model within the sequence as follows:

TABLE 39

| | | | | Re- | |
|---|---|---|---|---|---|
| Model | Description | E-value | Score | peats | Position |
| perox- | Peroxidase | 1.1e−40 | 148.6 | 1 | 726-1164 |
| idase | | | | | |
| Ig | Immunoglobulin | 6.2e−36 | 120.8 | 4 | 248-307 |
| | domain | | | | 344-400 |
| | | | | | 440-490 |
| | | | | | 525-582 |
| LRR | Leucine Rich Repeat | 2.3e−22 | 87.7 | 6 | 51-74 |
| | | | | | 75-98 |
| | | | | | 99-122 |
| | | | | | 123-146 |
| | | | | | 147-170 |
| | | | | | 171-195 |
| LRRCT | Leucine rich repeat | 9.1e−11 | 49.2 | 1 | 180-232 |
| | C-terminal domain | | | | |
| vwc | von Willebrand factor | 7e−08 | 39.6 | 1 | 1395-1450 |
| | type C domain | | | | |
| TILa | TILa domain | 0.023 | 12.0 | 1 | 1394-1447 |

Using the eMATRIX software package (Stanford University, Stanford, Calif.) (Wu et al., J. Comp. Biol., 6:219-235 (1999), herein incorporated by reference), the peroxidasin-like polypeptide of SEQ ID NO: 607 was determined to have following the eMATRIX domain hits. The results in Table 40 describe: the eMATRIX domain name, the corresponding p-value, Signature ID number, and the corresponding position of the domain within SEQ ID NO: 607:

TABLE 40

| | | Signature | |
|---|---|---|---|
| Name | p-value | ID NO | Position |
| ANIMAL HAEM | 3.118e−22 | PR00457E | 973-999 |
| PEROXIDASE | | | |
| SIGNATURE | | | |
| ANIMAL HAEM | 4.194e−21 | PR00457D | 948-968 |
| PEROXIDASE | | | |
| SIGNATURE | | | |
| ANIMAL HAEM | 1.675e−13 | PR00457C | 930-948 |
| PEROXIDASE | | | |
| SIGNATURE | | | |
| ANIMAL HAEM | 5.680e−13 | PR00457H | 1224-1238 |
| PEROXIDASE | | | |
| SIGNATURE | | | |
| ANIMAL HAEM | 4.750e−12 | PR00457F | 1026-1036 |
| PEROXIDASE | | | |
| SIGNATURE | | | |
| ANIMAL HAEM | 8.615e−12 | PR00457G | 1153-1173 |
| PEROXIDASE | | | |
| SIGNATURE | | | |
| VWFC domain | 3.250e−10 | BL01208B | 1412-1426 |
| proteins | | | |
| ANIMAL HAEM | 3.411e−10 | PR00457B | 778-793 |
| PEROXIDASE | | | |
| SIGNATURE | | | |
| Receptor tyrosine | 1.000e−09 | BL00240B | 325-348 |
| kinase class III | | | |
| proteins | | | |
| LEUCINE-RICH | 7.480e−09 | PR00019B | 73-86 |
| REPEAT | | | |
| SIGNATURE | | | |
| RECEPTOR FC | 7.677e−09 | PD01270A | 304-343 |
| IMMUNOGLOBULIN | | | |
| AFFIN. | | | |

The peroxidasin-like polypeptide of SEQ ID NO: 612 is an approximately 1439-amino acid protein with a predicted molecular mass of approximately 158 kDa unglycosylated. The initial methionine starts at position 145 of SEQ ID NO: 611 and the putative stop codon begins at position 4462 of SEQ ID NO: 611. A signal peptide of twenty-three residues (SEQ ID NO: 614) is predicted from approximately residue 1 to residue 23 of SEQ ID NO: 612. The extracellular portion is useful on its own. This can be confirmed by expression in mammalian cells and sequencing of the cleaved product. The signal peptide region was predicted using the Neural Network SignalP V1.1 program (Nielsen et al, Int. J. Neural Syst. 8:581-599 (1997)). One of skill in the art will recognize that the actual cleavage site may be different than that predicted by the computer program.

Using the TMpred program (Hofmann and Stoffel, Biol. Chem. 374:166 (1993), herein incorporated by reference), SEQ ID NO: 612 is predicted to have transmembrane domains at approximately residue 6 to residue 20, residue 561 to residue 576, and residue 1018 to residue 1036. Removal of one or more transmembrane domains renders fragments that can be useful on their own. One example is a fragment from residue 24 to residue 560 of SEQ ID NO: 612. One of skill in the art will recognize that the actual transmembrane domains may be different than that predicted by the computer program.

Protein database searches with the BLASTP algorithm (Altschul S. F. et al., J. Mol. Evol. 36:290-300 (1993) and Altschul S. F. et al., J. Mol. Biol. 21:403-10 (1990), herein incorporated by reference) indicate that SEQ ID NO: 612 is homologous to peroxidasin-like proteins.

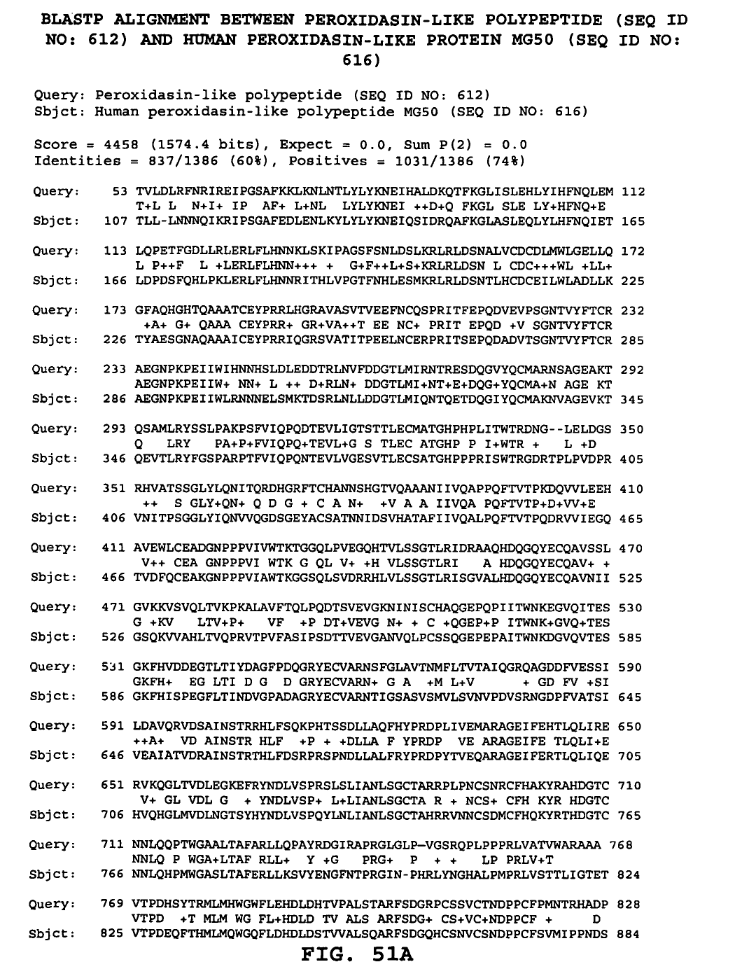

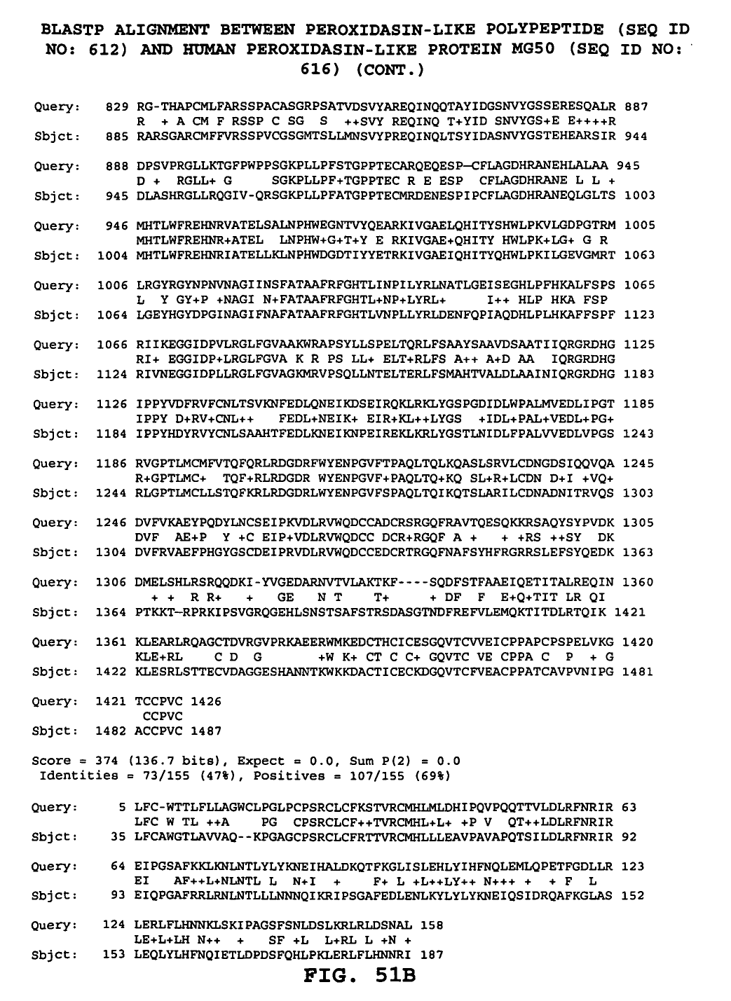

FIG. 51 shows the BLASTP amino acid sequence alignment between peroxidasin-like polypeptide SEQ ID NO: 612 and human peroxidasin-like protein (melanoma-associated antigen, MG50) (SEQ ID NO: 616), indicating that the two sequences share: 74% similarity and 60% identity over 1386 amino acid residues, and 69% similarity and 47% identity over a distinct 155 amino acid residues, wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine. Gaps are presented as dashes.

Using the Pfam software program (Sonnhammer et al., Nucleic Acids Res., 26:320-322 (1998) herein incorporated by reference) SEQ ID NO: 612 was examined for domains with homology to known conserved peptide domains. Table 41 shows the name of the Pfam model found, the description, the e-value, Pfam score, number of repeats, and position of the domain(s) within SEQ ID NO: 612 for the identified model within the sequence as follows:

TABLE 41

| | | | | Re- | |
|---|---|---|---|---|---|
| Model | Description | E-value | Score | peats | Position |
| perox- | Peroxidase | 1.1e−40 | 148.6 | 1 | 702-1140 |
| idase | | | | | |
| Ig | Immunoglobulin | 6.2e−36 | 120.8 | 4 | 224-283 |
| | domain | | | | 320-376 |
| | | | | | 416-466 |
| | | | | | 501-558 |
| LRR | Leucine Rich Repeat | 1.2e−18 | 75.4 | 5 | 51-74 |
| | | | | | 75-98 |
| | | | | | 99-122 |
| | | | | | 123-146 |
| | | | | | 147-171 |
| LRRCT | Leucine rich repeat | 9.1e−11 | 49.2 | 1 | 156-208 |
| | C-terminal domain | | | | |
| vwc | von Willebrand factor | 7e−08 | 39.6 | 1 | 1371-1426 |
| | type C domain | | | | |
| TILa | TILa domain | 0.023 | 12.0 | 1 | 1370-1423 |

Using the eMATRIX software package (Stanford University, Stanford, Calif.) (Wu et al., J. Comp. Biol., 6:219-235 (1999), herein incorporated by reference), the peroxidasin-like polypeptide of SEQ ID NO: 612 was determined to have following the eMATRIX domain hits. The results in Table 42 describe: the eMATRIX domain name, the corresponding p-value, Signature ID number, and the corresponding position of the domain within SEQ ID NO: 613:

TABLE 42

| | | Signature | |
|---|---|---|---|
| Name | p-value | ID NO | Position |
| ANIMAL HAEM | 3.118e−22 | PR00457E | 973-999 |
| PEROXIDASE | | | |
| SIGNATURE | | | |
| ANIMAL HAEM | 4.194e−21 | PR00457D | 948-968 |
| PEROXIDASE | | | |
| SIGNATURE | | | |
| ANIMAL HAEM | 1.675e−13 | PR00457C | 930-948 |
| PEROXIDASE | | | |
| SIGNATURE | | | |
| ANIMAL HAEM | 5.680e−13 | PR00457H | 1224-1238 |
| PEROXIDASE | | | |
| SIGNATURE | | | |
| ANIMAL HAEM | 4.750e−12 | PR00457F | 1026-1036 |
| PEROXIDASE | | | |
| SIGNATURE | | | |
| ANIMAL HAEM | 8.615e−12 | PR00457G | 1153-1173 |
| PEROXIDASE | | | |
| SIGNATURE | | | |
| VWFC domain | 3.250e−10 | BL01208B | 1412-1426 |
| proteins | | | |
| ANIMAL HAEM | 3.411e−10 | PR00457B | 778-793 |
| PEROXIDASE | | | |
| SIGNATURE | | | |
| Receptor tyrosine | 1.000e−09 | BL00240B | 325-348 |
| kinase class III | | | |
| proteins | | | |
| LEUCINE-RICH | 7.480e−09 | PR00019B | 73-86 |
| REPEAT | | | |
| SIGNATURE | | | |
| RECEPTOR FC | 7.677e−09 | PD01270A | 304-343 |
| IMMUNOGLOBULIN | | | |
| AFFIN. | | | |

Peroxidasin-like polypeptides are expected to play roles in phagocytosis and cell adhesion and possess peroxidase-like enzymatic activity. Additionally, peroxidasin-like polypeptides may serve as tumor markers and tumor-specific antigens for immunotherapy.

Immunotherapy provides a method of harnessing the immune system to treat various pathological states, including cancer, autoimmune disease, transplant rejection, hyperproliferative conditions, and allergic reactions.

Antibody therapy for cancer involves the use of antibodies, or antibody fragments, against a tumor antigen to target antigen-expressing cells. Antibodies, or antibody fragments, may have direct or indirect cytotoxic effects or may be conjugated or fused to cytotoxic moieties. Direct effects include the induction of apoptosis, the blocking of growth factor receptors, and anti-idiotype antibody formation. Indirect effects include antibody-dependent cell-mediated cytotoxicity (ADCC) and complement-mediated cellular cytotoxicity (CMCC). When conjugated or fused to cytotoxic moieties, the antibodies, or fragments thereof, provide a method of targeting the cytotoxicity towards the tumor antigen expressing cells. (Green, et al., Cancer Treatment Reviews, 26:269-286 (2000), incorporated herein by reference).

For example, Rituximab (Rituxan®) is a chimeric antibody directed against CD20, a B cell-specific surface molecule found on >95% of B-cell non-Hodgkin's lymphoma (Press, et al., Blood 69:584-591 (1987), incorporated herein by reference; Malony, et al., Blood 90:2188-2195 (1997), incorporated herein by reference). Rituximab induces ADCC and inhibits cell proliferation through apoptosis in malignant B cells in vitro (Maloney, et al., Blood 88:637a (1996), incorporated herein by reference). Rituximab is currently used as a therapy for advanced stage or relapsed low-grade non-Hodgkin's lymphoma, which has not responded to conventional therapy.

Active immunotherapy, whereby the host is induced to initiate an immune response against its own tumor cells can be achieved using therapeutic vaccines. One type of tumor-specific vaccine uses purified idiotype protein isolated from tumor cells, coupled to keyhole limpet hemocyanin (KLH) and mixed with adjuvant for injection into patients with low-grade follicular lymphoma (Hsu, et al., Blood 89:3129-3135 (1997), incorporated herein by reference). Another type of vaccine uses antigen-presenting cells (APCs), which present antigen to naïve T cells during the recognition and effector phases of the immune response. Dendritic cells, one type of APC, can be used in a cellular vaccine in which the dendritic cells are isolated from the patient, co-cultured with tumor antigen and then reinfused as a cellular vaccine (Hsu, et al., Nat. Med. 2:52-58 (1996), incorporated herein by reference). Immune responses can also be induced by injection of naked DNA. Plasmid DNA that expresses bicistronic mRNA encoding both the light and heavy chains of tumor idiotype proteins, such as those from B cell lymphoma, when injected into mice, are able to generate a protective, anti-tumor response (Singh, et al., Vaccine 20:1400-1411 (2002), incorporated herein by reference).

The peroxidasin-like polypeptides, polynucleotides, antibodies and other compositions of the invention are expected to be useful in providing therapeutic compositions and diagnostic methods for treating and identifying cancer, hyperproliferative disorders, auto-immune diseases, and organ transplant rejection.

4.10 Synaptic Associated 90/Postsynaptic Density Protein 95 kDa-Associated Protein-Like Polypeptides and Polynucleotides

Synaptic associated protein 90/postsynaptic density protein 95 kDa-associated proteins (SAPAPs) (Takeuchi et al., J. Biol. Chem. 272:11943-11951 (1997), herein incorporated by reference), also called GKAPs (Guanylate kinase-associated proteins) (Kim et al., J Cell Biol. 136:669-678 (1997) (Naisbitt et al., J Neurosci. 17:5687-5696 (1997), both herein incorporated by reference) or DAPs (hDLG-associated proteins) (Satoh et al., Genes Cells. 2:415-424 (1997), herein incorporated by reference), are major molecular constituents of postsynaptic densities. Pre- and postsynaptic specializations are formed gradually during brain development and in the adult nervous system contribute to regulate synaptic transmission. (Kawashima et al., FEBS Lett. 418:301-304 (1997), herein incorporated by reference). SAPAPs are associated with the postsynaptic density protein 95 kDa/synaptic associated protein 90 (PSD-95/SAP90) which belongs to the large family of synaptic membrane-associated guanylate kinases (MAGUKs). This class of proteins contains characteristic domains, which mediate protein/protein interactions, including PDZ, SH3, and guanylate kinase domains. These domains enable the MAGUKs to build scaffolds of synaptic components that include: a) ion channels and neurotransmitter receptors via their NH2-terminal PDZ domains (for example NMDA receptors and potassium channels) (Kim et al., J. Cell Biol. 136:669-678 (1997), herein incorporated by reference); b) intracellular signaling molecules; and c) cytoskeletal proteins (Naisbitt et al., J Neurosci. 17:5687-5696 (1997), herein incorporated by reference). Thus PSD-95 family proteins function as molecular anchors for coupling synaptic receptors and ion channels to downstream signaling molecules and cytoskeleton. The hypothesis that SAPAPs play a role in the molecular organization of synapses and neuronal cell signaling is suggested by the following observations: SAPAPs bind directly to a) the guanylate kinase domain of the postsynaptic density protein 95 (PSD-95) family, b) members of the dynein light chain family (Naisbitt et al., J Neurosci. 20:45244534 (2000), herein incorporated by reference), which are implicated in synaptic remodeling, and c) Shank, which is a protein that links different glutamate receptor complexes (NMDA and metabotropic) (Sangmi et al., J. Biol. Chem. 274:29510-29518 (1999), herein incorporated by reference). Thus SAPAPs may orchestrate functional interactions between metabotropic and ionotropic systems. This is relevant in the context of synaptic transmission and stabilization since SAPAPs also modulate NMDA channel conductance (Yamada et al., FEBS Lett. 458:295-298 (1999), herein incorporated by reference), interact with neuronal nitric oxide synthase (Haraguchi et al., Genes Cells. 5:905-911 (2000), herein incorporated by reference), neurofilaments (Hirao et al., Genes Cells. 5:203-210 (2000), herein incorporated by reference), and synaptic scaffolding molecule (S-SCAM; Hirao et al., J. Biol. Chem. 275:2966-2972 (2000), herein incorporated by reference). Thus, SAPAPs may be involved in the molecular organization of synapses and neuronal cell signaling.

Clones of the SAPAP family have been isolated (Boeckers et al., Biochem Biophys Res Commun. 264:247-252 (1999), herein incorporated by reference). SAP proteins are expressed not only in the synapse, but also in epithelial cells (Fujita and Kurachi, Biochem Biophys Res Commun. 269:1-6 (2000), herein incorporated by reference). Taken together, it is strongly suggested that various SAPAP proteins help SAPs perform specific functions in different tissues. Therefore, it is important to identify other members of this family of proteins.

The SAPAP-like polypeptide of SEQ ID NO: 630 is an approximately 979-amino acid protein with a predicted molecular mass of approximately 107.7-kDa unglycosylated. The initial methionine starts at position 1 of SEQ ID NO: 629 and the putative stop codon begins at position 2938 of SEQ ID NO: 629.

Protein database searches with the BLASTP algorithm (Altschul S. F. et al., J. Mol. Evol. 36:290-300 (1993) and Altschul S. F. et al., J. Mol. Biol. 21:403-10 (1990), herein incorporated by reference) indicate that SEQ ID NO: 630 is homologous to rat SAPAP (gi|17374684).

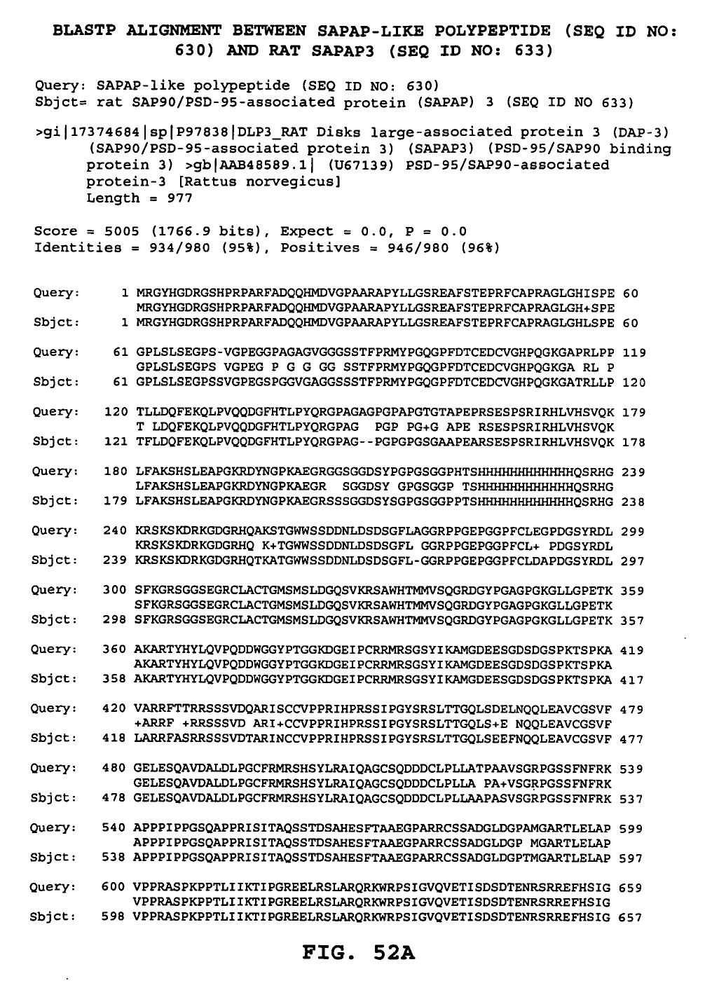

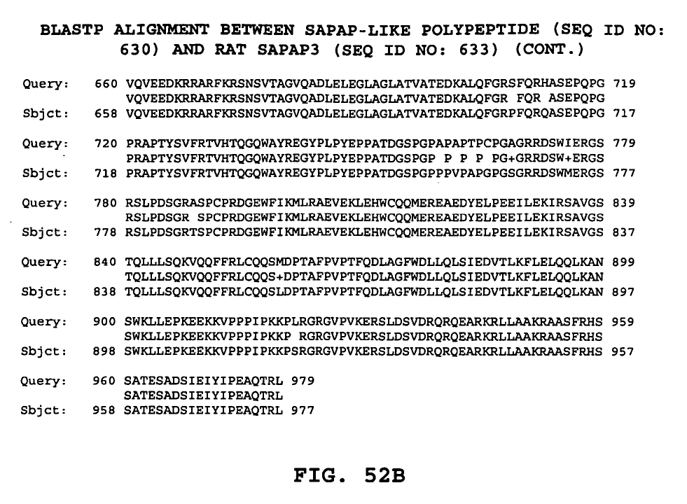

FIG. 52 shows a BLASTP amino acid sequence alignment between SAPAP-like polypeptide (SEQ ID NO: 630) and rat SAPAP3 (SEQ ID NO: 633), indicating that the two sequences share 96% similarity over amino acids 1-979 of SEQ ID NO: 630 and 95% identity over the same amino acids 1-979 of SEQ ID NO: 630, wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine. Gaps are presented as dashes.

Using the Pfam software program (Sonnhammer et al., Nucleic Acids Res., 26:320-322 (1998) herein incorporated by reference), SAPAP-like polypeptide of SEQ ID NO: 630 revealed its structural homology to Guanylate-kinase-associated protein (GKAP) corresponding to amino acids of 621-979 of the full length protein of SEQ ID NO: 630 that correspond to the Pfam domain and nucleotides of 1858-2937 the open reading frame of SEQ ID NO: 631 and is shown in Table 43. Further description of the Pfam models can be found at http://pfam.wustl.edu/.

TABLE 43

| SEQ | | | | Amino acid sequence |
|---|---|---|---|---|
| ID | | | | (start and |
| NO: | Domain | E-value | Score | end position) |
| 632 | Guanylate- | 7e−292 | 983.7 | ELRSLARQRKWRPSIGVQVET |
| | kinase- | | | ISDSDTENRSRREFHSIGVQV |
| | associated | | | EEDKRRARFKRSNSVTAGVQA |
| | protein | | | DLELEGLAGLATVATEDKALQ |
| | | | | FGRSFQRHASEPQPGPRAPTY |
| | | | | SVFRTVHTQGQWAYREGYPLP |
| | | | | YEPPATDGSPGPAPAPTPCPG |
| | | | | AGRRDSWIERGSRSLPDSGRA |
| | | | | SPCPRDGEWFIKMLRAEVEKL |
| | | | | EHWCQQMEREAEDYELPEEIL |
| | | | | EKIRSAVGSTQLLLSQKVQQF |
| | | | | FRLCQQSMDPTAFPVPTFQDL |
| | | | | AGFWDLLQLSIEDVTLKFLEL |
| | | | | QQLKANSWKLLEPKEEKKVPP |
| | | | | PIPKKPLRGRGVPVKERSLDS |
| | | | | VDRQRQEARKRLLAAKRAASF |
| | | | | RHSSATESADSIEIYIPEAQT |
| | | | | RL (621-979) |

Using eMATRIX software package (Stanford University, Stanford, Calif.) (Wu et al, J. Comp. Biol., 6:219-235 (1999), herein incorporated by reference in its entirety), SAPAP-like polypeptide of SEQ ID NO: 630 was determined to have following eMATRIX domain hits. The results in Table 44 describe: corresponding SEQ ID NO: in sequence listing, e-value, subtype, Accession number, name, position of the domain in the full-length protein, and the amino acid sequence, wherein A=Alanine, C=Cysteine, D=Aspartic Acid, E=Glutamic Acid, F—Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine.

TABLE 44

| | | | | | |
|---|---|---|---|---|---|
| SEQ | | | | | Amino Acid |
| ID | | | Accession | | Sequence (start |
| NO: | e-value | subtype | No. | Name | and end position) |

| | | | | | |
|---|---|---|---|---|---|
| 634 | 5.97e−11 | 5.92 | PR01256B | Otx 1 transcription | TSHHHHHHHH |
| | | | | factor signature II | HHH (221-233) |
| 635 | 7.51e−11 | 5.92 | PR01256B | Otx 1 transcription | GPHTSHHHHH |
| | | | | factor signature II | HHH (218-230) |
| 636 | 2.35e−10 | 5.92 | PR01256B | Otx 1 transcription | PHTSHHHHHH |
| | | | | factor signature II | HHH (219-231) |
| 637 | 2.11e−09 | 5.92 | PR01256B | Otx 1 transcription | HTSHHHHHHH |
| | | | | factor signature II | HHH (220-232) |
| 638 | 2.31−e09 | 5.92 | PR01256B | Otx 1 transcription | SHHHHHHHHH |
| | | | | factor signature II | HHH (222-234) |
| 639 | 2.62−e09 | 5.92 | PR01256B | Otx 1 transcription | GGPHTSHHHH |
| | | | | factor signature II | HHLH (217-229) |
| 640 | 3.14e−09 | 11.65 | IPB001541B | SUR2-type | HHHHHHHHHH |
| | | | | hydroxylase/desaturase | (223-232) |
| | | | | catalytic domain | |
| 641 | 3.14e−09 | 11.65 | IPB001541B | SUR2-type | HHHHHHHHHH |
| | | | | hydroxylase/desaturase | (224-233) |
| | | | | catalytic domain | |
| 642 | 3.14e−09 | 11.65 | IPB001541B | SUR2-type | HHHHHHHHHH |
| | | | | hydroxylase/desaturase | (225-234) |
| | | | | catalytic domain | |
| 643 | 6.57e−09 | 11.65 | IPB001541B | SUR2-type | HHHHHHHHHH |
| | | | | hydroxylase/desaturase | (222-231) |
| | | | | catalytic domain | |
| 644 | 2.29e−08 | 11.65 | IPB00154IB | SUR2-type | HHHRHHHQSR |
| | | | | hydroxylase/desaturase | (228-237) |
| | | | | catalytic domain | |
| 645 | 3.57e−08 | 11.65 | IPB001541B | SUR2-type | HHHHHHHHQS |
| | | | | hydroxylase/ | (227-236) |
| | | | | desaturase | |
| | | | | catalytic domain | |
| 646 | 6.60e−08 | 0.00 | PR00049D | Wilm's tumour | GSPGPAPAPTP |
| | | | | protein signature | CPGA (754-768) |
| | | | | IV | |
| 647 | 6.61e−08 | 9.10 | PR00334B | HMW kininogen | GGPHTSHHHH |
| | | | | signature H | HHHHHHHHQS |
| | | | | | RHGK (217-240) |
| 648 | 6.85e−08 | 5.92 | PR01256B | Otx 1 transcription | HHHHHHHHHH |
| | | | factor signature II | HHQ (223-235) | |
| 649 | 7.34e−08 | 14.85 | IPB002489C | Domain of unknown | RFCAPRAGLGH |
| | | | | function DUF14 | ISPEGPLSLSEG |
| | | | | | PSVGPEGGPAG |
| | | | | | (46-79) |
| 650 | 7.75e−08 | 11.65 | IPB001541B | SUR2-type | GPHTSHHHHH |
| | | | | hydroxylase/desaturase | (218-227) |
| | | | | catalytic domain | |
| 651 | 7.77e−08 | 3.45 | PR01131B | Connexin36 (Cx36) | GPKAEGRGGS |
| | | | | signature II | GGD (197-209) |
| 652 | 8.01e−08 | 24.91 | IPB000868B | Isochorismatase | HTSHHHHHHH |
| | | | | hydrolase family | HHHHHQSRHG |
| | | | | | KRS (220-242) |
| 653 | 8.32e−08 | 10.49 | PR01274A | Metalloprotease | TAFPVPTFQDL |
| | | | | inhibitor | AGFWDL |
| | | | | signature I | (862-878) |

The polypeptides of the invention may play a role in the formation and function of the nervous system, by regulating the molecular organization of synapses and neuronal cell signaling. For example, they could function as adapter proteins linking ion channels and other synaptic proteins to the subsynaptic cytoskeleton which is important for the localization and concentration of synaptic molecules to the postsynaptic membrane.

The polypeptides, polynucleotides, antibodies and other compositions of the invention are expected to be useful in treating the following disorders: Alzheimer's disease, anxiety, autism, brain injury, depression, epilepsy, Huntington's disease, mania, pain, Parkinsonism, Parkinson's disease, Schizophrenia, Tardive dyskinesia, myasthenia gravis, amyotrophic lateral sclerosis, episodic ataxia/myokymia, hyperkalemix periodic paralysis, hypokalemic periodic paralysis, Lamber-Eaton syndrome, paramyotonia congenita, Rasmussen's encephalitis, Startle disease, and seizure disorders, including neonatal seizure disorders and generally, learning and memory disorders.

4.11 Definitions

It must be noted that as used herein and in the appended claims, the singular forms “a”, “an” and “the” include plural references unless the context clearly dictates otherwise.

The term “active” refers to those forms of the polypeptide that retain the biologic and/or immunologic activities of any naturally occurring polypeptide. According to the invention, the terms “biologically active” or “biological activity” refer to a protein or peptide having structural, regulatory or biochemical functions of a naturally occurring molecule. Likewise “biologically active” or “biological activity” refers to the capability of the natural, recombinant or synthetic polypeptide of the invention, or any peptide thereof, to induce a specific biological response in appropriate animals or cells and to bind with specific antibodies.

The term “activated cells” as used in this application are those cells which are engaged in extracellular or intracellular membrane trafficking, including the export of secretory or enzymatic molecules as part of a normal or disease process.

The terms “complementary” or “complementarity” refer to the natural binding of polynucleotides by base pairing. For example, the sequence 5′-AGT-3′ binds to the complementary sequence 3′-TCA-5′. Complementarity between two single-stranded molecules may be “partial” such that only some of the nucleic acids bind or it may be “complete” such that total complementarity exists between the single stranded molecules. The degree of complementarity between the nucleic acid strands has significant effects on the efficiency and strength of the hybridization between the nucleic acid strands.

The term “embryonic stem cells (ES)” refers to a cell that can give rise to many differentiated cell types in an embryo or an adult, including the germ cells. The term “germ line stem cells (GSCs)” refers to stem cells derived from primordial stem cells that provide a steady and continuous source of germ cells for the production of gametes. The term “primordial germ cells (PGCs)” refers to a small population of cells set aside from other cell lineages particularly from the yolk sac, mesenteries, or gonadal ridges during embryogenesis that have the potential to differentiate into germ cells and other cells. PGCs are the source from which GSCs and ES cells are derived. The PGCs, the GSCs and the ES cells are capable of self-renewal. Thus these cells not only populate the germ line and give rise to a plurality of terminally differentiated cells that comprise the adult specialized organs, but are able to regenerate themselves. The term “totipotent” refers to the capability of a cell to differentiate into all of the cell types of an adult organism. The term “pluripotent” refers to the capability of a cell to differentiate into a number of differentiated cell types that are present in an adult organism. A pluripotent cell is restricted in its differentiation capability in comparison to a totipotent cell.

The term “expression modulating fragment,” EMF, means a series of nucleotides that modulates the expression of an operably linked ORF or another EMF.

As used herein, a sequence is said to “modulate the expression of an operably linked sequence” when the expression of the sequence is altered by the presence of the EMF. EMFs include, but are not limited to, promoters, and promoter modulating sequences (inducible elements). One class of EMFs is nucleic acid fragments which induce the expression of an operably linked ORF in response to a specific regulatory factor or physiological event.

The terms “nucleotide sequence” or “nucleic acid” or “polynucleotide” or “oligonculeotide” are used interchangeably and refer to a heteropolymer of nucleotides or the sequence of these nucleotides. These phrases also refer to DNA or RNA of genomic or synthetic origin which may be single-stranded or double-stranded and may represent the sense or the antisense strand, to peptide nucleic acid (PNA) or to any DNA-like or RNA-like material. In the sequences, A is adenine, C is cytosine, G is guanine, and T is thymine, while N is A, T, G, or C. It is contemplated that where the polynucleotide is RNA, the T (thymine) in the sequence herein may be replaced with U (uracil). Generally, nucleic acid segments provided by this invention may be assembled from fragments of the genome and short oligonucleotide linkers, or from a series of oligonucleotides, or from individual nucleotides, to provide a synthetic nucleic acid which is capable of being expressed in a recombinant transcriptional unit comprising regulatory elements derived from a microbial or viral operon, or a eukaryotic gene.

The terms “oligonucleotide fragment” or a “polynucleotide fragment”, “portion,” or “segment” or “probe” or “primer” are used interchangeably and refer to a sequence of nucleotide residues which are at least about 5 nucleotides, more preferably at least about 7 nucleotides, more preferably at least about 9 nucleotides, more preferably at least about 11 nucleotides and most preferably at least about 17 nucleotides. The fragment is preferably less than about 500 nucleotides, preferably less than about 200 nucleotides, more preferably less than about 100 nucleotides, more preferably less than about 50 nucleotides and most preferably less than 30 nucleotides. Preferably the probe is from about 6 nucleotides to about 200 nucleotides, preferably from about 15 to about 50 nucleotides, more preferably from about 17 to 30 nucleotides and most preferably from about 20 to 25 nucleotides. Preferably the fragments can be used in polymerase chain reaction (PCR), various hybridization procedures or microarray procedures to identify or amplify identical or related parts of mRNA or DNA molecules. A fragment or segment may uniquely identify each polynucleotide sequence of the present invention. Preferably the fragment comprises a sequence substantially similar to a portion of SEQ ID NO: 1-4, 6, 14, 16, 25-27, 29, 157-159, 161, 183-185, 187, 214, 216, 240, 242, 271, 273, 300-301, 303, 322, 324, 345-347, 349, 353-354, 356, 377, 379, 405-407, 409, 418-419, 421, 441-443, 485-486, 488, 503, 504, 506, 514-515, 517, 526-527, 529, 547, 549, 556, 558, 570-571, 573, 577-578, 580, 587, 589, 601, 603, 606, 608, 611, 613, 617, 619, 621, 623, 625, 627, 629, or 631.

Probes may, for example, be used to determine whether specific mRNA molecules are present in a cell or tissue or to isolate similar nucleic acid sequences from chromosomal DNA as described by Walsh et al. (Walsh, P. S. et al., PCR Methods Appl. 1:241-250 (1992)). They may be labeled by nick translation, Klenow fill-in reaction, PCR, or other methods well known in the art. Probes of the present invention, their preparation and/or labeling are elaborated in Sambrook, J. et al., 1989, Molecular Cloning: A Laboratory Manual, Cold Spring Harbor Laboratory, NY; or Ausubel, F. M. et al., 1989, Current Protocols in Molecular Biology, John Wiley & Sons, New York N.Y., both of which are incorporated herein by reference in their entirety.

The nucleic acid sequences of the present invention also include the sequence information from any of the nucleic acid sequences of SEQ ID NO: 1-4, 6, 14, 16, 25-27, 29, 157-159, 161, 183-185, 187, 214, 216, 240, 242, 271, 273, 300-301, 303, 322, 324, 345-347, 349, 353-354, 356, 377, 379, 405-407, 409, 418-419, 421, 441-443, 485-486, 488, 503, 504, 506, 514-515, 517, 526-527, 529, 547, 549, 556, 558, 570-571, 573, 577-578, 580, 587, 589, 601, 603, 606, 608, 611, 613, 617, 619, 621, 623, 625, 627, 629, or 631. The sequence information can be a segment of SEQ ID NO: 1-4, 6, 14, 16, 25-27, 29, 157-159, 161, 183-185, 187, 214, 216, 240, 242, 271, 273, 300-301, 303, 322, 324, 345-347, 349, 353-354, 356, 377, 379, 405-407, 409, 418-419, 421, 441-443, 485-486, 488, 503, 504, 506, 514-515, 517, 526-527, 529, 547, 549, 556, 558, 570-571, 573, 577-578, 580, 587, 589, 601, 603, 606, 608, 611, 613, 617, 619, 621, 623, 625, 627, 629, or 631 that uniquely identifies or represents the sequence information of SEQ ID NO: 1-4,6,14,16,25-27,29,157-159,161,183-185, 187, 214, 216, 240, 242, 271, 273, 300-301, 303, 322, 324, 345-347, 349, 353-354, 356, 377, 379, 405-407, 409, 418-419,421,441-443, 485-486, 488, 503, 504, 506, 514-515, 517, 526-527, 529, 547, 549, 556, 558, 570-571, 573, 577-578, 580, 587, 589, 601, 603, 606, 608, 611, 613, 617, 619, 621, 623, 625, 627, 629, or 631. One such segment can be a twenty-mer nucleic acid sequence because the probability that a twenty-mer is fully matched in the human genome is 1 in 300. In the human genome, there are three billion base pairs in one set of chromosomes. Because 4²⁰ possible twenty-mers exist, there are 300 times more twenty-mers than there are base pairs in a set of human chromosomes. Using the same analysis, the probability for a seventeen-mer to be fully matched in the human genome is approximately 1 in 5. When these segments are used in arrays for expression studies, fifteen-mer segments can be used. The probability that the fifteen-mer is fully matched in the expressed sequences is also approximately one in five because expressed sequences comprise less than approximately 5% of the entire genome sequence.

Similarly, when using sequence information for detecting a single mismatch, a segment can be a twenty-five mer. The probability that the twenty-five mer would appear in a human genome with a single mismatch is calculated by multiplying the probability for a full match (1÷4²⁵) times the increased probability for mismatch at each nucleotide position (3×25). The probability that an eighteen mer with a single mismatch can be detected in an array for expression studies is approximately one in five. The probability that a twenty-mer with a single mismatch can be detected in a human genome is approximately one in five.

The term “open reading frame,” ORF, means a series of nucleotide triplets coding for amino acids without any termination codons and is a sequence translatable into protein.

The terms “operably linked” or “operably associated” refer to functionally related nucleic acid sequences. For example, a promoter is operably associated or operably linked with a coding sequence if the promoter controls the transcription of the coding sequence. While operably linked nucleic acid sequences can be contiguous and in the same reading frame, certain genetic elements e.g. repressor genes are not contiguously linked to the coding sequence but still control transcription/translation of the coding sequence.

The term “pluripotent” refers to the capability of a cell to differentiate into a number of differentiated cell types that are present in an adult organism. A pluripotent cell is restricted in its differentiation capability in comparison to a totipotent cell.

The terms “polypeptide” or “peptide” or “amino acid sequence” refer to an oligopeptide, peptide, polypeptide, or protein sequence or fragment thereof and to naturally occurring or synthetic molecules. A polypeptide “fragment,” “portion,” or “segment” is a stretch of amino acid residues of at least about 5 amino acids, preferably at least about 7 amino acids, more preferably at least about 9 amino acids and most preferably at least about 17 or more amino acids. The peptide preferably is not greater than about 200 amino acids, more preferably less than 150 amino acids and most preferably less than 100 amino acids. Preferably the peptide is from about 5 to about 200 amino acids. To be active, any polypeptide must have sufficient length to display biological and/or immunological activity.

The term “naturally occurring polypeptide” refers to polypeptides produced by cells that have not been genetically engineered and specifically contemplates various polypeptides arising from post-translational modifications of the polypeptide including, but not limited to, acetylation, carboxylation, glycosylation, phosphorylation, lipidation and acylation.

The term “translated protein coding portion” means a sequence which encodes for the full length protein which may include any leader sequence or a processing sequence.

The term “mature protein coding sequence” refers to a sequence which encodes a peptide or protein without any leader/signal sequence. The “mature protein portion” refers to that portion of the protein without the leader/signal sequence. The peptide may have the leader sequences removed during processing in the cell or the protein may have been produced synthetically or using a polynucleotide only encoding for the mature protein coding sequence. It is contemplated that the mature protein portion may or may not include an initial methionine residue. The initial methionine is often removed during processing of the peptide.

The term “derivative” refers to polypeptides chemically modified by such techniques as ubiquitination, labeling (e.g., with radionuclides or various enzymes), covalent polymer attachment such as pegylation (derivatization with polyethylene glycol) and insertion or substitution by chemical synthesis of amino acids such as ornithine, which do not normally occur in human proteins.

The term “variant” (or “analog”) refers to any polypeptide differing from naturally occurring polypeptides by amino acid insertions, deletions, and substitutions, created using, e.g., recombinant DNA techniques. Guidance in determining which amino acid residues may be replaced, added or deleted without abolishing activities of interest, may be found by comparing the sequence of the particular polypeptide with that of homologous peptides and minimizing the number of amino acid sequence changes made in regions of high homology (conserved regions) or by replacing amino acids with consensus sequence.

Alternatively, recombinant variants encoding these same or similar polypeptides may be synthesized or selected by making use of the “redundancy” in the genetic code. Various codon substitutions, such as the silent changes which produce various restriction sites, may be introduced to optimize cloning into a plasmid or viral vector or expression in a particular prokaryotic or eukaryotic system. Mutations in the polynucleotide sequence may be reflected in the polypeptide or domains of other peptides added to the polypeptide to modify the properties of any part of the polypeptide, to change characteristics such as ligand-binding affinities, interchain affinities, or degradation/turnover rate.

Preferably, amino acid “substitutions” are the result of replacing one amino acid with another amino acid having similar structural and/or chemical properties, i.e., conservative amino acid replacements. “Conservative” amino acid substitutions may be made on the basis of similarity in polarity, charge, solubility, hydrophobicity, hydrophilicity, and/or the amphipathic nature of the residues involved. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid. “Insertions” or “deletions” are preferably in the range of about 1 to 20 amino acids, more preferably 1 to 10 amino acids. The variation allowed may be experimentally determined by systematically making insertions, deletions, or substitutions of amino acids in a polypeptide molecule using recombinant DNA techniques and assaying the resulting recombinant variants for activity.

Alternatively, where alteration of function is desired, insertions, deletions or non-conservative alterations can be engineered to produce altered polypeptides. Such alterations can, for example, alter one or more of the biological functions or biochemical characteristics of the polypeptides of the invention. For example, such alterations may change polypeptide characteristics such as ligand-binding affinities, interchain affinities, or degradation/turnover rate. Further, such alterations can be selected so as to generate polypeptides that are better suited for expression, scale up and the like in the host cells chosen for expression. For example, cysteine residues can be deleted or substituted with another amino acid residue in order to eliminate disulfide bridges.

The terms “purified” or “substantially purified” as used herein denotes that the indicated nucleic acid or polypeptide is present in the substantial absence of other biological macromolecules, e.g., polynucleotides, proteins, and the like. In one embodiment, the polynucleotide or polypeptide is purified such that it constitutes at least 95% by weight, more preferably at least 99% by weight, of the indicated biological macromolecules present (but water, buffers, and other small molecules, especially molecules having a molecular weight of less than 1000 daltons, can be present).

The term “isolated” as used herein refers to a nucleic acid or polypeptide separated from at least one other component (e.g., nucleic acid or polypeptide) present with the nucleic acid or polypeptide in its natural source. In one embodiment, the nucleic acid or polypeptide is found in the presence of (if anything) only a solvent, buffer, ion, or other components normally present in a solution of the same. The terms “isolated” and “purified” do not encompass nucleic acids or polypeptides present in their natural source.

The term “recombinant,” when used herein to refer to a polypeptide or protein, means that a polypeptide or protein is derived from recombinant (e.g., microbial, insect, or mammalian) expression systems. “Microbial” refers to recombinant polypeptides or proteins made in bacterial or fungal (e.g., yeast) expression systems. As a product, “recombinant microbial” defines a polypeptide or protein essentially free of native endogenous substances and unaccompanied by associated native glycosylation. Polypeptides or proteins expressed in most bacterial cultures, e.g., E. coli, will be free of glycosylation modifications; polypeptides or proteins expressed in yeast will have a glycosylation pattern in general different from those expressed in mammalian cells.

The term “recombinant expression vehicle or vector” refers to a plasmid or phage or virus or vector, for expressing a polypeptide from a DNA (RNA) sequence. An expression vehicle can comprise a transcriptional unit comprising an assembly of (1) a genetic element or elements having a regulatory role in gene expression, for example, promoters or enhancers, (2) a structural or coding sequence which is transcribed into mRNA and translated into protein, and (3) appropriate transcription initiation and termination sequences. Structural units intended for use in yeast or eukaryotic expression systems preferably include a leader sequence enabling extracellular secretion of translated protein by a host cell. Alternatively, where recombinant protein is expressed without a leader or transport sequence, it may include an amino terminal methionine residue. This residue may or may not be subsequently cleaved from the expressed recombinant protein to provide a final product.

The term “recombinant expression system” means host cells which have stably integrated a recombinant transcriptional unit into chromosomal DNA or carry the recombinant transcriptional unit extrachromosomally. Recombinant expression systems as defined herein will express heterologous polypeptides or proteins upon induction of the regulatory elements linked to the DNA segment or synthetic gene to be expressed. This term also means host cells which have stably integrated a recombinant genetic element or elements having a regulatory role in gene expression, for example, promoters or enhancers. Recombinant expression systems as defined herein will express polypeptides or proteins endogenous to the cell upon induction of the regulatory elements linked to the endogenous DNA segment or gene to be expressed. The cells can be prokaryotic or eukaryotic.

The term “secreted” includes a protein that is transported across or through a membrane, including transport as a result of signal sequences in its amino acid sequence when it is expressed in a suitable host cell. “Secreted” proteins include without limitation proteins secreted wholly (e.g., soluble proteins) or partially (e.g., receptors) from the cell in which they are expressed. “Secreted” proteins also include without limitation proteins that are transported across the membrane of the endoplasmic reticulum. “Secreted” proteins are also intended to include proteins containing non-typical signal sequences (e.g. Interleukin-1 Beta, see Krasney, P. A. and Young, P. R. Cytokine 4:134-143 (1992)) and factors released from damaged cells (e.g. Interleukin-1 Receptor Antagonist, see Arend, W. P. et. al. Annu. Rev. Immunol. 16:27-55 (1998)).

Where desired, an expression vector may be designed to contain a “signal or leader sequence” which will direct the polypeptide through the membrane of a cell. Such a sequence may be naturally present on the polypeptides of the present invention or provided from heterologous protein sources by recombinant DNA techniques.

The term “stringent” is used to refer to conditions that are commonly understood in the art as stringent. Stringent conditions can include highly stringent conditions (i.e., hybridization to filter-bound DNA in 0.5 M NaHPO₄, 7% sodium dodecyl sulfate (SDS), 1 mM EDTA at 65° C., and washing in 0.1×SSC/0.1% SDS at 68° C.), and moderately stringent conditions (ie., washing in 0.2×SSC/0.1% SDS at 42° C.). Other exemplary hybridization conditions are described herein in the examples.

In instances of hybridization of deoxyoligonucleotides, additional exemplary stringent hybridization conditions include washing in 6×SSC/0.05% sodium pyrophosphate at 37° C. (for 14-base oligonucleotides), 48° C. (for 17-base oligonucleotides), 55° C. (for 20-base oligonucleotides), and 60° C. (for 23-base oligonucleotides).

As used herein, “substantially equivalent” can refer both to nucleotide and amino acid sequences, for example a mutant sequence, that varies from a reference sequence by one or more substitutions, deletions, or additions, the net effect of which does not result in an adverse functional dissimilarity between the reference and subject sequences. Typically, such a substantially equivalent sequence varies from one of those listed herein by no more than about 35% (i.e., the number of individual residue substitutions, additions, and/or deletions in a substantially equivalent sequence, as compared to the corresponding reference sequence, divided by the total number of residues in the substantially equivalent sequence is about 0.35 or less). Such a sequence is said to have 65% sequence identity to the listed sequence. In one embodiment, a substantially equivalent, e.g., mutant, sequence of the invention varies from a listed sequence by no more than 30% (70% sequence identity); in a variation of this embodiment, by no more than 25% (75% sequence identity); and in a further variation of this embodiment, by no more than 20% (80% sequence identity) and in a further variation of this embodiment, by no more than 10% (90% sequence identity) and in a further variation of this embodiment, by no more that 5% (95% sequence identity). Substantially equivalent, e.g., mutant, amino acid sequences according to the invention preferably have at least 80% sequence identity with a listed amino acid sequence, more preferably at least 90% sequence identity. Substantially equivalent nucleotide sequence of the invention can have lower percent sequence identities, taking into account, for example, the redundancy or degeneracy of the genetic code. Preferably, nucleotide sequence has at least about 65% identity, more preferably at least about 75% identity, and most preferably at least about 95% identity. For the purposes of the present invention, sequences having substantially equivalent biological activity and substantially equivalent expression characteristics are considered substantially equivalent. For the purposes of determining equivalence, truncation of the mature sequence (e.g., via a mutation which creates a spurious stop codon) should be disregarded. Sequence identity may be determined, e.g., using the Jotun Hein method (Hein, J. Methods Enzymol. 183:626-645 (1990)). Identity between sequences can also be determined by other methods known in the art, e.g. by varying hybridization conditions.

The term “totipotent” refers to the capability of a cell to differentiate into all of the cell types of an adult organism.

The term “transformafion” means introducing DNA into a suitable host cell so that the DNA is replicable, either as an extrachromosomal element, or by chromosomal integration. The term “transfection” refers to the taking up of an expression vector by a suitable host cell, whether or not any coding sequences are in fact expressed. The term “infection” refers to the introduction of nucleic acids into a suitable host cell by use of a virus or viral vector.

As used herein, an “uptake modulating fragment,” UMF, means a series of nucleotides which mediate the uptake of a linked DNA fragment into a cell. UMFs can be readily identified using known UMFs as a target sequence or target motif with the computer-based systems described below. The presence and activity of a UMF can be confirmed by attaching the suspected UMF to a marker sequence. The resulting nucleic acid molecule is then incubated with an appropriate host under appropriate conditions and the uptake of the marker sequence is determined. As described above, a UMF will increase the frequency of uptake of a linked marker sequence.

Each of the above terms is meant to encompass all that is described for each, unless the context dictates otherwise.

4.12 Nucleic Acids of the Invention

The isolated polynucleotides of the invention include, but are not limited to a polynucleotide comprising any of the nucleotide sequences of SEQ ID NO: 1-4, 6, 14, 16, 25-27, 29, 157-159, 161, 183-185, 187, 214, 216, 240, 242, 271, 273, 300-301, 303, 322, 324, 345-347, 349, 353-354, 356, 377, 379, 405-407, 409, 418-419, 421, 441-443, 485-486, 488, 503, 504, 506, 514-515, 517, 526-527, 529, 547, 549, 556, 558, 570-571, 573, 577-578, 580, 587, 589, 601, 603, 606, 608, 611, 613, 617, 619, 621, 623, 625, 627, 629, or 631; a fragment of SEQ ID NO: 1-4, 6, 14, 16, 25-27, 29, 157-159, 161, 183-185, 187, 214, 216, 240, 242, 271, 273, 300-301, 303, 322, 324, 345-347, 349, 353-354, 356, 377, 379, 405-407, 409, 418-419, 421, 441-443, 485-486, 488, 503, 504, 506, 514-515, 517, 526-527, 529, 547, 549, 556, 558, 570-571, 573, 577-578, 580, 587, 589, 601, 603, 606, 608, 611, 613, 617, 619, 621, 623, 625, 627, 629, or 631; a polynucleotide comprising the full length protein coding sequence of SEQ ID NO: 1-4, 6, 14, 16, 25-27, 29, 157-159, 161, 183-185, 187, 214, 216, 240, 242, 271, 273, 300-301, 303, 322, 324, 345-347, 349, 353-354, 356, 377, 379, 405-407, 409, 418-419, 421, 441-443, 485-486, 488, 503, 504, 506, 514-515, 517, 526-527, 529, 547, 549, 556, 558, 570-571, 573, 577-578, 580, 587, 589, 601, 603, 606, 608, 611, 613, 617, 619, 621, 623, 625, 627, 629, or 631 (for example coding for SEQ ID NO: 5, 15, 28, 160, 186, 215, 241, 272, 302, 323, 348, 355, 378, 408, 420, 444, 487, 505, 516, 528, 542, 548, 557, 572, 579, 588, 602, 607, 612, 618, 622, 626, or 630); and a polynucleotide comprising the nucleotide sequence encoding the mature protein coding sequence of the polypeptides of any one of SEQ ID NO: 5, 7-13, 15, 17-24, 28, 30-156, 160, 162-182, 186, 188-213, 215, 217-239, 241, 243-270, 272, 274-299, 302, 304-321, 323, 325-344, 348, 350-352, 355, 357-376, 378, 380-401, 408, 410-414, 415, 420, 422-439, 444-480,482-484,487, 489-501, 505, 507-512, 516, 518-524, 528, 530-539, 542, 544-546, 548, 550-553, 557, 559-567, 572, 574, 576, 579, 581-584, 588, 590, 596, 602, 604-605, 607, 609-610, 612, 614-615, 618, 620, 622, 624, 626, 628, 630, 632, or 634-653. The polynucleotides of the present invention also include, but are not limited to, a polynucleotide that hybridizes under stringent conditions to (a) the complement of any of the nucleotides sequences of SEQ ID NO: 1-4, 6, 14, 16, 25-27, 29, 157-159, 161, 183-185, 187, 214, 216, 240, 242, 271, 273, 300-301, 303, 322, 324, 345-347, 349, 353-354, 356, 377, 379, 405-407, 409, 418-419, 421, 441-443, 485-486, 488, 503, 504, 506, 514-515, 517, 526-527, 529, 547, 549, 556, 558, 570-571, 573, 577-578, 580, 587, 589, 601, 603, 606, 608, 611, 613, 617, 619, 621, 623, 625, 627, 629, or 631; (b) a polynucleotide encoding any one of the polypeptides of SEQ ID NO: 5, 7-13, 15, 17-24, 28, 30-156, 160, 162-182, 186, 188-213, 215, 217-239, 241, 243-270, 272, 274-299, 302, 304-321, 323, 325-344, 348, 350-352, 355, 357-376, 378, 380-401, 408, 410-414, 415, 420, 422-439, 444-480, 482-484, 487, 489-501, 505, 507-512, 516, 518-524, 528, 530-539, 542, 544-546, 548, 550-553, 557, 559-567, 572, 574, 576, 579, 581-584, 588, 590, 596, 602, 604-605, 607, 609-610, 612, 614-615, 618, 620, 622, 624, 626, 628, 630, 632, or 634-653; (c) a polynucleotide which is an allelic variant of any polynucleotides recited above; (d) a polynucleotide which encodes a species homolog of any of the proteins recited above; or (e) a polynucleotide that encodes a polypeptide comprising a specific domain or truncation of the polypeptides of SEQ ID NO: 1-4, 6, 14, 16, 25-27, 29, 157-159, 161, 183-185, 187, 214, 216, 240, 242, 271, 273, 300-301, 303, 322, 324, 345-347, 349, 353-354, 356, 377, 379, 405-407, 409, 418-419, 421, 441-443, 485486, 488, 503, 504, 506, 514-515, 517, 526-527, 529, 547, 549, 556, 558, 570-571, 573, 577-578, 580, 587, 589, 601, 603, 606, 608, 611, 613, 617, 619, 621, 623, 625, 627, 629, or 631. Domains of interest may depend on the nature of the encoded polypeptide; e.g., domains in receptor-like polypeptides include ligand-binding, extracellular, transmembrane, or cytoplasmic domains, or combinations thereof; domains in immunoglobulin-like proteins include the variable immunoglobulin-like domains; domains in enzyme-like polypeptides include catalytic and substrate binding domains; and domains in ligand polypeptides include receptor-binding domains.

The polynucleotides of the invention include naturally occurring or wholly or partially synthetic DNA, e.g., cDNA and genomic DNA, and RNA, e.g., mRNA. The polynucleotides may include the entire coding region of the cDNA or may represent a portion of the coding region of the cDNA.

The present invention also provides genes corresponding to the cDNA sequences disclosed herein. The corresponding genes can be isolated in accordance with known methods using the sequence information disclosed herein. Such methods include the preparation of probes or primers from the disclosed sequence information for identification and/or amplification of genes in appropriate genomic libraries or other sources of genomic materials. Further 5′ and 3′ sequence can be obtained using methods known in the art. For example, full length cDNA or genomic DNA that corresponds to any of the polynucleotides of SEQ ID NO: 1-4, 6, 14, 16, 25-27, 29, 157-159, 161, 183-185, 187, 214, 216, 240, 242, 271, 273, 300-301, 303, 322, 324, 345-347, 349, 353-354, 356, 377, 379, 405-407, 409, 418-419, 421, 441-443, 485-486, 488, 503, 504, 506, 514-515, 517, 526-527, 529, 547, 549, 556, 558, 570-571, 573, 577-578, 580, 587, 589, 601, 603, 606, 608, 611, 613, 617, 619, 621, 623, 625, 627, 629, or 631 can be obtained by screening appropriate cDNA or genomic DNA libraries under suitable hybridization conditions using any of the polynucleotides of SEQ ID NO: 1-4,6,14,16,25-27,29,157-159,161,183-185, 187, 214, 216, 240, 242, 271, 273, 300-301, 303, 322, 324, 345-347, 349, 353-354, 356, 377, 379, 405-407, 409, 418-419, 421, 441-443, 485-486, 488, 503, 504, 506, 514-515, 517, 526-527, 529, 547, 549, 556, 558, 570-571, 573, 577-578, 580, 587, 589, 601, 603, 606, 608, 611, 613, 617, 619, 621, 623, 625, 627, 629, or 631 or a portion thereof as a probe. Alternatively, the polynucleotides of SEQ ID NO: 1-4,6,14,16,25-27,29,157-159,161,183-185, 187, 214, 216, 240, 242, 271, 273, 300-301, 303, 322, 324, 345-347, 349, 353-354, 356, 377, 379, 405-407, 409, 418-419, 421, 441-443, 485-486, 488, 503, 504, 506, 514-515, 517, 526-527, 529, 547, 549, 556, 558, 570-571, 573, 577-578, 580, 587, 589, 601, 603, 606, 608, 611, 613, 617, 619, 621, 623, 625, 627, 629, or 631 may be used as the basis for suitable primer(s) that allow identification and/or amplification of genes in appropriate genomic DNA or cDNA libraries.

The nucleic acid sequences of the invention can be assembled from ESTs and sequences (including cDNA and genomic sequences) obtained from one or more public databases, such as dbEST, gbpri, and UniGene. The EST sequences can provide identifying sequence information, representative fragment or segment information, or novel segment information for the full-length gene.

The polynucleotides of the invention also provide polynucleotides including nucleotide sequences that are substantially equivalent to the polynucleotides recited above. Polynucleotides according to the invention can have, e.g., at least about 65%, at least about 70%, at least about 75%, at least about 80%, 81%, 82%, 83%, 84%, 85%, 86%, 87%, 88%, or 89%, more typically at least about 90%, 91%, 92%, 93%, or 94% and even more typically at least about 95%, 96%, 97%, 98% or 99% sequence identity to a polynucleotide recited above.

Included within the scope of the nucleic acid sequences of the invention are nucleic acid sequence fragments that hybridize under stringent conditions to any of the nucleotide sequences of SEQ ID NO: 1-4, 6, 14, 16, 25-27, 29, 157-159, 161, 183-185, 187, 214, 216, 240, 242, 271, 273, 300-301, 303, 322, 324, 345-347, 349, 353-354, 356, 377, 379, 405-407, 409, 418-419, 421, 441-443, 485-486, 488, 503, 504, 506, 514-515, 517, 526-527, 529, 547, 549, 556, 558, 570-571, 573, 577-578,580,587, 589, 601, 603, 606, 608, 611, 613, 617, 619, 621, 623, 625, 627, 629, or 631, or complements thereof, which fragment is greater than about 5 nucleotides, preferably 7 nucleotides, more preferably greater than 9 nucleotides and most preferably greater than 17 nucleotides. Fragments of, e.g. 15, 17, or 20 nucleotides or more that are selective for (i.e. specifically hybridize to any one of the polynucleotides of the invention) are contemplated. Probes capable of specifically hybridizing to a polynucleotide can differentiate polynucleotide sequences of the invention from other polynucleotide sequences in the same family of genes or can differentiate human genes from genes of other species, and are preferably based on unique nucleotide sequences.

The sequences falling within the scope of the present invention are not limited to these specific sequences, but also include allelic and species variations thereof. Allelic and species variations can be routinely determined by comparing the sequence provided in SEQ ID NO: 1-4,6,14,16,25-27,29,157-159,161,183-185, 187, 214, 216, 240, 242, 271, 273, 300-301, 303, 322, 324, 345-347, 349, 353-354, 356, 377, 379, 405-407, 409, 418-419, 421, 441-443, 485-486, 488, 503, 504, 506, 514-515, 517, 526-527, 529, 547, 549, 556, 558, 570-571, 573, 577-578, 580, 587, 589, 601, 603, 606, 608, 611, 613, 617, 619, 621, 623, 625, 627, 629, or 631, a representative fragment thereof, or a nucleotide sequence at least 90% identical, preferably 95% identical, to SEQ ID NO: 1-4, 6, 14, 16, 25-27, 29, 157-159, 161, 183-185, 187, 214, 216, 240, 242, 271, 273, 300-301, 303, 322, 324, 345-347, 349, 353-354, 356, 377, 379, 405-407, 409, 418-419, 421, 441-443, 485-486, 488, 503, 504, 506, 514-515, 517, 526-527, 529, 547, 549, 556, 558, 570-571, 573, 577-578, 580, 587, 589, 601, 603, 606, 608, 611, 613, 617, 619, 621, 623, 625, 627, 629, or 631 with a sequence from another isolate of the same species. Furthermore, to accommodate codon variability, the invention includes nucleic acid molecules coding for the same amino acid sequences as do the specific ORFs disclosed herein. In other words, in the coding region of an ORF, substitution of one codon for another codon that encodes the same amino acid is expressly contemplated.

The nearest neighbor result for the nucleic acids of the present invention, including SEQ ID NO: 14, 6, 14, 16, 25-27, 29, 157-159, 161, 183-185, 187, 214, 216, 240, 242, 271, 273, 300-301, 303, 322, 324, 345-347, 349, 353-354, 356, 377, 379, 405-407, 409, 418-419, 421, 441-443, 485-486, 488, 503, 504, 506, 514-515, 517, 526-527, 529, 547, 549, 556, 558, 570-571, 573, 577-578, 580, 587, 589, 601, 603, 606, 608, 611, 613, 617, 619, 621, 623, 625, 627, 629, or 631, can be obtained by searching a database using an algorithm or a program. Preferably, a BLAST which stands for Basic Local Alignment Search Tool is used to search for local sequence alignments (Altshul, S. F., J. Mol. Evol. 36 290-300 (1993) and Altschul S. F., et al. J. Mol. Biol. 21:403-410 (1990)).

Species homologs (or orthologs) of the disclosed polynucleotides and proteins are also provided by the present invention. Species homologs may be isolated and identified by making suitable probes or primers from the sequences provided herein and screening a suitable nucleic acid source from the desired species.

The invention also encompasses allelic variants of the disclosed polynucleotides or proteins; that is, naturally-occurring alternative forms of the isolated polynucleotide which also encodes proteins which are identical, homologous or related to that encoded by the polynucleotides.

The nucleic acid sequences of the invention are further directed to sequences which encode variants of the described nucleic acids. These amino acid sequence variants may be prepared by methods known in the art by introducing appropriate nucleotide changes into a native or variant polynucleotide. There are two variables in the construction of amino acid sequence variants: the location of the mutation and the nature of the mutation. Nucleic acids encoding the amino acid sequence variants are preferably constructed by mutating the polynucleotide to encode an amino acid sequence that does not occur in nature. These nucleic acid alterations can be made at sites that differ in the nucleic acids from different species (variable positions) or in highly conserved regions (constant regions). Sites at such locations will typically be modified in series, e.g., by substituting first with conservative choices (e.g., hydrophobic amino acid to a different hydrophobic amino acid) and then with more distant choices (e.g., hydrophobic amino acid to a charged amino acid), and then deletions or insertions may be made at the target site. Amino acid sequence deletions generally range from about 1 to 30 residues, preferably about 1 to 10 residues, and are typically contiguous. Amino acid insertions include amino- and/or carboxyl-terminal fusions ranging in length from one to one hundred or more residues, as well as intrasequence insertions of single or multiple amino acid residues. Intrasequence insertions may range generally from about 1 to 10 amino residues, preferably from 1 to 5 residues. Examples of terminal insertions include the heterologous signal sequences necessary for secretion or for intracellular targeting in different host cells and sequences such as FLAG or poly-histidine sequences useful for purifying the expressed protein.

In a preferred method, polynucleotides encoding the novel amino acid sequences are changed via site-directed mutagenesis. This method uses oligonucleotide sequences to alter a polynucleotide to encode the desired amino acid variant, as well as sufficient adjacent nucleotides on both sides of the changed amino acid to form a stable duplex on either side of the site being changed. In general, the techniques of site-directed mutagenesis are well known to those of skill in the art and this technique is exemplified by publications such as, Edelman et al., DNA 2:183 (1983). A versatile and efficient method for producing site-specific changes in a polynucleotide sequence was published by Zoller and Smith, Nucleic Acids Res. 10:6487-6500 (1982). PCR may also be used to create amino acid sequence variants of the novel nucleic acids. When small amounts of template DNA are used as starting material, primer(s) that differs slightly in sequence from the corresponding region in the template DNA can generate the desired amino acid variant. PCR amplification results in a population of product DNA fragments that differ from the polynucleotide template encoding the polypeptide at the position specified by the primer. The product DNA fragments replace the corresponding region in the plasmid and this gives a polynucleotide encoding the desired amino acid variant.

A further technique for generating amino acid variants is the cassette mutagenesis technique described in Wells, et al., Gene 34:315 (1985); and other mutagenesis techniques well known in the art, such as, for example, the techniques in Sambrook, et al., supra, and Current Protocols in Molecular Biology, Ausubel, et al. Due to the inherent degeneracy of the genetic code, other DNA sequences which encode substantially the same or a functionally equivalent amino acid sequence may be used in the practice of the invention for the cloning and expression of these novel nucleic acids. Such DNA sequences include those which are capable of hybridizing to the appropriate novel nucleic acid sequence under stringent conditions.

Polynucleotides encoding preferred polypeptide truncations of the invention can be used to generate polynucleotides encoding chimeric or fusion proteins comprising one or more domains of the invention and heterologous protein sequences.

The polynucleotides of the invention additionally include the complement of any of the polynucleotides recited above. The polynucleotide can be DNA (genomic, cDNA, amplified, or synthetic) or RNA. Methods and algorithms for obtaining such polynucleotides are well known to those of skill in the art and can include, for example, methods for determining hybridization conditions that can routinely isolate polynucleotides of the desired sequence identities.

In accordance with the invention, polynucleotide sequences comprising the mature protein coding sequences, coding for any one of SEQ ID NO: 5, 15, 28, 160, 186, 215, 241, 272, 302, 323, 348, 355, 378, 408, 420, 444, 487, 505, 516, 528, 542, 548, 557, 572, 579, 588, 602, 607, 612, 618, 622, 626, or 630, or functional equivalents thereof, may be used to generate recombinant DNA molecules that direct the expression of that nucleic acid, or a functional equivalent thereof, in appropriate host cells. Also included are the cDNA inserts of any of the clones identified herein.

A polynucleotide according to the invention can be joined to any of a variety of other nucleotide sequences by well-established recombinant DNA techniques (see Sambrook, J. et al. (1989) Molecular Cloning: A Laboratory Manual, Cold Spring Harbor Laboratory, NY). Useful nucleotide sequences for joining to polynucleotides include an assortment of vectors, e.g., plasmids, cosmids, lambda phage derivatives, phagemids, and the like, that are well known in the art. Accordingly, the invention also provides a vector including a polynucleotide of the invention and a host cell containing the polynucleotide. In general, the vector contains an origin of replication functional in at least one organism, convenient restriction endonuclease sites, and a selectable marker for the host cell. Vectors according to the invention include expression vectors, replication vectors, probe generation vectors, and sequencing vectors. A host cell according to the invention can be a prokaryotic or eukaryotic cell and can be a unicellular organism or part of a multicellular organism.

The present invention further provides recombinant constructs comprising a nucleic acid having any of the nucleotide sequences of SEQ ID NO: 1-4, 6, 14, 16, 25-27, 29, 157-159, 161, 183-185, 187, 214, 216, 240, 242, 271, 273, 300-301, 303, 322, 324, 345-347, 349, 353-354, 356, 377, 379, 405-407, 409, 418-419, 421, 441-443, 485-486, 488, 503, 504, 506, 514-515, 517, 526-527, 529, 547, 549, 556, 558, 570-571, 573, 577-578, 580, 587, 589, 601, 603, 606, 608, 611, 613, 617, 619, 621, 623, 625, 627, 629, or 631 or a fragment thereof or any other polynucleotides of the invention. In one embodiment, the recombinant constructs of the present invention comprise a vector, such as a plasmid or viral vector, into which a nucleic acid having any of the nucleotide sequences of SEQ ID NO: 1-4, 6, 14, 16, 25-27, 29, 157-159, 161, 183-185, 187, 214, 216, 240, 242, 271, 273, 300-301, 303, 322, 324, 345-347, 349, 353-354, 356, 377, 379, 405-407, 409, 418-419, 421, 441-443, 485-486, 488, 503, 504, 506, 514-515, 517, 526-527, 529, 547, 549, 556, 558, 570-571, 573, 577-578, 580, 587, 589, 601, 603, 606, 608, 611, 613, 617, 619, 621, 623, 625, 627, 629, or 631 or a fragment thereof is inserted, in a forward or reverse orientation. In the case of a vector comprising one of the ORFs of the present invention, the vector may further comprise regulatory sequences, including for example, a promoter, operably linked to the ORF. Large numbers of suitable vectors and promoters are known to those of skill in the art and are commercially available for generating the recombinant constructs of the present invention. The following vectors are provided by way of example. Bacterial: pBs, phagescript, PsiX174, pBluescript SK, pBs KS, pNH8a, pNH16a, pNH18a, pNH46a (Stratagene); pTrc99A, pKK223-3, pKK233-3, pDR540, pRIT5 (Pharmacia). Eukaryotic: pWLneo, pSV2cat, pOG44, PXTI, pSG (Stratagene) pSVK3, pBPV, pMSG, and pSVL (Pharmacia).

The isolated polynucleotide of the invention may be operably linked to an expression control sequence such as the pMT2 or pED expression vectors disclosed in Kaufman et al., Nucleic Acids Res. 19:4485-4490 (1991), in order to produce the protein recombinantly. Many suitable expression control sequences are known in the art. General methods of expressing recombinant proteins are also known and are exemplified in R. Kaufman, Methods in Enzymology 185:537-566 (1990). As defined herein “operably linked” means that the isolated polynucleotide of the invention and an expression control sequence are situated within a vector or cell in such a way that the protein is expressed by a host cell which has been transformed (transfected) with the ligated polynucleotide/expression control sequence.

Promoter regions can be selected from any desired gene using CAT (chloramphenicol transferase) vectors or other vectors with selectable markers. Two appropriate vectors are pKK232-8 and pCM7. Particular named bacterial promoters include lacI, lacZ, T3, T7, gpt, lambda PR, and trc. Eukaryotic promoters include CMV immediate early, HSV thymidine kinase, early and late SV40, LTRs from retrovirus, and mouse metallothionein-I. Selection of the appropriate vector and promoter is well within the level of ordinary skill in the art. Generally, recombinant expression vectors will include origins of replication and selectable markers permitting transformation of the host cell, e.g., the ampicillin resistance gene of E. coli and S. cerevisiae TRP1 gene, and a promoter derived from a highly expressed gene to direct transcription of a downstream structural sequence. Such promoters can be derived from operons encoding glycolytic enzymes such as 3-phosphoglycerate kinase (PGK), a-factor, acid phosphatase, or heat shock proteins, among others. The heterologous structural sequence is assembled in appropriate phase with translation initiation and termination sequences, and preferably, a leader sequence capable of directing secretion of translated protein into the periplasmic space or extracellular medium. Optionally, the heterologous sequence can encode a fusion protein including an amino terminal identification peptide imparting desired characteristics, e.g., stabilization or simplified purification of expressed recombinant product. Useful expression vectors for bacterial use are constructed by inserting a structural DNA sequence encoding a desired protein together with suitable translation initiation and termination signals in operable reading phase with a functional promoter. The vector will comprise one or more phenotypic selectable markers and an origin of replication to ensure maintenance of the vector and to, if desirable, provide amplification within the host. Suitable prokaryotic hosts for transformation include E. coli, Bacillus subtilis, Salmonella typhimurium and various species within the genera Pseudomonas, Streptomyces, and Staphylococcus, although others may also be employed as a matter of choice.

As a representative but non-limiting example, useful expression vectors for bacterial use can comprise a selectable marker and bacterial origin of replication derived from commercially available plasmids comprising genetic elements of the well known cloning vector pBR322 (ATCC 37017). Such commercial vectors include, for example, pKK223-3 (Pharmacia Fine Chemicals, Uppsala, Sweden) and GEM 1 (Promega Biotech, Madison, Wis., USA). These pBR322 “backbone” sections are combined with an appropriate promoter and the structural sequence to be expressed. Following transformation of a suitable host strain and growth of the host strain to an appropriate cell density, the selected promoter is induced or derepressed by appropriate means (e.g., temperature shift or chemical induction) and cells are cultured for an additional period. Cells are typically harvested by centrifugation, disrupted by physical or chemical means, and the resulting crude extract retained for further purification.

Polynucleotides of the invention can also be used to induce immune responses. For example, as described in Fan, et al., Nat. Biotech. 17:870-872 (1999), incorporated herein by reference, nucleic acid sequences encoding a polypeptide may be used to generate antibodies against the encoded polypeptide following topical administration of naked plasmid DNA or following injection, and preferably intramuscular injection of the DNA. The nucleic acid sequences are preferably inserted in a recombinant expression vector and may be in the form of naked DNA.

4.12.1 Antisense Nucleic Acids

Another aspect of the invention pertains to isolated antisense nucleic acid molecules that can hybridize to or are complementary to the nucleic acid molecule comprising the nucleotide sequence of SEQ ID NO: 1-4, 6, 14, 16, 25-27,29,157-159, 161, 183-185, 187, 214, 216, 240, 242, 271, 273, 300-301, 303, 322, 324, 345-347, 349, 353-354, 356, 377, 379, 405-407, 409, 418-419, 421, 441-443, 485-486, 488, 503, 504, 506, 514-515, 517, 526-527, 529, 547, 549, 556, 558, 570-571, 573, 577-578, 580, 587, 589, 601, 603, 606, 608, 611, 613, 617, 619, 621, 623, 625, 627, 629, or 631, or fragments, analogs or derivatives thereof. An “antisense” nucleic acid comprises a nucleotide sequence that is complementary to a “sense” nucleic acid encoding a protein (e.g., complementary to the coding strand of a double-stranded cDNA molecule or complementary to an mRNA sequence). In specific aspects, antisense nucleic acid molecules are provided that comprise a sequence complementary to at least about 10, 25, 50, 100, 250 or 500 nucleotides or an entire coding strand, or to only a portion thereof. Nucleic acid molecules encoding fragments, homologs, derivatives and analogs of a protein of any of SEQ ID NO: 5, 7-13, 15, 17-24, 28, 30-156, 160, 162-182, 186, 188-213, 215, 217-239, 241, 243-270, 272, 274-299, 302, 304-321, 323, 325-344, 348, 350-352, 355, 357-376, 378, 380-401, 408, 410414, 415, 420, 422-439, 444-480, 482-484, 487, 489-501, 505, 507-512, 516, 518-524, 528, 530-539, 542, 544-546, 548, 550-553, 557, 559-567, 572, 574, 576, 579, 581-584, 588, 590, 596, 602, 604-605, 607, 609-610, 612, 614-615, 618, 620, 622, 624, 626, 628, 630, 632, or 634-653 or antisense nucleic acids complementary to a nucleic acid sequence of SEQ ID NO: 1-4, 6, 14, 16, 25-27, 29, 157-159,161,183-185, 187, 214, 216, 240, 242, 271, 273, 300-301,303, 322, 324, 345-347, 349, 353-354, 356, 377, 379, 405-407, 409, 418-419, 421, 441-443, 485-486, 488, 503, 504, 506, 514-515, 517, 526-527, 529, 547, 549, 556, 558, 570-571, 573, 577-578, 580, 587, 589, 601, 603, 606, 608, 611, 613, 617, 619, 621, 623, 625, 627, 629, or 631 are additionally provided.

In one embodiment, an antisense nucleic acid molecule is antisense to a “coding region” of the coding strand of a nucleotide sequence of the invention. The term “coding region” refers to the region of the nucleotide sequence comprising codons which are translated into amino acid residues. In another embodiment, the antisense nucleic acid molecule is antisense to a “conceding region” of the coding strand of a nucleotide sequence of the invention. The term “conceding region” refers to 5′ and 3′ sequences which flank the coding region that are not translated into amino acids (i.e., also referred to as 5′ and 3′ untranslated regions).

Given the coding strand sequences (e.g. SEQ ID NO: 1-4, 6, 14, 16, 25-27, 29, 157-159, 161, 183-185, 187, 214, 216, 240, 242, 271, 273, 300-301, 303, 322, 324, 345-347, 349, 353-354,356,377,379,405-407,409,418-419, 421, 441-443,485-486, 488, 503, 504, 506, 514-515, 517, 526-527, 529, 547, 549, 556, 558, 570-571, 573, 577-578, 580, 587, 589, 601, 603, 606, 608, 611, 613, 617, 619, 621, 623, 625, 627, 629, or 631) disclosed herein, antisense nucleic acids of the invention can be designed according to the rules of Watson and Crick or Hoogsteen base pairing. The antisense nucleic acid molecule can be complementary to the entire coding region of an mRNA of the invention, but more preferably is an oligonucleotide that is antisense to only a portion of the coding or noncoding region of an mRNA of the invention. For example, the antisense oligonucleotide can be complementary to the region surrounding the translation start site of an mRNA of the invention. An antisense oligonucleotide can be, for example, about 5, 10, 15, 20, 25, 30, 35, 40, 45, or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following section).

The antisense nucleic acid molecules of the invention are typically administered to a subject or generated in situ such that they hybridize with or bind to cellular mRNA and/or genomic DNA encoding a protein according to the invention to thereby inhibit expression of the protein (e.g., by inhibiting transcription and/or translation). The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient nucleic acid molecules, vector constructs in which the antisense nucleic acid molecule is placed under the control of a strong pol II or pol III promoter are preferred.

In yet another embodiment, the antisense nucleic acid molecule of the invention is an alpha-anomeric nucleic acid molecule. An alpha-anomeric nucleic acid molecule forms specific double-stranded hybrids with complementary RNA in which, contrary to the usual alpha-units, the strands run parallel to each other. See, e.g., Gaultier, et al., Nucl. Acids Res. 15:6625-6641 (1987). The antisense nucleic acid molecule can also comprise a 2′-o-methylribonucleotide (see, e.g., Inoue, et al. Nucl. Acids Res. 15:6131-6148 (1987)) or a chimeric RNA-DNA analogue (see, e.g., Inoue, et al., FEBS Lett. 215:327-330 (1987).

4.12.2 Ribozymes And PNA Moieties

Nucleic acid modifications include, by way of non-limiting example, modified bases, and nucleic acids whose sugar phosphate backbones are modified or derivatized. These modifications are carried out at least in part to enhance the chemical stability of the modified nucleic acid, such that they can be used, for example, as antisense binding nucleic acids in therapeutic applications in a subject.

In one embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes as described in Haselhoff and Gerlach, Nature 334: 585-591 (1988)) can be used to catalytically cleave mRNA transcripts of the invention to thereby inhibit translation of mRNA of the invention. A ribozyme having specificity for a nucleic acid of the invention can be designed based upon the nucleotide sequence of a cDNA disclosed herein (e.g. SEQ ID NO: 1-4, 6, 14, 16, 25-27, 29, 157-159, 161, 183-185, 187, 214, 216, 240, 242, 271, 273, 300-301, 303, 322, 324, 345-347, 349, 353-354, 356, 377, 379, 405-407, 409, 418-419, 421, 441-443, 485-486, 488, 503, 504, 506, 514-515, 517, 526-527, 529, 547, 549, 556, 558, 570-571, 573, 577-578, 580, 587, 589, 601, 603, 606, 608, 611, 613, 617, 619, 621, 623, 625, 627, 629, or 631). For example, a derivative of a Tetrahymena L-19 IVS RNA can be constructed in which the nucleotide sequence of the active site is complementary to the nucleotide sequence to be cleaved in an mRNA of the invention. See, e.g., U.S. Pat. No. 4,987,071 to Cech, et al. and U.S. Pat. No. 5,116,742 to Cech, et al. Stem cell growth factor-like mRNA can also be used to select a catalytic RNA having a specific ribonuclease activity from a pool of RNA molecules. See, e.g., Bartel, et al., Science 261:1411-1418 (1993).

Alternatively, gene expression can be inhibited by targeting nucleotide sequences complementary to the regulatory region (e.g., the promoter and/or enhancers of the gene relating to the invention) to form triple helical structures that prevent transcription of the gene in target cells. See, e.g., Helene, Anticancer Drug Des. 6:569-84 (1991); Helene, et al., Ann. N.Y. Acad. Sci. 660:27-36 (1992); Maher, Bioassays 14:807-15 (1992).

In various embodiments, the nucleic acids of the invention can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids. See, e.g., Hyrup, et al., Bioorg. Med. Chem. 4:5-23 (1996). As used herein, the terms “peptide nucleic acids” or “PNAs” refer to nucleic acid mimics (e.g., DNA mimics) in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup, et al., 1996. supra; Perry-O'Keefe, et al., Proc. Natl. Acad. Sci. USA 93:14670-14675 (1996).

PNAs of the invention can be used in therapeutic and diagnostic applications. For example, PNAs can be used as antisense or antigene agents for sequence-specific modulation of gene expression by, e.g., inducing transcription or translation arrest or inhibiting replication. PNAs of the invention can also be used, for example, in the analysis of single base pair mutations in a gene (e.g., PNA directed PCR clamping; as artificial restriction enzymes when used in combination with other enzymes, e.g., S1 nucleases (see, Hyrup, et al., 1996.supra); or as probes or primers for DNA sequence and hybridization (see, Hyrup, et al., 1996, supra; Perry-O'Keefe, et al., 1996. supra).

In another embodiment, PNAs of the invention can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of the invention can be generated that may combine the advantageous properties of PNA and DNA. Such chimeras allow DNA recognition enzymes (e.g., RNase H and DNA polymerases) to interact with the DNA portion while the PNA portion would provide high binding affinity and specificity. PNA-DNA chimeras can be linked using linkers of appropriate lengths selected in terms of base stacking, number of bonds between the nucleobases, and orientation (see, Hyrup, et al., 1996. supra). The synthesis of PNA-DNA chimeras can be performed as described in Hyrup, et al., 1996. Supra, et al., Nucl Acids Res 24:3357-3363 (1996). For example, a DNA chain can be synthesized on a solid support using standard phosphoramidite coupling chemistry, and modified nucleoside analogs, e.g., 5′-(4-methoxytrityl)amino-5′-deoxythymidine phosphoramidite, can be used between the PNA and the 5′ end of DNA. See, e.g., Mag, et al., Nucl Acid Res 17:5973-5988 (1989). PNA monomers are then coupled in a stepwise manner to produce a chimeric molecule with a 5′ PNA segment and a 3′ DNA segment. See, e.g., Finn, et al., 1996. supra. Alternatively, chimeric molecules can be synthesized with a 5′ DNA segment and a 3′ PNA segment. See, e.g., Petersen, et al., Bioorg. Med. Chem. Lett. 5:1119-11124 (1975).

In other embodiments, the oligonucleotide may include other appended groups such as peptides (e.g., for targeting host cell receptors in vivo), or agents facilitating transport across the cell membrane (see, e.g., Letsinger, et al., Proc. Natl. Acad. Sci. U.S.A. 86:6553-6556 (1989); Lemaitre, et al., Proc. Natl. Acad. Sci. USA 84:648-652 (1987); PCT Publication No. WO88/09810) or the blood-brain barrier (see, e.g., PCT Publication No. WO 89/10134). In addition, oligonucleotides can be modified with hybridization-triggered cleavage agents (see, e.g., Krol, et al., BioTechniques 6:958-976 (1988)) or intercalating agents (see, e.g., Zon, Pharm. Res. 5:539-549 (1988)). To this end, the oligonucleotide can be conjugated to another molecule, e.g., a peptide, a hybridization triggered cross-linking agent, a transport agent, a hybridization-triggered cleavage agent, and the like.

4.13 Hosts

The present invention further provides host cells genetically engineered to contain the polynucleotides of the invention. For example, such host cells may contain nucleic acids of the invention introduced into the host cell using known transformation, transfection or infection methods. The present invention still further provides host cells genetically engineered to express the polynucleotides of the invention, wherein such polynucleotides are in operative association with a regulatory sequence heterologous to the host cell which drives expression of the polynucleotides in the cell.

The host cell can be a higher eukaryotic host cell, such as a mammalian cell, a lower eukaryotic host cell, such as a yeast cell, or the host cell can be a prokaryotic cell, such as a bacterial cell. Introduction of the recombinant construct into the host cell can be effected by calcium phosphate transfection, DEAE, dextran mediated transfection, or electroporation (Davis, L. et al., Basic Methods in Molecular Biology (1986)). The host cells containing one of polynucleotides of the invention, can be used in conventional manners to produce the gene product encoded by the isolated fragment (in the case of an ORF) or can be used to produce a heterologous protein under the control of the EMF.

Any host/vector system can be used to express one or more of the ORFs of the present invention. These include, but are not limited to, eukaryotic hosts such as HeLa cells, Cv-1 cell, COS cells, and Sf9 cells, as well as prokaryotic host such as E. coli and B. subtilis. The most preferred cells are those which do not normally express the particular polypeptide or protein or which expresses the polypeptide or protein at low natural level. Mature proteins can be expressed in mammalian cells, yeast, bacteria, or other cells under the control of appropriate promoters. Cell-free translation systems can also be employed to produce such proteins using RNAs derived from the DNA constructs of the present invention. Appropriate cloning and expression vectors for use with prokaryotic and eukaryotic hosts are described by Sambrook, et al., in Molecular Cloning: A Laboratory Manual, Second Edition, Cold Spring Harbor, N.Y. (1989), the disclosure of which is hereby incorporated by reference.

Various mammalian cell culture systems can also be employed to express recombinant protein. Examples of mammalian expression systems include the COS-7 lines of monkey kidney fibroblasts, described by Gluzman, Cell 23:175 (1981), and other cell lines capable of expressing a compatible vector, for example, the C127, 3T3, CHO, HeLa and BHK cell tines. Mammalian expression vectors will comprise an origin of replication, a suitable promoter, and also any necessary ribosome binding sites, polyadenylation site, splice donor and acceptor sites, transcriptional termination sequences, and 5′ flanking nontranscribed sequences. DNA sequences derived from the SV40 viral genome, for example, SV40 origin, early promoter, enhancer, splice, and polyadenylation sites may be used to provide the required nontranscribed genetic elements. Recombinant polypeptides and proteins produced in bacterial culture are usually isolated by initial extraction from cell pellets, followed by one or more salting-out, aqueous ion exchange or size exclusion chromatography steps. Protein refolding steps can be used, as necessary, in completing configuration of the mature protein. Finally, high performance liquid chromatography (HPLC) can be employed for final purification steps. Microbial cells employed in expression of proteins can be disrupted by any convenient method, including freeze-thaw cycling, sonication, mechanical disruption, or use of cell lysing agents.

A number of types of cells may act as suitable host cells for expression of the protein. Mammalian host cells include, for example, monkey COS cells, Chinese Hamster Ovary (CHO) cells, human kidney 293 cells, human epidermal A431 cells, human Colo205 cells, 3T3 cells, CV-1 cells, other transformed primate cell lines, normal diploid cells, cell strains derived from in vitro culture of primary tissue, primary explants, HeLa cells, mouse L cells, BHK, HL-60, U937, HaK or Jurkat cells.

Alternatively, it may be possible to produce the protein in lower eukaryotes such as yeast or in prokaryotes such as bacteria. Potentially suitable yeast strains include Saccharomyces cerevisiae, Schizosaccharomyces pombe, Kluyveromyces strains, Candida albicans, or any yeast strain capable of expressing heterologous proteins. Potentially suitable bacterial strains include Escherichia coli, Bacillus subtilis, Salmonella typhimurium, or any bacterial strain capable of expressing heterologous proteins. If the protein is made in yeast or bacteria, it may be necessary to modify the protein produced therein, for example by phosphorylation or glycosylation of the appropriate sites, in order to obtain the functional protein. Such covalent attachments may be accomplished using known chemical or enzymatic methods.

In another embodiment of the present invention, cells and tissues may be engineered to express an endogenous gene comprising the polynucleotides of the invention under the control of inducible regulatory elements, in which case the regulatory sequences of the endogenous gene may be replaced by homologous recombination. As described herein, gene targeting can be used to replace a gene's existing regulatory region with a regulatory sequence isolated from a different gene or a novel regulatory sequence synthesized by genetic engineering methods. Such regulatory sequences may be comprised of promoters, enhancers, scaffold-attachment regions, negative regulatory elements, transcriptional initiation sites, regulatory protein binding sites or combinations of said sequences. Alternatively, sequences which affect the structure or stability of the RNA or protein produced may be replaced, removed, added, or otherwise modified by targeting, including polyadenylation signals, mRNA stability elements, splice sites, leader sequences for enhancing or modifying transport or secretion properties of the protein, or other sequences which alter or improve the function or stability of protein or RNA molecules.

The targeting event may be a simple insertion of the regulatory sequence, placing the gene under the control of the new regulatory sequence, e.g., inserting a new promoter or enhancer or both upstream of a gene. Alternatively, the targeting event may be a simple deletion of a regulatory element, such as the deletion of a tissue-specific negative regulatory element. Alternatively, the targeting event may replace an existing element; for example, a tissue-specific enhancer can be replaced by an enhancer that has broader or different cell-type specificity than the naturally occurring elements. Here, the naturally occurring sequences are deleted and new sequences are added. In all cases, the identification of the targeting event may be facilitated by the use of one or more selectable marker genes that are contiguous with the targeting DNA, allowing for the selection of cells in which the exogenous DNA has integrated into the host cell genome. The identification of the targeting event may also be facilitated by the use of one or more marker genes exhibiting the property of negative selection, such that the negatively selectable marker is linked to the exogenous DNA, but configured such that the negatively selectable marker flanks the targeting sequence, and such that a correct homologous recombination event with sequences in the host cell genome does not result in the stable integration of the negatively selectable marker. Markers useful for this purpose include the Herpes Simplex Virus thymidine kinase (TK) gene or the bacterial xanthine-guanine phosphoribosyl-transferase (gpt) gene.

The gene targeting or gene activation techniques which can be used in accordance with this aspect of the invention are more particularly described in U.S. Pat. No. 5,272,071 to Chappel; U.S. Pat. No. 5,578,461 to Sherwin et al.; International Application No. PCT/US92/09627 (WO93/09222) by Selden et al.; and International Application No. PCT/US90/06436 (WO91/06667) by Skoultchi et al., each of which is incorporated by reference herein in its entirety.

4.13.1 Chimeric and Fusion Proteins

The invention also provides chimeric or fusion proteins. As used herein, a “chimeric protein” or “fusion protein” of the invention comprises a polypeptide of the invention operatively linked to another polypeptide. Within a fusion protein of the invention, the polypeptide according to the invention can correspond to all or a portion of a protein according to the invention. In one embodiment, a fusion protein comprises at least one biologically active portion of a protein according to the invention. In another embodiment, a fusion protein comprises at least two biologically active portions of a protein according to the invention. In yet another embodiment, a fusion protein comprises at least three biologically active portions of a protein according to the invention. Within the fusion protein, the term “operatively-linked” is intended to indicate that the polypeptide according to the invention and the other polypeptide are fused in-frame with one another. The other polypeptide can be fused to the N-terminus or C-terminus of the polypeptide according to the invention. For example, in one embodiment a fusion protein comprises a polypeptide according to the invention operably linked to the extracellular domain of a second protein.

In one embodiment, the fusion protein is a GST-fusion protein in which the polypeptide sequences according to the invention are fused to the C-terminus of the GST (glutathione S-transferase) sequences. Such fusion proteins can facilitate the purification of recombinant polypeptides according to the invention. In another embodiment, the fusion protein is a protein according to the invention containing a heterologous signal sequence at its N-terminus. In certain host cells (e.g., mammalian host cells), expression and/or secretion of the polypeptide according to the invention can be increased through use of a heterologous signal sequence.

In yet another embodiment, the fusion protein is an immunoglobulin fusion protein in which the polypeptide sequences of the invention are fused to sequences derived from a member of the immunoglobulin protein family. The immunoglobulin fusion proteins of the invention can be incorporated into pharmaceutical compositions and administered to a subject to inhibit an interaction between a ligand and a protein according to the invention on the surface of a cell, to thereby suppress signal transduction mediated by the protein according to the invention in vivo. The immunoglobulin fusion proteins can be used to affect the bioavailability of a cognate ligand. Inhibition of the ligand/protein interaction can be useful therapeutically for both the treatment of proliferative and differentiative disorders, as well as modulating (e.g. promoting or inhibiting) cell survival. Moreover, the immunoglobulin fusion proteins of the invention can be used as immunogens to produce antibodies in a subject, to purify ligands, and in screening assays to identify molecules that inhibit the interaction of a polypeptide according to the invention with a ligand.

A chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g., Ausubel, et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). A nucleic acid encoding a polypeptide of the invention can be cloned into such an expression vector such that the fusion moiety is linked in-frame to the protein of the invention.

4.14 Polypeptides of the Invention

The isolated polypeptides of the invention include, but are not limited to, a polypeptide comprising: the amino acid sequence set forth as any one of SEQ ID NO: 5, 7-13, 15, 17-24,28,30-156, 160, 162-182, 186, 188-213, 215, 217-239, 241, 243-270, 272, 274-299, 302, 304-321, 323, 325-344, 348, 350-352, 355, 357-376, 378, 380-401, 408, 410-414, 415, 420, 422-439, 444-480, 482-484, 487, 489-501, 505, 507-512, 516, 518-524, 528, 530-539, 542, 544-546, 548, 550-553, 557, 559-567, 572, 574, 576, 579, 581-584, 588, 590, 596, 602, 604-605, 607, 609-610, 612, 614-615, 618, 620, 622, 624, 626, 628, 630, 632, or 634-653 or an amino acid sequence encoded by any one of the nucleotide sequences SEQ ID NO: 2-4, 6, 14, 16, 26-27, 29, 158-159, 161, 184-185, 187, 214, 216, 240, 242, 271, 273, 301, 303, 322, 324, 346-347, 349, 354, 356, 377, 379, 407, 409, 419, 421, 443, 486, 488, 504, 506, 515, 517, 527, 529, 541, 543, 547, 549, 556, 558, 571, 573, 578, 580, 587, 589, 601, 603, 606, 608, 611, 613, 617, 619, 621, 623, 625, 627, 630, or 631, or the corresponding full length or mature protein. Polypeptides of the invention also include polypeptides preferably with biological or immunological activity that are encoded by: (a) a polynucleotide having any one of the nucleotide sequences set forth in SEQ ID NO: 1-4, 6, 14, 16, 25-27, 29, 157-159, 161, 183-185, 187, 214, 216, 240, 242, 271, 273, 300-301, 303, 322, 324, 345-347, 349, 353-354, 356, 377, 379, 405-407, 409, 418-419, 421, 441-443, 485-486, 488, 503, 504, 506, 514-515, 517, 526-527, 529, 547, 549, 556, 558, 570-571, 573, 577-578,580,587,589, 601, 603, 606, 608, 611, 613, 617, 619, 621, 623, 625, 627, 629, or 631 or (b) polynucleotides encoding any one of the amino acid sequences set forth as SEQ ID NO: 5, 7-13, 15, 17-24, 28, 30-156, 160, 162-182, 186, 188-213, 215, 217-239, 241, 243-270, 272, 274-299, 302, 304-321, 323, 325-344, 348, 350-352, 355, 357-376, 378, 380-401, 408, 410-414, 415, 420, 422-439, 444-480, 482-484, 487, 489-501, 505, 507-512, 516, 518-524, 528, 530-539, 542, 544-546, 548, 550-553, 557, 559-567, 572, 574, 576, 579, 581-584, 588, 590, 596, 602, 604-605, 607, 609-610, 612, 614-615, 618, 620, 622, 624, 626, 628, 630, 632, or 634-653 or (c) polynucleotides that hybridize to the complement of the polynucleotides of either (a) or (b) under stringent hybridization conditions. The invention also provides biologically active or immunologically active variants of any of the amino acid sequences set forth as SEQ ID NO: 5, 7-13, 15, 17-24, 28, 30-156, 160, 162-182, 186, 188-213, 215, 217-239, 241, 243-270, 272, 274-299, 302, 304-321, 323, 325-344, 348, 350-352, 355, 357-376, 378, 380-401, 408, 410-414, 415, 420, 422-439, 444-480, 482-484, 487, 489-501, 505, 507-512, 516, 518-524, 528, 530-539, 542, 544-546, 548, 550-553, 557, 559-567, 572, 574, 576, 579, 581-584,588,590,596, 602, 604-605,607,609-610,612,614-615, 618, 620, 622, 624, 626, 628, 630, 632, or 634-653 or the corresponding full length or mature protein; and “substantial equivalents” thereof (e.g., with at least about 65%, at least about 70%, at least about 75%, at least about 80%, 81%, 82%, 83%, 84%, 85%, 86%, 87%, 88%, or 89%, more typically at least about 90%, 91%, 92%, 93%, or 94% and even more typically at least about 95%, 96%, 97%, 98% or 99%, most typically at least about 99% amino acid identity) that retain biological activity. Polypeptides encoded by allelic variants may have a similar, increased, or decreased activity compared to polypeptides comprising SEQ ID NO: 5, 7-13, 15, 17-24, 28, 30-156, 160, 162-182, 186, 188-213, 215, 217-239, 241, 243-270, 272, 274-299, 302, 304-321, 323, 325-344, 348, 350-352, 355, 357-376, 378, 380-401, 408, 410-414, 415, 420, 422-439, 444-480, 482-484, 487, 489-501, 505, 507-512, 516, 518-524, 528, 530-539, 542, 544-546, 548, 550-553, 557, 559-567, 572, 574, 576, 579, 581-584, 588, 590, 596, 602, 604-605, 607, 609-610, 612, 614-615, 618, 620, 622, 624, 626, 628, 630, 632, or 634-653.

Fragments of the proteins of the present invention which are capable of exhibiting biological activity are also encompassed by the present invention. Fragments of the protein may be in linear form or they may be cyclized using known methods, for example, as described in H. U. Saragovi, et al., Bio/Technology 10:773-778 (1992) and in R. S. McDowell, et al., J. Amer. Chem. Soc. 114:9245-9253 (1992), both of which are incorporated herein by reference. Such fragments may be fused to carrier molecules such as immunoglobulins for many purposes, including increasing the valency of protein binding sites.

The present invention also provides both full-length and mature forms (for example, without a signal sequence or precursor sequence) of the disclosed proteins. The protein coding sequence is identified in the sequence listing by translation of the disclosed nucleotide sequences. The mature form of such protein may be obtained by expression of a full-length polynucleotide in a suitable mammalian cell or other host cell. The sequence of the mature form of the protein is also determinable from the amino acid sequence of the full-length form. Where proteins of the present invention are membrane bound, soluble forms of the proteins are also provided. In such forms, part or all of the regions causing the proteins to be membrane bound are deleted so that the proteins are fully secreted from the cell in which it is expressed.

Protein compositions of the present invention may further comprise an acceptable carrier, such as a hydrophilic, e.g., pharmaceutically acceptable, carrier.

The present invention further provides isolated polypeptides encoded by the nucleic acid fragments of the present invention or by degenerate variants of the nucleic acid fragments of the present invention. By “degenerate variant” is intended nucleotide fragments which differ from a nucleic acid fragment of the present invention (e.g., an ORF) by nucleotide sequence but, due to the degeneracy of the genetic code, encode an identical polypeptide sequence. Preferred nucleic acid fragments of the present invention are the ORFs that encode proteins.

A variety of methodologies known in the art can be utilized to obtain any one of the isolated polypeptides or proteins of the present invention. At the simplest level, the amino acid sequence can be synthesized using commercially available peptide synthesizers. The synthetically-constructed protein sequences, by virtue of sharing primary, secondary or tertiary structural and/or conformational characteristics with proteins may possess biological properties in common therewith, including protein activity. This technique is particularly useful in producing small peptides and fragments of larger polypeptides. Fragments are useful, for example, in generating antibodies against the native polypeptide. Thus, they may be employed as biologically active or immunological substitutes for natural, purified proteins in screening of therapeutic compounds and in immunological processes for the development of antibodies.

The polypeptides and proteins of the present invention can alternatively be purified from cells which have been altered to express the desired polypeptide or protein. As used herein, a cell is said to be altered to express a desired polypeptide or protein when the cell, through genetic manipulation, is made to produce a polypeptide or protein which it normally does not produce or which the cell normally produces at a lower level. One skilled in the art can readily adapt procedures for introducing and expressing either recombinant or synthetic sequences into eukaryotic or prokaryotic cells in order to generate a cell which produces one of the polypeptides or proteins of the present invention.

The invention also relates to methods for producing a polypeptide comprising growing a culture of host cells of the invention in a suitable culture medium, and purifying the protein from the cells or the culture in which the cells are grown. For example, the methods of the invention include a process for producing a polypeptide in which a host cell containing a suitable expression vector that includes a polynucleotide of the invention is cultured under conditions that allow expression of the encoded polypeptide. The polypeptide can be recovered from the culture, conveniently from the culture medium, or from a lysate prepared from the host cells and further purified. Preferred embodiments include those in which the protein produced by such process is a full length or mature form of the protein.

In an alternative method, the polypeptide or protein is purified from bacterial cells which naturally produce the polypeptide or protein. One skilled in the art can readily follow known methods for isolating polypeptides and proteins in order to obtain one of the isolated polypeptides or proteins of the present invention. These include, but are not limited to, immunochromatography, HPLC, size-exclusion chromatography, ion-exchange chromatography, and immuno-affinity chromatography. See, e.g., Scopes, Protein Purification: Principles and Practice, Springer-Verlag (1994); Sambrook, et al., in Molecular Cloning: A Laboratory Manual; Ausubel et al., Current Protocols in Molecular Biology. Polypeptide fragments that retain biological/immunological activity include fragments comprising greater than about 100 amino acids, or greater than about 200 amino acids, and fragments that encode specific protein domains.

The purified polypeptides can be used in in vitro binding assays which are well known in the art to identify molecules which bind to the polypeptides. These molecules include but are not limited to, for e.g., small molecules, molecules from combinatorial libraries, antibodies or other proteins. The molecules identified in the binding assay are then tested for antagonist or agonist activity in in vivo tissue culture or animal models that are well known in the art. In brief, the molecules are titrated into a plurality of cell cultures or animals and then tested for either cell/animal death or prolonged survival of the animal/cells.

In addition, the peptides of the invention or molecules capable of binding to the peptides may be complexed with toxins, e.g., ricin or cholera, or with other compounds that are toxic to cells. The toxin-binding molecule complex is then targeted to a tumor or other cell by the specificity of the binding molecule for SEQ ID NO: 5, 7-13, 15, 17-24, 28, 30-156, 160, 162-182, 186, 188-213, 215, 217-239, 241, 243-270, 272, 274-299, 302, 304-321, 323, 325-344, 348, 350-352, 355, 357-376, 378, 380401, 408, 410-414, 415, 420, 422-439, 444-480, 482-484, 487, 489-501, 505, 507-512, 516, 518-524, 528, 530-539, 542, 544-546, 548, 550-553, 557, 559-567, 572, 574, 576, 579, 581-584, 588, 590, 596, 602, 604-605, 607, 609-610, 612, 614-615, 618, 620, 622, 624, 626, 628, 630, 632, or 634-653.

The protein of the invention may also be expressed as a product of transgenic animals, e.g., as a component of the milk of transgenic cows, goats, pigs, or sheep which are characterized by somatic or germ cells containing a nucleotide sequence encoding the protein.

The proteins provided herein also include proteins characterized by amino acid sequences similar to those of purified proteins but into which modification are naturally provided or deliberately engineered. For example, modifications, in the peptide or DNA sequence, can be made by those skilled in the art using known techniques. Modifications of interest in the protein sequences may include the alteration, substitution, replacement, insertion or deletion of a selected amino acid residue in the coding sequence. For example, one or more of the cysteine residues may be deleted or replaced with another amino acid to alter the conformation of the molecule. Techniques for such alteration, substitution, replacement, insertion or deletion are well known to those skilled in the art (see, e.g. U.S. Pat. No. 4,518,584). Preferably, such alteration, substitution, replacement, insertion or deletion retains the desired activity of the protein. Regions of the protein that are important for the protein function can be determined by various methods known in the art including the alanine-scanning method which involved systematic substitution of single or strings of amino acids with alanine, followed by testing the resulting alanine-containing variant for biological activity. This type of analysis determines the importance of the substituted amino acid(s) in biological activity. Regions of the protein that are important for protein function may be determined by the eMATRIX program.

Other fragments and derivatives of the sequences of proteins which would be expected to retain protein activity in whole or in part and are useful for screening or other immunological methodologies may also be easily made by those skilled in the art given the disclosures herein. Such modifications are encompassed by the present invention.

The protein may also be produced by operably linking the isolated polynucleotide of the invention to suitable control sequences in one or more insect expression vectors, and employing an insect expression system. Materials and methods for baculovirus/insect cell expression systems are commercially available in kit form from, e.g., Invitrogen, San Diego, Calif., U.S.A. (the MaxBat™ kit), and such methods are well known in the art, as described in Summers and Smith, Texas Agricultural Experiment Station Bulletin No. 1555 (1987), incorporated herein by reference. As used herein, an insect cell capable of expressing a polynucleotide of the present invention is “transformed.”

The protein of the invention may be prepared by culturing transformed host cells under culture conditions suitable to express the recombinant protein. The resulting expressed protein may then be purified from such culture (i.e., from culture medium or cell extracts) using known purification processes, such as gel filtration and ion exchange chromatography. Purification of the protein of the invention may also include an affinity column containing agents which will bind to the protein of the invention; one or more column steps over such affinity resins as concanavalin A-agarose, heparin-toyopearl™ or Cibacrom blue 3GA Sepharose™; one or more steps involving hydrophobic interaction chromatography using such resins as phenyl ether, butyl ether, or propyl ether; or immunoaffinity chromatography.

Alternatively, the protein of the invention may also be expressed in a form which will facilitate purification. For example, it may be expressed as a fusion protein, such as those of maltose binding protein (MBP), glutathione-S-transferase (GST) or thioredoxin (TRX), or as a His tag. Kits for expression and purification of such fusion proteins are commercially available from New England BioLab (Beverly, Mass.), Pharmacia (Piscataway, N.J.) and Invitrogen, respectively. The protein of the invention can also be tagged with an epitope and subsequently purified by using a specific antibody directed to such epitope. One such epitope (“FLAG®”) is commercially available from Kodak (New Haven, Conn.).

Finally, one or more reverse-phase high performance liquid chromatography (RP-HPLC) steps employing hydrophobic RP-HPLC media, e.g., silica gel having pendant methyl or other aliphatic groups, can be employed to further purify the protein of the invention. Some or all of the foregoing purification steps, in various combinations, can also be employed to provide a substantially homogeneous isolated recombinant protein. The protein thus purified is substantially free of other mammalian proteins and is defined in accordance with the present invention as an “isolated protein.”

The polypeptides of the invention include analogs (variants). This embraces fragments of the polypeptides of the invention, as well polypeptides of the invention which comprise one or more amino acids deleted, inserted, or substituted. Also, analogs of the polypeptides of the invention embrace fusions of the polypeptides of the invention or modifications of the polypeptides of the invention, wherein the polypeptide or analog of the invention is fused to another moiety or moieties, e.g., targeting moiety or another therapeutic agent. Such analogs may exhibit improved properties such as activity and/or stability. Examples of moieties which may be fused to the polypeptide or an analog of the invention include, for example, targeting moieties which provide for the delivery of polypeptides of the invention to neurons, e.g., antibodies to central nervous system, or antibodies to receptor and ligands expressed on neuronal cells. Other moieties which may be fused to polypeptides of the invention include therapeutic agents which are used for treatment, for example antidepressant drugs or other medications for neurological disorders. Also, polypeptides of the invention may be fused to neuron growth modulators, and other chemokines for targeted delivery.

4.14.1 Determining Polypeptide and Polynucleotide Identity and Similarity

Preferred identity and/or similarity are designed to give the largest match between the sequences tested. Methods to determine identity and similarity are codified in computer programs including, but are not limited to, the GCG program package, including GAP (Devereux, J., et al., Nucl. Acids Res. 12:387 (1984); Genetics Computer Group, University of Wisconsin, Madison, Wis., herein incorporated by reference), BLASTP, BLASTN, BLASTX, FASTA (Altschul, S. F. et al., J. Molec. Biol. 215:403-410 (1990), PSI-BLAST (Altschul S. F. et al., Nucl. Acids Res. 25:3389-3402, herein incorporated by reference), the eMatrix software (Wu et al., J. Comp. Biol., 6:219-235 (1999), herein incorporated by reference), eMotif software (Nevill-Manning et al, ISMB-97, 4:202-209, herein incorporated by reference), the GeneAtlas software (Molecular Simulations Inc. (MSI), San Diego, Calif.) (Sanchez and Sali, Proc. Natl. Acad. Sci. USA, 95:13597-13602 (1998); Kitson D H, et al, (2000) “Remote homology detection using structural modeling—an evaluation” Submitted; Fischer and Eisenberg, Protein Sci. 5:947-955 (1996)), and the Kyte-Doolittle hydrophobocity prediction algorithm (J. Mol Biol, 157:105-31 (1982), incorporated herein by reference). The BLAST programs are publicly available from the National Center for Biotechnology Information (NCBI) and other sources (BLAST Manual, Altschul, S., et al. NCB NLM NIH Bethesda, Md. 20894; Altschul, S., et al., J. Mol. Biol. 215:403-410 (1990).

4.15 Gene Therapy

Mutations in the gene encoding the polypeptide of the invention may result in loss of normal function of the encoded protein. The invention thus provides gene therapy to restore normal activity of the polypeptides of the invention; or to treat disease states involving polypeptides of the invention. Delivery of a functional gene encoding polypeptides of the invention to appropriate cells is effected ex vivo, in situ, or in vivo by use of vectors, and more particularly viral vectors (e.g., adenovirus, adeno-associated virus, or a retrovirus), or ex vivo by use of physical DNA transfer methods (e.g., liposomes or chemical treatments). See, for example, Anderson, Nature, 392(Suppl.):25-20 (1998). For additional reviews of gene therapy technology see Friedmann, Science, 244:1275-1281 (1989); Verma, Scientific American: 68-84 (1990); and Miller, Nature, 357:455-460 (1992). Introduction of any one of the nucleotides of the present invention or a gene encoding the polypeptides of the present invention can also be accomplished with extrachromosomal substrates (transient expression) or artificial chromosomes (stable expression). Cells may also be cultured ex vivo in the presence of proteins of the present invention in order to proliferate or to produce a desired effect on or activity in such cells. Treated cells can then be introduced in vivo for therapeutic purposes. Alternatively, it is contemplated that in other human disease states, preventing the expression of or inhibiting the activity of polypeptides of the invention will be useful in treating the disease states. It is contemplated that antisense therapy or gene therapy could be applied to negatively regulate the expression of polypeptides of the invention.

Other methods inhibiting expression of a protein include the introduction of antisense molecules to the nucleic acids of the present invention, their complements, or their translated RNA sequences, by methods known in the art. Further, the polypeptides of the present invention can be inhibited by using targeted deletion methods, or the insertion of a negative regulatory element such as a silencer, which is tissue specific.

The present invention still further provides cells genetically engineered in vivo to express the polynucleotides of the invention, wherein such polynucleotides are in operative association with a regulatory sequence heterologous to the host cell which drives expression of the polynucleotides in the cell. These methods can be used to increase or decrease the expression of the polynucleotides of the present invention.

Knowledge of DNA sequences provided by the invention allows for modification of cells to permit, increase, or decrease, expression of endogenous polypeptide. Cells can be modified (e.g., by homologous recombination) to provide increased polypeptide expression by replacing, in whole or in part, the naturally occurring promoter with all or part of a heterologous promoter so that the cells express the protein at higher levels. The heterologous promoter is inserted in such a manner that it is operatively linked to the desired protein encoding sequences. See, for example, PCT International Publication No. WO 94/12650, PCT International Publication No. WO 92/20808, and PCT International Publication No. WO 91/09955. It is also contemplated that, in addition to heterologous promoter DNA, amplifiable marker DNA (e.g., ada, dhfr, and the multifunctional CAD gene which encodes carbamyl phosphate synthase, aspartate transcarbamylase, and dihydroorotase) and/or intron DNA may be inserted along with the heterologous promoter DNA. If linked to the desired protein coding sequence, amplification of the marker DNA by standard selection methods results in co-amplification of the desired protein coding sequences in the cells.

In another embodiment of the present invention, cells and tissues may be engineered to express an endogenous gene comprising the polynucleotides of the invention under the control of inducible regulatory elements, in which case the regulatory sequences of the endogenous gene may be replaced by homologous recombination. As described herein, gene targeting can be used to replace a gene's existing regulatory region with a regulatory sequence isolated from a different gene or a novel regulatory sequence synthesized by genetic engineering methods. Such regulatory sequences may be comprised of promoters, enhancers, scaffold-attachment regions, negative regulatory elements, transcriptional initiation sites, regulatory protein binding sites or combinations of said sequences. Alternatively, sequences which affect the structure or stability of the RNA or protein produced may be replaced, removed, added, or otherwise modified by targeting. These sequences include polyadenylation signals, mRNA stability elements, splice sites, leader sequences for enhancing or modifying transport or secretion properties of the protein, or other sequences which alter or improve the function or stability of protein or RNA molecules.

The targeting event may be a simple insertion of the regulatory sequence, placing the gene under the control of the new regulatory sequence, e.g., inserting a new promoter or enhancer or both upstream of a gene. Alternatively, the targeting event may be a simple deletion of a regulatory element, such as the deletion of a tissue-specific negative regulatory element. Alternatively, the targeting event may replace an existing element; for example, a tissue-specific enhancer can be replaced by an enhancer that has broader or different cell-type specificity than the naturally occurring elements. Here, the naturally occurring sequences are deleted and new sequences are added. In all cases, the identification of the targeting event may be facilitated by the use of one or more selectable marker genes that are contiguous with the targeting DNA, allowing for the selection of cells in which the exogenous DNA has integrated into the cell genome. The identification of the targeting event may also be facilitated by the use of one or more marker genes exhibiting the property of negative selection, such that the negatively selectable marker is linked to the exogenous DNA, but configured such that the negatively selectable marker flanks the targeting sequence, and such that a correct homologous recombination event with sequences in the host cell genome does not result in the stable integration of the negatively selectable marker. Markers useful for this purpose include the Herpes Simplex Virus thymidine kinase (TK) gene or the bacterial xanthine-guanine phosphoribosyl-transferase (gpt) gene.

The gene targeting or gene activation techniques which can be used in accordance with this aspect of the invention are more particularly described in U.S. Pat. No. 5,272,071 to Chappel; U.S. Pat. No. 5,578,461 to Sherwin et al.; International Application No. PCT/US92/09627 (WO93/09222) by Selden et al.; and International Application No. PCT/US90/06436 (WO91/06667) by Skoultchi et al., each of which is incorporated by reference herein in its entirety.

4.16 Transgenic Animals

In preferred methods to determine biological functions of the polypeptides of the invention in vivo, one or more genes provided by the invention are either over expressed or inactivated in the germ line of animals using homologous recombination (Capecchi, Science 244:1288-1292 (1989)). Animals in which the gene is over expressed, under the regulatory control of exogenous or endogenous promoter elements, are known as transgenic animals. Animals in which an endogenous gene has been inactivated by homologous recombination are referred to as “knockout” animals. Knockout animals, preferably non-human mammals, can be prepared as described in U.S. Pat. No. 5,557,032, incorporated herein by reference. Transgenic animals are useful to determine the roles polypeptides of the invention play in biological processes, and preferably in disease states. Transgenic animals are useful as model systems to identify compounds that modulate lipid metabolism. Transgenic animals, preferably non-human mammals, are produced using methods as described in U.S. Pat. No. 5,489,743 and PCT Publication No. WO94/28122, incorporated herein by reference.

Transgenic animals can be prepared wherein all or part of a promoter of the polynucleotides of the invention is either activated or inactivated to alter the level of expression of the polypeptides of the invention. Inactivation can be carried out using homologous recombination methods described above. Activation can be achieved by supplementing or even replacing the homologous promoter to provide for increased protein expression. The homologous promoter can be supplemented by insertion of one or more heterologous enhancer elements known to confer promoter activation in a particular tissue.

The polynucleotides of the present invention also make possible the development, through, e.g., homologous recombination or knock out strategies, of animals that fail to express functional polypeptides of the invention or that express a variant of the polypeptides of the invention. Such animals are useful as models for studying the in vivo activities of polypeptides of the invention as well as for studying modulators of the polypeptides of the invention.

4.17 Uses and Biological Activity

The polynucleotides and proteins of the present invention are expected to exhibit one or more of the uses or biological activities (including those associated with assays cited herein) identified herein. Uses or activities described for proteins of the present invention may be provided by administration or use of such proteins or of polynucleotides encoding such proteins (such as, for example, in gene therapies or vectors suitable for introduction of DNA). The mechanism underlying the particular condition or pathology will dictate whether the polypeptides of the invention, the polynucleotides of the invention or modulators (activators or inhibitors) thereof would be beneficial to the subject in need of treatment. Thus, “therapeutic compositions of the invention” include compositions comprising isolated polynucleotides (including recombinant DNA molecules, cloned genes and degenerate variants thereof) or polypeptides of the invention (including full length protein, mature protein and truncations or domains thereof), or compounds and other substances that modulate the overall activity of the target gene products, either at the level of target gene/protein expression or target protein activity. Such modulators include polypeptides, analogs, (variants), including fragments and fusion proteins, antibodies and other binding proteins; chemical compounds that directly or indirectly activate or inhibit the polypeptides of the invention (identified, e.g., via drug screening assays as described herein); antisense polynucleotides and polynucleotides suitable for triple helix formation; and in particular antibodies or other binding partners that specifically recognize one or more epitopes of the polypeptides of the invention.

The polypeptides of the present invention may likewise be involved in cellular activation or in one of the other physiological pathways described herein.

4.17.1 Research Uses and Utilities

The polynucleotides provided by the present invention can be used by the research community for various purposes. The polynucleotides can be used to express recombinant protein for analysis, characterization or therapeutic use; as markers for tissues in which the corresponding protein is preferentially expressed (either constitutively or at a particular stage of tissue differentiation or development or in disease states); as molecular weight markers on gels; as chromosome markers or tags (when labeled) to identify chromosomes or to map related gene positions; to compare with endogenous DNA sequences in patients to identify potential genetic disorders; as probes to hybridize and thus discover novel, related DNA sequences; as a source of information to derive PCR primers for genetic fingerprinting; as a probe to “subtract-out” known sequences in the process of discovering other novel polynucleotides; for selecting and making oligomers for attachment to a “gene chip” or other support, including for examination of expression patterns; to raise anti-protein antibodies using DNA immunization techniques; and as an antigen to raise anti-DNA antibodies or elicit another immune response. Where the polynucleotide encodes a protein which binds or potentially binds to another protein (such as, for example, in a receptor-ligand interaction), the polynucleotide can also be used in interaction trap assays (such as, for example, that described in Gyuris et al., Cell 75:791-803 (1993)) to identify polynucleotides encoding the other protein with which binding occurs or to identify inhibitors of the binding interaction.

The polypeptides provided by the present invention can similarly be used in assays to determine biological activity, including in a panel of multiple proteins for high-throughput screening; to raise antibodies or to elicit another immune response; as a reagent (including the labeled reagent) in assays designed to quantitatively determine levels of the protein (or its receptor) in biological fluids; as markers for tissues in which the corresponding polypeptide is preferentially expressed (either constitutively or at a particular stage of tissue differentiation or development or in a disease state); and, of course, to isolate correlative receptors or ligands. Proteins involved in these binding interactions can also be used to screen for peptide or small molecule inhibitors or agonists of the binding interaction.

The polypeptides of the invention are also useful for making antibody substances that are specifically immunoreactive with proteins according to the invention. Antibodies and portions thereof (e.g., Fab fragments) which bind to the polypeptides of the invention can be used to identify the presence of such polypeptides in a sample. Such determinations are carried out using any suitable immunoassay format, and any polypeptide of the invention that is specifically bound by the antibody can be employed as a positive control.

Any or all of these research utilities are capable of being developed into reagent grade or kit format for commercialization as research products.

Methods for performing the uses listed above are well known to those skilled in the art. References disclosing such methods include without limitation “Molecular Cloning: A Laboratory Manual”, 2d ed., Cold Spring Harbor Laboratory Press, Sambrook, J., E. F. Fritsch and T. Maniatis eds., 1989, and “Methods in Enzymology: Guide to Molecular Cloning Techniques”, Academic Press, Berger, S. L. and A. R. Kimmel eds., 1987.

4.17.2 Cytokine and Cell Proliferation/Differentiation Activity

A polypeptide of the present invention may exhibit activity relating to cytokine, cell proliferation (either inducing or inhibiting) or cell differentiation (either inducing or inhibiting) activity or may induce production of other cytokines in certain cell populations. A polynucleotide of the invention can encode a polypeptide exhibiting such attributes. Many protein factors discovered to date, including all known cytokines, have exhibited activity in one or more factor-dependent cell proliferation assays, and hence the assays serve as a convenient confirmation of cytokine activity. The activity of therapeutic compositions of the present invention is evidenced by any one of a number of routine factor dependent cell proliferation assays for cell lines including, without limitation, 32D, DA2, DA1G, T10, B9, B9/11, BaF3, MC9/G, M+(preB M+), 2E8, RB5, DA1, 123, T1165, HT2, CTLL2, TF-1, Mo7e, CMK, HUVEC, and Caco. Therapeutic compositions of the invention can be used in the following:

Assays for T-cell or thymocyte proliferation include without limitation those described in: Current Protocols in Immunology, Ed by J. E. Coligan, A. M. Kruisbeek, D. H. Margulies, E. M. Shevach, W. Strober, Pub. Greene Publishing Associates and Wiley-Interscience (Chapter 3, In Vitro assays for Mouse Lymphocyte Function 3.1-3.19; Chapter 7, Immunologic studies in Humans); Takai, et al., J. Immunol. 137:3494-3500 (1986); Bertagnolli, et al., J. Immunol. 145:1706-1712 (1990); Bertagnolli, et al., Cellular Immunology 133:327-341 (1991); Bertagnolli, et al., J. Immunol. 149:3778-3783 (1992); Bowman, et al., J. Immunol. 152:1756-1761 (1994).

Assays for cytokine production and/or proliferation of spleen cells, lymph node cells or thymocytes include, without limitation, those described in: Polyclonal T cell stimulation, Kruisbeek, A. M. and Shevach, E. M. In Current Protocols in Immunology. J. E. e.a. Coligan eds. Vol 1 pp. 3.12.1-3.12.14, John Wiley and Sons, Toronto. 1994; and Measurement of mouse and human interferon-γ, Schreiber, R. D. In Current Protocols in Immunology. J. E. e.a. Coligan eds. Vol 1 pp. 6.8.1-6.8.8, John Wiley and Sons, Toronto. 1994.

Assays for proliferation and differentiation of hematopoietic and lymphopoietic cells include, without limitation, those described in: Measurement of Human and Murine Interleukin 2 and Interleukin 4, Bottomly, K., Davis, L. S. and Lipsky, P. E. In Current Protocols in Immunology. J. E. e.a. Coligan eds. Vol 1 pp. 6.3.1-6.3.12, John Wiley and Sons, Toronto. 1991; deVries, et al., J. Exp. Med. 173:1205-1211 (1991); Moreau, et al., Nature 336:690-692 (1988); Greenberger, et al., Proc. Natl. Acad. Sci. U.S.A. 80:2931-2938 (1983); Measurement of mouse and human interleukin 6—Nordan, R. In Current Protocols in Immunology. J. E. Coligan eds. Vol 1 pp. 6.6.1-6.6.5, John Wiley and Sons, Toronto. 1991; Smith, et al., Proc. Natl. Aced. Sci. U.S.A. 83:1857-1861 (1986); Measurement of human Interleukin 11—Bennett, F., Giannotti, J., Clark, S. C. and Turner, K. J. In Current Protocols in Immunology. J. E. Coligan eds. Vol 1 pp. 6.15.1 John Wiley and Sons, Toronto. 1991; Measurement of mouse and human Interleukin 9-Ciarletta, A., Giannotti, J., Clark, S. C. and Turner, K. J. In Current Protocols in Immunology. J. E. Coligan eds. Vol 1 pp. 6.13.1, John Wiley and Sons, Toronto. 1991.

Assays for T-cell clone responses to antigens (which will identify, among others, proteins that affect APC-T cell interactions as well as direct T-cell effects by measuring proliferation and cytokine production) include, without limitation, those described in: Current Protocols in Immunology, Ed by J. E. Coligan, A. M. Kruisbeek, D. H. Margulies, E. M. Shevach, W Strober, Pub. Greene Publishing Associates and Wiley-Interscience (Chapter 3, In Vitro assays for Mouse Lymphocyte Function; Chapter 6, Cytokines and their cellular receptors; Chapter 7, Immunologic studies in Humans); Weinberger, et al., Proc. Natl. Acad. Sci. USA 77:6091-6095 (1980); Weinberger, et al., Eur. J. Immun. 11:405-411 (1981); Takai, et al., J. Immunol. 137:3494-3500 (1986); Takai, et al., J. Immunol. 140:508-512 (1988).

4.17.3 Stem Cell Growth Factor Activity

A polypeptide of the present invention may exhibit stem cell growth factor activity and be involved in the proliferation, differentiation and survival of pluripotent and totipotent stem cells including primordial germ cells, embryonic stem cells, hematopoietic stem cells and/or germ line stem cells. Administration of the polypeptide of the invention to stem cells in vivo or ex vivo may maintain and expand cell populations in a totipotential or pluripotential state which would be useful for re-engineering damaged or diseased tissues, transplantation, and manufacture of bio-pharmaceuticals and the development of bio-sensors. The ability to produce large quantities of human cells has important working applications for the production of human proteins which currently must be obtained from non-human sources or donors, implantation of cells to treat diseases such as Parkinson's, Alzheimer's and other neurodegenerative diseases; tissues for grafting such as bone marrow, skin, cartilage, tendons, bone, muscle (including cardiac muscle), blood vessels, cornea, neural cells, gastrointestinal cells and others; and organs for transplantation such as kidney, liver, pancreas (including islet cells), heart and lung.

It is contemplated that multiple different exogenous growth factors and/or cytokines may be administered in combination with the polypeptide of the invention to achieve the desired effect, including any of the growth factors listed herein, other stem cell maintenance factors, and specifically including stem cell factor (SCF), leukemia inhibitory factor (LIF), Flt-3 ligand (Flt-3L), any of the interleukins, recombinant soluble IL-6 receptor fused to IL-6, macrophage inflammatory protein 1-alpha (MIP-1-alpha), G-CSF, GM-CSF, thrombopoietin (TPO), platelet factor 4 (PF-4), platelet-derived growth factor (PDGF), neural growth factors and basic fibroblast growth factor (bFGF).

Since totipotent stem cells can give rise to virtually any mature cell type, expansion of these cells in culture will facilitate the production of large quantities of mature cells. Techniques for culturing stem cells are known in the art and administration of polypeptides of the invention, optionally with other growth factors and/or cytokines, is expected to enhance the survival and proliferation of the stem cell populations. This can be accomplished by direct administration of the polypeptide of the invention to the culture medium. Alternatively, stroma cells transfected with a polynucleotide that encodes for the polypeptide of the invention can be used as a feeder layer for the stem cell populations in culture or in vivo. Stromal support cells for feeder layers may include embryonic bone marrow fibroblasts, bone marrow stromal cells, fetal liver cells, or cultured embryonic fibroblasts (see U.S. Pat. No. 5,690,926).

Stem cells themselves can be transfected with a polynucleotide of the invention to induce autocrine expression of the polypeptide of the invention. This will allow for generation of undifferentiated totipotential/pluripotential stem cell lines that are useful as is or that can then be differentiated into the desired mature cell types. These stable cell lines can also serve as a source of undifferentiated totipotential/pluripotential mRNA to create cDNA libraries and templates for polymerase chain reaction experiments. These studies would allow for the isolation and identification of differentially expressed genes in stem cell populations that regulate stem cell proliferation and/or maintenance.

Expansion and maintenance of totipotent stem cell populations will be useful in the treatment of many pathological conditions. For example, polypeptides of the present invention may be used to manipulate stem cells in culture to give rise to neuroepithelial cells that can be used to augment or replace cells damaged by illness, autoimmune disease, accidental damage or genetic disorders. The polypeptide of the invention may be useful for inducing the proliferation of neural cells and for the regeneration of nerve and brain tissue, i.e. for the treatment of central and peripheral nervous system diseases and neuropathies, as well as mechanical and traumatic disorders which involve degeneration, death or trauma to neural cells or nerve tissue. Furthermore, these cells can be cultured in vitro to form other differentiated cells, such as skin tissue that can be used for transplantation. In addition, the expanded stem cell populations can also be genetically altered for gene therapy purposes and to decrease host rejection of replacement tissues after grafting or implantation.

Expression of the polypeptide of the invention and its effect on stem cells can also be manipulated to achieve controlled differentiation of the stem cells into more differentiated cell types. A broadly applicable method of obtaining pure populations of a specific differentiated cell type from undifferentiated stem cell populations involves the use of a cell-type specific promoter driving a selectable marker. The selectable marker allows only cells of the desired type to survive. For example, stem cells can be induced to differentiate into cardiomyocytes (Wobus et al., Differentiation, 48:173-182 (1991); Klug, et al., J. Clin. Invest., 98:216-224 (1998)) or skeletal muscle cells (Browder, L. W. In: Principles of Tissue Engineering eds. Lanza, et al., Academic Press (1997)). Alternatively, directed differentiation of stem cells can be accomplished by culturing the stem cells in the presence of a differentiation factor such as retinoic acid and an antagonist of the polypeptide of the invention which would inhibit the effects of endogenous stem cell factor activity and allow differentiation to proceed.

In vitro cultures of stem cells can be used to determine if the polypeptide of the invention exhibits stem cell growth factor activity. Stem cells are isolated from any one of various cell sources (including hematopoietic stem cells and embryonic stem cells) and cultured on a feeder layer, as described by Thompson, et al. Proc. Natl. Acad. Sci, U.S.A., 92:7844-7848 (1995), in the presence of the polypeptide of the invention alone or in combination with other growth factors or cytokines. The ability of the polypeptide of the invention to induce stem cells proliferation is determined by colony formation on semi-solid support e.g. as described by Bernstein, et al., Blood, 77: 2316-2321 (1991).

4.17.4 Hematopoiesis Regulating Activity

A polypeptide of the present invention may be involved in regulation of hematopoiesis and, consequently, in the treatment of myeloid or lymphoid cell disorders. Even marginal biological activity in support of colony forming cells or of factor-dependent cell lines indicates involvement in regulating hematopoiesis, e.g. in supporting the growth and proliferation of erythroid progenitor cells alone or in combination with other cytokines, thereby indicating utility, for example, in treating various anemias or for use in conjunction with irradiation/chemotherapy to stimulate the production of erythroid precursors and/or erythroid cells; in supporting the growth and proliferation of myeloid cells such as granulocytes and monocytes/macrophages (i.e., traditional colony stimulating factor activity) useful, for example, in conjunction with chemotherapy to prevent or treat consequent myelo-suppression; in supporting the growth and proliferation of megakaryocytes and consequently of platelets thereby allowing prevention or treatment of various platelet disorders such as thrombocytopenia, and generally for use in place of or complimentary to platelet transfusions; and/or in supporting the growth and proliferation of hematopoietic stem cells which are capable of maturing to any and all of the above-mentioned hematopoietic cells and therefore find therapeutic utility in various stem cell disorders (such as those usually treated with transplantation, including, without limitation, aplastic anemia and paroxysmal nocturnal hemoglobinuria), as well as in repopulating the stem cell compartment post irradiation/chemotherapy, either in vivo or ex vivo (i.e., in conjunction with bone marrow transplantation or with peripheral progenitor cell transplantation (homologous or heterologous)) as normal cells or genetically manipulated for gene therapy.

Therapeutic compositions of the invention can be used in the following:

Suitable assays for proliferation and differentiation of various hematopoietic lines are cited above.

Assays for embryonic stem cell differentiation (which will identify, among others, proteins that influence embryonic differentiation hematopoiesis) include, without limitation, those described in: Johansson, et al. Cellular Biology 15:141-15 (1995); Keller, et al., Mol. Cell. Biol. 13:473486 (1993); McClanahan, et al., Blood 81:2903-2915 (1993).

Assays for stem cell survival and differentiation (which will identify, among others, proteins that regulate lympho-hematopoiesis) include, without limitation, those described in: Methylcellulose colony forming assays, Freshney, M. G. In Culture of Hematopoietic Cells. R. I. Freshney, et al. eds. Vol pp. 265-268, Wiley-Liss, Inc., New York, N.Y. 1994; Hirayama, et al., Proc. Natl. Acad. Sci. USA 89:5907-5911 (1992); Primitive hematopoietic colony forming cells with high proliferative potential, McNiece, I. K. and Briddell, R. A. In Culture of Hematopoietic Cells. R. I. Freshney, et al. eds. Vol pp. 23-39, Wiley-Liss, Inc., New York, N.Y. 1994; Neben, et al., Experimental Hematology 22:353-359 (1994); Cobblestone area forming cell assay, Ploemacher, R. E. In Culture of Hematopoietic Cells. R. I. Freshney, et al. eds. Vol pp. 1-21, Wiley-Liss, Inc., New York, N.Y. 1994; Long term bone marrow cultures in the presence of stromal cells, Spooncer, E., Dexter, M. and Allen, T. In Culture of Hematopoietic Cells. R. I. Freshney, et al. eds. Vol pp. 163-179, Wiley-Liss, Inc., New York, N.Y. 1994; Long term culture initiating cell assay, Sutherland, H. J. In Culture of Hematopoietic Cells. R. I. Freshney, et al. eds. Vol pp. 139-162, Wiley-Liss, Inc., New York, N.Y. 1994.

4.17.5 Tissue Growth Activity

A polypeptide of the present invention also may be involved in bone, cartilage, tendon, ligament and/or nerve tissue growth or regeneration, as well as in wound healing and tissue repair and replacement, and in healing of burns, incisions and ulcers.

A polypeptide of the present invention which induces cartilage and/or bone growth in circumstances where bone is not normally formed has application in the healing of bone fractures and cartilage damage or defects in humans and other animals. Compositions of a polypeptide, antibody, binding partner, or other modulator of the invention may have prophylactic use in closed as well as open fracture reduction and also in the improved fixation of artificial joints. De novo bone formation induced by an osteogenic agent contributes to the repair of congenital, trauma induced, or oncologic resection induced craniofacial defects, and also is useful in cosmetic plastic surgery.

A polypeptide of this invention may also be involved in attracting bone-forming cells, stimulating growth of bone-forming cells, or inducing differentiation of progenitors of bone-forming cells. Treatment of osteoporosis, osteoarthritis, bone degenerative disorders, or periodontal disease, such as through stimulation of bone and/or cartilage repair or by blocking inflammation or processes of tissue destruction (collagenase activity, osteoclast activity, etc.) mediated by inflammatory processes may also be possible using the composition of the invention.

Another category of tissue regeneration activity that may involve the polypeptide of the present invention is tendon/ligament formation. Induction of tendon/ligament-like tissue or other tissue formation in circumstances where such tissue is not normally formed has application in the healing of tendon or ligament tears, deformities and other tendon or ligament defects in humans and other animals. Such a preparation employing a tendon/ligament-like tissue inducing protein may have prophylactic use in preventing damage to tendon or ligament tissue, as well as use in the improved fixation of tendon or ligament to bone or other tissues, and in repairing defects to tendon or ligament tissue. De novo tendon/ligament-like tissue formation induced by a composition of the present invention contributes to the repair of congenital, trauma induced, or other tendon or ligament defects of other origin, and is also useful in cosmetic plastic surgery for attachment or repair of tendons or ligaments. The compositions of the present invention may provide environment to attract tendon- or ligament-forming cells, stimulate growth of tendon- or ligament-forming cells, induce differentiation of progenitors of tendon- or ligament-forming cells, or induce growth of tendon/ligament cells or progenitors ex vivo for return in vivo to effect tissue repair. The compositions of the invention may also be useful in the treatment of tendinitis, carpal tunnel syndrome and other tendon or ligament defects. The compositions may also include an appropriate matrix and/or sequestering agent as a carrier as is well known in the art.

The compositions of the present invention may also be useful for proliferation of neural cells and for regeneration of nerve and brain tissue, i.e. for the treatment of central and peripheral nervous system diseases and neuropathies, as well as mechanical and traumatic disorders, which involve degeneration, death or trauma to neural cells or nerve tissue. More specifically, a composition of the invention may be used in the treatment of diseases of the peripheral nervous system, such as peripheral nerve injuries, peripheral neuropathy and localized neuropathies, and central nervous system diseases, such as Alzheimer's, Parkinson's disease, Huntington's disease, amyotrophic lateral sclerosis, and Shy-Drager syndrome. Further conditions which may be treated in accordance with the present invention include mechanical and traumatic disorders, such as spinal cord disorders, head trauma and cerebrovascular diseases such as stroke. Peripheral neuropathies resulting from chemotherapy or other medical therapies may also be treatable using a composition of the invention.

Compositions of the invention may also be useful to promote better or faster closure of non-healing wounds, including without limitation pressure ulcers, ulcers associated with vascular insufficiency, surgical and traumatic wounds, and the like.

Compositions of the present invention may also be involved in the generation or regeneration of other tissues, such as organs (including, for example, pancreas, liver, intestine, kidney, skin, and endothelium), muscle (smooth, skeletal or cardiac) and vascular (including vascular endothelium) tissue, or for promoting the growth of cells comprising such tissues. Part of the desired effects may be by inhibition or modulation of fibrotic scarring may allow normal tissue to regenerate. A polypeptide of the present invention may also exhibit angiogenic activity.

A composition of the present invention may also be useful for gut protection or regeneration and treatment of lung or liver fibrosis, reperfusion injury in various tissues, and conditions resulting from systemic cytokine damage.

A composition of the present invention may also be useful for promoting or inhibiting differentiation of tissues described above from precursor tissues or cells; or for inhibiting the growth of tissues described above.

Therapeutic compositions of the invention can be used in the following:

Assays for tissue generation activity include, without limitation, those described in: International Patent Publication No. WO95/16035 (bone, cartilage, tendon); International Patent Publication No. WO95/05846 (nerve, neuronal); International Patent Publication No. WO91/07491 (skin, endothelium).

Assays for wound healing activity include, without limitation, those described in: Winter, Epidermal Wound Healing, pp. 71-112 (Maibach, H. I. and Rovee, D. T., eds.), Year Book Medical Publishers, Inc., Chicago, as modified by Eaglstein and Mertz, J. Invest. Dermatol 71:382-84 (1978).

4.17.6 Immune Function Stimulating or Suppressing Activity

A polypeptide of the present invention may also exhibit immune stimulating or immune suppressing activity, including without limitation the activities for which assays are described herein. A polynucleotide of the invention can encode a polypeptide exhibiting such activities. A protein may be useful in the treatment of various immune deficiencies and disorders (including severe combined immunodeficiency (SCID)), e.g., in regulating (up or down) growth and proliferation of T and/or B lymphocytes, as well as effecting the cytolytic activity of NK cells and other cell populations. These immune deficiencies may be genetic or be caused by viral (e.g., HIV) as well as bacterial or fungal infections, or may result from autoimmune disorders. More specifically, infectious diseases causes by viral, bacterial, fungal or other infection may be treatable using a protein of the present invention, including infections by HIV, hepatitis viruses, herpes viruses, mycobacteria, Leishmania spp., malaria spp. and various fungal infections such as candidiasis. Of course, in this regard, proteins of the present invention may also be useful where a boost to the immune system generally may be desirable, i.e., in the treatment of cancer.

Autoimmune disorders which may be treated using a protein of the present invention include, for example, connective tissue disease, multiple sclerosis, systemic lupus erythematosus, rheumatoid arthritis, autoimmune pulmonary inflammation, Guillain-Barre syndrome, autoimmune thyroiditis, insulin dependent diabetes mellitis, myasthenia gravis, graft-versus-host disease and autoimmune inflammatory eye disease. Such a protein (or antagonists thereof, including antibodies) of the present invention may also to be useful in the treatment of allergic reactions and conditions (e.g., anaphylaxis, serum sickness, drug reactions, food allergies, insect venom allergies, mastocytosis, allergic rhinitis, hypersensitivity pneumonitis, urticaria, angioedema, eczema, atopic dermatitis, allergic contact dermatitis, erythema multiforme, Stevens-Johnson syndrome, allergic conjunctivitis, atopic keratoconjunctivitis, venereal keratoconjunctivitis, giant papillary conjunctivitis and contact allergies), such as asthma (particularly allergic asthma) or other respiratory problems. Other conditions, in which immune suppression is desired (including, for example, organ transplantation), may also be treatable using a protein (or antagonists thereof) of the present invention. The therapeutic effects of the polypeptides or antagonists thereof on allergic reactions can be evaluated by in vivo animals models such as the cumulative contact enhancement test (Lastbom, et al., Toxicology 125: 59-66 (1998)), skin prick test (Hoffmann, et al., Allergy 54: 446-54 (1999)), guinea pig skin sensitization test (Vohr, et al., Arch. Toxocol. 73: 501-9), and murine local lymph node assay (Kimber, et al., J. Toxicol. Environ. Health 53: 563-79).

Using the proteins of the invention it may also be possible to modulate immune responses, in a number of ways. Down regulation may be in the form of inhibiting or blocking an immune response already in progress or may involve preventing the induction of an immune response. The functions of activated T cells may be inhibited by suppressing T cell responses or by inducing specific tolerance in T cells, or both. Immunosuppression of T cell responses is generally an active, non-antigen-specific, process which requires continuous exposure of the T cells to the suppressive agent. Tolerance, which involves inducing non-responsiveness or anergy in T cells, is distinguishable from immunosuppression in that it is generally antigen-specific and persists after exposure to the tolerizing agent has ceased. Operationally, tolerance can be demonstrated by the lack of a T cell response upon reexposure to specific antigen in the absence of the tolerizing agent.

Down regulating or preventing one or more antigen functions (including without limitation B lymphocyte antigen functions (such as, for example, B7)), e.g., preventing high level lymphokine synthesis by activated T cells, will be useful in situations of tissue, skin and organ transplantation and in graft-versus-host disease (GVHD). For example, blockage of T cell function should result in reduced tissue destruction in tissue transplantation. Typically, in tissue transplants, rejection of the transplant is initiated through its recognition as foreign by T cells, followed by an immune reaction that destroys the transplant. The administration of a therapeutic composition of the invention may prevent cytokine synthesis by immune cells, such as T cells, and thus acts as an immunosuppressant. Moreover, a lack of costimulation may also be sufficient to anergize the T cells, thereby inducing tolerance in a subject. Induction of long-term tolerance by B lymphocyte antigen-blocking reagents may avoid the necessity of repeated administration of these blocking reagents. To achieve sufficient immunosuppression or tolerance in a subject, it may also be necessary to block the function of a combination of B lymphocyte antigens.

The efficacy of particular therapeutic compositions in preventing organ transplant rejection or GVHD can be assessed using animal models that are predictive of efficacy in humans. Examples of appropriate systems which can be used include allogeneic cardiac grafts in rats and xenogeneic pancreatic islet cell grafts in mice, both of which have been used to examine the immunosuppressive effects of CTLA4Ig fusion proteins in vivo as described in Lenschow, et al., Science 257:789-792 (1992) and Turka, et al., Proc. Natl. Acad. Sci USA, 89:11102-11105 (1992). In addition, murine models of GVHD (see Paul ed., Fundamental Immunology, Raven Press, New York, 1989, pp. 846-847) can be used to determine the effect of therapeutic compositions of the invention on the development of that disease.

Blocking antigen function may also be therapeutically useful for treating autoimmune diseases. Many autoimmune disorders are the result of inappropriate activation of T cells that are reactive against self tissue and which promote the production of cytokines and autoantibodies involved in the pathology of the diseases. Preventing the activation of autoreactive T cells may reduce or eliminate disease symptoms. Administration of reagents which block stimulation of T cells can be used to inhibit T cell activation and prevent production of autoantibodies or T cell-derived cytokines which may be involved in the disease process. Additionally, blocking reagents may induce antigen-specific tolerance of autoreactive T cells which could lead to long-term relief from the disease. The efficacy of blocking reagents in preventing or alleviating autoimmune disorders can be determined using a number of well-characterized animal models of human autoimmune diseases. Examples include murine experimental autoimmune encephalitis, systemic lupus erythematosus in MRL/lpr/lpr mice or NZB hybrid mice, murine autoimmune collagen arthritis, diabetes mellitus in NOD mice and BB rats, and murine experimental myasthenia gravis (see Paul ed., Fundamental Immunology, Raven Press, New York, 1989, pp. 840-856).

Upregulation of an antigen function (e.g., a B lymphocyte antigen function), as a means of up regulating immune responses, may also be useful in therapy. Upregulation of immune responses may be in the form of enhancing an existing immune response or eliciting an initial immune response. For example, enhancing an immune response may be useful in cases of viral infection, including systemic viral diseases such as influenza, the common cold, and encephalitis.

Alternatively, anti-viral immune responses may be enhanced in an infected patient by removing T cells from the patient, costimulating the T cells in vitro with viral antigen-pulsed APCs either expressing a peptide of the present invention or together with a stimulatory form of a soluble peptide of the present invention and reintroducing the in vitro activated T cells into the patient. Another method of enhancing anti-viral immune responses would be to isolate infected cells from a patient, transfect them with a nucleic acid encoding a protein of the present invention as described herein such that the cells express all or a portion of the protein on their surface, and reintroduce the transfected cells into the patient. The infected cells would now be capable of delivering a costimulatory signal to, and thereby activate, T cells in vivo.

A polypeptide of the present invention may provide the necessary stimulation signal to T cells to induce a T cell mediated immune response against the transfected tumor cells. In addition, tumor cells which lack MHC class I or MHC class II molecules, or which fail to reexpress sufficient mounts of MHC class I or MHC class II molecules, can be transfected with nucleic acid encoding all or a portion of (e.g., a cytoplasmic-domain truncated portion) of an MHC class 1 alpha chain protein and P2 microglobulin protein or an MHC class II alpha chain protein and an MHC class II beta chain protein to thereby express MHC class I or MHC class II proteins on the cell surface. Expression of the appropriate class I or class II MHC in conjunction with a peptide having the activity of a B lymphocyte antigen (e.g., B7-1, B7-2, B7-3) induces a T cell mediated immune response against the transfected tumor cell. Optionally, a gene encoding an antisense construct which blocks expression of an MHC class II associated protein, such as the invariant chain, can also be cotransfected with a DNA encoding a peptide having the activity of a B lymphocyte antigen to promote presentation of tumor associated antigens and induce tumor specific immunity. Thus, the induction of a T cell mediated immune response in a human subject may be sufficient to overcome tumor-specific tolerance in the subject.

The activity of a protein of the invention may, among other means, be measured by the following methods:

Suitable assays for thymocyte or splenocyte cytotoxicity include, without limitation, those described in: Current Protocols in Immunology, Ed by J. E. Coligan, A. M. Kruisbeek, D. H. Margulies, E. M. Shevach, W. Strober, Pub. Greene Publishing Associates and Wiley-Interscience (Chapter 3, In Vitro assays for Mouse Lymphocyte Function 3.1-3.19; Chapter 7, Immunologic studies in Humans); Herrmann, et al., Proc. Natl. Acad. Sci. USA 78:2488-2492 (1981); Herrmann, et al., J. Immunol. 128:1968-1974 (1982); Handa, et al., J. Immunol. 135:1564-1572 (1985); Takai, et al., I. Immunol. 137:3494-3500 (1986); Takai, et al., J. Immunol. 140:508-512 (1988); Bowman, et al., J. Virology 61:1992-1998; Bertagnolli, et al., Cellular Immunology 133:327-341 (1991); Brown, et al., J. Immunol. 153:3079-3092 (1994).

Assays for T-cell-dependent immunoglobulin responses and isotype switching (which will identify, among others, proteins that modulate T-cell dependent antibody responses and that affect Th1/Th2 profiles) include, without limitation, those described in: Maliszewski, J. Immunol. 144:3028-3033 (1990); and Assays for B cell function: In vitro antibody production, Mond, J. J. and Brunswick, M. In Current Protocols in Immunology. J. E. e.a. Coligan eds. Vol 1 pp. 3.8.1-3.8.16, John Wiley and Sons, Toronto. 1994.

Mixed lymphocyte reaction (MLR) assays (which will identify, among others, proteins that generate predominantly Th1 and CTL responses) include, without limitation, those described in: Current Protocols in Immunology, Ed by J. E. Coligan, A. M. Kruisbeek, D. H. Margulies, E. M. Shevach, W. Strober, Pub. Greene Publishing Associates and Wiley-Interscience (Chapter 3, In Vitro assays for Mouse Lymphocyte Function 3.1-3.19; Chapter 7, Immunologic studies in Humans); Takai, et al., J. Immunol. 137:3494-3500 (1986); Takai, et al., J. Immunol. 140:508-512 (1988); Bertagnolli, et al., J. Immunol. 149:3778-3783 (1992).

Dendritic cell-dependent assays (which will identify, among others, proteins expressed by dendritic cells that activate naive T-cells) include, without limitation, those described in: Guery et al., J. Immunol. 134:536-544 (1995); Inaba et al., J. Exp. Med. 173:549-559 (1991); Macatonia, et al., J. Immunol. 154:5071-5079 (1995); Porgador, et al., J. Exp. Med. 182:255-260 (1995); Nair, et al., J Virology 67:4062-4069 (1993); Huang, et al., Science 264:961-965 (1994); Macatonia, et al., J. Exp. Med. 169:1255-1264 (1989); Bhardwaj, et al., J. Clin. Invest. 94:797-807 (1994); and Inaba, et al., J. Exp. Med. 172:631-640 (1990).

Assays for lymphocyte survival/apoptosis (which will identify, among others, proteins that prevent apoptosis after superantigen induction and proteins that regulate lymphocyte homeostasis) include, without limitation, those described in: Darzynkiewicz et al., Cytometry 13:795-808 (1992); Gorczyca, et al., Leukemia 7:659-670 (1993); Gorczyca, et al., Cancer Res. 53:1945-1951 (1993); Itoh, et al., Cell 66:233-243 (1991); Zacharchuk, J. Immunol. 145:4037-4045 (1990); Zamai, et al., Cytometry 14:891-897 (1993); Gorczyca, et al., Int. J. Oncol. 1:639-648 (1992).

Assays for proteins that influence early steps of T-cell commitment and development include, without limitation, those described in: Antica, et al., Blood 84:111-117 (1994); Fine, et al., Cell. Immunol. 155:111-122, (1994); Galy, et al., Blood 85:2770-2778 (1995); Toki, et al., Proc. Nat. Acad. Sci. USA 88:7548-7551 (1991).

4.17.7 Chemotactic/Chemokinetic Activity

A polypeptide of the present invention may be involved in chemotactic or chemokinetic activity for mammalian cells, including, for example, monocytes, fibroblasts, neutrophils, T-cells, mast cells, eosinophils, epithelial and/or endothelial cells. A polynucleotide of the invention can encode a polypeptide exhibiting such attributes. Chemotactic and chemokinetic receptor activation can be used to mobilize or attract a desired cell population to a desired site of action. Chemotactic or chemokinetic compositions (e.g. proteins, antibodies, binding partners, or modulators of the invention) provide particular advantages in treatment of wounds and other trauma to tissues, as well as in treatment of localized infections. For example, attraction of lymphocytes, monocytes or neutrophils to tumors or sites of infection may result in improved immune responses against the tumor or infecting agent.

A protein or peptide has chemotactic activity for a particular cell population if it can stimulate, directly or indirectly, the directed orientation or movement of such cell population. Preferably, the protein or peptide has the ability to directly stimulate directed movement of cells. Whether a particular protein has chemotactic activity for a population of cells can be readily determined by employing such protein or peptide in any known assay for cell chemotaxis.

Therapeutic compositions of the invention can be used in the following:

Assays for chemotactic activity (which will identify proteins that induce or prevent chemotaxis) consist of assays that measure the ability of a protein to induce the migration of cells across a membrane as well as the ability of a protein to induce the adhesion of one cell population to another cell population. Suitable assays for movement and adhesion include, without limitation, those described in: Current Protocols in Immunology, Ed by J. E. Coligan, A. M. Kruisbeek, D. H. Marguiles, E. M. Shevach, W. Strober, Pub. Greene Publishing Associates and Wiley-Interscience (Chapter 6.12, Measurement of alpha and beta Chemokines 6.12.1-6.12.28; Taub, et al. J. Clin. Invest. 95:1370-1376 (1995); Lind, et al. APMIS 103:140-146 (1995); Muller, et al Eur. J. Immunol. 25:1744-1748; Gruber, et al. J. Immunol. 152:5860-5867 (1994); Johnston, et al. J. Immunol. 153:1762-1768 (1994).

4.17.8 Activin/Inhibin Activity

A polypeptide of the present invention may also exhibit activin- or inhibin-related activities. A polynucleotide of the invention may encode a polypeptide exhibiting such characteristics. Inhibins are characterized by their ability to inhibit the release of follicle stimulating hormone (FSH), while activins and are characterized by their ability to stimulate the release of follicle stimulating hormone (FSH). Thus, a polypeptide of the present invention, alone or in heterodimers with a member of the inhibin family, may be useful as a contraceptive based on the ability of inhibins to decrease fertility in female mammals and decrease spermatogenesis in male mammals. Administration of sufficient amounts of other inhibins can induce infertility in these mammals. Alternatively, the polypeptide of the invention, as a homodimer or as a heterodimer with other protein subunits of the inhibin group, may be useful as a fertility inducing therapeutic, based upon the ability of activin molecules in stimulating FSH release from cells of the anterior pituitary. See, for example, U.S. Pat. No. 4,798,885. A polypeptide of the invention may also be useful for advancement of the onset of fertility in sexually immature mammals, so as to increase the lifetime reproductive performance of domestic animals such as, but not limited to, cows, sheep and pigs.

The activity of a polypeptide of the invention may, among other means, be measured by the following methods.

Assays for activin/inhibin activity include, without limitation, those described in: Vale et al., Endocrinology 91:562-572 (1972); Ling et al., Nature 321:779-782 (1986); Vale et al., Nature 321:776-779 (1986); Mason et al., Nature 318:659-663 (1985); Forage et al., Proc. Natl. Acad. Sci. USA 83:3091-3095 (1986).

4.17.9 Hemostatic and Thrombolytic Activity

A polypeptide of the invention may also be involved in hemostatis or thrombolysis or thrombosis. A polynucleotide of the invention can encode a polypeptide exhibiting such attributes. Compositions may be useful in treatment of various coagulation disorders (including hereditary disorders, such as hemophilias) or to enhance coagulation and other hemostatic events in treating wounds resulting from trauma, surgery or other causes. A composition of the invention may also be useful for dissolving or inhibiting formation of thromboses and for treatment and prevention of conditions resulting therefrom (such as, for example, infarction of cardiac and central nervous system vessels (e.g., stroke).

Therapeutic compositions of the invention can be used in the following:

Assay for hemostatic and thrombolytic activity include, without limitation, those described in: Linet, et al., J. Clin. Pharmacol. 26:131-140 (1986); Burdick, et al., Thrombosis Res. 45:413-419 (1987); Humphrey, et al., Fibrinolysis 5:71-79 (1991); Schaub, Prostaglandins 35:467-474 (1988).

4.17.10 Cancer Diagnosis and Therapy

Polypeptides of the invention may be involved in cancer cell generation, proliferation or metastasis. Detection of the presence or amount of polynucleotides or polypeptides of the invention may be useful for the diagnosis and/or prognosis of one or more types of cancer. For example, the presence or increased expression of a polynucleotide/polypeptide of the invention may indicate a hereditary risk of cancer, a precancerous condition, or an ongoing malignancy. Conversely, a defect in the gene or absence of the polypeptide may be associated with a cancer condition. Identification of single nucleotide polymorphisms associated with cancer or a predisposition to cancer may also be useful for diagnosis or prognosis.

Cancer treatments promote tumor regression by inhibiting tumor cell proliferation, inhibiting angiogenesis (growth of new blood vessels that is necessary to support tumor growth) and/or prohibiting metastasis by reducing tumor cell motility or invasiveness. Therapeutic compositions of the invention may be effective in adult and pediatric oncology including in solid phase tumors/malignancies, locally advanced tumors, human soft tissue sarcomas, metastatic cancer, including lymphatic metastases, blood cell malignancies including multiple myeloma, acute and chronic leukemias, and lymphomas, head and neck cancers including mouth cancer, larynx cancer and thyroid cancer, lung cancers including small cell carcinoma and non-small cell cancers, breast cancers including small cell carcinoma and ductal carcinoma, gastrointestinal cancers including esophageal cancer, stomach cancer, colon cancer, colorectal cancer and polyps associated with colorectal neoplasia, pancreatic cancers, liver cancer, urologic cancers including bladder cancer and prostate cancer, malignancies of the female genital tract including ovarian carcinoma, uterine (including endometrial) cancers, and solid tumor in the ovarian follicle, kidney cancers including renal cell carcinoma, brain cancers including intrinsic brain tumors, neuroblastoma, astrocytic brain tumors, gliomas, metastatic tumor cell invasion in the central nervous system, bone cancers including osteomas, skin cancers including malignant melanoma, tumor progression of human skin keratinocytes, squamous cell carcinoma, basal cell carcinoma, hemangiopericytoma and Karposi's sarcoma.

Polypeptides, polynucleotides, or modulators of polypeptides of the invention (including inhibitors and stimulators of the biological activity of the polypeptide of the invention) may be administered to treat cancer. Therapeutic compositions can be administered in therapeutically effective dosages alone or in combination with adjuvant cancer therapy such as surgery, chemotherapy, radiotherapy, thermotherapy, and laser therapy, and may provide a beneficial effect, e.g. reducing tumor size, slowing rate of tumor growth, inhibiting metastasis, or otherwise improving overall clinical condition, without necessarily eradicating the cancer.

The composition can also be administered in therapeutically effective amounts as a portion of an anti-cancer cocktail. An anti-cancer cocktail is a mixture of the polypeptide or modulator of the invention with one or more anti-cancer drugs in addition to a pharmaceutically acceptable carrier for delivery. The use of anti-cancer cocktails as a cancer treatment is routine. Anti-cancer drugs that are well known in the art and can be used as a treatment in combination with the polypeptide or modulator of the invention include: Actinomycin D, Aminoglutethimide, Asparaginase, Bleomycin, Busulfan, Carboplatin, Carmustine, Chlorambucil, Cisplatin (cis-DDP), Cyclophosphamide, Cytarabine HCl (Cytosine arabinoside), Dacarbazine, Dactinomycin, Daunorubicin HCl, Doxorubicin HCl, Estramustine phosphate sodium, Etoposide (V16-213), Floxuridine, 5-Fluorouracil (5-Fu), Flutamide, Hydroxyurea (hydroxycarbamide), Ifosfamide, Interferon Alpha-2a, Interferon Alpha-2b, Leuprolide acetate (LHRH-releasing factor analog), Lomustine, Mechlorethamine HCl (nitrogen mustard), Melphalan, Mercaptopurine, Mesna, Methotrexate (MTX), Mitomycin, Mitoxantrone HCl, Octreotide, Plicamycin, Procarbazine HCl, Streptozocin, Tamoxifen citrate, Thioguanine, Thiotepa, Vinblastine sulfate, Vincristine sulfate, Amsacrine, Azacitidine, Hexamethylmelamine, Interleukin-2, Mitoguazone, Pentostatin, Semustine, Teniposide, and Vindesine sulfate.

In addition, therapeutic compositions of the invention may be used for prophylactic treatment of cancer. There are hereditary conditions and/or environmental situations (e.g. exposure to carcinogens) known in the art that predispose an individual to developing cancers. Under these circumstances, it may be beneficial to treat these individuals with therapeutically effective doses of the polypeptide of the invention to reduce the risk of developing cancers.

In vitro models can be used to determine the effective doses of the polypeptide of the invention as a potential cancer treatment. These in vitro models include proliferation assays of cultured tumor cells, growth of cultured tumor cells in soft agar (see Freshney, (1987) Culture of Animal Cells: A Manual of Basic Technique, Wily-Liss, New York, N.Y. Ch 18 and Ch 21), tumor systems in nude mice as described in Giovanella, et al., J. Natl. Can. Inst., 52: 921-30 (1974), mobility and invasive potential of tumor cells in Boyden Chamber assays as described in Pilkington, et al., Anticancer Res., 17: 4107-9 (1997), and angiogenesis assays such as induction of vascularization of the chick chorioallantoic membrane or induction of vascular endothelial cell migration as described in Ribatta, et al., Intl. J. Dev. Biol., 40: 1189-97 (1999) and Li, et al., Clin. Exp. Metastasis, 17:423-9 (1999), respectively. Suitable tumor cells lines are available, e.g. from American Type Tissue Culture Collection catalogs.

4.17.11 Receptor/Ligand Activity

A polypeptide of the present invention may also demonstrate activity as receptor, receptor ligand or inhibitor or agonist of receptor/ligand interactions. A polynucleotide of the invention can encode a polypeptide exhibiting such characteristics. Examples of such receptors and ligands include, without limitation, cytokine receptors and their ligands, receptor kinases and their ligands, receptor phosphatases and their ligands, receptors involved in cell-cell interactions and their ligands (including without limitation, cellular adhesion molecules (such as selectins, integrins and their ligands) and receptor/ligand pairs involved in antigen presentation, antigen recognition and development of cellular and humoral immune responses. Receptors and ligands are also useful for screening of potential peptide or small molecule inhibitors of the relevant receptor/ligand interaction. A protein of the present invention (including, without limitation, fragments of receptors and ligands) may themselves be useful as inhibitors of receptor/ligand interactions.

The activity of a polypeptide of the invention may, among other means, be measured by the following methods:

Suitable assays for receptor-ligand activity include without limitation those described in: Current Protocols in Immunology, Ed by J. E. Coligan, A. M. Kruisbeek, D. H. Margulies, E. M. Shevach, W. Strober, Pub. Greene Publishing Associates and Wiley-Interscience (Chapter 7.28, Measurement of Cellular Adhesion under static conditions 7.28.1-7.28.22), Takai, et al., Proc. Natl. Acad. Sci. USA 84:6864-6868 (1987); Bierer, et al., J. Exp. Med. 168:1145-1156 (1988); Rosenstein, et al., J. Exp. Med. 169:149-160 (1989); Stoltenborg, et al., J. Immunol. Methods 175:59-68 (1994); Stitt, et al., Cell 80:661-670 (1995).

By way of example, the polypeptides of the invention may be used as a receptor for a ligand(s) thereby transmitting the biological activity of that ligand(s). Ligands may be identified through binding assays, affinity chromatography, dihybrid screening assays, BIAcore assays, gel overlay assays, or other methods known in the art.

Studies characterizing drugs or proteins as agonist or antagonist or partial agonists or a partial antagonist require the use of other proteins as competing ligands. The polypeptides of the present invention or ligand(s) thereof may be labeled by being coupled to radioisotopes, calorimetric molecules or a toxin molecules by conventional methods. (“Guide to Protein Purification” Murray P. Deutscher (ed) Methods in Enzymology Vol. 182 (1990) Academic Press, Inc. San Diego). Examples of radioisotopes include, but are not limited to, tritium and carbon-14. Examples of calorimetric molecules include, but are not limited to, fluorescent molecules such as fluorescamine, or rhodamine or other colorimetric molecules. Examples of toxins include, but are not limited, to ricin.

4.17.12 Drug Screening

This invention is particularly useful for screening chemical compounds by using the novel polypeptides or binding fragments thereof in any of a variety of drug screening techniques. The polypeptides or fragments employed in such a test may either be free in solution, affixed to a solid support, borne on a cell surface or located intracellularly. One method of drug screening utilizes eukaryotic or prokaryotic host cells which are stably transformed with recombinant nucleic acids expressing the polypeptide or a fragment thereof. Drugs are screened against such transformed cells in competitive binding assays. Such cells, either in viable or fixed form, can be used for standard binding assays. One may measure, for example, the formation of complexes between polypeptides of the invention or fragments and the agent being tested or examine the diminution in complex formation between the novel polypeptides and an appropriate cell line, which are well known in the art.

Sources for test compounds that may be screened for ability to bind to or modulate (i.e., increase or decrease) the activity of polypeptides of the invention include (1) inorganic and organic chemical libraries, (2) natural product libraries, and (3) combinatorial libraries comprised of either random or mimetic peptides, oligonucleotides or organic molecules.

Chemical libraries may be readily synthesized or purchased from a number of commercial sources, and may include structural analogs of known compounds or compounds that are identified as “hits” or “leads” via natural product screening.

The sources of natural product libraries are microorganisms (including bacteria and fingi), animals, plants or other vegetation, or marine organisms, and libraries of mixtures for screening may be created by: (1) fermentation and extraction of broths from soil, plant or marine microorganisms or (2) extraction of the organisms themselves. Natural product libraries include polyketides, non-ribosomal peptides, and (non-naturally occurring) variants thereof. For a review, see Science 282:63-68 (1998).

Combinatorial libraries are composed of large numbers of peptides, oligonucleotides or organic compounds and can be readily prepared by traditional automated synthesis methods, PCR, cloning or proprietary synthetic methods. Of particular interest are peptide and oligonucleotide combinatorial libraries. Still other libraries of interest include peptide, protein, peptidomimetic, multiparallel synthetic collection, recombinatorial, and polypeptide libraries. For a review of combinatorial chemistry and libraries created therefrom, see Myers, Curr. Opin. Biotechnol. 8:701-707 (1997). For reviews and examples of peptidomimetic libraries, see Al-Obeidi et al., Mol. Biotechnol, 9:205-23 (1998); Hruby, et al., Curr Opin Chem Biol, 1:114-19 (1997); Dorner, et al., Bioorg Med Chem, 4:709-15 (1996) (alkylated dipeptides).

Identification of modulators through use of the various libraries described herein permits modification of the candidate “hit” (or “lead”) to optimize the capacity of the “hit” to bind a polypeptide of the invention. The molecules identified in the binding assay are then tested for antagonist or agonist activity in in vivo tissue culture or animal models that are well known in the art. In brief, the molecules are titrated into a plurality of cell cultures or animals and then tested for either cell/animal death or prolonged survival of the animal/cells.

The binding molecules thus identified may be complexed with toxins, e.g., ricin or cholera, or with other compounds that are toxic to cells such as radioisotopes. The toxin-binding molecule complex is then targeted to a tumor or other cell by the specificity of the binding molecule for a polypeptide of the invention. Alternatively, the binding molecules may be complexed with imaging agents for targeting and imaging purposes.

4.17.13 Assay for Receptor Activity

The invention also provides methods to detect specific binding of a polypeptide e.g. a ligand or a receptor. The invention also provides methods to detect specific binding of a polypeptide of the invention to a binding partner polypeptide, and in particular a ligand polypeptide. Ligands useful in binding assays of this type include, for example Nogo-A, Nogo-B, Nogo-C, and Nogo-66 or related protein for NgRHy, and other binding partner/receptors for other polypeptides of the invention identified using assays well known and routinely practiced in the art.

In one embodiment, receptor activity of the polypeptides of the invention is determined using a method that involves (1) forming a mixture comprising a polypeptide of the invention, and/or its agonists and antagonists (or agonist or antagonist drug candidates) and/or antibodies specific for the polypeptides of the invention; (2) incubating the mixture under conditions whereby, but for the presence of said polypeptide of the invention and/or agonists and antagonists (or agonist or antagonist drug candidates) and/or antibodies specific for the polypeptides of the invention, the ligand binds to the receptor; and (3) detecting the presence or absence of specific binding of the polypeptide of the invention to its ligand.

The art provides numerous assays particularly useful for identifying previously unknown binding partners for receptor polypeptides of the invention. For example, expression cloning using mammalian or bacterial cells, or dihybrid screening assays can be used to identify polynucleotides encoding binding partners. As another example, affinity chromatography with the appropriate immobilized polypeptide of the invention can be used to isolate polypeptides that recognize and bind polypeptides of the invention. There are a number of different libraries used for the identification of compounds, and in particular small molecules, that modulate (i.e., increase or decrease) biological activity of a polypeptide of the invention. Ligands for receptor polypeptides of the invention can also be identified by adding exogenous ligands, or cocktails of ligands to two cells populations that are genetically identical except for the expression of the receptor of the invention: one cell population expresses the receptor of the invention whereas the other does not. The response of the two cell populations to the addition of ligands(s) is then compared. Alternatively, an expression library can be co-expressed with the polypeptide of the invention in cells and assayed for an autocrine response to identify potential ligand(s). As still another example, BIAcore assays, gel overlay assays, or other methods known in the art can be used to identify binding partner polypeptides, including, (1) organic and inorganic chemical libraries, (2) natural product libraries, and (3) combinatorial libraries comprised of random peptides, oligonucleotides or organic molecules.

The role of downstream intracellular signaling molecules in the signaling cascade of the polypeptide of the invention can be determined. For example, a chimeric protein in which the cytoplasmic domain of the polypeptide of the invention is fused to the extracellular portion of a protein, whose ligand has been identified, is produced in a host cell. The cell is then incubated with the ligand specific for the extracellular portion of the chimeric protein, thereby activating the chimeric receptor. Known downstream proteins involved in intracellular signaling can then be assayed for expected modifications i.e. phosphorylation. Other methods known to those in the art can also be used to identify signaling molecules involved in receptor activity.

4.17.14 Leukemia

Leukemia and related disorders may be treated or prevented by administration of a therapeutic that promotes or inhibits function of the polynucleotides and/or polypeptides of the invention. Such leukemias and related disorders include but are not limited to acute leukemia, acute lymphocytic leukemia, acute myelocytic leukemia, myeloblastic, promyelocytic, myelomonocytic, monocytic, erythroleukemia, chronic leukemia, chronic myelocytic (granulocytic) leukemia and chronic lymphocytic leukemia (for a review of such disorders, see Fishman, et al., 1985, Medicine, 2d Ed., J.B. Lippincott Co., Philadelphia).

4.17.15 Nervous System Disorders

Nervous system disorders, involving cell types which can be tested for efficacy of intervention with compounds that modulate the activity of the polynucleotides and/or polypeptides of the invention, and which can be treated upon thus observing an indication of therapeutic utility, include but are not limited to nervous system injuries, and diseases or disorders which result in either a disconnection of axons, a diminution or degeneration of neurons, or demyelination. Nervous system lesions which may be treated in a patient (including human and non-human mammalian patients) according to the invention include but are not limited to the following lesions of either the central (including spinal cord, brain) or peripheral nervous systems:
(i) traumatic lesions, including lesions caused by physical injury or associated with surgery, for example, lesions which sever a portion of the nervous system, or compression injuries;
(ii) ischemic lesions, in which a lack of oxygen in a portion of the nervous system results in neuronal injury or death, including cerebral infarction or ischemia, or spinal cord infarction or ischemia;
(iii) infectious lesions, in which a portion of the nervous system is destroyed or injured as a result of infection, for example, by an abscess or associated with infection by human immunodeficiency virus, herpes zoster, or herpes simplex virus or with Lyme disease, tuberculosis, syphilis;
(iv) degenerative lesions, in which a portion of the nervous system is destroyed or injured as a result of a degenerative process including but not limited to degeneration associated with Parkinson's disease, Alzheimer's disease, Huntington's chorea, or amyotrophic lateral sclerosis;
(v) lesions associated with nutritional diseases or disorders, in which a portion of the nervous system is destroyed or injured by a nutritional disorder or disorder of metabolism including but not limited to, vitamin B12 deficiency, folic acid deficiency, Wernicke disease, tobacco-alcohol amblyopia, Marchiafava-Bignami disease (primary degeneration of the corpus callosum), and alcoholic cerebellar degeneration;
(vi) neurological lesions associated with systemic diseases including but not limited to diabetes (diabetic neuropathy, Bell's palsy), systemic lupus erythematosus, carcinoma, or sarcoidosis;
(vii) lesions caused by toxic substances including alcohol, lead, or particular neurotoxins; and
(viii) demyelinated lesions in which a portion of the nervous system is destroyed or injured by a demyelinating disease including but not limited to multiple sclerosis, monophasic demyelination, encephalomyelitis, panencephalaitis, Marchiafava-Bignami disease, Spongy degeneration, Alexander's disease, Canavan's disease, metachromatic leukodystrophy, Krabbe's disease, human immunodeficiency virus-associated myelopathy, transverse myelopathy or various etiologies, progressive multifocal leukoencephalopathy, Guillain-Barre Syndrome, and central pontine myelinolysis.

Therapeutics which are useful according to the invention for treatment of a nervous system disorder may be selected by testing for biological activity in promoting the survival or differentiation of neurons. For example, and not by way of limitation, therapeutics which elicit any of the following effects may be useful according to the invention:
(i) increased survival time of neurons in culture;
(ii) increased sprouting of neurons in culture or in vivo;
(iii) increased production of a neuron-associated molecule in culture or in vivo, e.g., choline acetyltransferase or acetylcholinesterase with respect to motor neurons; or
(iv) decreased symptoms of neuron dysfunction in vivo.

Such effects may be measured by any method known in the art. In preferred, nonlimiting embodiments, increased survival of neurons may be measured by the method set forth in Arakawa et al. (J. Neurosci. 10:3507-3515 (1990)); increased sprouting of neurons may be detected by methods set forth in Pestronk, et al. (Exp. Neurol. 70:65-82 (1980)) or Brown, et al. (Ann. Rev. Neurosci. 4:17-42 (1981)); increased production of neuron-associated molecules may be measured by bioassay, enzymatic assay, antibody binding, Northern blot assay, etc., depending on the molecule to be measured; and motor neuron dysfunction may be measured by assessing the physical manifestation of motor neuron disorder, e.g., weakness, motor neuron conduction velocity, or functional disability.

In specific embodiments, motor neuron disorders that may be treated according to the invention include but are not limited to disorders such as infarction, infection, exposure to toxin, trauma, surgical damage, degenerative disease or malignancy that may affect motor neurons as well as other components of the nervous system, as well as disorders that selectively affect neurons such as amyotrophic lateral sclerosis, and including but not limited to progressive spinal muscular atrophy, progressive bulbar palsy, primary lateral sclerosis, infantile and juvenile muscular atrophy, progressive bulbar paralysis of childhood (Fazio-Londe syndrome), poliomyelitis and the post polio syndrome, and Hereditary Motorsensory Neuropathy (Charcot-Marie-Tooth Disease).

4.17.16 Other Activities

A polypeptide of the invention may also exhibit one or more of the following additional activities or effects: inhibiting the growth, infection or function of, or killing, infectious agents, including, without limitation, bacteria, viruses, fungi and other parasites; effecting (suppressing or enhancing) bodily characteristics, including, without limitation, height, weight, hair color, eye color, skin, fat to lean ratio or other tissue pigmentation, or organ or body part size or shape (such as, for example, breast augmentation or diminution, change in bone form or shape); effecting biorhythms or circadian cycles or rhythms; effecting the fertility of male or female subjects; effecting the metabolism, catabolism, anabolism, processing, utilization, storage or elimination of dietary fat, lipid, protein, carbohydrate, vitamins, minerals, co-factors or other nutritional factors or component(s); effecting behavioral characteristics, including, without limitation, appetite, libido, stress, cognition (including cognitive disorders), depression (including depressive disorders) and violent behaviors; providing analgesic effects or other pain reducing effects; promoting differentiation and growth of embryonic stem cells in lineages other than hematopoietic lineages; hormonal or endocrine activity; in the case of enzymes, correcting deficiencies of the enzyme and treating deficiency-related diseases; treatment of hyperproliferative disorders (such as, for example, psoriasis); immunoglobulin-like activity (such as, for example, the ability to bind antigens or complement); and the ability to act as an antigen in a vaccine composition to raise an immune response against such protein or another material or entity which is cross-reactive with such protein.

4.17.17 Identification of Polymorphisms

The demonstration of polymorphisms makes possible the identification of such polymorphisms in human subjects and the pharmacogenetic use of this information for diagnosis and treatment. Such polymorphisms may be associated with, e.g., differential predisposition or susceptibility to various disease states (such as disorders involving inflammation or immune response) or a differential response to drug administration, and this genetic information can be used to tailor preventive or therapeutic treatment appropriately. For example, the existence of a polymorphism associated with a predisposition to inflammation or autoimmune disease makes possible the diagnosis of this condition in humans by identifying the presence of the polymorphism.

Polymorphisms can be identified in a variety of ways known in the art which all generally involve obtaining a sample from a patient, analyzing DNA from the sample, optionally involving isolation or amplification of the DNA, and identifying the presence of the polymorphism in the DNA. For example, PCR may be used to amplify an appropriate fragment of genomic DNA which may then be sequenced. Alternatively, the DNA may be subjected to allele-specific oligonucleotide hybridization (in which appropriate oligonucleotides are hybridized to the DNA under conditions permitting detection of a single base mismatch) or to a single nucleotide extension assay (in which an oligonucleotide that hybridizes immediately adjacent to the position of the polymorphism is extended with one or more labeled nucleotides). In addition, traditional restriction fragment length polymorphism analysis (using restriction enzymes that provide differential digestion of the genomic DNA depending on the presence or absence of the polymorphism) may be performed. Arrays with nucleotide sequences of the present invention can be used to detect polymorphisms. The array can comprise modified nucleotide sequences of the present invention in order to detect the nucleotide sequences of the present invention. In the alternative, any one of the nucleotide sequences of the present invention can be placed on the array to detect changes from those sequences.

Alternatively a polymorphism resulting in a change in the amino acid sequence could also be detected by detecting a corresponding change in amino acid sequence of the protein, e.g., by an antibody specific to the variant sequence.

4.17.18 Arthritis and Inflammation

The immunosuppressive effects of the compositions of the invention against rheumatoid arthritis are determined in an experimental animal model system. The experimental model system is adjuvant induced arthritis in rats, and the protocol is described by J. Holoshitz, et al., Science, 219:56 (1983), or by B. Waksman, et al., Int. Arch. Allergy Appl. Immunol., 23:129 (1963). Induction of the disease can be caused by a single injection, generally intradermally, of a suspension of killed Mycobacterium tuberculosis in complete Freund's adjuvant (CFA). The route of injection can vary, but rats may be injected at the base of the tail with an adjuvant mixture. The polypeptide is administered in phosphate buffered solution (PBS) at a dose of about 1-5 mg/kg. The control consists of administering PBS only.

The procedure for testing the effects of the test compound would consist of intradermally injecting killed Mycobacterium tuberculosis in CFA followed by immediately administering the test compound and subsequent treatment every other day until day 24. At 14, 15, 18, 20, 22, and 24 days after injection of Mycobacterium CFA, an overall arthritis score may be obtained as described by J. Holoskitz above. An analysis of the data would reveal that the test compound would have a dramatic affect on the swelling of the joints as measured by a decrease of the arthritis score.

Compositions of the present invention may also exhibit other anti-inflammatory activity. The anti-inflammatory activity may be achieved by providing a stimulus to cells involved in the inflammatory response, by inhibiting or promoting cell-cell interactions (such as, for example, cell adhesion), by inhibiting or promoting chemotaxis of cells involved in the inflammatory process, inhibiting or promoting cell extravasation, or by stimulating or suppressing production of other factors which more directly inhibit or promote an inflammatory response. Compositions with such activities can be used to treat inflammatory conditions including chronic or acute conditions), including without limitation intimation associated with infection (such as septic shock, sepsis or systemic inflammatory response syndrome (SIRS)), ischemia-reperfusion injury, endotoxin lethality, arthritis, complement-mediated hyperacute rejection, nephritis, cytokine or chemokine-induced lung injury, inflammatory bowel disease, Crohn's disease or resulting from over production of cytokines such as TNF or IL-1. Compositions of the invention may also be useful to treat anaphylaxis and hypersensitivity to an antigenic substance or material. Compositions of this invention may be utilized to prevent or treat conditions such as, but not limited to, sepsis, acute pancreatitis, endotoxin shock, cytokine induced shock, rheumatoid arthritis, chronic inflammatory arthritis, pancreatic cell damage from diabetes mellitus type 1, graft versus host disease, inflammatory bowel disease, inflamation associated with pulmonary disease, other autoimmune disease or inflammatory disease, or in the prevention of premature labor secondary to intrauterine infections.

4.17.19 Nutritional Uses

Polynucleotides and polypeptides of the present invention can also be used as nutritional sources or supplements. Such uses include without limitation use as a protein or amino acid supplement, use as a carbon source, use as a nitrogen source and use as a source of carbohydrate. In such cases the polypeptide or polynucleotide of the invention can be added to the feed of a particular organism or can be administered as a separate solid or liquid preparation, such as in the form of powder, pills, solutions, suspensions or capsules. In the case of microorganisms, the polypeptide or polynucleotide of the invention can be added to the medium in or on which the microorganism is cultured. Additionally, the polypeptides of the invention can be used as markers, and as a food supplement. Protein food supplements are well known and the formulation of suitable food supplements including polypeptides of the invention is within the level of skill in the food preparation art.

4.17.20 Metabolic Disorders

A polynucleotide and polypeptide of the invention may also be involved in the prevention, diagnosis and management of metabolic disorders involving carbohydrates, lipids, amino acids, vitamins etc., including but not limited to diabetes mellitus, obesity, aspartylglusomarinuria, carbohydrate deficient glycoprotein syndrome (CDGS), cystinosis, diabetes insipidus, Fabry, fatty acid metabolism disorders, galactosemia, Gaucher, glucose-6-phosphate dehydrogenase (G6PD), glutaric aciduria, Hurler, Hurler-Scheie, Hunter, hypophosphatemia, 1-cell, Krabbe, lactic acidosis, long chain 3 hydroxyacyl CoA dehydrogenase deficiency (LCHAD), lysosomal storage diseases, mannosidosis, maple syrup urine, Maroteaux-Lamy, metachromatic leukodystrophy, mitochondrial Morquio, mucopolysaccharidosis, neuro-metabolic, Niemann-Pick, organic acidemias, purine, phenylketonuria (PKU), Pompe, porphyria, pseudo-Hurler, pyruvate dehydrogenase deficiency, Sandhoff, Sanfilippo, Scheie, Sly, Tay-Sachs, trimethylaminuria (Fish-Malodor syndrome), urea cycle conditions, vitamin D deficiency rickets and related complications involving different organs including but not limited to liver, heart, kidney, eye, brain, muscle development etc. Hereditary and/or environmental factors known in the art can predispose an individual to developing metabolic disorders and conditions resulting therefrom. Under these circumstances, it maybe beneficial to treat these individual with therapeutically effective doses of the polypeptide of the invention to reduce the risk of developing the disorder. Examples of such disorders include diabetes mellitus, obesity and cardiovascular disease. Further, polynucleotide sequences encoding the invention may be used in Southern or Northern analysis, dot blot, or other membrane-based technologies; in PCR technologies; or in dip stick, pin, ELISA or chip assays utilizing fluids or tissues from patient biopsies to detect altered expression of the polynucleotides of the invention. Such qualitative or quantitative methods are well known in the art.

4.7.21 Cardiovascular Disease and Therapy

Polypeptides and polynucleotides of the invention may also be involved in the prevention, diagnosis and management of cardiovascular disorders such as coronary artery disease, atherosclerosis and hyper- and hypolipoproteinemia, hypertension, angina pectoris, myocardial infarction, congestive heart failure, cardiac arrythmias including paroxysmal arrythmias, restenosis after angioplasty, aortic aneurysm and related complications involving various organs including but not limited to kidney, eye, brain, heart etc. Polypeptides of the invention may also have direct and indirect effects on myocardial contractility, electrical activity of the heart, atrial fibrillation, atrial fluter, anomalous atrio-ventricular pathways, sino-atrial dysfunction, vascular insufficiency and arterial embolism. Hereditary and/or environmental factors known in the art can predispose an individual to developing metabolic disorders and conditions resulting therefrom. Under these circumstances, it maybe beneficial to treat these individual with therapeutically effective doses of the polypeptide of the invention to reduce the risk of developing the disorder. Examples of such disorders include but are not limited to coronary artery disease, atherosclerosis, hyper- and hypolipoproteinemia, hypertension, angina pectoris, myocardial infarction, cardiac arrythmias including paroxysmal arrythmias, diabetes mellitus, inflammatory glomerulonephritis, ischemic renal failure, extracellular matrix accumulation, fibrosis, hypertension, coronary vasoconstriction, ischemic heart disease, and lesions occurring in brain disorders such as stroke, trauma, infarcts, aneurysms.

The polynucleotide sequences encoding the invention may be used in Southern or Northern analysis, dot blot, or other membrane-based technologies; in PCR technologies; or in dip stick, pin, ELISA or chip assays utilizing fluids or tissues from patient biopsies to detect altered expression of the polynucleotides of the invention. Such qualitative or quantitative methods are well known in the art.

4.18 Therapeutic Methods

The compositions (including polypeptide fragments, analogs, variants and antibodies or other binding partners or modulators including antisense polynucleotides) of the invention have numerous applications in a variety of therapeutic methods. Examples of therapeutic applications include, but are not limited to, those exemplified herein.

4.18.1 Example

One embodiment of the invention is the administration of an effective amount of the polypeptides of the invention or other composition of the invention to individuals affected by a disease or disorder that can be modulated by regulating the peptides of the invention. While the mode of administration is not particularly important, parenteral administration is preferred. An exemplary mode of administration is to deliver an intravenous bolus. The dosage of polypeptides of the invention or other composition of the invention will normally be determined by the prescribing physician. It is to be expected that the dosage will vary according to the age, weight, condition and response of the individual patient. Typically, the amount of polypeptide administered per dose will be in the range of about 0.01 μg/kg to 100 mg/kg of body weight, with the preferred dose being about 0.1 μg/kg to 10 mg/kg of patient body weight. For parenteral administration, polypeptides of the invention will be formulated in an injectable form combined with a pharmaceutically acceptable parenteral vehicle. Such vehicles are well known in the art and examples include water, saline, Ringer's solution, dextrose solution, and solutions consisting of small amounts of the human serum albumin. The vehicle may contain minor amounts of additives that maintain the isotonicity and stability of the polypeptide or other active ingredient. The preparation of such solutions is within the skill of the art.

4.19 Pharmaceutical Formulations and Routes of Administration

A protein or other composition of the present invention (from whatever source derived, including without limitation from recombinant and non-recombinant sources and including antibodies and other binding partners of the polypeptides of the invention) may be administered to a patient in need, by itself, or in pharmaceutical compositions where it is mixed with suitable carriers or excipient(s) at doses to treat or ameliorate a variety of disorders. Such a composition may optionally contain (in addition to protein or other active ingredient and a carrier) diluents, fillers, salts, buffers, stabilizers, solubilizers, and other materials well known in the art. The term “pharmaceutically acceptable” means a non-toxic material that does not interfere with the effectiveness of the biological activity of the active ingredient(s). The characteristics of the carrier will depend on the route of administration. The pharmaceutical composition of the invention may also contain cytokines, lymphokines, or other hematopoietic factors such as M-CSF, GM-CSF, TNF, IL-1, IL-2, IL-3, IL-4, IL-5, IL-6, IL-7, IL-8, IL-9, IL-10, IL-11, IL-12, IL-13, IL-14, IL-15, IFN, TNF0, TNF1, TNF2, G-CSF, Meg-CSF, thrombopoietin, stem cell factor, and erythropoietin. In further compositions, proteins of the invention may be combined with other agents beneficial to the treatment of the disease or disorder in question. These agents include various growth factors such as epidermal growth factor (EGF), platelet-derived growth factor (PDGF), transforming growth factors (TGF-α and TGF-β), insulin-like growth factor (IGF), as well as cytokines described herein.

The pharmaceutical composition may further contain other agents which either enhance the activity of the protein or other active ingredient or complement its activity or use in treatment. Such additional factors and/or agents may be included in the pharmaceutical composition to produce a synergistic effect with protein or other active ingredient of the invention, or to minimize side effects. Conversely, protein or other active ingredient of the present invention may be included in formulations of the particular clotting factor, cytokine, lymphokine, other hematopoietic factor, thrombolytic or anti-thrombotic factor, or anti-inflammatory agent to minimize side effects of the clotting factor, cytokine, lymphokine, other hematopoietic factor, thrombolytic or anti-thrombotic factor, or antiinflammatory agent (such as IL-1Ra, IL-1 Hy1, IL-1 Hy2, anti-TNF, corticosteroids, immunosuppressive agents). A protein of the present invention may be active in multimers (e.g., heterodimers or homodimers) or complexes with itself or other proteins. As a result, pharmaceutical compositions of the invention may comprise a protein of the invention in such multimeric or complexed form.

As an alternative to being included in a pharmaceutical composition of the invention including a first protein, a second protein or a therapeutic agent may be concurrently administered with the first protein (e.g., at the same time, or at differing times provided that therapeutic concentrations of the combination of agents is achieved at the treatment site). Techniques for formulation and administration of the compounds of the instant application may be found in “Remington's Pharmaceutical Sciences,” Mack Publishing Co., Easton, Pa., latest edition. A therapeutically effective dose further refers to that amount of the compound sufficient to result in amelioration of symptoms, e.g., treatment, healing, prevention or amelioration of the relevant medical condition, or an increase in rate of treatment, healing, prevention or amelioration of such conditions. When applied to an individual active ingredient, administered alone, a therapeutically effective dose refers to that ingredient alone. When applied to a combination, a therapeutically effective dose refers to combined amounts of the active ingredients that result in the therapeutic effect, whether administered in combination, serially or simultaneously.

In practicing the method of treatment or use of the present invention, a therapeutically effective amount of protein or other active ingredient of the present invention is administered to a mammal having a condition to be treated. Protein or other active ingredient of the present invention may be administered in accordance with the method of the invention either alone or in combination with other therapies such as treatments employing cytokines, lymphokines or other hematopoietic factors. When co-administered with one or more cytokines, lymphokines or other hematopoietic factors, protein or other active ingredient of the present invention may be administered either simultaneously with the cytokine(s), lymphokine(s), other hematopoietic factor(s), thrombolytic or anti-thrombotic factors, or sequentially. If administered sequentially, the attending physician will decide on the appropriate sequence of administering protein or other active ingredient of the present invention in combination with cytokine(s), lymphokine(s), other hematopoietic factor(s), thrombolytic or anti-thrombotic factors.

4.19.1 Routes of Administration

Suitable routes of administration may, for example, include oral, rectal, transmucosal, or intestinal administration; parenteral delivery, including intramuscular, subcutaneous, intramedullary injections, as well as intrathecal, direct intraventricular, intravenous, intraperitoneal, intranasal, or intraocular injections. Administration of protein or other active ingredient of the present invention used in the pharmaceutical composition or to practice the method of the present invention can be carried out in a variety of conventional ways, such as oral ingestion, inhalation, topical application or cutaneous, subcutaneous, intraperitoneal, parenteral or intravenous injection. Intravenous administration to the patient is preferred.

Alternately, one may administer the compound in a local rather than systemic manner, for example, via injection of the compound directly into arthritic joints or in fibrotic tissue, often in a depot or sustained release formulation. In order to prevent the scarring process frequently occurring as complication of glaucoma surgery, the compounds may be administered topically, for example, as eye drops. Furthermore, one may administer the drug in a targeted drug delivery system, for example, in a liposome coated with a specific antibody, targeting, for example, arthritic or fibrotic tissue. The liposomes will be targeted to and taken up selectively by the afflicted tissue.

The polypeptides of the invention are administered by any route that delivers an effective dosage to the desired site of action. The determination of a suitable route of administration and an effective dosage for a particular indication is within the level of skill in the art. Preferably for wound treatment, one administers the therapeutic compound directly to the site. Suitable dosage ranges for the polypeptides of the invention can be extrapolated from these dosages or from similar studies in appropriate animal models. Dosages can then be adjusted as necessary by the clinician to provide maximal therapeutic benefit.

4.19.2 Compositions/Formulations

Pharmaceutical compositions for use in accordance with the present invention thus may be formulated in a conventional manner using one or more physiologically acceptable carriers comprising excipients and auxiliaries which facilitate processing of the active compounds into preparations which can be used pharmaceutically. These pharmaceutical compositions may be manufactured in a manner that is itself known, e.g., by means of conventional mixing, dissolving, granulating, dragee-making, levigating, emulsifying, encapsulating, entrapping or lyophilizing processes. Proper formulation is dependent upon the route of administration chosen. When a therapeutically effective amount of protein or other active ingredient of the present invention is administered orally, protein or other active ingredient of the present invention will be in the form of a tablet, capsule, powder, solution or elixir. When administered in tablet form, the pharmaceutical composition of the invention may additionally contain a solid carrier such as a gelatin or an adjuvant. The tablet, capsule, and powder contain from about 5 to 95% protein or other active ingredient of the present invention, and preferably from about 25 to 90% protein or other active ingredient of the present invention. When administered in liquid form, a liquid carrier such as water, petroleum, oils of animal or plant origin such as peanut oil, mineral oil, soybean oil, or sesame oil, or synthetic oils may be added. The liquid form of the pharmaceutical composition may further contain physiological saline solution, dextrose or other saccharide solution, or glycols such as ethylene glycol, propylene glycol or polyethylene glycol. When administered in liquid form, the pharmaceutical composition contains from about 0.5 to 90% by weight of protein or other active ingredient of the present invention, and preferably from about 1 to 50% protein or other active ingredient of the present invention.

When a therapeutically effective amount of protein or other active ingredient of the present invention is administered by intravenous, cutaneous or subcutaneous injection, protein or other active ingredient of the present invention will be in the form of a pyrogen-free, parenterally acceptable aqueous solution. The preparation of such parenterally acceptable protein or other active ingredient solutions, having due regard to pH, isotonicity, stability, and the like, is within the skill in the art. A preferred pharmaceutical composition for intravenous, cutaneous, or subcutaneous injection should contain, in addition to protein or other active ingredient of the present invention, an isotonic vehicle such as Sodium Chloride Injection, Ringer's Injection, Dextrose Injection, Dextrose and Sodium Chloride Injection, Lactated Ringer's Injection, or other vehicle as known in the art. The pharmaceutical composition of the present invention may also contain stabilizers, preservatives, buffers, antioxidants, or other additives known to those of skill in the art. For injection, the agents of the invention may be formulated in aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration. For buccal administration, the compositions may take the form of tablets or lozenges formulated in conventional manner.

For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch. The compounds may be formulated for parenteral administration by injection, e.g., by bolus injection or continuous infusion. Formulations for injection may be presented in unit dosage form, e.g., in ampules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides. In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

A pharmaceutical carrier for the hydrophobic compounds of the invention is a cosolvent system comprising benzyl alcohol, a nonpolar surfactant, a water-miscible organic polymer, and an aqueous phase. The co-solvent system may be the VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD:5W) consists of VPD diluted 1:1 with a 5% dextrose in water solution. This co-solvent system dissolves hydrophobic compounds well, and itself produces low toxicity upon systemic administration. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of polysorbate 80; the fraction size of polyethylene glycol may be varied; other biocompatible polymers may replace polyethylene glycol, e.g. polyvinyl pyrrolidone; and other sugars or polysaccharides may substitute for dextrose. Alternatively, other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various types of sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein or other active ingredient stabilization may be employed.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols. Many of the active ingredients of the invention may be provided as salts with pharmaceutically compatible counter ions. Such pharmaceutically acceptable base addition salts are those salts which retain the biological effectiveness and properties of the free acids and which are obtained by reaction with inorganic or organic bases such as sodium hydroxide, magnesium hydroxide, ammonia, trialkylamine, dialkylamine, monoalkylamine, dibasic amino acids, sodium acetate, potassium benzoate, triethanol amine and the like.

The pharmaceutical composition of the invention may be in the form of a complex of the protein(s) or other active ingredient of present invention along with protein or peptide antigens. The protein and/or peptide antigen will deliver a stimulatory signal to both B and T lymphocytes. B lymphocytes will respond to antigen through their surface immunoglobulin receptor. T lymphocytes will respond to antigen through the T cell receptor (TCR) following presentation of the antigen by MHC proteins. MHC and structurally related proteins including those encoded by class I and class II MHC genes on host cells will serve to present the peptide antigen(s) to T lymphocytes. The antigen components could also be supplied as purified MHC-peptide complexes alone or with co-stimulatory molecules that can directly signal T cells. Alternatively antibodies able to bind surface immunoglobulin and other molecules on B cells as well as antibodies able to bind the TCR and other molecules on T cells can be combined with the pharmaceutical composition of the invention.

The pharmaceutical composition of the invention may be in the form of a liposome in which protein of the present invention is combined, in addition to other pharmaceutically acceptable carriers, with amphipathic agents such as lipids which exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithins, phospholipids, saponin, bile acids, and the like. Preparation of such liposomal formulations is within the level of skill in the art, as disclosed, for example, in U.S. Pat. Nos. 4,235,871; 4,501,728; 4,837,028; and 4,737,323, all of which are incorporated herein by reference.

The amount of protein or other active ingredient of the present invention in the pharmaceutical composition of the present invention will depend upon the nature and severity of the condition being treated, and on the nature of prior treatments which the patient has undergone. Ultimately, the attending physician will decide the amount of protein or other active ingredient of the present invention with which to treat each individual patient. Initially, the attending physician will administer low doses of protein or other active ingredient of the present invention and observe the patient's response. Larger doses of protein or other active ingredient of the present invention may be administered until the optimal therapeutic effect is obtained for the patient, and at that point the dosage is not increased further. It is contemplated that the various pharmaceutical compositions used to practice the method of the present invention should contain about 0.01 μg to about 100 mg (preferably about 0.1 μg to about 10 mg, more preferably about 0.1 μg to about 1 mg) of protein or other active ingredient of the present invention per kg body weight. For compositions of the present invention which are useful for bone, cartilage, tendon or ligament regeneration, the therapeutic method includes administering the composition topically, systematically, or locally as an implant or device. When administered, the therapeutic composition for use in this invention is, of course, in a pyrogen-free, physiologically acceptable form. Further, the composition may desirably be encapsulated or injected in a viscous form for delivery to the site of bone, cartilage or tissue damage. Topical administration may be suitable for wound healing and tissue repair. Therapeutically useful agents other than a protein or other active ingredient of the invention which may also optionally be included in the composition as described above, may alternatively or additionally, be administered simultaneously or sequentially with the composition in the methods of the invention. Preferably for bone and/or cartilage formation, the composition would include a matrix capable of delivering the protein-containing or other active ingredient-containing composition to the site of bone and/or cartilage damage, providing a structure for the developing bone and cartilage and optimally capable of being reabsorbed into the body. Such matrices may be formed of materials presently in use for other implanted medical applications.

The choice of matrix material is based on biocompatibility, biodegradability, mechanical properties, cosmetic appearance and interface properties. The particular application of the compositions will define the appropriate formulation. Potential matrices for the compositions may be biodegradable and chemically defined calcium sulfate, tricalcium phosphate, hydroxyapatite, polylactic acid, polyglycolic acid and polyanhydrides. Other potential materials are biodegradable and biologically well-defined, such as bone or dermal collagen. Further matrices are comprised of pure proteins or extracellular matrix components. Other potential matrices are nonbiodegradable and chemically defined, such as sintered hydroxyapatite, bioglass, aluminates, or other ceramics. Matrices may be comprised of combinations of any of the above mentioned types of material, such as polylactic acid and hydroxyapatite or collagen and tricalcium phosphate. The bioceramics may be altered in composition, such as in calcium-aluminate-phosphate and processing to alter pore size, particle size, particle shape, and biodegradability. Presently preferred is a 50:50 (mole weight) copolymer of lactic acid and glycolic acid in the form of porous particles having diameters ranging from 150 to 800 microns. In some applications, it will be useful to utilize a sequestering agent, such as carboxymethyl cellulose or autologous blood clot, to prevent the protein compositions from disassociating from the matrix.

A preferred family of sequestering agents is cellulosic materials such as alkylcelluloses (including hydroxyalkylcelluloses), including methylcellulose, ethylcellulose, hydroxyethylcellulose, hydroxypropylcellulose, hydroxypropylmethylcellulose, and carboxymethylcellulose, the most preferred being cationic salts of carboxymethylcellulose (CMC). Other preferred sequestering agents include hyaluronic acid, sodium alginate, poly(ethylene glycol), polyoxyethylene oxide, carboxyvinyl polymer and poly(vinyl alcohol). The amount of sequestering agent useful herein is 0.5-20 wt %, preferably 1-10 wt % based on total formulation weight, which represents the amount necessary to prevent desorption of the protein from the polymer matrix and to provide appropriate handling of the composition, yet not so much that the progenitor cells are prevented from infiltrating the matrix, thereby providing the protein the opportunity to assist the osteogenic activity of the progenitor cells. In further compositions, proteins or other active ingredient of the invention may be combined with other agents beneficial to the treatment of the bone and/or cartilage defect, wound, or tissue in question. These agents include various growth factors such as epidermal growth factor (EGF), platelet derived growth factor (PDGF), transforming growth factors (TGF-α and TGF-β), and insulin-like growth factor (IGF).

The therapeutic compositions are also presently valuable for veterinary applications. Particularly domestic animals and thoroughbred horses, in addition to humans, are desired patients for such treatment with proteins or other active ingredient of the present invention. The dosage regimen of a protein-containing pharmaceutical composition to be used in tissue regeneration will be determined by the attending physician considering various factors which modify the action of the proteins, e.g., amount of tissue weight desired to be formed, the site of damage, the condition of the damaged tissue, the size of a wound, type of damaged tissue (e.g., bone), the patient's age, sex, and diet, the severity of any infection, time of administration and other clinical factors. The dosage may vary with the type of matrix used in the reconstitution and with inclusion of other proteins in the pharmaceutical composition. For example, the addition of other known growth factors, such as IGF I (insulin like growth factor I), to the final composition, may also effect the dosage. Progress can be monitored by periodic assessment of tissue/bone growth and/or repair, for example, X-rays, histomorphometric determinations and tetracycline labeling.

Polynucleotides of the present invention can also be used for gene therapy. Such polynucleotides can be introduced either in vivo or ex vivo into cells for expression in a mammalian subject. Polynucleotides of the invention may also be administered by other known methods for introduction of nucleic acid into a cell or organism (including, without limitation, in the form of viral vectors or naked DNA). Cells may also be cultured ex vivo in the presence of proteins of the present invention in order to proliferate or to produce a desired effect on or activity in such cells. Treated cells can then be introduced in vivo for therapeutic purposes.

4.19.3 Effective Dosage

Pharmaceutical compositions suitable for use in the present invention include compositions wherein the active ingredients are contained in an effective amount to achieve its intended purpose. More specifically, a therapeutically effective amount means an amount effective to prevent development of or to alleviate the existing symptoms of the subject being treated. Determination of the effective amount is well within the capability of those skilled in the art, especially in light of the detailed disclosure provided herein. For any compound used in the method of the invention, the therapeutically effective dose can be estimated initially from appropriate in vitro assays. For example, a dose can be formulated in animal models to achieve a circulating concentration range that can be used to more accurately determine useful doses in humans. For example, a dose can be formulated in animal models to achieve a circulating concentration range that includes the IC₅₀ as determined in cell culture (i.e., the concentration of the test compound which achieves a half-maximal inhibition of the protein's biological activity). Such information can be used to more accurately determine useful doses in humans.

A therapeutically effective dose refers to that amount of the compound that results in amelioration of symptoms or a prolongation of survival in a patient. Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio between LD₅₀ and ED₅₀. Compounds which exhibit high therapeutic indices are preferred. The data obtained from these cell culture assays and animal studies can be used in formulating a range of dosage for use in human. The dosage of such compounds lies preferably within a range of circulating concentrations that include the ED₅₀ with little or no toxicity. The dosage may vary within this range depending upon the dosage form employed and the route of administration utilized. The exact formulation, route of administration and dosage can be chosen by the individual physician in view of the patient's condition. See, e.g., Fingl et al., 1975, in “The Pharmacological Basis of Therapeutics”, Ch. 1 p.1. Dosage amount and interval may be adjusted individually to provide plasma levels of the active moiety which are sufficient to maintain the desired effects, or minimal effective concentration (MEC). The MEC will vary for each compound but can be estimated from in vitro data. Dosages necessary to achieve the MEC will depend on individual characteristics and route of administration. However, HPLC assays or bioassays can be used to determine plasma concentrations.

Dosage intervals can also be determined using MEC value. Compounds should be administered using a regimen which maintains plasma levels above the MEC for 10-90% of the time, preferably between 30-90% and most preferably between 50-90%. In cases of local administration or selective uptake, the effective local concentration of the drug may not be related to plasma concentration.

An exemplary dosage regimen for polypeptides or other compositions of the invention will be in the range of about 0.01 μg/kg to 100 mg/kg of body weight daily, with the preferred dose being about 0.1 μg/kg to 25 mg/kg of patient body weight daily, varying in adults and children. Dosing may be once daily, or equivalent doses may be delivered at longer or shorter intervals.

The amount of composition administered will, of course, be dependent on the subject being treated, on the subject's age and weight, the severity of the affliction, the manner of administration and the judgment of the prescribing physician.

4.19.4 Packaging

The compositions may, if desired, be presented in a pack or dispenser device which may contain one or more unit dosage forms containing the active ingredient. The pack may, for example, comprise metal or plastic foil, such as a blister pack. The pack or dispenser device may be accompanied by instructions for administration. Compositions comprising a compound of the invention formulated in a compatible pharmaceutical carrier may also be prepared, placed in an appropriate container, and labeled for treatment of an indicated condition.

4.20 Antibodies

Also included in the invention are antibodies to proteins, or fragments of proteins of the invention. The term “antibody” as used herein refers to immunoglobulin molecules and immunologically active portions of immunoglobulin (Ig) molecules, i.e., molecules that contain an antigen-binding site that specifically binds (immunoreacts with) an antigen. Such antibodies include, but are not limited to, polyclonal, monoclonal, chimeric, single chain, F_{ab}, F_{ab′} and F_{(ab′)2} fragments, and an F_{ab} expression library. In general, an antibody molecule obtained from humans relates to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species.

An isolated related protein of the invention may be intended to serve as an antigen, or a portion or fragment thereof, and additionally can be used as an immunogen to generate antibodies that immunospecifically bind the antigen, using standard techniques for polyclonal and monoclonal antibody preparation. The full-length protein can be used or, alternatively, the invention provides antigenic peptide fragments of the antigen for use as immunogens. An antigenic peptide fragment comprises at least 6 amino acid residues of the amino acid sequence of the full length protein, such as an amino acid sequence shown in SEQ ID NO: 5, 7-13, 15, 17-24, 28, 30-156, 160, 162-182, 186, 188-213, 215, 217-239, 241, 243-270, 272, 274-299, 302, 304-321, 323, 325-344, 348, 350-352, 355, 357-376, 378, 380-401,408,410-414, 415, 420, 422-439, 444-480,482-484, 487, 489-501, 505, 507-512, 516, 518-524, 528, 530-539, 542, 544-546, 548, 550-553, 557, 559-567, 572, 574, 576, 579, 581-584, 588, 590, 596, 602, 604-605, 607, 609-610, 612, 614-615, 618, 620, 622, 624, 626, 628, 630, 632, or 634-653, or Tables 2-44 and encompasses an epitope thereof such that an antibody raised against the peptide forms a specific immune complex with the full length protein or with any fragment that contains the epitope. Preferably, the antigenic peptide comprises at least 10 amino acid residues, or at least 15 amino acid residues, or at least 20 amino acid residues, or at least 30 amino acid residues. Preferred epitopes encompassed by the antigenic peptide are regions of the protein that are located on its surface; commonly these are hydrophilic regions.

In certain embodiments of the invention, at least one epitope encompassed by the antigenic peptide is a surface region of the protein, e.g., a hydrophilic region. A hydrophobicity analysis of the human related protein sequence will indicate which regions of a related protein are particularly hydrophilic and, therefore, are likely to encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, Proc. Nat. Acad. Sci. USA 78: 3824-3828 (1981); Kyte and Doolittle, J. Mol. Biol. 157: 105-142 (1982), each of which is incorporated herein by reference in its entirety. Antibodies that are specific for one or more domains within an antigenic protein, or derivatives, fragments, analogs or homologs thereof, are also provided herein.

A protein of the invention, or a derivative, fragment, analog, homolog or ortholog thereof, may be utilized as an immunogen in the generation of antibodies that immunospecifically bind these protein components.

The term “specific for” indicates that the variable regions of the antibodies of the invention recognize and bind polypeptides of the invention exclusively (i.e., able to distinguish the polypeptide of the invention from other similar polypeptides despite sequence identity, homology, or similarity found in the family of polypeptides), but may also interact with other proteins (for example, S. aureus protein A or other antibodies in ELISA techniques) through interactions with sequences outside the variable region of the antibodies, and in particular, in the constant region of the molecule. Screening assays to determine binding specificity of an antibody of the invention are well known and routinely practiced in the art. For a comprehensive discussion of such assays, see Harlow et al. (Eds), Antibodies A Laboratory Manual; Cold Spring Harbor Laboratory; Cold Spring Harbor, N.Y. (1988), Chapter 6. Antibodies that recognize and bind fragments of the polypeptides of the invention are also contemplated, provided that the antibodies are first and foremost specific for, as defined above, full-length polypeptides of the invention. As with antibodies that are specific for full length polypeptides of the invention, antibodies of the invention that recognize fragments are those which can distinguish polypeptides from the same family of polypeptides despite inherent sequence identity, homology, or similarity found in the family of proteins.

Antibodies of the invention are useful for, for example, therapeutic purposes (by modulating activity of a polypeptide of the invention), diagnostic purposes to detect or quantitate a polypeptide of the invention, as well as purification of a polypeptide of the invention. Kits comprising an antibody of the invention for any of the purposes described herein are also comprehended. In general, a kit of the invention also includes a control antigen for which the antibody is immunospecific. The invention further provides a hybridoma that produces an antibody according to the invention. Antibodies of the invention are useful for detection and/or purification of the polypeptides of the invention.

Monoclonal antibodies binding to the protein of the invention may be useful diagnostic agents for the immunodetection of the protein. Neutralizing monoclonal antibodies binding to the protein may also be useful therapeutics for both conditions associated with the protein and also in the treatment of some forms of cancer where abnormal expression of the protein is involved. In the case of cancerous cells or leukemic cells, neutralizing monoclonal antibodies against the protein may be useful in detecting and preventing the metastatic spread of the cancerous cells, which may be mediated by the protein.

The labeled antibodies of the present invention can be used for in vitro, in vivo, and in situ assays to identify cells or tissues in which a fragment of the polypeptide of interest is expressed. The antibodies may also be used directly in therapies or other diagnostics. The present invention further provides the above-described antibodies immobilized on a solid support. Examples of such solid supports include plastics such as polycarbonate, complex carbohydrates such as agarose and Sepharose®, acrylic resins and such as polyacrylamide and latex beads. Techniques for coupling antibodies to such solid supports are well known in the art (Weir, D. M. et al., “Handbook of Experimental Immunology” 4th Ed., Blackwell Scientific Publications, Oxford, England, Chapter 10 (1986); Jacoby, W. D. et al., Meth. Enzym. 34 Academic Press, N.Y. (1974)). The immobilized antibodies of the present invention can be used for in vitro, in vivo, and in situ assays as well as for immuno-affinity purification of the proteins of the present invention.

Various procedures known within the art may be used for the production of polyclonal or monoclonal antibodies directed against a protein of the invention, or against derivatives, fragments, analogs homologs or orthologs thereof (see, for example, Antibodies: A Laboratory Manual, Harlow E, and Lane D, 1988, Cold Spring Harbor Laboratory Press, Cold Spring Harbor, N.Y., incorporated herein by reference). Some of these antibodies are discussed below.

4.20.1 Polyclonal Antibodies

For the production of polyclonal antibodies, various suitable host animals (e.g., rabbit, goat, mouse or other mammal) may be immunized by one or more injections with the native protein, a synthetic variant thereof, or a derivative of the foregoing. An appropriate immunogenic preparation can contain, for example, the naturally occurring immunogenic protein, a chemically synthesized polypeptide representing the immunogenic protein, or a recombinantly expressed immunogenic protein. Furthermore, the protein may be conjugated to a second protein known to be immunogenic in the mammal being immunized. Examples of such immunogenic proteins include but are not limited to keyhole limpet hemocyanin, serum albumin, bovine thyroglobulin, and soybean trypsin inhibitor. The preparation can further include an adjuvant. Various adjuvants used to increase the immunological response include, but are not limited to, Freund's (complete and incomplete), mineral gels (e.g., aluminum hydroxide), surface-active substances (e.g., lysolecithin, pluronic polyols, polyanions, peptides, oil emulsions, dinitrophenol, etc.), adjuvants usable in humans such as Bacille Calmette-Guerin and Corynebacterium parvum, or similar immunostimulatory agents. Additional examples of adjuvants that can be employed include MPL-TDM adjuvant (monophosphoryl Lipid A, synthetic trehalose dicorynomycolate).

The polyclonal antibody molecules directed against the immunogenic protein can be isolated from the mammal (e.g., from the blood) and further purified by well known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

4.20.2 Monoclonal Antibodies

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen-binding site capable of immunoreacting with a particular epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, Marcel Dekker, Inc., New York, (1987) pp. 51-63).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). Preferably, antibodies having a high degree of specificity and a high binding affinity for the target antigen are isolated.

After the desired hybridoma cells are identified, the clones can be subcloned by limiting dilution procedures and grown by standard methods. Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; Morrison, Nature 368:812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

4.20.3 Humanized Antibodies

The antibodies directed against the protein antigens of the invention can further comprise humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., Nature, 321:522-525 (1986); Riechmann, et al., Nature, 332:323-327 (1988); Verhoeyen, et al., Science, 239:1534-1536 (1988)), by substituting rodent CDRs or CDR sequences for the corresponding sequences of a human antibody. (See also U.S. Pat. No. 5,225,539). In some instances, Fv framework residues of the human immunoglobulin are replaced by corresponding non-human residues. Humanized antibodies can also comprise residues that are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin (Jones et al., 1986; Riechmann et al., 1988; and Presta, Curr. Op. Struct. Biol., 2:593-596 (1992)).

4.20.4 Human Antibodies

Fully human antibodies relate to antibody molecules in which essentially the entire sequences of both the light chain and the heavy chain, including the CDRs, arise from human genes. Such antibodies are termed “human antibodies” or “fully human antibodies” herein. Human monoclonal antibodies can be prepared by the trioma technique; the human B-cell hybridoma technique (see Kozbor, et al., Immunol Today 4: 72 (1983)) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al., Proc Natl Acad Sci USA 80: 2026-2030 (1983)) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

In addition, human antibodies can also be produced using additional techniques, including phage display libraries (Hoogenboom and Winter, J. Mol. Biol., 227:381 (1991); Marks et al., J. Mol. Biol., 222:581(1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (Bio/Technology 10:779-783 (1992)); Lonberg et al. (Nature 368:856-859 (1994)); Morrison (Nature 368:812-13 (1994)); Fishwild et al (Nature Biotechnology, 14:845-51 (1996)); Neuberger (Nature Biotechnology, 14:826 (1996)); and Lonberg and Huszar (Intern. Rev. Immunol. 13:65-93 (1995)).

Human antibodies may additionally be produced using transgenic nonhuman animals which are modified so as to produce fully human antibodies rather than the animal's WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse™ as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No. 5,939,598. It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

A method for producing an antibody of interest, such as a human antibody, is disclosed in U.S. Pat. No. 5,916,771. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen, and a correlative method for selecting an antibody that binds immunospecifically to the relevant epitope with high affinity, are disclosed in PCT publication WO 99/53049.

4.20.5 Fab Fragments and Single Chain Antibodies

According to the invention, techniques can be adapted for the production of single-chain antibodies specific to an antigenic protein of the invention (see e.g., U.S. Pat. No. 4,946,778). In addition, methods can be adapted for the construction of F_{ab} expression libraries (see e.g., Huse, et al., Science 246:1275-1281 (1989)) to allow rapid and effective identification of monoclonal F_{ab} fragments with the desired specificity for a protein or derivatives, fragments, analogs or homologs thereof. Antibody fragments that contain the idiotypes to a protein antigen may be produced by techniques known in the art including, but not limited to: (i) an F_{(ab)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

4.20.6 Bispecific Antibodies

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published 13 May 1993, and in Traunecker et al., EMBO J., 10:3655-3659 (1991).

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are cotransfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full-length antibodies or antibody fragments (e.g. F(ab′)₂ bispecific antibodies). Techniques for generating bispecific antibodies from antibody fragments have been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Additionally, Fab′ fragments can be directly recovered from E. coli and chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol. 148:1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994).

Antibodies with more than two valencies are contemplated. For example, trispecific antibodies can be prepared. Tutt et al., J. Immunol. 147:60 (1991).

Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g. CD2, CD3, CD28, or B7), or Fc receptors for IgG (FcγR), such as FcγRI (CD64), FcγRII (CD32) and FcγRIII (CD16) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA. Another bispecific antibody of interest binds the protein antigen described herein and further binds tissue factor (TF).

4.20.7 Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

4.20.8 Effector Function Engineering

It can be desirable to modify the antibody of the invention with respect to effector function, so as to enhance, e.g., the effectiveness of the antibody in treating cancer. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). See Caron et al., J. Exp Med., 176: 1191-1195 (1992) and Shopes, J. Immunol., 148: 2918-2922 (1992). Homodimeric antibodies with enhanced anti-tumor activity can also be prepared using heterobifunctional cross-linkers as described in Wolff et al. Cancer Research, 53: 2560-2565 (1993). Alternatively, an antibody can be engineered that has dual Fc regions and can thereby have enhanced complement lysis and ADCC capabilities. See Stevenson et al., Anti-Cancer Drug Design, 3: 219-230 (1989).

4.20.9 Immunoconjugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol)propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis(p-azidobenzoyl)hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

In another embodiment, the antibody can be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g., avidin) that is in turn conjugated to a cytotoxic agent.

4.21 Computer Readable Sequences

In one application of this embodiment, a nucleotide sequence of the present invention can be recorded on computer readable media. As used herein, “computer readable media” refers to any medium which can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. A skilled artisan can readily appreciate how any of the presently known computer readable mediums can be used to create a manufacture comprising computer readable medium having recorded thereon a nucleotide sequence of the present invention. As used herein, “recorded” refers to a process for storing information on computer readable medium. A skilled artisan can readily adopt any of the presently known methods for recording information on computer readable medium to generate manufactures comprising the nucleotide sequence information of the present invention.

A variety of data storage structures are available to a skilled artisan for creating a computer readable medium having recorded thereon a nucleotide sequence of the present invention. The choice of the data storage structure will generally be based on the means chosen to access the stored information. In addition, a variety of data processor programs and formats can be used to store the nucleotide sequence information of the present invention on computer readable medium. The sequence information can be represented in a word processing text file, formatted in commercially-available software such as WordPerfect and Microsoft Word, or represented in the form of an ASCII file, stored in a database application, such as DB2, Sybase, Oracle, or the like. A skilled artisan can readily adapt any number of data processor structuring formats (e.g. text file or database) in order to obtain computer readable medium having recorded thereon the nucleotide sequence information of the present invention.

By providing any of the nucleotide sequences SEQ ID NO: 1-4, 6, 14, 16, 25-27, 29, 157-159, 161, 183-185, 187, 214, 216, 240, 242, 271, 273, 300-301, 303, 322, 324, 345-347, 349, 353-354, 356, 377, 379, 405-407, 409, 418-419, 421, 441-443, 485-486, 488, 503, 504, 506, 514-515, 517, 526-527, 529, 547, 549, 556, 558, 570-571, 573, 577-578, 580, 587, 589, 601, 603, 606, 608, 611, 613, 617, 619, 621, 623, 625, 627, 629, or 631 or a representative fragment thereof; or a nucleotide sequence at least 95% identical to any of the nucleotide sequences of SEQ ID NO: 1-4, 6, 14, 16, 25-27, 29, 157-159, 161, 183-185, 187, 214, 216, 240, 242, 271, 273, 300-301, 303, 322, 324, 345-347, 349, 353-354, 356, 377, 379, 405-407, 409, 418-419, 421, 441-443, 485-486, 488, 503, 504, 506, 514-515, 517, 526-527, 529, 547, 549, 556, 558, 570-571, 573, 577-578, 580, 587, 589, 601, 603, 606, 608, 611, 613, 617, 619, 621, 623, 625, 627, 629, or 631 in computer readable form, a skilled artisan can routinely access the sequence information for a variety of purposes. Computer software is publicly available which allows a skilled artisan to access sequence information provided in a computer readable medium. The examples which follow demonstrate how software which implements the BLAST (Altschul et al., J. Mol. Biol. 215:403-410 (1990)) and BLAZE (Brutlag et al., Comp. Chem. 17:203-207 (1993)) search algorithms on a Sybase system is used to identify open reading frames (ORFs) within a nucleic acid sequence. Such ORFs may be protein-encoding fragments and may be useful in producing commercially important proteins such as enzymes used in fermentation reactions and in the production of commercially useful metabolites.

As used herein, “a computer-based system” refers to the hardware means, software means, and data storage means used to analyze the nucleotide sequence information of the present invention. The minimum hardware means of the computer-based systems of the present invention comprises a central processing unit (CPU), input means, output means, and data storage means. A skilled artisan can readily appreciate that any one of the currently available computer-based systems are suitable for use in the present invention. As stated above, the computer-based systems of the present invention comprise a data storage means having stored therein a nucleotide sequence of the present invention and the necessary hardware means and software means for supporting and implementing a search means. As used herein, “data storage means” refers to memory which can store nucleotide sequence information of the present invention, or a memory access means which can access manufactures having recorded thereon the nucleotide sequence information of the present invention.

As used herein, “search means” refers to one or more programs which are implemented on the computer-based system to compare a target sequence or target structural motif with the sequence information stored within the data storage means. Search means are used to identify fragments or regions of a known sequence which match a particular target sequence or target motif. A variety of known algorithms are disclosed publicly and a variety of commercially available software for conducting search means are and can be used in the computer-based systems of the present invention. Examples of such software include, but are not limited to, Smith-Waterman, MacPattern (EMBL), BLASTN and BLASTA (NPOLYPEPTIDEIA). A skilled artisan can readily recognize that any one of the available algorithms or implementing software packages for conducting homology searches can be adapted for use in the present computer-based systems. As used herein, a “target sequence” can be any nucleic acid or amino acid sequence of six or more nucleotides or two or more amino acids. A skilled artisan can readily recognize that the longer a target sequence is, the less likely a target sequence will be present as a random occurrence in the database. The most preferred sequence length of a target sequence is from about 10 to 100 amino acids, or from about 30 to 300 nucleotide residues. However, it is well recognized that searches for commercially important fragments, such as sequence fragments involved in gene expression and protein processing, may be of shorter length.

As used herein, “a target structural motif,” or “target motif,” refers to any rationally selected sequence or combination of sequences in which the sequence(s) are chosen based on a three-dimensional configuration which is formed upon the folding of the target motif There are a variety of target motifs known in the art. Protein target motifs include, but are not limited to, enzyme active sites and signal sequences. Nucleic acid target motifs include, but are not limited to, promoter sequences, hairpin structures and inducible expression elements (protein binding sequences).

4.22 Triple Helix Formation

In addition, the fragments of the present invention, as broadly described, can be used to control gene expression through triple helix formation or antisense DNA or RNA, both of which methods are based on the binding of a polynucleotide sequence to DNA or RNA. Polynucleotides suitable for use in these methods are usually 20 to 40 bases in length and are designed to be complementary to a region of the gene involved in transcription (triple helix—see Lee et al., Nucl. Acids Res. 6:3073 (1979); Cooney et al., Science 15241:456 (1988); and Dervan et al., Science 251:1360 (1991)) or to the mRNA itself (antisense—Olmno, J. Neurochem. 56:560 (1991); Oligodeoxynucleotides as Antisense Inhibitors of Gene Expression, CRC Press, Boca Raton, Fla. (1988)). Triple helix-formation optimally results in a shut-off of RNA transcription from DNA, while antisense RNA hybridization blocks translation of an mRNA molecule into polypeptide. Both techniques have been demonstrated to be effective in model systems. Information contained in the sequences of the present invention is necessary for the design of an antisense or triple helix oligonucleotide.

4.23 Diagnostic Assays and Kits

The present invention further provides methods to identify the presence or expression of one of the ORFs of the present invention, or homolog thereof, in a test sample, using a nucleic acid probe or antibodies of the present invention, optionally conjugated or otherwise associated with a suitable label.

In general, methods for detecting a polynucleotide of the invention can comprise contacting a sample with a compound that binds to and forms a complex with the polynucleotide for a period sufficient to form the complex, and detecting the complex, so that if a complex is detected, a polynucleotide of the invention is detected in the sample. Such methods can also comprise contacting a sample under stringent hybridization conditions with nucleic acid primers that anneal to a polynucleotide of the invention under such conditions, and amplifying annealed polynucleotides, so that if a polynucleotide is amplified, a polynucleotide of the invention is detected in the sample.

In general, methods for detecting a polypeptide of the invention can comprise contacting a sample with a compound that binds to and forms a complex with the polypeptide for a period sufficient to form the complex, and detecting the complex, so that if a complex is detected, a polypeptide of the invention is detected in the sample.

In detail, such methods comprise incubating a test sample with one or more of the antibodies or one or more of the nucleic acid probes of the present invention and assaying for binding of the nucleic acid probes or antibodies to components within the test sample.

Conditions for incubating a nucleic acid probe or antibody with a test sample vary. Incubation conditions depend on the format employed in the assay, the detection methods employed, and the type and nature of the nucleic acid probe or antibody used in the assay. One skilled in the art will recognize that any one of the commonly available hybridization, amplification or immunological assay formats can readily be adapted to employ the nucleic acid probes or antibodies of the present invention. Examples of such assays can be found in Chard, T., An Introduction to Radioimmunoassay and Related Techniques, Elsevier Science Publishers, Amsterdam, The Netherlands (1986); Bullock, G. R. et al., Techniques in Immunocytochemistry, Academic Press, Orlando, Fla. Vol. 1 (1982), Vol. 2 (1983), Vol. 3 (1985); Tijssen, P., Practice and Theory of immunoassays: Laboratory Techniques in Biochemistry and Molecular Biology, Elsevier Science Publishers, Amsterdam, The Netherlands (1985). The test samples of the present invention include cells, protein or membrane extracts of cells, or biological fluids such as sputum, blood, serum, plasma, or urine. The test sample used in the above-described method will vary based on the assay format, nature of the detection method and the tissues, cells or extracts used as the sample to be assayed. Methods for preparing protein extracts or membrane extracts of cells are well known in the art and can be readily be adapted in order to obtain a sample which is compatible with the system utilized.

In another embodiment of the present invention, kits are provided which contain the necessary reagents to carry out the assays of the present invention. Specifically, the invention provides a compartment kit to receive, in close confinement, one or more containers which comprises: (a) a first container comprising one of the probes or antibodies of the present invention; and (b) one or more other containers comprising one or more of the following: wash reagents, reagents capable of detecting presence of a bound probe or antibody.

In detail, a compartment kit includes any kit in which reagents are contained in separate containers. Such containers include small glass containers, plastic containers or strips of plastic or paper. Such containers allows one to efficiently transfer reagents from one compartment to another compartment such that the samples and reagents are not cross-contaminated, and the agents or solutions of each container can be added in a quantitative fashion from one compartment to another. Such containers will include a container which will accept the test sample, a container which contains the antibodies used in the assay, containers which contain wash reagents (such as phosphate buffered saline, Tris-buffers, etc.), and containers which contain the reagents used to detect the bound antibody or probe. Types of detection reagents include labeled nucleic acid probes, labeled secondary antibodies, or in the alternative, if the primary antibody is labeled, the enzymatic, or antibody binding reagents which are capable of reacting with the labeled antibody. One skilled in the art will readily recognize that the disclosed probes and antibodies of the present invention can be readily incorporated into one of the established kit formats which are well known in the art.

4.24 Medical Imaging

The novel polypeptides and binding partners of the invention are useful in medical imaging of sites expressing the molecules of the invention (e.g., where the polypeptide of the invention is involved in the immune response, for imaging sites of inflammation or infection). See, e.g., Kunkel et al., U.S. Pat. No. 5,413,778. Such methods involve chemical attachment of a labeling or imaging agent, administration of the labeled polypeptide to a subject in a pharmaceutically acceptable carrier, and imaging the labeled polypeptide in vivo at the target site.

4.25 Screening Assays

Using the isolated proteins and polynucleotides of the invention, the present invention further provides methods of obtaining and identifying agents which bind to a polypeptide encoded by an ORF corresponding to any of the nucleotide sequences set forth in SEQ ID NO: 1-4, 6, 14, 16, 25-27, 29, 157-159, 161, 183-185, 187, 214, 216, 240, 242, 271, 273, 300-301, 303, 322, 324, 345-347, 349, 353-354, 356, 377, 379, 405-407, 409, 418-419,421,441-443, 485-486, 488, 503, 504, 506, 514-515, 517, 526-527, 529, 547, 549, 556, 558, 570-571,573,577-578,580,587,589, 601, 603, 606, 608, 611, 613, 617, 619, 621, 623, 625, 627, 629, or 631, or bind to a specific domain of the polypeptide encoded by the nucleic acid. In detail, said method comprises the steps of:
(a) contacting an agent with an isolated protein encoded by an ORF of the present invention, or nucleic acid of the invention; and
(b) determining whether the agent binds to said protein or said nucleic acid.

In general, therefore, such methods for identifying compounds that bind to a polynucleotide of the invention can comprise contacting a compound with a polynucleotide of the invention for a time sufficient to form a polynucleotide/compound complex, and detecting the complex, so that if a polynucleotide/compound complex is detected, a compound that binds to a polynucleotide of the invention is identified.

Likewise, in general, therefore, such methods for identifying compounds that bind to a polypeptide of the invention can comprise contacting a compound with a polypeptide of the invention for a time sufficient to form a polypeptide/compound complex, and detecting the complex, so that if a polypeptide/compound complex is detected, a compound that binds to a polynucleotide of the invention is identified.

Methods for identifying compounds that bind to a polypeptide of the invention can also comprise contacting a compound with a polypeptide of the invention in a cell for a time sufficient to form a polypeptide/compound complex, wherein the complex drives expression of a receptor gene sequence in the cell, and detecting the complex by detecting reporter gene sequence expression, so that if a polypeptide/compound complex is detected, a compound that binds a polypeptide of the invention is identified.

Compounds identified via such methods can include compounds which modulate the activity of a polypeptide of the invention (that is, increase or decrease its activity, relative to activity observed in the absence of the compound). Alternatively, compounds identified via such methods can include compounds which modulate the expression of a polynucleotide of the invention (that is, increase or decrease expression relative to expression levels observed in the absence of the compound). Compounds, such as compounds identified via the methods of the invention, can be tested using standard assays well known to those of skill in the art for their ability to modulate activity/expression.

The agents screened in the above assay can be, but are not limited to, peptides, carbohydrates, vitamin derivatives, or other pharmaceutical agents. The agents can be selected and screened at random or rationally selected or designed using protein modeling techniques.

For random screening, agents such as peptides, carbohydrates, pharmaceutical agents and the like are selected at random and are assayed for their ability to bind to the protein encoded by the ORF of the present invention. Alternatively, agents may be rationally selected or designed. As used herein, an agent is said to be “rationally selected or designed” when the agent is chosen based on the configuration of the particular protein. For example, one skilled in the art can readily adapt currently available procedures to generate peptides, pharmaceutical agents and the like, capable of binding to a specific peptide sequence, in order to generate rationally designed antipeptide peptides, for example see Hurby et al., Application of Synthetic Peptides: Antisense Peptides,” In Synthetic Peptides, A User's Guide, W.H. Freeman, NY (1992), pp. 289-307, and Kaspczak et al, Biochemistry 28:9230-8 (1989), or pharmaceutical agents, or the like.

In addition to the foregoing, one class of agents of the present invention, as broadly described, can be used to control gene expression through binding to one of the ORFs or EMFs of the present invention. As described above, such agents can be randomly screened or rationally designed/selected. Targeting the ORF or EMF allows a skilled artisan to design sequence specific or element specific agents, modulating the expression of either a single ORF or multiple ORFs which rely on the same EMF for expression control. One class of DNA binding agents are agents which contain base residues which hybridize or form a triple helix formation by binding to DNA or RNA. Such agents can be based on the classic phosphodiester, ribonucleic acid backbone, or can be a variety of sulfhydryl or polymeric derivatives which have base attachment capacity.

Agents suitable for use in these methods usually contain 20 to 40 bases and are designed to be complementary to a region of the gene involved in transcription (triple helix—see Lee et al., Nucl. Acids Res. 6:3073 (1979); Cooney et al., Science 241:456 (1988); and Dervan et al., Science 251:1360 (1991)) or to the mRNA itself (antisense—Okano, J. Neurochem. 56:560 (1991); Oligodeoxynucleotides as Antisense Inhibitors of Gene Expression, CRC Press, Boca Raton, Fla. (1988)). Triple helix-formation optimally results in a shut-off of RNA transcription from DNA, while antisense RNA hybridization blocks translation of an mRNA molecule into polypeptide. Both techniques have been demonstrated to be effective in model systems. Information contained in the sequences of the present invention is necessary for the design of an antisense or triple helix oligonucleotide and other DNA binding agents.

Agents which bind to a protein encoded by one of the ORFs of the present invention can be used as a diagnostic agent. Agents which bind to a protein encoded by one of the ORFs of the present invention can be formulated using known techniques to generate a pharmaceutical composition.

4.26 Use of Nucleic Acids as Probes

Another aspect of the subject invention is to provide for polypeptide-specific nucleic acid hybridization probes capable of hybridizing with naturally occurring nucleotide sequences. The hybridization probes of the subject invention may be derived from any of the nucleotide sequences SEQ ID NO: 1-4, 6, 14, 16, 25-27, 29, 157-159, 161, 183-185, 187, 214, 216, 240, 242, 271, 273, 300-301, 303, 322, 324, 345-347, 349, 353-354, 356, 377, 379, 405-407, 409, 418-419, 421, 441-443, 485-486, 488, 503, 504, 506, 514-515, 517, 526-527, 529, 547, 549, 556, 558, 570-571, 573, 577-578, 580, 587, 589, 601, 603, 606, 608, 611, 613, 617, 619, 621, 623, 625, 627, 629, or 631. Because the corresponding gene is only expressed in a limited number of tissues, a hybridization probe derived from of any of the nucleotide sequences SEQ ID NO: 1-4, 6, 14, 16, 25-27, 29, 157-159, 161, 183-185, 187, 214, 216, 240, 242, 271, 273, 300-301, 303, 322, 324, 345-347, 349, 353-354, 356, 377, 379, 405-407, 409, 418-419, 421, 441-443, 485-486, 488, 503, 504, 506, 514-515, 517, 526-527, 529, 547, 549, 556, 558, 570-571, 573, 577-578, 580, 587, 589, 601, 603, 606, 608, 611, 613, 617, 619, 621, 623, 625, 627, 629, or 631 can be used as an indicator of the presence of RNA of cell type of such a tissue in a sample.

Any suitable hybridization technique can be employed, such as, for example, in situ hybridization. PCR as described in U.S. Pat. Nos. 4,683,195 and 4,965,188 provides additional uses for oligonucleotides based upon the nucleotide sequences. Such probes used in PCR may be of recombinant origin, may be chemically synthesized, or a mixture of both. The probe will comprise a discrete nucleotide sequence for the detection of identical sequences or a degenerate pool of possible sequences for identification of closely related genomic sequences.

Other means for producing specific hybridization probes for nucleic acids include the cloning of nucleic acid sequences into vectors for the production of mRNA probes. Such vectors are known in the art and are commercially available and may be used to synthesize RNA probes in vitro by means of the addition of the appropriate RNA polymerase as T7 or SP6 RNA polymerase and the appropriate radioactively labeled nucleotides. The nucleotide sequences may be used to construct hybridization probes for mapping their respective genomic sequences. The nucleotide sequence provided herein may be mapped to a chromosome or specific regions of a chromosome using well known genetic and/or chromosomal mapping techniques. These techniques include in situ hybridization, linkage analysis against known chromosomal markers, hybridization screening with libraries or flow-sorted chromosomal preparations specific to known chromosomes, and the like. The technique of fluorescent in situ hybridization of chromosome spreads has been described, among other places, in Verma et al (1988) Human Chromosomes: A Manual of Basic Techniques, Pergamon Press, New York N.Y.

Fluorescent in situ hybridization of chromosomal preparations and other physical chromosome mapping techniques may be correlated with additional genetic map data. Examples of genetic map data can be found in the 1994 Genome Issue of Science (265:1981f). Correlation between the location of a nucleic acid on a physical chromosomal map and a specific disease (or predisposition to a specific disease) may help delimit the region of DNA associated with that genetic disease. The nucleotide sequences of the subject invention may be used to detect differences in gene sequences between normal, carrier or affected individuals.

4.27 Preparation of Support Bound Oligonucleotides

Oligonucleotides, i.e., small nucleic acid segments, may be readily prepared by, for example, directly synthesizing the oligonucleotide by chemical means, as is commonly practiced using an automated oligonucleotide synthesizer.

Support bound oligonucleotides may be prepared by any of the methods known to those of skill in the art using any suitable support such as glass, polystyrene or Teflon. One strategy is to precisely spot oligonucleotides synthesized by standard synthesizers. Immobilization can be achieved using passive adsorption (Inouye & Hondo, J. Clin Microbiol 28:1462-72 (1990)); using UV light (Nagata et al., 1985; Dahlen et al., 1987; Morrissey & Collins, Mol. Cell Probes 3:189-207 (1989)) or by covalent binding of base modified DNA (Keller et al., 1988; 1989); all references being specifically incorporated herein.

Another strategy that may be employed is the use of the strong biotin-streptavidin interaction as a linker. For example, Broude et al. Proc. Natl. Acad. Sci USA 91:3072-6 (1994) describe the use of biotinylated probes, although these are duplex probes, that are immobilized on streptavidin-coated magnetic beads. Streptavidin-coated beads may be purchased from Dynal, Oslo. Of course, this same linking chemistry is applicable to coating any surface with streptavidin. Biotinylated probes may be purchased from various sources, such as, e.g., Operon Technologies (Alameda, Calif.).

Nunc Laboratories (Naperville, Ill.) is also selling suitable material that could be used. Nunc Laboratories have developed a method by which DNA can be covalently bound to the microwell surface termed Covalink NH. CovaLink NH is a polystyrene surface grafted with secondary amino groups (>NH) that serve as bridge-heads for further covalent coupling. CovaLink Modules may be purchased from Nunc Laboratories. DNA molecules may be bound to CovaLink exclusively at the 5′-end by a phosphoramidate bond, allowing immobilization of more than 1 pmol of DNA (Rasmussen et al., Anal Biochem 198:138-42 (1991)).

The use of CovaLink NH strips for covalent binding of DNA molecules at the 5′-end has been described (Rasmussen et al., 1991). In this technology, a phosphoramidate bond is employed (Chu et al., Nucleic Acids 11:6513-29 (1983)). This is beneficial as immobilization using only a single covalent bond is preferred. The phosphoramidate bond joins the DNA to the CovaLink NH secondary amino groups that are positioned at the end of spacer arms covalently grafted onto the polystyrene surface through a 2 nm long spacer arm. To link an oligonucleotide to CovaLink NH via an phosphoramidate bond, the oligonucleotide terminus must have a 5′-end phosphate group. It is, perhaps, even possible for biotin to be covalently bound to CovaLink and then streptavidin used to bind the probes.

More specifically, the linkage method includes dissolving DNA in water (7.5 ng/ul) and denaturing for 10 min. at 95° C. and cooling on ice for 10 min. Ice-cold 0.1 M 1-methylimidazole, pH 7.0 (1-MeIm₇), is then added to a final concentration of 10 mM 1-MeIm₇. A ss DNA solution is then dispensed into CovaLink NH strips (75 ul/well) standing on ice.

Carbodiimide 0.2 M 1-ethyl-3-(3-dimethylaminopropyl)-carbodiimide (EDC), dissolved in 10 mM 1-MeIm₇, is made fresh and 25 ul added per well. The strips are incubated for 5 hours at 50° C. After incubation the strips are washed using, e.g., Nunc-Immuno Wash; first the wells are washed 3 times, then they are soaked with washing solution for 5 min., and finally they are washed 3 times (where in the washing solution is 0.4 N NaOH, 0.25% SDS heated to 50° C.).

It is contemplated that a further suitable method for use with the present invention is that described in PCT Patent Application WO 90/03382 (Southern & Maskos), incorporated herein by reference. This method of preparing an oligonucleotide bound to a support involves attaching a nucleoside 3′-reagent through the phosphate group by a covalent phosphodiester link to aliphatic hydroxyl groups carried by the support. The oligonucleotide is then synthesized on the supported nucleoside and protecting groups removed from the synthetic oligonucleotide chain under standard conditions that do not cleave the oligonucleotide from the support. Suitable reagents include nucleoside phosphoramidite and nucleoside hydrogen phosphorate.

An on-chip strategy for the preparation of DNA probe for the preparation of DNA probe arrays may be employed. For example, addressable laser-activated photodeprotection may be employed in the chemical synthesis of oligonucleotides directly on a glass surface, as described by Fodor et al. Science 251:767-73 (1991)), incorporated herein by reference. Probes may also be immobilized on nylon supports as described by Van Ness et al. Nucleic Acids Res. 19:3345-50 (1991); or linked to Teflon using the method of Duncan & Cavalier, Anal Biochem 169:104-8 (1988); all references being specifically incorporated herein.

To link an oligonucleotide to a nylon support, as described by Van Ness et al. (1991), requires activation of the nylon surface via alkylation and selective activation of the 5′-amine of oligonucleotides with cyanuric chloride.

One particular way to prepare support bound oligonucleotides is to utilize the light-generated synthesis described by Pease et al., Proc. Natl. Acad. Sci USA 91:5022-6 (1994). These authors used current photolithographic techniques to generate arrays of immobilized oligonucleotide probes (DNA chips). These methods, in which light is used to direct the synthesis of oligonucleotide probes in high-density, miniaturized arrays, utilize photolabile 5′-protected N-acyl-deoxynucleoside phosphoramidites, surface linker chemistry and versatile combinatorial synthesis strategies. A matrix of 256 spatially defined oligonucleotide probes may be generated in this manner.

4.28 Preparation of Nucleic Acid Fragments

The nucleic acids may be obtained from any appropriate source, such as cDNAs, genomic DNA, chromosomal DNA, microdissected chromosome bands, cosmid or YAC inserts, and RNA, including mRNA without any amplification steps. For example, Sambrook et al. (1989) describes three protocols for the isolation of high molecular weight DNA from mammalian cells (p. 9.14-9.23).

DNA fragments may be prepared as clones in M13, plasmid or lambda vectors and/or prepared directly from genomic DNA or cDNA by PCR or other amplification methods. Samples may be prepared or dispensed in multiwell plates. About 100-1000 ng of DNA samples may be prepared in 2-500 ml of final volume.

The nucleic acids would then be fragmented by any of the methods known to those of skill in the art including, for example, using restriction enzymes as described at 9.24-9.28 of Sambrook et al. (1989), shearing by ultrasound and NaOH treatment.

Low pressure shearing is also appropriate, as described by Schriefer et al. Nucleic Acids Res. 18:7455-6 (1990). In this method, DNA samples are passed through a small French pressure cell at a variety of low to intermediate pressures. A lever device allows controlled application of low to intermediate pressures to the cell. The results of these studies indicate that low-pressure shearing is a useful alternative to sonic and enzymatic DNA fragmentation methods.

One particularly suitable way for fragmenting DNA is contemplated to be that using the two base recognition endonuclease, CviJI, described by Fitzgerald et al. Nucleic Acids Res. 20:3753-62 (1992). These authors described an approach for the rapid fragmentation and fractionation of DNA into particular sizes that they contemplated to be suitable for shotgun cloning and sequencing.

The restriction endonuclease CviJI normally cleaves the recognition sequence PuGCPy between the G and C to leave blunt ends. Atypical reaction conditions, which alter the specificity of this enzyme (CviJI**), yield a quasi-random distribution of DNA fragments form the small molecule pUC19 (2688 base pairs). Fitzgerald et al. (1992) quantitatively evaluated the randomness of this fragmentation strategy, using a CviJI** digest of pUC19 that was size fractionated by a rapid gel filtration method and directly ligated, without end repair, to a lac Z minus M13 cloning vector. Sequence analysis of 76 clones showed that CviJI** restricts pyGCPy and PuGCPu, in addition to PuGCPy sites, and that new sequence data is accumulated at a rate consistent with random fragmentation.

As reported in the literature, advantages of this approach compared to sonication and agarose gel fractionation include: smaller amounts of DNA are required (0.2-0.5 μg instead of 2-5 μg); and fewer steps are involved (no preligation, end repair, chemical extraction, or agarose gel electrophoresis and elution are needed).

Irrespective of the manner in which the nucleic acid fragments are obtained or prepared, it is important to denature the DNA to give single stranded pieces available for hybridization. This is achieved by incubating the DNA solution for 2-5 minutes at 80-90° C. The solution is then cooled quickly to 2° C. to prevent renaturation of the DNA fragments before they are contacted with the chip. Phosphate groups must also be removed from genomic DNA by methods known in the art.

4.29 Preparation of DNA Arrays

Arrays may be prepared by spotting DNA samples on a support such as a nylon membrane. Spotting may be performed by using arrays of metal pins (the positions of which correspond to an array of wells in a microtiter plate) to repeated by transfer of about 20 nl of a DNA solution to a nylon membrane. By offset printing, a density of dots higher than the density of the wells is achieved. One to 25 dots may be accommodated in 1 mm², depending on the type of label used. By avoiding spotting in some preselected number of rows and columns, separate subsets (subarrays) may be formed. Samples in one subarray may be the same genomic segment of DNA (or the same gene) from different individuals, or may be different, overlapped genomic clones. Each of the subarrays may represent replica spotting of the same samples. In one example, a selected gene segment may be amplified from 64 patients. For each patient, the amplified gene segment may be in one 96-well plate (all 96 wells containing the same sample). A plate for each of the 64 patients is prepared. By using a 96-pin device, all samples may be spotted on one 8×12 cm membrane. Subarrays may contain 64 samples, one from each patient. Where the 96 subarrays are identical, the dot span may be 1 mm² and there may be a 1 mm space between subarrays.

Another approach is to use membranes or plates (available from NUNC, Naperville, Ill.) which may be partitioned by physical spacers e.g. a plastic grid molded over the membrane, the grid being similar to the sort of membrane applied to the bottom of multiwell plates, or hydrophobic strips. A fixed physical spacer is not preferred for imaging by exposure to flat phosphor-storage screens or x-ray films.

The present invention is illustrated in the following examples. Upon consideration of the present disclosure, one of skill in the art will appreciate that many other embodiments and variations may be made in the scope of the present invention. Accordingly, it is intended that the broader aspects of the present invention not be limited to the disclosure of the following examples. The present invention is not to be limited in scope by the exemplified embodiments which are intended as illustrations of single aspects of the invention, and compositions and methods which are functionally equivalent are within the scope of the invention. Indeed, numerous modifications and variations in the practice of the invention are expected to occur to those skilled in the art upon consideration of the present preferred embodiments. Consequently, the only limitations which should be placed upon the scope of the invention are those which appear in the appended claims.

All references cited within the body of the instant specification are hereby incorporated by reference in their entirety.

### 5. EXAMPLES

### Example 1

### Isolation Of SEQ ID NO: 1, 25, 157, 183, 300, 345, 353, 405, 441, 486, 504, 515, 527, 571, 578, 630, and 632 from a cDNA Library of Human Cells

The novel nucleic acids of SEQ ID NO: 1, 25, 157, 183, 300, 345, 353, 405, 441, 486, 504, 515, 527, 571, 578, 630, and 632 were obtained from various human cDNA libraries using standard PCR, sequencing by hybridization sequence signature analysis, and Sanger sequencing techniques. The inserts of the library were amplified with PCR using primers specific for vector sequences flanking the inserts. These samples were spotted onto nylon membranes and interrogated with oligonucleotide probes to give sequence signatures. The clones were clustered into groups of similar or identical sequences, and single representative clones were selected from each group for gel sequencing. The 5′ sequence of the amplified inserts were then deduced using the reverse M13 sequencing primer in a typical Sanger sequencing protocol. PCR products were purified and subjected to fluorescent dye terminator cycle sequencing. Single-pass gel sequencing was done using a 377 Applied Biosystems (ABI) sequencer. These inserts were identified as a novel sequence not previously obtained from this library and not previously reported in public databases. These sequences are designated as SEQ ID NO: 1, 25, 157, 183, 300, 345, 353, 405, 441, 486, 504, 515, 527, 571, 578, 630, and 632 in the attached sequence listing.

### Example 2

### Assemblage of SEQ ID NO: 2, 3, 26, 158, 184, 299, or 346

The novel nucleic acids (SEQ ID NO: 2, 3, 26, 158, 184, 299, or 346) of the invention were assembled from sequences that were obtained from various cDNA libraries by methods described in Example 1 above, and in some cases obtained from one or more public databases. The final sequence was assembled using the EST sequence as seed. Then a recursive algorithm was used to extend the seed into an extended assemblage, by pulling additional sequences from different databases (ie. Hyseq's database containing EST sequences, dbEST, gb pri, and UniGene) that belong to this assemblage. The algorithm terminated when there was no additional sequences from the above databases that would extend the assemblage. Inclusion of component sequences into the assemblage was based on a BLASTN hit to the extending assemblage with BLAST score greater than 300 and percent identity greater than 95%.

The nearest neighbor results for the assembled contigs were obtained by a FASTA search against Genpept, using FASTXY algorithm. FASTXY is an improved version of FASTA alignment which allows in-codon frame shifts. The nearest neighbor results showed the closest homologue for each assemblage from Genpept (and contain the translated amino acid sequences for which the assemblages encodes). The nearest neighbor results are set forth in Table 45 below:

TABLE 45

| | | | Smith- | |
|---|---|---|---|---|
| SEQ ID | Accession | | Waterman | |
| NO: | No. | Description | Score | % Identity |
| 2 | U27838 | Mus musculus glycosyl- | 418 | 29.216 |
| | | phosphatidyl-inositol- | | |
| | | anchored protein homolog | | |
| 3 | M19419 | Mus musculus proline-rich | 244 | 36.683 |
| | | salivary protein | | |
| 158 | X53556 | Bos taurus type X collagen | 657 | 42.963 |
| 184 | L23982 | Homo sapiens collagen | 521 | 46.226 |
| | | type VII | | |
| 346 | AF095737 | Homo sapiens unknown | 366 | 68.085 |

The predicted amino acid sequences for SEQ ID NO: 2, 3, 26, 158, 184, 299, or 346 was obtained by using a software program called FASTY (available from http://fasta:bioch.virginia.edu) which selects a polypeptide based on a comparison of translated novel polynucleotide to known polynucleotides (W. R. Pearson, Methods in Enzymology, 183:63-98 (1990), incorporated herein by reference). The results for SEQ ID NO: 2, 3, 26, 158, 184, 299, or 346 are shown in Table 46 below, wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine, X=Unknown, *=Stop Codon, /=possible nucleotide deletion, \=possible nucleotide insertion:

TABLE 46

| | | | |
|---|---|---|---|
| | Predicted | | |
| | beginning | Predicted end | |
| | nucleotide | nucleotide | |
| | location | location | |
| | corresponding | corresponding | |
| | to first amino | to first amino | |
| SEQ | acid residue of | acid residue of | |
| ID | amino acid | amino acid | Amino acid segment containing |
| NO: | sequence | sequence | signal peptide |

| | | | |
|---|---|---|---|
| 2 | 3 | 2456 | FDTYRGLPSISNGNYSQLQFQAREYSGAPYSQ |
| | | | RISMTTVSVAWKVLSGKIGEGAEGNCKCVISE |
| | | | GAWAVCPTQPCGKAKPDKHLKDLLSKLLNSG |
| | | | YFESIPVPKNAKEKEVPLEEEMLIQSEKKTQLS |
| | | | KTESVKESESLMEFAQPEIQPQEFLNRRYMTE |
| | | | VDYSNXQGEEQPWEADYARKPNLPKRWDML |
| | | | TEPDGQEKKQESFKSWEASGKHQEVSKPAVS |
| | | | LEQRKQDTSKLRSTLPEEQKKQEISKSKPSPSQ |
| | | | WKQDTPKSKAGYVQEEHKKQETPKLWPVQL |
| | | | QKEQDPKKQTPKSWTPSMQSEQNTTKSWTTP |
| | | | MCEEQDSKQPETPKSWENNVESQKIHSLTSQS |
| | | | QISPKSWGVATASLWNDQLLPRKLNTEPKDVP |
| | | | /IACASA*GFLPLQPPFRRI/HVLRKEKLQDLMT |
| | | | QIQGTCNFMQESVLDFDKPSSAIPTSQPPSATP |
| | | | G*PRRHLKEQNLS\VKVLFFQGAVT\VFNVNAP |
| | | | LPPRKEQEIKIESPYSPGYNQSFTTASTQTPPQC |
| | | | QLPSIIHVEQTVHSQETANYHPDGTIQVSNGSL |
| | | | AFYPAQTNVFPRPTQPFVNSRGSVRGCTRGGR |
| | | | LITNSYRSPGGYKGFDTYRGLPSISNGNYSQLQ |
| | | | FQAREYSGAPYSQRDNFQQCYKRGGTSGGPR |
| | | | ANSRAGWSDSSQVSSPERDNETFNSGDSGQG |
| | | | DSRSMTPVDVPVTNPAATILPVHVYPLPQQMR |
| | | | VAFSAARTSNLAPGTLDQPEVFDLLLNNLGETF |
| | | | DLQLGRFNCPVNGTYVFIFHMLKLAVNVIPLY |
| | | | VNLMKNEEVLVSAYANDGAPDHETASNHAIL |
| | | | QLFQGDQIWLRLHRGAIYGSSW |
| | | | (SEQ ID NO: 23) |
| 3 | 39 | 599 | GASPNQGQNRPHARQRAPPQ/G/PPGEPERRAP |
| | | | LPSGHGEPCRHRPPPFPQPP/AGTQKPLLQGPG |
| | | | GG*PAENAPTAALGSPAPPRGCQAAPPPRSGA |
| | | | GRPDLPTLAGPRPAPA\PPPSAAPPPPPSGAPSR/ |
| | | | PAAGRQRLSGVSSGPSLGWW*VGRGRGLPAF |
| | | | AQIAGHQVGPRRRRTPAGRKPRSPAGPR |
| | | | (SEQ ID NO: 24) |
| 26 | 202 | 2471 | FDSAVLSSINVMAVLPGPLQLLGVLLTISLSSIR |
| | | | LIQAGAYYGIIKPLPPQIPPQMPPQIPQYQPLGQ |
| | | | QVPHMPLAKDGLAMGKEMPHLQYGKEYPHL |
| | | | PQYMIKEIQPAPRMGKEAVPKKGKEIPLASLRG |
| | | | EQGPRGEPGPRGPPGPPGLPGHGIPGIXGKPGP |
| | | | QGYPGVGKPGMPGMPGKPGAMGMPGAKGEI |
| | | | GQKGEIGPMGLP*PQGPPGPHGLPGIGKPGGPG |
| | | | LPGQPGPKGDRGPKGLPGPQGLRGPKGDKGF |
| | | | GMPGAPGVKGPPGMHGPPGPVGLPGVGKPGV |
| | | | TGFPGP\QGPLGK\PGAPGEPGPQGPIGVPGVQ |
| | | | GPPGIPGIGKPGQDG\IPGQPGFPGGKGEQGLP |
| | | | GLPGPPGLPGIGKPGFPGPKGDRGMGGVPGAL |
| | | | GPRGEKGPIGAPGIGGPPGEPGLPGIPGPMGPP |
| | | | GAIGFPGPKGEGGIVGPQGPPGPKGEPGLQGFP |
| | | | GKPGFLGEVGPPGMRGFPGPIGPKGEHGQKG |
| | | | VPGLPGVPGLLGPKGEPGIPGDQGLQGPPGWG |
| | | | IGGPSGPIGPPGIPGPKGEPGLPGPPGFPGIGKP |
| | | | GVAGLHGPPGKPGALGPQGQPGLPGPPGPPGP |
| | | | PGPPAVMPPTPPPQGEYLPDMGLGIDGVKPPH |
| | | | AYGAKKGKNGGPAYEMPAFTABLTAPFPPVG |
| | | | APVKFNKLLYNGRQNYNPQTGIFTCEVPGVY |
| | | | YFAYHVHCKGGNVWVALFKNNEPVMYTYDE |
| | | | YKKGFLDQASGSAVLLLRPGDRVFLQMPSEQ |
| | | | AAGLYAGQYVHSSFSGYLLYPM |
| | | | (SEQ ID NO: 156) |
| 158 | 142 | 1058 | SSKTPAVGRSCEQEPKMFVLLYVTSFAICASG |
| | | | QPRGNQLKGENYSPRYICSLPGLPGPPGPPGAN |
| | | | GSPGPHGRIGLPGRDGRDGRKGEKGEKGTAG |
| | | | LRGKTGPLGLAGEKGDQGETGKKGPIGPEGE |
| | | | KGEVGPIGPPGPKGDRGEQGDPGLPGVCRCGS |
| | | | IVLKSAFSVGITTSYPEERLPIIFNKVLFNEGEH |
| | | | YNPATGKFICAFPGIYYFSYDITLANKHLAIGL |
| | | | VHNGQYRIKTFDANTGNHDVASGSTVIYLQPE |
| | | | DEVWLEIFFTDQNGLFSDPGWADSLFSGFLLY |
| | | | VDTDYLDSISEDDEL (SEQ ID NO: 182) |
| 184 | 739 | 794 | ASFLLQMCP*GPVQSLSSEP*GSGGFCLPLKSA |
| | | | QGT*T/PQDTCRQGHPGLPGNPGHNGLPGRDG |
| | | | RDGAKGDKGDAGEPGRPGSPGKDGTSGEKGE |
| | | | RGADGKVEAKGIKGDQGS\*GSPGKHGPKGLA |
| | | | GPMGEKGLRGETGPQGQKGNXGDVGPTGPE |
| | | | GPRGNIGPLGPTGLPGPMGPIGKPGPKGEAGPT |
| | | | GPQGEPGVRGIRGWKGDRGEKGKIGETLVLP |
| | | | KSAFTVGLTVLSKFPSSDVPIKFDKIHIT |
| | | | (SEQ ID NO: 299) |
| 346 | 2471 | 2985 | ETSLERERLSFCTGSRTTRSAELKAVGFEAALQ |
| | | | EVITPEVVPASQSEAYQTLRQNQAQVHNFFFF |
| | | | WGGDSPTLSPRLECSSMSAHCNLRLPGSSNSP |
| | | | TSASRVAGTTGACRHARLIFCILVEMGFHRVA |
| | | | QAGRELLSSANPPTSASQSAGITGMSHHAQPS |
| | | | SQLLISSCC (SEQ ID NO: 352) |

### Example 3

### Assemblage of SEQ ID NO: 4, 14, 27, 159, 185, 214, 240, or 271

The novel nucleic acids (SEQ ID NO: 4, 14, 27, 159, 185, 214, 240, or 271) of the invention were assembled from sequences that were obtained from cDNA libraries by methods described in Example 1 above, and in some cases obtained from one or more public databases. The final sequences were assembled using the EST sequences as seed. Then a recursive algorithm was used to extend the seed into an extended assemblage, by pulling additional sequences from different databases (i.e. Hyseq's database containing EST sequences, dbEST, gb pri, and UniGene) that belong to this assemblage. The algorithm terminated when there was no additional sequences from the above databases that would extend the assemblage. Inclusion of component sequences into the assemblage was based on a BLASTN hit to the extending assemblage with BLAST score greater than 300 and percent identity greater than 95%.

Using PHRAP (Univ. of Washington) or CAP4 (Paracel), a full-length gene cDNA sequence and its corresponding protein sequence were generated from the assemblage. Any frame shifts and incorrect sop codons were corrected by hand editing. During editing, the sequence was checked using FASTY and BLAST against Genbank (i.e. dbEST, gb pri, UniGene, Genpept). Other computer programs which may have been used in the editing process were phredPhrap and Consed (University of Washington) and ed-ready, ed-ext and cg-zip-2 (Hyseq, Inc.). The full-length nucleotide sequences are shown in the Sequence Listing as SEQ ID NO: 4, 14, 27, 159, 185, 214, 240, or 271; and the full-length amino acid sequences are shown in the sequence listing as SEQ ID NO: 5, 15, 28, 160, 186, 215, 241, or 272.

Further annotation of SEQ ID NO: 4, or 14 can be found in U.S. patent application Ser. No. 09/598,075 filed Jun. 20, 2000 (attorney docket no. 787); herein incorporated by reference in its entirety.

Further annotation of SEQ ID NO: 27 can be found in U.S. patent application Ser. No. 09/620,312 filed Jul. 19, 2000 (attorney docket no. 784); herein incorporated by reference in its entirety.

Further annotation of SEQ ID NO: 159 can be found in U.S. patent application Ser. No. 09/728,952 filed Nov. 30, 2000 (attorney docket no. 799); herein incorporated by reference in its entirety.

Further annotation of SEQ ID NO: SEQ ID NO: 185, 214, 240, or 271 can be found in U.S. Provisional patent application Ser. No. 60/306,971 filed Jul. 21, 2001 (attorney docket no. 805); herein incorporated by reference in its entirety.

### Example 4

### Assemblage of SEQ ID NO: 301, 322, 347, 354, or 377

The novel nucleic acid (SEQ ID NO: 301, 322, 347, 354, or 377) of the invention were assembled from sequences that was obtained from a cDNA library by methods described in Example 1 above, and in some cases obtained from one or more public databases. The final sequence was assembled using the EST sequences as seed. Then a recursive algorithm was used to extend the seed into an extended assemblage, by pulling additional sequences from different databases (i.e. Hyseq's database containing EST sequences, dbEST, gb pri, and UniGene) that belong to this assemblage. The algorithm terminated when there was no additional sequences from the above databases that would extend the assemblage. Inclusion of component sequences into the assemblage was based on a BLASTN hit to the extending assemblage with BLAST score greater than 300 and percent identity greater than 95%.

Using PHRAP (Univ. of Washington) or CAP4 (Paracel), a full-length gene cDNA sequence and its corresponding protein sequence were generated from the assemblage. Any frame shifts and incorrect sop codons were corrected by hand editing. During editing, the sequence was checked using FASTY and BLAST against Genbank (ie. dbEST, gb pri, UniGene, Genpept). Other computer programs which may have been used in the editing process were phredPhrap and Consed (University of Washington) and ed-ready, ed-ext and cg-zip-2 (Hyseq, Inc.). The full-length nucleotide sequences are shown in the Sequence Listing as SEQ ID NO: 301, 322, 347, 354, or 377; and the full-length amino acid sequences are shown in the sequence listing as SEQ ID NO: 302, 323, 348, 355, or 378.

### Example 5

### Assemblage of SEQ ID NO: 406

The novel nucleic acid (SEQ ID NO: 406) of the invention was assembled from sequences that were obtained from various cDNA libraries by methods described in Example 1 above, and in some cases obtained from one or more public databases. The final sequence was assembled using the EST sequence as seed. Then a recursive algorithm was used to extend the seed into an extended assemblage, by pulling additional sequences from different databases (i.e. Hyseq's database containing EST sequences, dbEST, gb pri, and UniGene) that belong to this assemblage. The algorithm terminated when there was no additional sequences from the above databases that would extend the assemblage. Inclusion of component sequences into the assemblage was based on a BLASTN hit to the extending assemblage with BLAST score greater than 300 and percent identity greater than 95%.

The nearest neighbor result for the assembled contigs were obtained by a FASTA search against Genpept, using FASTXY algorithm. FASTXY is an improved version of FASTA alignment which allows in-codon frame shifts. The nearest neighbor result showed the closest homologue for each assemblage from Genpept (and contains the translated amino acid sequences for which the assemblage encodes). The nearest neighbor result is set forth in Table 47 below:

TABLE 47

| | | | Smith- | |
|---|---|---|---|---|
| SEQ ID | Accession | | Waterman | |
| NO: | No. | Description | Score | % Identity |
| 406 | X89015 | Homo sapiens leupin | 996 | 44.162 |

The predicted amino acid sequences for SEQ ID NO: 406 were obtained by using a software program called FASTY (available from http://fasta:bioch.virginia.edu) which selects a polypeptide based on a comparison of translated novel polynucleotide to known polynucleotides (W. R. Pearson, Methods in Enzymology, 183:63-98 (1990), incorporated herein by reference). The results for SEQ ID NO: 406 are shown in Table 48 below wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine, X=Unknown, *=Stop Codon, /=possible nucleotide deletion, \=possible nucleotide insertion:

TABLE 48

| | | |
|---|---|---|
| Predicted | | |
| beginning | Predicted end | |
| nucleotide | nucleotide | |
| location | location | |
| corresponding | corresponding | |
| to first amino | to first amino | |
| acid residue of | acid residue of | |
| amino acid | amino acid | Amino acid segment containing |
| sequence | sequence | signal peptide |

| | | |
|---|---|---|
| 1 | 851 | MRASPLEGNSGKNTDSSNITQKPELVPPDLWT |
| | | HPERLATPQQTPTELQESFASTLETTSLISNNLL |
| | | LKMSQSKATVEGLKEAKLGWSLSPGPTHLVL |
| | | TEPHQ1VISFTMDSLVTANTKFCFDLFQEIGKD |
| | | DRHKNIFFSPLSLSAALGMVRLGARSDSAHQI |
| | | DEVLHFNEFSQNESKEPDPCLKSNKQKVLADS |
| | | SLEGQKKTTEPLDQQAGSLNNESGLVSCYFGQ |
| | | LLSKLDRIKTDYTLSIANRLYGEQEFPICQEYL |
| | | DGVIQFYHTTIESVDFQKNPEKSRQEINFWVEC |
| | | QSQGKIKELFSKDAINAIETVLVLVNAVYFKAK |
| | | WETYFDHENTVDAPFWLNANENKSVKMMTQ |
| | | KGLYRJGFIEEVKAQILEMRYTKGKLSMFVLL |
| | | PSHSKDNLKGLEELERKITYEKMVAWSSSEN |
| | | MSEESVVLSFPRFTLEDSYDLNSILQDMGITDI |
| | | FDETRADLTGISPSPNLYLSKIIHKTFVEVDEN |
| | | GTQAAAATGAVVSESSKNSHLWLAPFMHPAQ |
| | | AGVKRSAAGIVDGWPPYAPLSAFWPPECSAM |
| | | TTDTSNSHILFGVSLFPLELPPVVQGGHAVFLQK |
| | | AGLEQTKEMALFSIRDEIDTDVSLELLTAFEES |
| | | CQLHVA (SEQ ID NO: 415) |

### Example 6

### Assemblage of SEQ ID NO: 407

The novel nucleic acid (SEQ ID NO: 407) of the invention were assembled from sequences that was obtained from a cDNA library by methods described in Example 1 above, and in some cases obtained from one or more public databases. The final sequence was assembled using the EST sequences as seed. Then a recursive algorithm was used to extend the seed into an extended assemblage, by pulling additional sequences from different databases (i.e. Hyseq's database containing EST sequences, dbEST, gb pri, and UniGene) that belong to this assemblage. The algorithm terminated when there was no additional sequences from the above databases that would extend the assemblage. Inclusion of component sequences into the assemblage was based on a BLASTN hit to the extending assemblage with BLAST score greater than 300 and percent identity greater than 95%.

Using PHRAP (Univ. of Washington) or CAP4 (Paracel), a full-length gene cDNA sequence and its corresponding protein sequence were generated from the assemblage. Any frame shifts and incorrect sop codons were corrected by hand editing. During editing, the sequence was checked using FASTY and BLAST against Genbank (i.e. dbEST, gb pri, UniGene, Genpept). Other computer programs which may have been used in the editing process were phredPhrap and Consed (University of Washington) and ed-ready, ed-ext and cg-zip-2 (Hyseq, Inc.).

### Example 7

### Identification of SEQ ID NO: 419

Assembly of the novel nucleotide sequence of SEQ ID NO: 419 was accomplished using a contig sequence SEQ ID NO: 418 as a seed. The seed was extended by gel sequencing (377 Applied Biosystems (ABI) sequencer) using primers to extend the 3′ end (primer extension). The DNA from the full-length clone was then isolated, sonicated and recloned for gel sequencing. Each fragment was sequenced by gel sequencing (377 Applied Biosystems (ABI) sequencer) and the sequences were assembled to arrive at the complete sequence.

A polypeptide (SEQ ID NO: 420) was predicted to be encoded by SEQ ID NO: 419 as set forth below. The polypeptide was predicted using a software program called BLASTX which selects a polypeptide based on a comparison of the translated novel polynucleotide to known polynucleotides. The initial methionine starts at position 1217 of SEQ ID NO: 419 and the putative stop codon, TGA, begins at position 2414 of the nucleotide sequence SEQ ID NO: 419.

### Example 8

### Assemblage of SEQ ID NO: 442

A polypeptide was predicted to be encoded by SEQ ID NO: 442 as set forth below. The polypeptide was predicted using a software program called FASTY (available from http://fasta.bioch.virginia.edu) which selects a polypeptide based on a comparison of translated novel polynucleotide to known polypeptides (W. R. Pearson, Methods in Enzymology, 183:63-98 (1990), herein incorporated by reference). The results for SEQ ID NO: 442 are shown in Table 49 below, wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine, X=Unknown, *=Stop Codon, /=possible nucleotide deletion, \=possible nucleotide insertion:

TABLE 49

| | | |
|---|---|---|
| Predicted | Predicted | |
| beginning | end | |
| nucleotide | nucleotide | |
| location | location | |
| correspond- | correspond- | |
| ing to first | ing to last | |
| amino acid | amino acid | |
| residue of | residue of | |
| amino acid | amino acid | Amino acid segment containing |
| segment | segment | signal peptide |

| | | |
|---|---|---|
| 3 | 385 | PPGPKGDQGDEGKEGRPGIPGLPGLRGLPGERGT |
| | | PGLPGPKGNDGKLGATGPMGMRGFKGDRGPKG |
| | | EKGEKGDRAGDASGVEAPMMIRLVNGSGPHEG |
| | | RVEVYHDRRWGTVCDDGWDKXDGDVVCRM |
| | | (SEQ ID NO: 480) |

### Example 9

### Assemblage of SEQ ID NO: 443 and 444

Assembly of novel nucleotide sequence of SEQ ID NO: 443 was accomplished by using an EST sequence SEQ ID NO: 441 as a seed. The seed was extended by gel sequencing (377 Applied Biosystems (ABI) sequencer) using primers to extend the 3′ end (primer extension). A portion of the 5′ end was extended by using primers and 5′ RACE on a reverse transcribed cDNA mixture prepared from mRNAs from Invitrogen that included adult brain, kidney, heart, liver, lung, placenta, small intestine, and uterus, as well as fetal brain, heart, kidney, liver, lung, muscle, and skin; mRNAs from Clontech that included adult adrenal gland, bone marrow, lymph node, pituitary gland, spinal cord, spleen, thyroid gland, thymus, and trachea, as well as the MOLT-4 leukemia cell line; and mRNAs from Biochain that included adult esophagus, and fetal umbilical cord. The resulting sequence was used to conduct a BLASTN alignment against GENSCAN (Stanford University, Burge, C. and Karlin, S. (1997) Prediction of complete gene structures in human genomic DNA. J. Mol. Biol. 268, 78-94) predicted genes from the Human Genome Project database to arrive at the final 5′ complete DNA sequence.

A polypeptide (SEQ ID NO: 444) was predicted to be encoded by SEQ ID NO: 443 as set forth below. The polypeptide was predicted using a software program called BLASTX which selects a polypeptide based on a comparison of translated novel polynucleotide to known polynucleotides. The initial methionine starts at position 417 of SEQ ID NO: 443 and the putative stop codon, TGA, begins at position 1902 of the nucleotide sequence.

### Example 10

### Assemblage of SEQ ID NO: 486, 504, 515, or 527

The novel nucleic acids (SEQ ID NO: 486, 504, 515, or 527) of the invention were assembled from sequences that were obtained from various cDNA libraries by methods described in Example 1 above, and in some cases obtained from one or more public databases. The final sequence was assembled using the EST sequence as seed. Then a recursive algorithm was used to extend the seed into an extended assemblage, by pulling additional sequences from different databases (i.e. Hyseq's database containing EST sequences, dbEST, gb pri, and UniGene) that belong to this assemblage. The algorithm terminated when there was no additional sequences from the above databases that would extend the assemblage. Inclusion of component sequences into the assemblage was based on a BLASTN hit to the extending assemblage with BLAST score greater than 300 and percent identity greater than 95%.

The nearest neighbor results for the assembled contigs were obtained by a FASTA search against Genpept, using FASTXY algorithm. FASTXY is an improved version of FASTA alignment which allows in-codon frame shifts. The nearest neighbor results showed the closest homologue for each assemblage from Genpept (and contain the translated amino acid sequences for which the assemblages encodes). The nearest neighbor results are set forth in Table 50 below:

TABLE 50

| | | | Smith- | |
|---|---|---|---|---|
| SEQ ID | Accession | | Waterman | |
| NO: | No. | Description | Score | % Identity |
| 486 | AAW29667 | Homo sapiens DL185_1 | 3969 | 60.629 |
| | | clone secreted protein | | |
| 504 | AK009118 | Mus musculus | 1089 | 74.797 |
| | | putativeprotein | | |
| 515 | AB052620 | Mus musculus DDM36 | 1374 | 40.830 |
| 527 | U35371 | Rattus norvegicus neural | 2822 | 91.391 |
| | | cell adhesion protein | | |
| | | precursor BIG-2 | | |

The predicted amino acid sequences for SEQ ID NO: 486, 504, 515, or 527 were predicted as set forth below. The polypeptides were predicted using a software program called BLASTX which selects a polypeptide based on a comparison of the translated novel polynucleotide to known polynucleotides. The initial methionine of SEQ ID NO: 487 starts at position 178 of SEQ ID NO: 486 and the putative stop codon, TGA, begins at position 3262 of the nucleotide sequence SEQ ID NO: 486. The initial methionine of SEQ ID NO: 506 starts at position 17 of SEQ ID NO: 504 and the putative stop codon, TGA, begins at position 707 of the nucleotide sequence SEQ ID NO: 504. The initial methionine of SEQ ID NO: 516 starts at position 1 of SEQ ID NO: 505 and the putative stop codon TAG, begins at position 2000 of the nucleotide sequence SEQ ID NO: 505. The initial methionine of SEQ ID NO: 528 starts at position 117 of SEQ ID NO: 527 and the putative stop codon TGA, begins at position 3249 of the nucleotide sequence SEQ ID NO: 527.

### Example 11

### Assemblage of SEQ ID NO: 547 or 556

The novel nucleic acids (SEQ ID NO: 547 or 556) of the invention were assembled from sequences that were obtained from cDNA libraries by methods described in Example 1 above, and in some cases obtained from one or more public databases. The final sequences were assembled using the EST sequences as seed. Then a recursive algorithm was used to extend the seed into an extended assemblage, by pulling additional sequences from different databases (i.e. Hyseq's database containing EST sequences, dbEST, gb pri, and UniGene) that belong to this assemblage. The algorithm terminated when there was no additional sequences from the above databases that would extend the assemblage. Inclusion of component sequences into the assemblage was based on a BLASTN hit to the extending assemblage with BLAST score greater than 300 and percent identity greater than 95%.

Using PHRAP (Univ. of Washington) or CAP4 (Paracel), a full-length gene cDNA sequence and its corresponding protein sequence were generated from the assemblage. Any frame shifts and incorrect sop codons were corrected by hand editing. During editing, the sequence was checked using FASTY and BLAST against Genbank (i.e. dbEST, gb pri, UniGene, Genpept). Other computer programs which may have been used in the editing process were phredphrap and Consed (University of Washington) and ed-ready, ed-ext and cg-zip-2 (Hyseq, Inc.). The full-length nucleotide sequences are shown in the Sequence Listing as SEQ ID NO: 547 or 556; and the full-length amino acid sequences are shown in the sequence listing as SEQ ID NO: 548 or 557.

### Example 12

### Assemblage of SEQ ID NO: 571 or 578

The novel nucleic acids (SEQ ID NO: 571 or 578) of the invention were assembled from sequences that were obtained from various cDNA libraries by methods described in Example 1 above, and in some cases obtained from one or more public databases. The final sequence was assembled using the EST sequence as seed. Then a recursive algorithm was used to extend the seed into an extended assemblage, by pulling additional sequences from different databases (i.e. Hyseq's database containing EST sequences, dbEST, gb pri, and UniGene) that belong to this assemblage. The algorithm terminated when there was no additional sequences from the above databases that would extend the assemblage. Inclusion of component sequences into the assemblage was based on a BLASTN hit to the extending assemblage with BLAST score greater than 300 and percent identity greater than 95%.

The nearest neighbor results for the assembled contigs were obtained by a FASTA search against Genpept, using FASTXY algorithm. FASTXY is an improved version of FASTA alignment which allows in-codon frame shifts. The nearest neighbor results showed the closest homologue for each assemblage from Genpept (and contain the translated amino acid sequences for which the assemblages encodes). The nearest neighbor results are set forth in Table 51 below:

TABLE 51

| | | | Smith- | |
|---|---|---|---|---|
| SEQ ID | Accession | | Waterman | % |
| NO: | No. | Description | Score | Identity |
| 573 | X83006 | Homo sapiens neutrophils | 208 | 40 |
| | | gelatinase associated | | |
| | | lipocalin | | |
| 578 | AAY91653 | Human secreted protein | 606 | 100 |
| | | sequence encoded by gene | | |
| | | 62 SEQ ID NO 326 | | |

The predicted amino acid sequences for SEQ ID NO: 571 or 578 were predicted as set forth below. The polypeptides were predicted using a software program called BLASTX which selects a polypeptide based on a comparison of the translated novel polynucleotide to known polynucleotides. The initial methionine of SEQ ID NO: 572 starts at position 192 of SEQ ID NO: 571 and the putative stop codon, TGA, begins at position 660 of the nucleotide sequence SEQ ID NO: 571. The initial methionine of SEQ ID NO: 579 starts at position 128 of SEQ ID NO: 578 and the putative stop codon, TGA, begins at position 727 of the nucleotide sequence SEQ ID NO: 578.

### Example 13

### Assemblage of SEQ ID NO: 587 and 589

The novel nucleic acid (SEQ ID NO: 587 and 589) of the invention were assembled from sequences that was obtained from a cDNA library by methods described in Example 1 above, and in some cases obtained from one or more public databases. The final sequence was assembled using the EST sequences as seed. Then a recursive algorithm was used to extend the seed into an extended assemblage, by pulling additional sequences from different databases (ie. Hyseq's database containing EST sequences, dbEST, gb pri, and UniGene) that belong to this assemblage. The algorithm terminated when there was no additional sequences from the above databases that would extend the assemblage. Inclusion of component sequences into the assemblage was based on a BLASTN hit to the extending assemblage with BLAST score greater than 300 and percent identity greater than 95%.

Using PHRAP (Univ. of Washington) or CAP4 (Paracel), a full-length gene cDNA sequence and its corresponding protein sequence were generated from the assemblage. Any frame shifts and incorrect stop codons were corrected by hand editing. During editing, the sequence was checked using FASTY and BLAST against Genbank (i.e. dbEST0627_Hs, gb pri124_Hs, UniGene124, Genpept124). Other computer programs which may have been used in the editing process were phredPhrap and Consed (University of Washington) and ed-ready, ed-ext and cg-zip-2 (Hyseq, Inc.). The full-length nucleotide sequences are shown in the Sequence Listing as SEQ ID NO: 587 and 589; and the full-length amino acid sequences are shown in the sequence listing as SEQ ID NO: 587.

### Example 14

### Assemblage of SEQ ID NO: 601, 606, and 611

The novel nucleic acids (SEQ ID NO: 601, 606, and 611) of the invention were assembled from sequences that were obtained from cDNA libraries by methods described in Example 1 above, and in some cases obtained from one or more public databases. The final sequences were assembled using the EST sequences as seed. Then a recursive algorithm was used to extend the seed into an extended assemblage, by pulling additional sequences from different databases (i.e. Hyseq's database containing EST sequences, dbEST, gb pri, and UniGene) that belong to this assemblage. The algorithm terminated when there was no additional sequences from the above databases that would extend the assemblage. Inclusion of component sequences into the assemblage was based on a BLASTN hit to the extending assemblage with BLAST score greater than 300 and percent identity greater than 95%.

Using PHRAP (Univ. of Washington) or CAP4 (Paracel), a full-length gene cDNA sequence and its corresponding protein sequence were generated from the assemblage. Any frame shifts and incorrect sop codons were corrected by hand editing. During editing, the sequence was checked using FASTY and BLAST against Genbank (i.e. dbEST, gb pri, UniGene, Genpept). Other computer programs which may have been used in the editing process were phredPhrap and Consed (University of Washington) and ed-ready, ed-ext and cg-zip-2 (Hyseq, Inc.). The full-length nucleotide sequences are shown in the Sequence Listing as SEQ ID NO: 601, 606, and 611; and the full-length amino acid sequences are shown in the sequence listing as SEQ ID NO: 602, 607, and 612.

### Example 15

### Assemblage of SEQ ID NO: 629 and 631

The novel nucleic acid (SEQ ID NO: 629 and 631) of the invention were assembled from sequences that was obtained from a cDNA library by methods described in Example 1 above, and in some cases obtained from one or more public databases. The final sequence was assembled using the EST sequences as seed. Then a recursive algorithm was used to extend the seed into an extended assemblage, by pulling additional sequences from different databases (i.e. Hyseq's database containing EST sequences013001, dbEST013001 HS, gb pri126_HS_cd, and UniGene126) that belong to this assemblage. The algorithm terminated when there was no additional sequences from the above databases that would extend the assemblage. Inclusion of component sequences into the assemblage was based on a BLASTN hit to the extending assemblage with BLAST score greater than 300 and percent identity greater than 95%.

Using PHRAP (Univ. of Washington) or CAP4 (Paracel), a fill-length gene cDNA sequence and its corresponding protein sequence were generated from the assemblage. Any frame shifts and incorrect sop codons were corrected by hand editing. During editing, the sequence was checked using FASTY and BLAST against Genbank (i.e. dbEST013001_HS, gb pri126_HS_cd, UniGene126, Genpept127). Other computer programs which may have been used in the editing process were phredPhrap and Consed (University of Washington) and ed-ready, ed-ext and cg-zip-2 (Hyseq, Inc.). The full-length nucleotide sequences are shown in the Sequence Listing as SEQ ID NO: 629 and 631; and the full-length amino acid sequences are shown in the sequence listing as SEQ ID NO: 630 and 632.

### Example 16

### Tissue Expression Analysis and Chromosomal Location of SEQ ID NO: 4, 14, 27, 159, 186, 214, 240, 271, 301, 322, 347, 354, or 377

By checking Hyseq proprietary database established from screening by hybridization, SEQ ID NO: 4, or 14 is found to be expressed in following human tissue/cell cDNA (see Table 52):

TABLE 52

| Library | No. of Positive | Total No. of Clones | |
|---|---|---|---|
| Name | Clones | in the Library | Tissue Origin |
| BMD001 | 13 | 342599 | bone marrow |
| ABD003 | 3 | 83268 | adult brain |
| FLS001 | 30 | 555770 | fetal liver-spleen |
| AKD001 | 5 | 176438 | adult kidney |
| LUC001 | 5 | 210372 | leukocytes |
| ATS001 | 2 | 26744 | testis |
| AKT002 | 7 | 149669 | adult kidney |
| AOV001 | 22 | 259409 | adult ovary |
| IB2002 | 21 | 265743 | infant brain |
| LGT002 | 7 | 158948 | lung tumor |
| HFB001 | 5 | 74494 | fetal brain |
| IBS001 | 3 | 33191 | infant brain |
| LPC001 | 8 | 97546 | lymphocyte |
| PIT004 | 5 | 120274 | pituitary gland |
| SPC001 | 2 | 61905 | whole organ |
| THM001 | 4 | 113947 | thymus |
| THR001 | 2 | 124110 | thyroid gland |
| ADR002 | 5 | 90185 | adrenal gland |
| CVX001 | 7 | 125473 | cervix |
| THA002 | 1 | 32817 | thalamus |
| FUC001 | 1 | 125570 | umbilical cord |
| SIN001 | 2 | 142562 | whole organ |
| ABR001 | 3 | 30163 | adult brain |
| FLG001 | 2 | 28154 | whole organ |
| BLD001 | 3 | 29386 | bladder |
| FSK001 | 5 | 127263 | fetal skin |
| CLN001 | 3 | 28708 | colon |
| REC001 | 1 | 28337 | rectum |
| SPLc01 | 2 | 110573 | spleen |
| FLG003 | 1 | 27360 | fetal lung |
| NTU001 | 4 | 37055 | neuronal cells |
| NTD001 | 5 | 35080 | induced neuronal cells |
| NTR001 | 3 | 34629 | retinoic acid-induced |
| | | | neuronal cells |
| ABR006 | 1 | 108204 | adult brain |
| FBR004 | 1 | 27560 | fetal brain |
| FBR006 | 8 | 151893 | fetal brain |
| ABR008 | 14 | 145661 | adult brain |
| FLS002 | 58 | 709733 | fetal liver-spleen |
| IB2003 | 14 | 201294 | infant brain |
| ADP001 | 2 | 37287 | cultured preadipocytes |
| ADP002 | 1 | 32855 | cultured preadipocytes |
| FLV002 | 2 | 32865 | fetal liver |
| BMD002 | 1 | 75816 | bone marrow |
| DIA002 | 1 | 40119 | diaphragm |
| FLV004 | 3 | 74491 | fetal liver |
| FKD002 | 1 | 33111 | fetal kidney |
| FSK002 | 1 | 72628 | fetal skin |
| FLS003 | 9 | 187791 | fetal liver-spleen |
| HMP001 | 3 | 71425 | macrophage |
| FLG004 | 1 | 41090 | fetal lung |
| BMD008 | 1 | 44770 | bone marrow |
| DGD001 | 1 | 91971 | lymphocyte |
| DGD004 | 1 | 91423 | lymphocytes |
| STM001 | 2 | 181899 | bone marrow |
| OBE01 | 3 | 132217 | adipocytes |

SEQ ID NO: 4, or 14 were further analyzed for their presence in the public dbEST database and their tissue source. SEQ ID NO: 4 or 14 were found to be expressed in following tissues: Gessler Wilms tumor, colon, Stratagene hNT neuron, Fibroblasts, senescent, Stratagene endothelial cell 937223, Soares breast 2NbHBst, Stratagene lung carcinoma 937218, Soares fetal liver spleen 1NFLS, Soares_parathyroid_tumor_NbHPA, total brain, Soares_NhHMPu_S1, Soares_fetal_heart_NbHH19W, liver, Soares infant brain 1NIB, Jurkat T-cells, cochlea, Ovary, and Testis tumor.

The gene corresponding to SEQ ID NO: 4 or 14 was mapped to human chromosome 12p11-37.2 by BLAST analysis with human genome sequences.

By checking Hyseq proprietary database established from screening by hybridization, SEQ ID NO: 27 is found to be expressed in following human tissue/cell cDNA (see Table 53):

TABLE 53

| | | Total No. of | |
|---|---|---|---|
| | No. of Positive | Clones in the | |
| Library Name | Clones | Library | Tissue Origin |
| LGT002 | 5 | 158948 | lung tumor |
| MMG001 | 1 | 131991 | mammary gland |
| PIT004 | 1 | 120274 | pituitary gland |
| THR001 | 5 | 124110 | thyroid gland |
| ADR002 | 2 | 90185 | adrenal gland |
| TRC001 | 1 | 23820 | trachea |
| FUC001 | 17 | 125570 | umbilical cord |
| FLG001 | 1 | 28154 | whole organ |
| FSK001 | 1 | 127263 | fetal skin |
| ADP001 | 1 | 37287 | adipocytes |
| ADP002 | 7 | 32855 | adipocytes |
| PLA003 | 1 | 80877 | placenta |
| FKD002 | 1 | 33111 | fetal kidney |
| FSK002 | 1 | 72628 | fetal skin |
| FHR001 | 2 | 108446 | fetal heart |
| FLG004 | 1 | 41090 | fetal lung |
| OBE01 | 5 | 132217 | adipocytes |

SEQ ID NO: 27 was further analyzed for their presence in the public dbEST database and their tissue source. SEQ D NO: 27 was found to be expressed in following tissues: Bone, poorly differentiated adeno, Fibroblasts, senescent, melanocyte, colon tumor RER+, Soares_NhHu_S1, bone marrow stroma, 2 pooled tumors (clear cell, Soares ovary tumor NbHOT, cochlea.

The gene corresponding to SEQ ID NO: 27 was mapped to chromosome 3 by BLAST analysis with human genome sequences.

By checking Hyseq proprietary database established from screening by hybridization, SEQ ID NO: 159 is found to be expressed in following human tissue/cell cDNA (see Table 54):

TABLE 54

| | | Total No. of | |
|---|---|---|---|
| | No. of Positive | Clones in the | |
| Library Name | Clones | Library | Tissue Origin |
| FLS001 | 1 | 555770 | fetal liver-spleen |
| AKD001 | 3 | 176438 | adult kidney |
| AOV001 | 9 | 259409 | adult ovary |
| CVX001 | 2 | 125473 | adult cervix |
| FLG001 | 1 | 28154 | fetal lung |
| SPLc01 | 1 | 110573 | spleen |
| FKD002 | 2 | 33111 | fetal kidney |

SEQ ID NO: 159 was further analyzed for their presence in the public dbEST database and their tissue source. SEQ ID NO: 159 was found to be expressed in following tissues: Soares_NhHMPu_S1, NCI_CGAP_Sub6.

The gene corresponding to SEQ ID NO: 159 was mapped to human chromosome 4 by BLAST analysis with human genome sequences.

By checking Hyseq proprietary database established from screening by hybridization, SEQ ID NO: 186, 214, 240, or 271 is found to be expressed in following human tissue/cell cDNA (see Table 55):

TABLE 55

| | | Total No. of | |
|---|---|---|---|
| | No. of Positive | Clones in the | |
| Library Name | Clones | Library | Tissue Origin |
| FLS001 | 1 | 555770 | fetal liver-spleen |
| FMS001 | 1 | 32743 | Fetal muscle |
| FSK001 | 1 | 127263 | Fetal skin |
| FMS002 | 6 | 40223 | Fetal muscle |
| FHR001 | 4 | 108446 | Fetal heart |

SEQ ID NO: 186, 214, 240, or 271 was further analyzed for their presence in the public dbEST database and their tissue source. SEQ ID NO: 186, 214, 240, or 271 was found to be expressed in following tissues:
HEMBB1, head_normal, MAGE resequences, MAGM, bone marrow, larynx tumor, high grade preneoplastic lesion, NCI_CGAP_Sub7, NIH_MGC_{—}87, NIH_MGC_{—}91, Soares_NFL_T_GBC_S1, Soares_testis_NHT.

The gene corresponding to SEQ ID NO: 186, 214, 240, or 271 was mapped to human chromosome 13 by BLAST analysis with human genome sequences.

By checking Hyseq proprietary database established from screening by hybridization, SEQ ID NO: 301, or 322 is found to be expressed in following human tissue/cell cDNA (see Table 56):

TABLE 56

| | | Total No. of | |
|---|---|---|---|
| | No. of Positive | Clones in the | |
| Library Name | Clones | Library | Tissue Origin |
| FLS001 | 1 | 555770 | Fetal liver-spleen |
| LUC001 | 1 | 210372 | leukocytes |
| AKT002 | 1 | 149669 | adult kidney |
| IB2002 | 2 | 265743 | infant brain |
| HFB001 | 3 | 74494 | fetal brain |
| SPC001 | 1 | 61905 | whole organ |
| NTR001 | 1 | 34629 | retinoic acid-induced |
| | | | neuronal cells |
| STM001 | 1 | 181899 | bone marrow |

SEQ ID NO: 301, or 322 was further analyzed for their presence in the public dbEST database and their tissue source. SEQ ID NO: 301, or 322 was found to be expressed in following tissues: 2 pooled tumors, HTC, and Soares fetal liver spleen 1NFLS S1.

The gene corresponding to SEQ ID NO: 301 or 322 was mapped to human chromosome 18 by BLAST analysis with human genome sequences.

By checking Hyseq proprietary database established from screening by hybridization, SEQ ID NO: 347 is found to be expressed in following human tissue/cell cDNA (see Table 57):

TABLE 57

| | | Total No. of | |
|---|---|---|---|
| | No. of Positive | Clones in the | |
| Library Name | Clones | Library | Tissue Origin |
| BMD001 | 2 | 342599 | bone marrow |
| ABD003 | 16 | 83268 | adult brain |
| FLS001 | 2 | 555770 | fetal liver-spleen |
| AKD001 | 2 | 176438 | adult kidney |
| LUC001 | 3 | 210372 | leukocytes |
| LUC003 | 3 | 30296 | leukocytes |
| ALV001 | 1 | 30866 | young liver |
| ATS001 | 1 | 26744 | testis |
| ASP001 | 1 | 32114 | adult spleen |
| APL001 | 1 | 31936 | placenta |
| ABT004 | 732 | 31910 | adult brain |
| AKT002 | 2 | 149669 | adult kidney |
| ALV002 | 10 | 144402 | adult liver |
| AOV001 | 5 | 259409 | ovary |
| IB2002 | 1276 | 265743 | infant brain |
| LGT002 | 16 | 158948 | adult lung |
| MMG001 | 8 | 131991 | mammary gland |
| HFB001 | 38 | 74494 | fetal brain |
| FBT002 | 1 | 35745 | fetal brain |
| IBM002 | 99 | 13952 | infant brain |
| IBS001 | 182 | 33191 | infant brain |
| LPC001 | 3 | 97546 | lymphocyte |
| PIT004 | 3 | 120274 | pituitary gland |
| SPC001 | 1705 | 61905 | whole organ |
| THR001 | 1 | 124110 | thyroid gland |
| MEL004 | 17 | 30503 | melanoma |
| ADR002 | 3 | 90185 | adrenal gland |
| CVX001 | 4 | 125473 | cervix |
| PRT001 | 2 | 28649 | whole organ |
| THA002 | 591 | 32817 | thalamus |
| TRC001 | 1 | 23820 | trachea |
| FBR001 | 1 | 28664 | fetal brain |
| FUC001 | 8 | 125570 | umbilical cord |
| SKM001 | 1 | 28327 | whole organ |
| SIN001 | 6 | 142562 | whole organ |
| ABR001 | 241 | 30163 | adult brain |
| FLG001 | 2 | 28154 | whole organ |
| BLD001 | 43 | 29386 | bladder |
| FMS001 | 4 | 32743 | fetal muscle |
| FSK001 | 8 | 127263 | fetal skin |
| CLN001 | 4 | 28708 | colon |
| REC001 | 3 | 28337 | rectum |
| SPLc01 | 13 | 110573 | spleen |
| FLG003 | 8 | 27360 | fetal lung |
| THMc02 | 17 | 96791 | thymus |
| NTU001 | 2 | 37055 | neuronal cells |
| NTR001 | 2 | 34629 | retinoic acid-induced |
| | | | neuronal cells |
| ABR006 | 365 | 108204 | adult brain |
| FBR004 | 2 | 27560 | fetal brain |
| FBR006 | 351 | 151893 | fetal brain |
| ABR008 | 11420 | 145661 | adult brain |
| FLS002 | 4 | 709733 | fetal liver-spleen |
| IB2003 | 1108 | 201294 | infant brain |
| ADP001 | 2 | 37287 | cultured preadipocytes |
| FLV002 | 11 | 32865 | fetal liver |
| PLA003 | 2 | 80877 | placenta |
| FLV004 | 2 | 74491 | fetal liver |
| ESO002 | 2 | 36840 | esophagus |
| FSK002 | 4 | 72628 | fetal skin |
| FMS002 | 7 | 40223 | fetal muscle |
| FHR001 | 7 | 108446 | fetal heart |
| FLS003 | 4 | 187791 | fetal liver-spleen |
| HMP001 | 10 | 71425 | macrophage |
| FLG004 | 1 | 41090 | fetal lung |
| ABR016 | 57 | 45716 | brain |
| BMD008 | 3 | 44770 | bone marrow |
| LYN001 | 2 | 44025 | lymph node |
| STM001 | 3 | 181899 | bone marrow |

SEQ ID NO: 347 was further analyzed for their presence in the public dbEST database and their tissue source. SEQ ID NO: 347 was found to be expressed in following tissues: Soares_total_fetus_Nb2HF8_{—}9w, head_neck, kidney tumor, colon tumor RER+, Soares_fetal_heart_NbHH19W, head_neck, pooled germ cell tumors, kidney, subtracted, 2 pooled tumors (clear cell type), colon tumor RER+, malignant melanoma, metastatic to lymph node, LTI_NFL006_PL2, cervix carcinoma cell line, bone marrow cell line, melanotic melanoma, carcinoid, Pineal gland II.

The gene corresponding to SEQ ID NO: 347 was mapped to human chromosome 18p11.3 by BLAST analysis with human genome sequences.

By checking Hyseq proprietary database established from screening by hybridization, SEQ ID NO: 354, or 377 is found to be expressed in following human tissue/cell cDNA (see Table 58):

TABLE 58

| | | Total No. of | |
|---|---|---|---|
| | No. of Positive | Clones in the | |
| Library Name | Clones | Library | Tissue Origin |
| ALV002 | 1 | 144402 | adult liver |
| FBR006 | 1 | 151893 | fetal brain |
| FKD002 | 1 | 33111 | fetal kidney |
| FSK002 | 1 | 72628 | fetal skin |

SEQ ID NO: 354 or 377 was further analyzed for their presence in the public dbEST database and their tissue source. SEQ ID NO: 354 or 377 was found to be expressed in following tissues: Neuroblastoma cells.

The gene corresponding to SEQ D NO: 354 or 377 was mapped to chromosome 12 by BLAST analysis with human genome sequences.

### Example 17

### Chromosomal Localization of SEQ ID NO: 240

To determine the chromosomal localization of SEQ ID NO: 240, gene specific PCR primers (5′-AAGCCTGGTCCCAAAGGAGA-3′ and 5′-GGTGTGGCGGATTTTTAAACTCT-3′) were screened against the NIGMS human/rodent somatic cell hybrid mapping panel #2. PCR amplification of the 423 nt product was performed using the following conditions; an initial denaturation at 94° C. for 3 min, followed by 5 cycles of 30 s at 94° C., 30 sec at 68° C. and 1 min at 72° C., followed by 5 cycles of 30 s at 94° C., 30 sec at 64° C. and 1 min at 72° C., followed by 20 cycles of 30 s at 94° C., 30 sec at 60° C. and 1 min at 72° C. followed by an extension of 10 min at 72° C. All products were separated by 3% agarose gel electrophoresis and visualized via ethidium bromide staining. SEQ ID NO: 240 was mapped to chromosome 13.

### Example 18

### Tissue Expression Analysis and Chromosomal Localization of 407

By checking Hyseq proprietary database established from screening by hybridization, SEQ ID NO: 407 was found to be expressed in following human tissue/cell cDNA (see Table 59):

TABLE 59

| | No. of Positive | Total No. of Clones | |
|---|---|---|---|
| Library Name | Clones | in the Library | Tissue Origin |
| FSK001 | 1 | 127263 | fetal skin |
| FSK002 | 2 | 72628 | fetal skin |

SEQ ID NO: 407 was further analyzed for their presence in the public dbEST database and their tissue source. SEQ ID NO: 407 was found to be expressed in following tissues: Soares_NhHMPu_S1.

The gene corresponding to SEQ ID NO: 407 was mapped to human chromosome 18 by BLAST analysis with human genome sequences.

### Example 19

### Tissue Expression Analysis and Chromosomal Localization of 486, 504 and 527

By checking Hyseq proprietary database established from screening by hybridization, SEQ ID NO: 486, is found to be expressed in following human tissue/cell cDNA (see Table 60):

TABLE 60

| | No. of Positive | Total No. of Clones | |
|---|---|---|---|
| Library Name | Clones | in the Library | Tissue Origin |
| AKD001 | 1 | 176438 | Adult kidney |
| HFB001 | 1 | 74494 | Fetal brain |
| PIT004 | 2 | 120274 | Pituitary gland |
| SPC001 | 1 | 61905 | Spinal cord |
| FUC001 | 1 | 125570 | Umbilical cord |
| SIN001 | 1 | 142562 | Small intestine |
| FBR004 | 1 | 27560 | Fetal brain |
| IB2003 | 1 | 201294 | Infant brain |

SEQ ID NO: 486 was further analyzed for their presence in the public dbEST database and their tissue source. SEQ ID NO: 486 was found to be expressed in following tissues: pituitary, NIH_MGC_{—}114 adult brain, NIH_MGC_{—}121 fetal brain, Soares multiple sclerosis lesions, hypothalamus, Athersys RAGE library, Clontech human aorta polyA+ mRNA, NCI_CGAP_Kid11 subtracted kidney, Morton fetal cochlea, NIH_MGC_{—}120 adult pancreas spleen, Soares_fetal_lung_NbHL19W, NCI_CGAP_Utlwell-differentiated endometrial adenocarcinoma, 7 pooled tumors, NT, and NCI_CGAP_Pr28 subtracted prostate.

The gene corresponding to SEQ ID NO: 486 was mapped to human chromosome 3p by BLAST analysis with human genome sequences.

By checking Hyseq proprietary database established from screening by hybridization, SEQ ID NO: 504 is found to be expressed in following human tissue/cell cDNA (see Table 61):

TABLE 61

| | | Total No. of | |
|---|---|---|---|
| | No. of Positive | Clones in the | |
| Library Name | Clones | Library | Tissue Origin |
| FKD002 | 1 | 33111 | Fetal kidney |

SEQ ID NO: 504 was further analyzed for its presence in the public dbEST database and its tissue source. SEQ ID NO: 504 was found to be expressed in following tissues: normal colon and adult colon kidney stomach.

By checking Hyseq proprietary database established from screening by hybridization, SEQ ID NO: 514 is found to be expressed in following human tissue/cell cDNA (see Table 62):

TABLE 62

| | | Total No. of | |
|---|---|---|---|
| | No. of Positive | Clones in the | |
| Library Name | Clones | Library | Tissue Origin |
| FKD001 | 1 | 127263 | Fetal skin |

SEQ ID NO: 514 was further analyzed for its presence in the public dbEST database and its tissue source. SEQ ID NO: 514 was found to be expressed in following tissues: Soares_NFL_T_G testis, B-cell and fetal lung.

By checking Hyseq proprietary database established from screening by hybridization, SEQ ID NO: 527 is found to be expressed in following human tissue/cell cDNA (see Table 63):

TABLE 63

| | | Total No. of | |
|---|---|---|---|
| | No. of Positive | Clones in the | |
| Library Name | Clones | Library | Tissue Origin |
| PIT004 | 1 | 120274 | Pituitary gland |
| THM001 | 1 | 113947 | Thymus |
| CVX001 | 1 | 125473 | Cervix |
| SIN001 | 1 | 142562 | Whole organ |
| IB2003 | 1 | 201294 | Infant brain |

SEQ ID NO: 527 was further analyzed for its presence in the public dbEST database and its tissue source. SEQ ID NO: 527 was found to be expressed in following tissues: normal nervous, testis, NIH_MGC_{—}85 lymph, lymphoma, NIH_MGC_{—}97 testis cell line, NCI_CGAP_Skn3 skin, Schiller oligodendroglioma, and Soares_NFL_T_G tesis, B-cell and fetal lung.

### Example 20

### Tissue Expression of 547

By checking Hyseq proprietary database established from screening by hybridization, SEQ ID NO: 547 is found to be expressed in the following human tissue/cell cDNA and shown in Table 64:

TABLE 64

| | | Total No. of | |
|---|---|---|---|
| | | Clones in the | No. of Positive |
| Library Name | Tissue Origin | Library | Clones |
| APL001 | placenta | 31936 | 3963 |
| PIT004 | pituitary gland | 120274 | 10496 |
| PLA003 | placenta | 80877 | 4991 |
| FLS001 | fetal liver-spleen | 555770 | 8220 |
| FLS003 | fetal liver-spleen | 187791 | 2072 |
| FLV001 | fetal liver | 33189 | 46 |
| ALN001 | lymph node | 27965 | 17 |
| ABT004 | adult brain | 31910 | 19 |
| FKD001 | fetal kidney | 31293 | 16 |
| FLS002 | fetal liver-spleen | 709733 | 211 |
| LUC003 | leukocytes | 30296 | 8 |
| OBE01 | adipocytes/Obesity | 132217 | 31 |
| FBR004 | fetal brain | 27560 | 6 |
| BMD001 | bone marrow | 342599 | 56 |
| ADP002 | adipocytes | 32855 | 4 |
| THM001 | thymus | 113947 | 10 |
| THMc02 | thymus | 96791 | 8 |
| FSK002 | fetal skin | 72628 | 5 |
| ASP001 | adult spleen | 32114 | 2 |
| THR001 | thyroid gland | 124110 | 7 |
| DGD004 | lymphocytes/Meyloma | 91423 | 5 |
| FLV004 | fetal liver | 74491 | 4 |
| ADP001 | adipocytes | 37287 | 2 |
| AKD001 | adult kidney | 176438 | 9 |
| FMS002 | fetal muscle | 40223 | 2 |
| FUC001 | umbilical cord | 125570 | 6 |
| AHR001 | adult heart | 130524 | 6 |
| ABR016 | brain | 45716 | 2 |
| BMD002 | bone marrow | 75816 | 3 |
| AOV001 | ovary | 259409 | 10 |
| ABD003 | adult brain | 83268 | 3 |
| BLD001 | bladder | 29386 | 1 |
| ABR001 | adult brain | 30163 | 1 |
| DGD001 | lymphocyte/Burkitt's | 91971 | 3 |
| | lymphoma | | |
| ALV001 | young liver | 30866 | 1 |
| SPC001 | whole organ | 61905 | 2 |
| THA002 | thalamus | 32817 | 1 |
| NTR001 | neuron | 34629 | 1 |
| NTD001 | neuron | 35080 | 1 |
| ABR006 | adult brain | 108204 | 3 |
| MMG001 | mammary gland | 131991 | 3 |
| ADR002 | adrenal gland | 90185 | 2 |
| STM001 | bone marrow | 181899 | 4 |
| LPC001 | lymphocyte | 97546 | 2 |
| IB2003 | infant brain | 201294 | 4 |
| FBR006 | fetal brain | 151893 | 3 |
| CVX001 | cervix | 125473 | 2 |
| FSK001 | fetal skin | 127263 | 2 |
| BB2002 | infant brain | 265743 | 4 |
| ABR008 | adult brain | 145661 | 2 |
| HFB001 | fetal brain | 74494 | 1 |
| AKT002 | adult kidney | 149669 | 2 |
| LUC001 | leukocytes | 210372 | 2 |
| SIN001 | whole organ | 142562 | 1 |
| ALV002 | adult liver | 144402 | 1 |
| LGT002 | lung tumor | 158948 | 1 |
| EDT001 | endothelial cells | 177809 | 1 |

### Example 21

### Tissue Expression Analysis of 571 and 578

By checking Hyseq proprietary database established from screening by hybridization, SEQ ID NO: 571 and 578 are found to be expressed in following human tissue/cell cDNA (see Table 65):

TABLE 65

| | No. of Positive | Total No. of Clones | |
|---|---|---|---|
| Library Name | Clones | in the Library | Tissue Origin |
| AKT002 | 2 | 149669 | Adult kidney |
| THR001 | 2 | 124110 | Thyroid gland |
| CVX001 | 1 | 125473 | Cervix |
| EDT001 | 1 | 177809 | Endothelial cells |
| SPLc01 | 1 | 110573 | (Spleen) |
| THMc02 | 1 | 96791 | Thymus |
| ABR006 | 7 | 108204 | Adult brain |
| ABR008 | 1 | 145661 | Adult brain |
| ALV003 | 1 | 34611 | Adult liver |
| FLV002 | 1 | 32865 | Fetal liver |
| PLA003 | 1 | 80877 | Placenta |
| FSK002 | 1 | 72628 | Fetal skin |
| FMS002 | 1 | 40223 | Fetal muscle |
| FHR001 | 2 | 108446 | Fetal heart |
| SIP002 | 17 | 179333 | Mixed tissue |
| SIP005 | 2 | 37621 | Mixed tissue |

### Example 22

### Tissue Expression Analysis of 587 and 589

By checking Hyseq proprietary database established from screening by hybridization, SEQ ID NO: 587 or 589 is found to be expressed in following human tissue/cell cDNA (see Table 66).

TABLE 66

| Library | Library | Number of | Total | |
|---|---|---|---|---|
| number | Name | clones | clones | Tissue |
| 71 | ADR002 | 1 | 90185 | Adrenal gland |
| 118 | SPLc01 | 1 | 110573 | spleen |

Expression of SEQ ID 587 and 589 was also found in the lung tumor library (LGT002).

SEQ ID NO: 587 was further analyzed for its presence in the public dbEST database and its tissue source. SEQ ID NO: 587 was found to be expressed in following tissues: T colon, Soares_placenta_{—}8 to 9 weeks_{—}2NbHP8 to 9W, NIH_MGC_{—}90 Liver tumor cell line made from adenocarcinoma tissue and Soares_NFL_T_G_testis, B-cell and fetal lung. Further information on these libraries may be obtained at http://image.llnl.gov/image/html/humlib_info.shtml.

### Example 23

### Chromosomal Localization of SEQ ID NO: 587

By running Hyseq's proprietary software program that maps SEQ ID NO: 587 to the human genome, SEQ ID NO: 587 was mapped to chromosome 1p22.2-31.1. To confirm the chromosomal localization of SEQ ID NO: 587, gene specific PCR primers (5′ATGGCACATCGTGATTCTGAG 3 and 5′-TTAGCAGAACTTTAGC-3′) were screened against the NIGMS human/rodent somatic cell hybrid mapping panel #2. PCR amplification of the 423 nt product was performed using the following conditions; an initial denaturation at 94° C. for 3 min, followed by 5 cycles of 30 s at 94° C., 30 sec at 68° C. and 1 min at 72° C., followed by 5 cycles of 30 s at 94° C., 30 sec at 64° C. and 1 min at 72° C., followed by 20 cycles of 30 s at 94° C., 30 sec at 60° C. and 1 min at 72° C. followed by an extension of 10 min at 72° C. All products were separated by 3% agarose gel electrophoresis and visualized via ethidium bromide staining. SEQ ID NO: 587 was mapped to chromosome 1.

### Example 24

### Tissue Expression Analysis of 601, 606, and 611

By checking Hyseq proprietary database established from screening by hybridization, SEQ ID NO: 601 is found to be expressed in the following human tissue/cell cDNA (see Table 67):

TABLE 67

| | | Total No. of | |
|---|---|---|---|
| | | Clones in the | No. of Positive |
| Library Name | Tissue Origin | Library | Clones |
| SIN001 | whole organ | 142562 | 3802 |
| BMD002 | bone marrow | 75816 | 714 |
| SPLc01 | spleen | 110573 | 356 |
| STO001 | whole organ | 26894 | 307 |
| REC001 | rectum | 28337 | 244 |
| TRC001 | trachea | 23820 | 235 |
| BMD001 | bone marrow | 342599 | 213 |
| SAL001 | whole organ | 37753 | 152 |
| CLN001 | colon | 28708 | 92 |
| CLN001 | colon | 28708 | 92 |
| CVX001 | cervix | 125473 | 85 |
| THM001 | thymus | 113947 | 51 |
| THMc02 | thymus | 96791 | 38 |
| THR001 | Thyroid gland | 124110 | 27 |
| PRT001 | whole organ | 28649 | 22 |
| LPC001 | lymphocyte | 97546 | 20 |
| ASP001 | adult spleen | 32114 | 18 |
| AKT002 | adult kidney | 149669 | 17 |
| FLG001 | whole organ | 28154 | 17 |
| BLD001 | bladder | 29386 | 15 |
| ESO002 | esophagus | 36840 | 12 |
| ABR016 | brain | 45716 | 11 |
| LUC001 | leukocytes | 210372 | 10 |
| FUC001 | umbilical cord | 125570 | 10 |
| UTR001 | uterus | 29595 | 7 |
| PIT004 | pituitary gland | 120274 | 6 |
| PIT004 | pituitary gland | 120274 | 6 |
| FSK001 | fetal skin | 127263 | 4 |
| FLS002 | fetal liver-spleen | 709733 | 4 |
| ALG001 | adult lung | 28271 | 3 |
| HFB001 | fetal brain | 74494 | 3 |
| SPC001 | whole organ | 61905 | 3 |
| HMP001 | marrow | 71425 | 3 |
| FLS001 | fetal liver spleen | 555770 | 2 |
| ALV002 | adult liver | 144402 | 2 |
| LGT002 | lung tumor | 158948 | 2 |
| ABR006 | adult brain | 108204 | 2 |
| PLA003 | placenta | 80877 | 2 |
| AHR001 | adult heart | 130524 | 1 |
| MMG001 | mammary gland | 131991 | 1 |
| ADR002 | adrenal gland | 90185 | 1 |
| THA002 | thalamus | 32817 | 1 |
| SKM001 | whole organ | 28327 | 1 |
| NTD001 | neuron | 35080 | 1 |
| FBR004 | fetal brain | 27560 | 1 |
| FBR006 | fetal brain | 151893 | 1 |
| ALV003 | adult liver | 34611 | 1 |
| FKD002 | fetal kidney | 33111 | 1 |
| FSK002 | fetal liver-spleen | 187791 | 1 |

By checking Hyseq proprietary database established from screening by hybridization, SEQ ID NO: 606 and 611 are found to be identically expressed in the following human tissue/cell cDNA (see Table 68):

TABLE 68

| | | Total No. of | |
|---|---|---|---|
| | | Clones in the | No. of Positive |
| Library Name | Tissue Origin | Library | Clones |
| SIN001 | whole organ | 142562 | 3802 |
| BMD002 | bone marrow | 75816 | 714 |
| SPLc01 | spleen | 110573 | 356 |
| THMc02 | thymus | 96791 | 338 |
| STO001 | whole organ | 26894 | 307 |
| REC001 | rectum | 28337 | 244 |
| TRC001 | trachea | 23820 | 235 |
| BMD001 | bone marrow | 342599 | 213 |
| SAL001 | whole organ | 37753 | 152 |
| LYN001 | lymph node | 44025 | 112 |
| BMD008 | bone marrow | 44770 | 104 |
| CLN001 | colon | 28708 | 92 |
| CVX001 | cervix | 125473 | 85 |
| THM001 | thymus | 113947 | 51 |
| THR001 | thyroid gland | 124110 | 27 |
| PRT001 | whole organ | 28649 | 22 |
| LPC001 | lymphocyte | 97546 | 20 |
| ASP001 | adult spleen | 32114 | 18 |
| AKT002 | adult kidney | 149669 | 17 |
| FLG001 | fetal lung | 28154 | 17 |
| BLD001 | bladder | 29386 | 15 |
| ESO002 | esophagus | 36840 | 12 |
| ABR016 | brain | 45716 | 11 |
| LUC001 | leukocytes | 210372 | 10 |
| FUC001 | umbilical cord | 125570 | 10 |
| UTR001 | uterus | 29595 | 7 |
| PIT004 | pituitary gland | 120274 | 6 |
| FSK002 | fetal skin | 72628 | 5 |
| STM001 | bone marrow | 181899 | 4 |
| DGD004 | lymphocytes | 91423 | 4 |
| FLS002 | fetal liver-spleen | 709733 | 4 |
| FSK001 | fetal skin | 127263 | 4 |
| SPC001 | whole organ | 61905 | 3 |
| HFB001 | fetal brain | 74494 | 3 |
| HMP001 | hematopoetic cells | 71425 | 3 |
| ALG001 | adult lung | 28271 | 3 |
| FLS001 | fetal liver-spleen | 555770 | 2 |
| LGT002 | lung tumor | 158948 | 2 |
| ALV002 | adult liver | 144402 | 2 |
| FHR001 | fetal heart | 108446 | 2 |
| PLA003 | placenta | 80877 | 2 |
| ABR006 | adult brain | 108204 | 2 |
| SKM001 | whole organ | 28327 | 1 |
| THA002 | thalamus | 32817 | 1 |
| ADR002 | adrenal gland | 90185 | 1 |
| DGD001 | lymphocyte | 91971 | 1 |
| MMG001 | mammary gland | 131991 | 1 |
| AHR001 | adult heart | 130524 | 1 |
| FLS003 | fetal liver-spleen | 187791 | 1 |
| FKD002 | fetal kidney | 33111 | 1 |
| ALV003 | adult liver | 34611 | 1 |
| FBR006 | fetal brain | 151893 | 1 |
| FBR004 | fetal brain | 27560 | 1 |
| NTD001 | neuron | 35080 | 1 |

### Example 25

### Tissue Expression Analysis and Chromosomal Location of SEQ ID NO: 629

By checking Hyseq proprietary database established from screening by hybridization, SEQ ID NO: 629 is found to be expressed in following human tissue/cell cDNA (see Table 69):

TABLE 69

| | No. of Positive | Total No. of Clones | |
|---|---|---|---|
| Library Name | Clones | in the Library | Tissue Origin |
| BMD001 | 145 | 342599 | bone marrow |
| ABD003 | 96 | 83268 | adult brain |
| FLS001 | 506 | 555770 | fetal liver-spleen |
| AKD001 | 421 | 176438 | adult kidney |
| LUC001 | 245 | 210372 | leukocytes |
| LUC003 | 31 | 30296 | leukocytes |
| ALV001 | 10 | 30866 | young liver |
| ATS001 | 34 | 26744 | testis |
| ASP001 | 78 | 32114 | adult spleen |
| ALG001 | 35 | 28271 | adult lung |
| AHR001 | 328 | 130524 | adult heart |
| APL001 | 6 | 31936 | placenta |
| FKD001 | 34 | 31293 | fetal kidney |
| ALN001 | 34 | 27965 | lymph node |
| ABT004 | 4 | 31910 | adult brain |
| AKT002 | 50 | 149669 | adult kidney |
| ALV002 | 24 | 144402 | adult liver |
| AOV001 | 124 | 259409 | ovary |
| IB2002 | 4 | 265743 | infant brain |
| LGT002 | 59 | 158948 | lung tumor |
| MMG001 | 82 | 131991 | mammary gland |
| AB3001 | 1 | 1565 | adult brain |
| HFB001 | 30 | 74494 | fetal brain |
| FLV001 | 11 | 33189 | fetal liver |
| FBT002 | 8 | 35745 | fetal brain |
| LPC001 | 17 | 97546 | lymphocyte |
| PIT004 | 70 | 120274 | pituitary gland |
| SPC001 | 37 | 61905 | whole organ |
| THM001 | 56 | 113947 | thymus |
| THR001 | 85 | 124110 | thyroid gland |
| MEL004 | 24 | 30503 | melanoma |
| ADR002 | 41 | 90185 | adrenal gland |
| CVX001 | 81 | 125473 | cervix |
| PRT001 | 19 | 28649 | whole organ |
| STO001 | 18 | 26894 | whole organ |
| THA002 | 12 | 32817 | thalamus |
| TRC001 | 20 | 23820 | trachea |
| UTR001 | 7 | 29595 | uterus |
| FBR001 | 9 | 28664 | fetal brain |
| FUC001 | 46 | 125570 | umbilical cord |
| SKM001 | 13 | 28327 | whole organ |
| SAL001 | 10 | 37753 | whole organ |
| SIN001 | 55 | 142562 | whole organ |
| ABR001 | 13 | 30163 | adult brain |
| FLG001 | 12 | 28154 | whole organ |
| BLD001 | 8 | 29386 | bladder |
| FMS001 | 17 | 32743 | fetal muscle |
| FSK001 | 27 | 127263 | fetal skin |
| EDT001 | 30 | 177809 | endothelial cells |
| CLN001 | 2 | 28708 | colon |
| REC001 | 2 | 28337 | rectum |
| SPLc01 | 8 | 110573 | adult spleen |
| FLG003 | 3 | 27360 | fetal lung |
| THMc02 | 3 | 96791 | thymus |
| NTU001 | 8 | 37055 | neuronal cells |
| NTD001 | 3 | 35080 | neuron |
| NTR001 | 7 | 34629 | neuron |
| ABR006 | 3 | 108204 | adult brain |
| FBR004 | 3 | 27560 | fetal brain |
| FBR006 | 7 | 151893 | fetal brain |
| ABR008 | 1 | 145661 | adult brain |
| FLS002 | 52 | 709733 | fetal liver-spleen |
| IB2003 | 1 | 201294 | infant brain |
| ADP001 | 14 | 37287 | adipocytes |
| ADP002 | 10 | 32855 | adipocytes |
| LFB001 | 1 | 41616 | lung, fibroblast |
| ALV003 | 10 | 34611 | adult liver |
| FLV002 | 13 | 32865 | fetal liver |
| BMD002 | 35 | 75816 | bone marrow |
| DIA002 | 4 | 40119 | diaphragm |
| PLA003 | 2 | 80877 | placenta |
| FLV004 | 5 | 74491 | fetal liver |
| FKD002 | 7 | 33111 | fetal kidney |
| ESO002 | 6 | 36840 | esophagus |
| FSK002 | 5 | 72628 | fetal skin |
| FMS002 | 11 | 40223 | fetal muscle |
| FHR001 | 22 | 108446 | fetal heart |
| FLS003 | 171 | 187791 | fetal liver-spleen |
| HMP001 | 13 | 71425 | macrophage |
| FLG004 | 14 | 41090 | fetal lung |
| ABR016 | 3 | 45716 | brain |
| SUP002 | 28 | 179333 | mix 16 tissues |
| SUP005 | 1 | 37621 | mix 16 tissues |
| SUP007 | 14 | 43646 | mix 16 tissues |
| BMD008 | 12 | 44770 | bone marrow |
| LYN001 | 19 | 44025 | lymph node |
| SUP008 | 1 | 37997 | mix |
| SUP014 | 3 | 46740 | mixed |
| SUP015 | 8 | 46850 | mixed |
| DGD001 | 264 | 91971 | lymphocyte |
| DGD004 | 25 | 91423 | lymphocytes |
| STM001 | 292 | 181899 | bone marrow |
| OBE01 | 48 | 132217 | adipocytes |

SEQ ID NO: 629 was further analyzed for their presence in the public dbEST database and their tissue source. SEQ ID NO: 629 was found to be expressed in following tissues: NIH_MGC_{—}119, NIH_MGC_{—}7, NCI_CGAP_GC6, Soares_testis, NCI_CGAP_Brn25, and NCI_CGAP_Brn35. Further description of the tissue source can be found at http://image.llnl.gov/image/html/humlib_info.shtml.

SEQ ID NO: 629 was mapped to human chromosome 1p34.1-35.3 by BLAST analysis with human genome sequences.

### Example 26

### Three-Dimensional Structure of 407

The GeneAtlas software package (Molecular Simulations Inc. (MSI), San Diego, Calif.) was used to predict the three-dimensional structure models of SEQ ID NO: 407. Models were generated by (1) PSI-BLAST which is the multiple alignment sequence profile-based searching developed by Altschul et al, (Nucl. Acids. Res. 25:3389-3408 (1997), (2) High Throughput Modeling (MSI) which is an automated sequence and structure searching procedure (Accelrys, Burlington, Mass.), and (3) SeqFold which is a fold recognition method described by Fischer and Eisenberg (J. Mol. Biol. 209:779-791 (1998)). This analysis was carried out, in part, by comparing the Serpin-like amino acid sequence (SEQ ID NO: 407) with the known NMR (nuclear magnetic resonance) and x-ray crystal three-dimensional structures of stem cell factors as templates. The best structural model predictions for Serpin-like polypeptides, SEQ ID NO: 407, were each based on the stem cell factor templates and the results are summarized below, where “PDB ID”, the Protein DataBase (PDB) identifier given to template structure; “Chain ID”, identifier of the subcomponent of the PDB template structure; “Compound Information”, information of the PDB template structure and/or its subcomponents; “PDB Function Annotation” gives function of the PDB template as annotated by the PDB files (Berman et al., Nucl. Acids Res. 28:235-242 (2000), herein incorporated by reference); start and end amino acid position of the protein sequence aligned; PSI-BLAST score, the verify score, the SeqFold score, and the Potential(s) of Mean Force (PMF) score. The verify score is produced by GeneAtlas™ software (MSI), is based on Dr. Eisenberg's Profile-3D threading program developed in Dr. David Eisenberg's laboratory (U.S. Pat. No. 5,436,850 and Luthy, Bowie, and Eisenberg, Nature, 356:83-85 (1992)) and a publication by R. Sanchez and A. Sali, Proc. Natl. Acad. Sci. USA, 95:13597-12502. The verify score produced by GeneAtlas normalizes the verify score for proteins with different lengths so that a unified cutoff can be used to select good models as follows:
Verify score (normalized)=(raw score−½ high score)/(½ high score)

The PFM score, produced by GeneAtlas™ software (MSI), is a composite scoring function that depends in part on the compactness of the model, sequence identity in the alignment used to build the model, pairwise and surface mean force potentials (MFP). As given in Table 70, a verify score between 0 and 1.0, with 1 being the best, represents a good model. Similarly, a PMF score between 0 and 1.0, with 1 being the best, represents a good model. A SeqFold™ score of more than 50 is considered significant. A good model may also be determined by one of skill in the art based all the information in Table 70 taken in totality.

TABLE 70

| SEQ | | | | | Start | End | PSI- | | | |
|---|---|---|---|---|---|---|---|---|---|---|
| ID | PDB | Chain | Compound | | amino | amino | BLAST | Verify | PMF | SeqFold |
| NO: | ID | ID | Information | PDB Function Annotation | acid | acid | score | score | score | score |
| 407 | 1ova | | Serpin | _1ova_ovalbumin_(eggalbumin) | 3 | 425 | 5.1e−61 | 0.81 | 1.00 | |

The overall topology of SEQ ID NO: 407 was similar to ov-serpins subfamily. It exhibited several β-sheets and α-helices. The amino acids from residues 82 through 101 add an insertion of 20 amino acids in one of the loop regions in the structure of SEQ ID NO: 407. This is likely important to the function of the protein as loop segments are usually on the surface of proteins, and often provide interfaces for protein-protein interaction binding sites and enzymatic active sites.

### Example 27

### Three-Dimensional Structure of SEQ ID NO: 572

The GeneAtlas™ software package (Molecular Simulations, Inc. (MSI), San Diego, Calif.) was used to predict the three-dimensional structure model of NGALHy1 polypeptide (SEQ ID NO: 572). Models were generated by (1) PSI-BLAST which is the multiple alignment sequence profile-based searching developed by Altschul et al. (Nucl. Acids Res. 25:3389-3408 (1997)), (2) High Throughput Modeling (MSI) which is an automated sequence and structure searching procedure (Accelrys, Burlington, Mass.), and (3) SeqFold which is a fold recognition method described by Fischer and Eisenberg (J. Mol. Biol. 209:779-791 (1998)). This analysis was carried out, in part, by comparing the NGALHy1 amino acid sequence (SEQ ID NO: 572) with the known NMR (nuclear magnetic resonance) and x-ray crystal three-dimensional structure of NGAL (SEQ ID NO: 586) as template. The best structural model prediction for NGALHy1 was based on the NGAL template and the results are summarized below, wherein “PDB ID” is the Protein DataBase (PDB) identifier given to template structure; “Chain ID” is the identifier of the subcomponent of the PDB template structure; “Compound Information” is the information of the PDB template structure and/or its subcomponents; “PDB Function Annotation” gives function of the PDB template as annotated by the PDB files (Berman et al., Nucl. Acids Res. 28:235-242 (2000), herein incorporated by reference); start and end amino acid position of the protein sequence aligned; PSI-BLAST score, the verify score, the SeqFold score, and the Potential(s) of Mean Force (PMF) score. The verify score is produced by GeneAtlas™ software (MSI), is based on Dr. Eisenberg's Profile-3D threading program developed in Dr. David Eisenberg's laboratory (U.S. patent Ser. No. 09/5,436,850; Luthy et al., Nature 356:83-85 (1992); Sanchez and Sali, Proc. Natl. Acad. Sci. USA 95:13597-13602 (1998)). The verify score produced by GeneAtlas™ normalizes the verify score for proteins with different lengths so that a unified cutoff can be used to select good models as follows:
Verify score (normalized)=(raw score− 1/2 high score)/( 1/2 high score)

The PMF score, produced by GeneAtlas™ software (MSI), is a composite scoring function that depends in part on the compactness of the model, sequence identity in the alignment used to build the model, pairwise and surface mean force potentials (MFP). As given in Table 71 below, a verify score between 0 and 1.0, with 1 being the best, represents a good model. Similarly, a PMF score between 0 and 1.0 with 1 being the best, represents a good model. A good model may also be determined by one of skill in the art based on all the information in Table 71 taken in totality.

TABLE 71

| | | | | Start | End | PSI | | |
|---|---|---|---|---|---|---|---|---|
| SEQ ID | PDB | Compound | PDB Function | Amino | Amino | Blast | Verify | PMF |
| NO | ID | Information | Annotation | Acid | Acid | Score | score | Score |
| 572 | 1 | NGAL | Sugar binding protein. | 32 | 155 | 3.2e−24 | 0.4 | 0.9 |
| | | | crystal structure of | | | | | |
| | | | human neutrophils | | | | | |
| | | | gelatinase associated | | | | | |
| | | | lipocalin monomer. | | | | | |
| | | | NGAL; neutrophils, | | | | | |
| | | | NGAL, lipocalin | | | | | |

### Example 28

### Expression Analysis of SEQ ID NO: 240

First strand human cDNA libraries from multiple tissues were screened with gene specific primers for SEQ ID NO: 240 (5′-CGATGCAGGAGAACCAGGAC-3′ and 5′-CCTCAGGACCAGTGGGACC-3′). The commercial panels (Clontech) screened were: Panel I (heart, brain, placenta, lung, liver, skeletal muscle, kidney and pancreas), Panel II (Spleen, thymus, prostate, testis, ovary, small intestine, colon and adipocyte from a marathon ready cDNA library), immune panel (spleen, lymph node, thymus, tonsil, bone marrow, fetal liver, peripheral blood leukocyte) and a blood fraction panel (mononuclear, resting CD8+, resting CD4+, resting CD14+, resting CD19+, activated mononuclear cells, activated CD4+ and activated CD8+). PCR was performed for a total of 30 cycles using the following conditions: an initial denaturation at 94° C. for 3 min, followed by 5 cycles of 30 s at 94° C., 30 sec at 68° C. and 1 min at 72° C., followed by 5 cycles of 30 s at 94° C., 30 sec at 64° C. and 1 min at 72° C., followed by 20 cycles of 30 s at 94° C., 30 sec at 60° C. and 1 min at 72° C. followed by an extension of 10 min at 72° C. The amplification product was detected by analysis on agarose gels stained with ethidium bromide. The SEQ ID NO: 240 was expressed in a human adipose tissue cDNA library.

### Example 29

### Cellular Localization of SEQ ID NO: 241

SEQ ID NO: 240 specific primers corresponding to the translational start region and the carboxy-terminal region, excluding the stop codon of the SEQ ID NO: 240 sequence, were used (5′-TATAAGCTTATGAGGATCTGGTGGCTTCTG-3′ and 5′-AATCTCAGACGGGCTGCTGAACAGAAGG-3′). PCR amplification of the 883 nt product was performed using the following conditions; an initial denaturation at 94° C. for 3 min, followed by 5 cycles of 30 s at 94° C., 30 sec at 66° C. and 1 min at 72° C., followed by 5 cycles of 30 s at 94° C., 30 sec at 62° C. and 1 min at 72° C., followed by 20 cycles of 30 s at 94° C., 30 sec at 58° C. and 1 min at 72° C. followed by an extension of 10 min at 72° C. These primers generated a fragment of DNA corresponding to the entire coding region of the SEQ ID NO: 240, flanked by HINDIII and XHOI sites. The PCR product was digested accordingly to generate overhang ends that were ligated to the HINDIII and XHOI sites of PCDNA3.1/myc-His(+)A (Invitrogen). The resultant mammalian expression plasmid (AQL1/myc-His) allows for expression of the AQL1 coding sequence fused in-frame with the myc-6His epitope at the carboxy terminus.

The mammalian expression vector was transfected into COS-7 cells. Briefly, cells in a 10 cm dish with 8 ml of medium were incubated with 16 μl of Fugene-6 and 4 μg of DNA for 12 h. The medium was then replaced with serum-free DMEM and incubated for an additional 48 h prior to harvesting. After the conditioned medium was collected from transfected COS-7 cells, cells were washed twice with PBS and then scrapped from plates. Upon centrifugation, the cells were resuspended in PBS containing 0.5 μg/ml leupeptin, 0.7 μg/ml pepstatin, and 0.2 μg/ml aprotinin. After a brief sonication, the cytosolic fraction was separated from the insoluble membrane fraction by centrifugation. Purification of proteins from the cytosolic and from the media took place at 4 C in the presence of 100 μl of Ni-NTA resin (Qiagen). The resin was washed twice with 50 mM Tris-HCl (pH 7.5), 300 mM NaCl, and 5 mM imidazole.

To determine the cellular localization of the AQL1/myc-His tagged protein, Western blot analysis was performed on cytosolic, membrane, and medium fractions using an anti-myc antibody. AQL1/myc-His tagged protein was detected primarily in the medium (85%), but some protein was also detected in the cytosolic (10%) and membrane (5%) fractions. The predicted molecular mass of the tagged AQL1/myc-His tagged protein is 38 kDa. However, the approximate 44 kDa electrophoretic mobility suggests that AQL1/myc-His tagged protein is post-translationaly modified.

### Example 30

### A. Expression of Full-Length Polypeptides of the Invention in Cells

Chinese Hamster Ovary (CHO) cells or other suitable cell types are grown in DMEM (ATCC) and 10% fetal bovine serum (FBS) (Gibco) to 70% confluence. Prior to transfection, the media is changed to DMEM and 0.5% FBS. Cells are transfected with cDNAs for SEQ ID NO: 5, 7-13, 15, 17-24, 28, 30-156, 160, 162-182, 186, 188-213, 215, 217-239, 241, 243-270, 272, 274-299, 302, 304-321, 323, 325-344, 348, 350-352, 355, 357-376, 378, 380-401, 408, 410-414, 415, 420, 422-439, 444-480, 482-484, 487, 489-501, 505, 507-512, 516, 518-524, 528, 530-539, 542, 544-546, 548, 550-553, 557, 559-567, 572, 574, 576, 579, 581-584, 588, 590, 596, 602, 604-605, 607, 609-610, 612, 614-615, 618, 620, 622, 624, 626, 628, 630, 632, or 634-653 or with pBGal vector by the FuGENE-6 transfection reagent (Boehringer). In summary, 4 μl of FuGENE-6 is diluted in 100 μl of DMEM and incubated for 5 min. Then, this is added to 1 μl of DNA and incubated for 15 min before adding it to a 35 mm dish of CHO cells. The CHO cells are incubated at 37° C. with 5% CO₂. After 24 h, media and cell lysates are collected, centrifuged and dialyzed against assay buffer (15 mM Tris pH 7.6, 134 mM NaCl, 5 mM glucose, 3 mM CaCl₂ and MgCl₂).

### B. Expression Study Using Polynucleotides of the Invention

The expression of SEQ ID NO: 1-4, 6, 14, 16, 25-27, 29, 157-159, 161, 183-185, 187, 214, 216, 240, 242, 271, 273, 300-301, 303, 322, 324, 345-347, 349, 353-354, 356, 377, 379, 405-407, 409, 418-419, 421, 441-443, 485-486, 488, 503, 504, 506, 514-515, 517, 526-527, 529, 547, 549, 556, 558, 570-571, 573, 577-578, 580, 587, 589, 601, 603, 606, 608, 611, 613, 617, 619, 621, 623, 625, 627, 629, or 631 in various tissues is analyzed using a semi-quantitative polymerase chain reaction-based technique. Human cDNA libraries are used as sources of expressed genes from tissues of interest (adult bladder, adult brain, adult heart, adult kidney, adult lymph node, adult liver, adult lung, adult ovary, adult placenta, adult rectum, adult spleen, adult testis, bone marrow, thymus, thyroid gland, fetal kidney, fetal liver, fetal liver-spleen, fetal skin, fetal brain, fetal leukocyte and macrophage). Gene-specific primers are used to amplify portions of SEQ ID NO: 1-4, 6, 14, 16, 25-27, 29, 157-159, 161, 183-185, 187, 214, 216, 240, 242, 271, 273, 300-301, 303, 322, 324, 345-347, 349, 353-354, 356, 377, 379, 405-407, 409, 418-419, 421, 441-443, 485-486, 488, 503, 504, 506, 514-515, 517, 526-527, 529, 547, 549, 556, 558, 570-571, 573, 577-578, 580, 587, 589, 601, 603, 606, 608, 611, 613, 617, 619, 621, 623, 625, 627, 629, or 631 sequence from the samples. Amplified products are separated on an agarose gel, transferred and chemically linked to a nylon filter. The filter is then hybridized with a radioactively labeled (³³P-dCTP) double-stranded probe generated from SEQ ID NO: 1-4, 6, 14, 16, 25-27, 29, 157-159, 161, 183-185, 187, 214, 216, 240, 242, 271, 273, 300-301, 303, 322, 324, 345-347, 349, 353-354, 356, 377, 379, 405-407, 409, 418-419,421,441-443, 485-486,488,503,504,506,514-515, 517, 526-527, 529, 547, 549, 556, 558, 570-571, 573, 577-578, 580, 587, 589, 601, 603, 606, 608, 611, 613, 617, 619, 621, 623, 625, 627, 629, or 631 using a Klenow polymerase, random-prime method. The filters are washed (high stringency) and used to expose a phosphorimaging screen for several hours. Bands indicate the presence of cDNA including SEQ ID NO: 1-4,6,14,16,25-27,29,157-159,161,183-185, 187, 214, 216, 240, 242, 271, 273, 300-301, 303, 322, 324, 345-347, 349, 353-354, 356, 377, 379, 405-407, 409, 418-419, 421, 441-443, 485-486, 488, 503, 504, 506, 514-515, 517, 526-527, 529, 547, 549, 556, 558, 570-571,573,577-578, 580, 587, 589, 601, 603, 606, 608, 611, 613, 617, 619, 621, 623, 625, 627, 629, or 631 sequences in a specific library, and thus mRNA expression in the corresponding cell type or tissue.

### Example 31

### Expression of Full-Length Polypeptides of the Invention in E. coli

SEQ ID NO: 5, 15, 28, 160, 186, 215, 241, 272, 302, 323, 348, 355, 378, 408, 420, 444, 487, 505, 516, 528, 542, 548, 557, 572, 579, 588, 602, 607, 612, 618, 622, 626, or 630 is expressed in E. Coli by subcloning the entire coding region into a prokaryotic expression vector. The expression vector (pQE16) used is from the QIAexpression® prokaryotic protein expression system (QIAGEN). The features of this vector that make it useful for protein expression include: an efficient promoter (phage T5) to drive transcription, expression control provided by the lac operator system, which can be induced by addition of IPTG (isopropyl-β-D-thiogalactopyranoside), and an encoded histidine, His6, tag comprising a stretch of 6 histidine amino acid residues which can bind very tightly to a nickel atom. The vector can be used to express a recombinant protein with a His6 tag fused to its carboxyl terminus, allowing rapid and efficient purification using Ni-coupled affinity columns.

PCR is used to amplify the coding region which is then ligated into digested pQE16 vector. The ligation product is transformed by electroporation into electrocompetent E. coli cells (strain M15 [pREP4] from QIAGEN), and the transformed cells are plated on ampicillin-containing plates. Colonies are screened for the correct insert in the proper orientation using a PCR reaction employing a gene-specific primer and a vector-specific primer. Positives are then sequenced to ensure correct orientation and sequence. To express the polypeptide of the invention, a colony containing a correct recombinant clone is inoculated into L-Broth containing 100 μg/ml of ampicillin, 25 μg/ml of kanamycin, and the culture is allowed to grow overnight at 37° C. The saturated culture is then diluted 20-fold in the same medium and allowed to grow to an optical density at 600 nm of 0.5. At this point, IPTG is added to a final concentration of 1 mM to induce protein expression. The culture is allowed to grow for 5 more hours, and then the cells are harvested by centrifugation at 3000×g for 15 minutes.

The resultant pellet is lysed using a mild, nonionic detergent in 20 mM Tris HCl (pH 7.5) (B-PER™ Reagent from Pierce), or by sonication until the turbid cell suspension turned translucent. The lysate obtained is further purified using a nickel-containing column (Ni-NTA spin column from QIAGEN) under non-denaturing conditions. Briefly, the lysate is brought up to 300 mM NaCl and 10 mM imidazole and centrifuged at 700×g through the spin column to allow the His-tagged recombinant protein to bind to the nickel column. The column is then washed twice with Wash Buffer (50 mM NaH₂PO₄, pH 8.0; 300 mM NaCl; 20 mM imidazole) and is eluted with Elution Buffer (50 mM NaH₂PO₄, pH 8.0; 300 mM NaCl; 250 mM imidazole). All the above procedures are performed at 4° C. The presence of a purified protein of the predicted size is confirmed with SDS-PAGE.

### Example 32

### Expression and Purification of Polypeptides of the Invention from Insect Cells

Polypeptides of the invention are expressed in insect cells as follows:

An open reading frame expressing a polypeptide of the invention is cloned by PCR into a pIB/V5-His TOPO TA cloning vector (Invitrogen Corporation) either with a Myc/His tag or without any tags. Insect cells (High Five TM, Invitrogen) are transfected with the plasmid DNA containing the tagged or untagged version of the polypeptide of the invention by using the InsectSelect™ System (Invitrogen). The expression of the polypeptide of the invention is determined by transient expression. The medium containing an expressed polypeptide of the invention is separated on SDS-PAGE and the expressed polypeptide of the invention is identified by Western blot analysis. For large-scale production of a polypeptide of the invention, resistant cells are expanded into flasks containing Ultimate InsectSerum-Free medium (Invitrogen). The cells are shaken at ˜100 mph at 27° C. for 4 days. The conditioned media containing the protein for purification are collected by centrifugation.

### Example 33

### Production of Antibodies Specific to the Polypeptides of the Invention

Cells expressing a polypeptide of the invention are identified using antibodies specific to the polypeptide of the invention. Polyclonal antibodies are produced by DNA vaccination or by injection of peptide antigens into rabbits or other hosts. An animal, such as a rabbit, is immunized with a peptide from the extracellular region of the polypeptide of the invention conjugated to a carrier protein, such as BSA (bovine serum albumin) or KLH (keyhole limpet hemocyanin). The rabbit is initially immunized with conjugated peptide in complete Freund's adjuvant, followed by a booster shot every two weeks with injections of conjugated peptide in incomplete Freund's adjuvant. Antibodies of the invention are affinity purified from rabbit serum using a peptide of the invention coupled to Affi-Gel 10 (BioRad), and stored in phosphate-buffered saline (PBS) with 0.1% sodium azide. To determine that the polyclonal antibodies are specific for the polypeptide of the invention, an expression vector encoding the polypeptide of the invention is introduced into mammalian cells. Western blot analysis of protein extracts of non-transfected cells and the cells expressing the polypeptide of the invention is performed using the polyclonal antibody sample as the primary antibody and a horseradish peroxidase-labeled anti-rabbit antibody as the secondary antibody. Detection of a band corresponding to the molecular weight of the polypeptide of the invention in the cells expressing the polypeptide of the invention and lack thereof in the control cells indicates that the polyclonal antibodies are specific for said polypeptide of the invention.

Monoclonal antibodies are produced by injecting mice with a peptide of the invention, with or without adjuvant. Subsequently, the mouse is boosted every 2 weeks until an appropriate immune response has been identified (typically 1-6 months), at which point the spleen is removed. The spleen is minced to release splenocytes, which are fused (in the presence of polyethylene glycol) with murine myeloma cells. The resulting cells (hybridomas) are grown in culture and selected for antibody production by clonal selection. The antibodies are secreted into the culture supernatant, facilitating the screening process, such as screening by an enzyme-linked immunosorbent assay (ELISA). Alternatively, humanized monoclonal antibodies are produced either by engineering a chimeric murine/human monoclonal antibody in which the murine-specific antibody regions are replaced by the human counterparts and produced in mammalian cells, or by using transgenic “knock out” mice in which the native antibody genes have been replaced by human antibody genes and immunizing the transgenic mice as described above.

### Example 33

### Multiplex Analysis of Protein Phosphorylation and Cytokine/Chemokine Activation After Treatment with Polypeptides of the Invention

### A. Secretion Levels of the Polypeptide of the Invention

The full-length open reading frame of the polypeptide of the invention (i.e. SEQ ID NO: 2-4, 6, 14, 16, 26-27, 29, 158-159, 161, 184-185, 187, 214, 216, 240, 242, 271, 273, 301, 303, 322, 324, 346-347, 349, 354, 356, 377, 379, 407, 409, 419, 421, 443, 486, 488, 504, 506, 515, 517, 527, 529, 541, 543, 547, 549, 556, 558, 571, 573, 578, 580, 587, 589, 601, 603, 606, 608, 611, 613, 617, 619, 621, 623, 625, 627, 630, or 631) is cloned into the mammalian expression vector pcDNA3.1/V5-His-Topo (Invitrogen, Carlsbad, Calif.) to generate a C-terminal V5-His tagged expression construct. The resulting plasmid is transiently transfected into COS7L cells using the Fugene-6 transfection reagent (Roche Biosciences). The presence of the V5-His tagged protein is determined in both culture supernatant and cell lysate by Western blotting using anti-V5 antibodies and chemiluminescence visualization. The percent secretion is determined by comparing the amount of protein in the supernatant to the amount of protein in the cell lysate.

### B. Detection of Intracellular Protein Phosphorylation

The assay described below, a Bio-Plex (Bio-Rad, Hercules, Calif.) phosphorylation assay, is one of several methods employed for measuring protein phosphorylation in order to assess potential functions of secreted proteins in the particular cell type tested. Briefly, purified antibodies against various protein kinases, JNK, p38MAPK, erk, Stat3, and IκBα, are conjugated to microsphere sets according to the manufacturer's protocol. Culture supernatant from COS7L cells, transiently transfected with an expression plasmid containing a V5-His tagged fusion protein of the polypeptide of the invention (see Example 33A), is harvested and 10 μl of the culture supernatant is added to a panel of target cell lines for 15 min at 37° C. Cells are lysed and the lysate is clarified. The conjugated microspheres are incubated with 25 μl of cell lysate in a final volume of 50 μl in a 96-well plate overnight at room temperature with constant shaking. After incubation, the microspheres are washed with Tris buffered saline (TBS) containing 0.02% Tween-20 (TBST). Protein phosphorylation is detected by incubating the microspheres with 25 μl of a mixture of biotinylated antibodies against the phosphorylated forms of the protein kinases, for example, anti-phospho-Stat3, in TBST containing 5% mouse serum at room temperature for 30 min with constant shaking. The microspheres are washed with TBST and further incubated with 2 μg/ml of streptavidin-phycoerythrin (PE). The resulting microspheres with the reaction complex are analyzed using the Luminex Reader (Luminex Co., Austin, Tex.).

### C. Detection of Cytokine/Chemokine Levels

Cytokine and chemokine levels are determined using the assay described below, the Luminex Multi-plex bead assay, which is very similar to a typical sandwich ELISA assay, but utilizes Luminex microspheres conjugated to anti-cytokine and anti-chemokine antibodies (Vignali, J. Immunol. Methods 243:243-255 (2000), herein incorporated by reference). Briefly, purified antibodies against a variety of cytokines and chemokines are conjugated to microsphere sets (Luminex Co., Austin, Tex.) according to the manufacturer's protocol. Culture supernatant from COS7L cells, transiently transfected with an expression plasmid containing a V5-His tagged fusion protein of the invention (see Example 33A), is harvested and 25 μl of the culture supernatant is added to a panel of target cell lines and incubated overnight at 37° C. Condition media is then harvested. The conjugated microspheres are incubated with 50 μl in a 96-well filter plate at room temperature for 30 min with constant shaking. After incubation, the microspheres are washed and incubated with 50 μl (1 μg/ml) of biotinylated anti-cytokine or anti-chemokine antibodies in phosphate buffered saline (PBS) containing 0.5% Tween-20, 0.2% BSA, 5% mouse serum at room temperature for 30 min. The microspheres are washed and further incubated with 2 μg/ml of Streptavidin-PE. The resulting microspheres with the reaction complex are analyzed using the Luminex Reader (Luminex Co., Austin, Tex.).

### Example 34

### Calcium Mobilization Assay

Many extracellular signals to intracellular targets are mediated by increases in free calcium levels in the cytoplasm. Calcium mobilization from intracellular stores can be detected in many cell types by loading the cells with a Ca²⁺ sensitive indicator such as fura-2-AM. The increase in fluorescence is detected by a fluorescence plate reader. Cells will be incubated in media containing 5 μM Fura-2 AM, 5 μM Pluronic F-127 for 30 min. After the addition of adiponectin-like protein the Fura-2 intensity will be monitored approximately every 20 sec by a fluorescent plate reader (Molecular Dynamics) and compared to the intensity of cells with basal calcium levels.

### Example 35

### Fatty Acid Oxidation Assay

The oxidation of palmitate or oleate in culture C2Cl2 skeletal muscle cells (ATCC; CRL-1772) upon exposure to AQL1 protein is measured according to published procedures (Barger et al., J. Clin. Invest. 105:1723-1730 (2000)). In summary, nearly confluent C2C12 myocytes are kept in differentiation medium (DMEM, 2.5% horse serum) for 7 days, at which time formation of myotubes is maximal. [1-¹⁴C]oleic acid (1 μCi/ml) is added to the cells and incubated for 90 minutes at 37° C. in the absence/presence of adiponectin-like protein. In some of the assays a proteolytically cleaved adiponectin-like protein (cleaved between lysine 190-glycine 191) may be employed. During the experiment the C2C12 cells are incubated in a closed system containing Whatman paper to collect the ¹⁴CO₂ gas released during fatty acid oxidation. After the incubation the Whatman paper is removed and the amount of ¹⁴C radioactivity is determined by liquid scintillation counting.

### Example 36

### Macrophage Phagocytosis Assay

Human macrophages are incubated in the presence/absence of adiponectin-like protein for 24 hours at 37° C. in 96-well plates. Fluobrite fluorescent-microspheres (0.75 G; Polyscience, Warrington, Pa.) are added to each well, followed by one hour incubation at 37° C. Nonadherent latex beads are removed by gentle washing and the cells are incubated for an additional 30 minutes to complete phagocytosis. The cells are harvested by short-time treatment with EDTA and trypsin and washed vigorously three times with PBS to remove noningested beads. The amount of ingested beads will be measured with a FACScan.

### Example 37

### Glucose Uptake Assay

The adiponectin-like proteins influence carbohydrate and lipid metabolism. One of the ways by which the adiponectin-like proteins affect the development of insulin resistence is by altering glucose metabolism. To evaluate the effect of the polypeptides of the invention on glucose uptake. differentiated rat L6 myotube cells are cultured in 96-well plate for a minimum of 5 days in DMEM with 3% horse serum. The cells are incubated in 100 μl serum free media containing 25 mM glucose at 37 C in 5% CO₂ with or without adiponectin-homolog proteins of SEQ ID NO: 2 or 8 at a concentration of 30 μg/ml for 4-5 hours, followed by a subsequent incubation with insulin (100 nM) for 1 hour. The cells are then washed with serum containing media twice to remove glucose. The cells are further incubated with 10 μM [1,2-³H]2-deoxyglucose in 50 μl HBS for 20 min at 30 C. The overlayed media is removed and the cells are washed twice with 2001 μl of HBS buffer to remove the excess 2-Deoxy-D-[1-³H]2-glucose from the cells. The cells are lysed with 100 μl of 1 M NaOH by incubation for 30 min. The supernatants from the cells are collected and stored. 5 μl of supernatant is transferred to a 96-well plate for radioactive counting in the 96-well scintillation counter for measuring the ³H uptake by the cells. The ³H uptake by cells reflects the glucose uptake induced by adiponectin by the cells. (Sarabia et. al., Biochem Cell Biol 68:536-542 (1990); Yu et al., J. Biol. Chem. 276: 19994-19998 (2001)).

### Example 38

### Effects on Neuronal Growth In Vitro and In Vivo

### A. Fibroblast Spreading Assays

Mouse NIH 3T3 cells are cultured and assayed for spreading behavior in DMEM containing 10% FCS, usually to a maximum of 70-80% confluency. Subconfluent 3T3 cells are plated for 1 h in serum-containing media before fixation and staining with rhodamine-phalloidin. Glass coverslips are precoated with poly-L lysine, washed, and coated with PBS containing Nogo peptides, the soluble ectodomain of NgR, the soluble ectodomain of NgRHy, or anti-NgRHy antibodies, the soluble domain of neural IgCAM-like polypepides (i.e. SEQ ID NO: 501, 512, or 539), neural IgCAM-like peptides (i.e. SEQ ID NO: 490, 508, 519, or 531), or anti-neural IgCAM-like antibodies. Appropriate concentration of protein for coating will be predetermined in separate assays. The protein drops are allowed to dry, the slides washed in PBS, and then fixed in 1% glutaraldehyde in PBS or 4% paraformaldehyde in PBS.

### B. Growth Cone Collapse Assays

For the assessment of growth cone collapse, chick DRGs from embryonic day 7 (E7) are explanted onto laminin-coated chamber slides in F12 medium with 10 ng/ml nerve growth factor and 10% fetal bovine serum for 20 h. Phosphate-buffered saline (PBS) solutions containing 1 mM DTT with or without the soluble ectodomain of NgRHy, NgRm or neural IgCAM-like polypeptides are added to the explants (225 μl) and incubated at 37° C. for one hour. For each explant, all growth cones were scored as collapsed or fan-shaped (Igarashi et al., Science 259: 77.1993). For E7-E15 cultures, the origin of neuronal cells can be assessed by staining with anti-neurofilament antibodies and the O4 antibody for detection of oligodendrocytes. Neurites are traced by observation of rhodamine-phalloidin staining of F-actin in processes.

In the above assays, neurite outgrowth or inhibition of growth cone collapse may increase after the addition of the NgRHy or neural IgCAM-like polypeptide as compared to cultures lacking added peptide or cultured in the presence of Nogo protein. This indicates that NgRHy or neural IgCAM-like peptide acts as an antagonist to the endogenous NgR protein and inhibits the effects of Nogo proteins on preventing neural growth.

### C. Neuronal Co-Culture Assays

Embryonic DRG neurons are co-cultured with adult oligodendrocytes (Oudega et al., Neuroscience 100: 873-883. 2000) in the presence of the soluble ectodomain of NgRHy or neural IgCAM-like polypeptides or antibodies specific for NgRHy or neural IgCAM-like polypeptides to determine the antagonistic effects of NgRHy or neural IgCAM-like peptides and antibodies.

Adult oligodendrocytes are isolated from rat spinal cord and cultured in DMEM/F12 with 0.5 μg NGF, 15 nM selenium, 1 mg transferrin, 0.5 μg insulin-like growth factor-1 and 2.5% heat-inactivated bovine serum in collagen coated Aclar hats (2×10⁴ cell/hat). After 4 days the oligodendrocytes are incubated in either plain DMEM or DMEM containing NgRHy or neural IgCAM-like polypeptides (at a pre-determined optimal concentration) for 30 min at 37° C. Following this incubation, 75% of the media is replaced by a suspension of DRG neurons (10⁴ cells/hat) isolated as described above. The co-cultures are maintained in neurobasal medium [B27 suppl. (Gibco BRL), containing 50 ng/ml partially purified NGF and 50 ug/ml ascorbic acid] at 37° C. 5% CO₂ for 72 h. The cultures are rinsed in L15 media with 10% normal goat serum and stained with mouse O1 anti-oligodendrocyte antibody. The oligodendrocytes are visualized using a rhodamine-conjugated goat-anti-mouse antibody. The cells are then fixed in 4% paraformaldehyde in PBS and permeabilized with 0.2% Triton X-100. To visualize axons, the co-culture is stained with an anti-neurofilament antibody.

The effects of NgRHy or neural IgCAM-like peptides and antibodies on DRG neurite growth can be quantified in a 2.0×0.5 mm strip by measuring the length of neurites that are touched an oligodendrocyte. An increase in neurite outgrowth in the co-culture in the presence of NgRHy or neural IgCAM-like peptides or antibodies demonstrates that NgRHy or neural IgCAM-like peptides or antibodies can act as antagonists to the endogenous receptor and prevent the inhibition of neural growth mediated by the oligodendrocytes, indicating NgRHy or neural IgCAM-like peptides and antibodies can be an effective treatment in vivo for the promotion of neural regeneration.

### D. Neurite Outgrowth Assays

For the assessment of neurite outgrowth, chick DRGs from embryonic day 7 (E7) are explanted onto laminin-coated chamber slides in F12 medium with 10 ng/ml nerve growth factor and 10% fetal bovine serum for 20 h. Phosphate-buffered saline (PBS) solutions containing 1 mM DTT with or without the soluble domain of neural IgCAM-like polypeptides are added to the explants (225 μl) and incubated at 37° C. for one hour. For each explant, all neurite are scored as collapsed or fan-shaped (Igarashi et al., Science 259: 77. 1993). For E7-E15 cultures, the origin of neuronal cells can be assessed by staining with anti-neurofilament antibodies and the O4 antibody for detection of oligodendrocytes. Neurites are traced by observation of rhodamine-phalloidin staining of F-actin in processes.

In the above assays, neurite outgrowth may increase after the addition of the neural IgCAM-like polypeptide as compared to cultures lacking added peptide indicating that neural IgCAM-like peptides enhance neurite outgrowth.

### Example 39

### Assessment of Binding Partners for NgRHy

The NgRHy polynucleotide sequence can be transfected into host cells as described previously. For instance, NgRHy is transfected into either COS cells or 3T3 fibroblasts and the binding of NgRHy to the Nogo protein and its isoforms are assessed by means well-known in the art. For example, the Nogo protein is labeled with a detectable label such as alkaline phosphatase (Fournier et al., supra) or conjugated to biotin molecules or a fluorophore such as fluoroisothiocyanate (FITC) or phycoerythrin (PE). The binding of labeled Nogo protein to cells expressing NgRHy is assessed by an appropriate detection method based on the labeled Nogo protein. These methods include staining for the presence of Nogo on cells bound to a coverslip or through flow cytometric analysis of the fluorophore-conjugated Nogo protein to the NgRHy-expressing cells.

The NgRHy of the invention can also be expressed in neurons such as embryonic DRG and retinal neurons which demonstrate a weak Nogo-66 response, in order to assess the ability of NgRHy to mediate Nogo activity (Fournier et al., supra). Infection of neurons with HSV containing NgRHy is carried out according to Takahashi et al, Nature Neurosci. 1: 487-493. 1998. cDNA of the invention can be inserted into an plasmid such as pHSV-PrpUC containing the immediate early promoter of HSV and an HSV packaging site. The plasmid is transfected into the HSV packaging cell line 2-2 which is infected after 24 hrs with a replication deficient HSV, such as the IE2 deletion mutant 5dl1.2. Recombinant viral stocks are amplified by sequential rounds of infection. Viral stocks are added to neuronal cultures at a concentration of approximately 106 plaque forming units (PFU)/ml 24 hours before analysis of neuronal culture. Neuronal growth is assayed as described previously.

### Example 40

### Effect of Neural IgCAM-Like Polypeptides on Astrocyte Cell Proliferation

Primary astrocytes are trypsinized and transferred to 96-well plates at a density of 2×10⁵ cells/ml. After cell attachment for 24 h, the culture medium is exchanged for serum-free medium. After 48 h, neural IgCAM-like polypeptides are added. After 12 h, [³H]thymidine is added (10 μCi/ml) and the incubation proceeds for another 12 h. Incorporation of [³H]thymidine is measured and cells are harvested onto glass filters using an automatic cell harvester (Packard, Meriden, Conn.). The incorporated radioactivity is measured using a liquid scintillation counter.

### Example 41

### Radioimmunoassay for NGAL-Like Activity

To measure the serum levels of NGAL-like polypeptides in normal and disease states, a radioimmunoassay specific for NGAL-like polypeptides is used (see Xu et al., J. Immunol. Meth. 171:245-252 (1994), herein incorporated by reference). Briefly, blood is drawn from patients and granules are prepared from buffy coats of granulocytes. An equal volume of 2% Dextran T-500 in phosphate buffered saline (PBS) without magnesium or calcium is added to the buffy coats for 1 h at room temperature. The granulocytes are sedimented and spun through 0.34 M sucrose. The cells are homogenized in a Potter-Elvehjem homogenizer and mixed with an equal volume of 0.34 M sucrose and 0.3 M NaCl, clarified at 450×g and then sedimented at 10,000×g. Granulocytes are extracted with 0.05 M acetic acid, pH 4.5 for 1 h at 4° C. and then an equal volume of 0.4 M sodium acetate, pH 4.0 is added and incubated for 3 h at 4° C., after which the granules are released into the supernatant (spin at 12,000×g) and concentrated using an Amicon YM-10 membrane.

NGAL-like polypeptides are radiolabeled with ¹²⁵I according to the chloramines-T method and purified using gel filtration (Hunter et al., Nature 194:495 (1962), herein incorporated by reference). 50 μl of the sample or standard is sequentially mixed with 50 μl of [¹²⁵I]-NGALHy1 or [¹²⁵ ]-NGALHy2 (8 μl/L), 50 μl anti-NGAL-like antibody [diluted 1:3800 in assay buffer (0.05 M sodium phosphate, pH 7.4 containing 0.08 M NaCl, 0.01 M Na-EDTA, 0.2% BSA, 0.02% NaN₃, 0.2% CTAB (N-cetyl-N,N,N-trimethylammonium bromide), 0.5% Tween-20)] and incubated for 3 h at room temperature. The mixture is further incubated with 2 ml anti-rabbit IgG-Sepharose for 30 min at room temperature. The NGAL-like-antibody complexes bound to separose are pelleted at 4000 rpm, 10 min and the amount of [¹²⁵I]-NGALHy1 or [¹²⁵I]-NGALHy2 is counted in a gamma counter.

### Example 42

### Anti-Microbial Activity of NGAL-Like Polypeptides

A standard halo assay is used to determine the inhibitory effect of NGAL-like polypeptides on microbial cell growth by measuring the size of the zone of exclusion (see Bjorck et al., Nature 337:385-386 (1989), herein incorporated by reference). This assay can be performed with both bacterial and yeast strains. For example, Streptococci are plated on 0.8% purified agar in Todd-Hewitt broth containing 4% sheep's blood. Sterile filter paper discs are impregnated with either an NGAL-like polypeptide solution (at various concentrations) or antibiotics, such as 0.1 μg benzylpenicillin or 0.2 IU bacitracin, or 1% dimethyl sulfoxide (DMSO) as a control and placed on the plate. The plates are incubated at 37° C. for 1-2 days and the sizes of the zones of exclusion (“halos”) are measured, the larger the halo, the greater the inhibition of growth.

### Example 43

### NGAL-Like Polypeptide Effects on Cell Proliferation

### A. Lymphoproliferation Assay

To examine the effect of NGAL-like polypeptides on proliferative responses, a mitogen-induced lymphoproliferation assay is done (see Cheresh et al., Immunology 51:541-548 (1984), herein incorporated by reference). Mononuclear cells are isolated from blood samples. In a multi-well culture dish, a variety of mitogens are added (one mitogen per well) such as 0.01 mg/ml Concanavalin A (ConA), 0.1 mg/ml phytohaemagglutinin-P, 0.5 mg/ml pokeweed mitogen, or as a control 0.01 ml growth medium. To each well is added 1×10⁵ mononuclear cells in 0.19 ml growth medium with 10% normal human serum with or without NGAL-like polypeptides and incubated for 72 h at 37° C. with 5% CO₂. 18 h before harvesting, the cells are pulsed with [³H]-thymidine (50 μCi/ml). Cells are harvested and collected on glass fiber filter paper and lysed with deionized water. Incorporated thymidine is measured in a scintillation counter. Data is presented as counts per minute of experimental cultures with mitogen minus counts per minute of control cultures without mitogen.

### B. Redistribution of Cell Surface Receptors

Another proliferative response is the redistribution of cell surface receptors such as the ConA receptor and cell surface immunoglobulin (sIg) molecules. This effect is measured with a capping assay (see Cheresh et al., Immunology 51:541-548 (1984), herein incorporated by reference). Mononuclear cells (2×10⁶) are incubated for 1 h at 37° C. with 0.2 ml normal human serum with or without NGAL-like polypeptides. Cells are washed with Hank's buffered salt solution without magnesium or calcium (HBSS) and resuspended in 0.2 ml fluorescein (FITC)-conjugated ConA (15 μg/ml) in HBSS- and incubated for various times at 37° C. Cells are fixed with 4% paraformaldehyde, washed with PBS, pH 7.4 containing 1 mg/ml BSA and placed on a microscope slide. The number of caps is determined using a Nikon Optiphot microscope equipped with epifluoresence. A minimum of 200 cells is counted and capped cells are defined to be cells with uniform fluorescence over less than or equal to one-third of the cell membrane. Alternatively, cells are incubated with 0.1 ml FITC-conjugated goat-anti-human Ig (100 μg/ml) in Media 199 for 1 h at 4° C., washed with Media 199 and incubated at room temperature for 10 min. Cells are fixed in 4% paraformaldehyde, washed with Media 199 containing 0.1% gelatin and mounted on microscope slides. Cells are counted for capping as stated above. Capping is not required for lymphocyte activation, but inhibition of capping may affect other events involving receptor mobility that may be necessary for triggering lymphoproliferative responses. Thus, a decrease in capping is associated with an inhibition of lymphoid activation as well as cell proliferation.

### Example 44

### NGAL-Like Polypeptide-Dependent Modulation of Matrix Metalloproteinase Activity

To monitor the effect of NGAL-like polypeptides on matrix metalloproteinase (MMP) activity, recombinant versions of NGALHy1, NGALHy2, and MMPs, such as MMP-9, are used in an MMP activity assay (see Yan et al., J. Biol. Chem. 276:37258-37265 (2001), herein incorporated by reference). MMP-9 is diluted in gelatinase buffer (50 mM Tris-HCl, pH 7.0, containing 5 mM CaCl₂, 1 μM ZnCl₂) to 0.1 μM and incubated at 37° C. for various times. Aliquots of MMP-9 (10 ng) are collected at different time points and subjected to gelatin zymography (Braunhut and Moses, J. Biol. Chem. 269:13472-13479 (1994), herein incorporated by reference). Briefly, Type 1 gelatin is added to the standard Laemmli acrylamide gel mixture at 1 mg/ml. Samples are mixed 3:1 with the substrate gel sample buffer (10% SDS, 4% sucrose, 0.25 M Tris-HCl, pH 6.8, and 0.1% bromphenol blue) and loaded onto the gel without boiling. After electrophoresis, gels are soaked in 2.5% Triton X-100 for 30 min and rinsed and incubated overnight at 37° C. in substrate buffer (50 mM Tris-HCl, pH 8, 5 mM CaCl₂, and 0.02% NaN₃). Gels are stained in 0.5% Coomassie Blue for 15-30 min and destained in water. MMP activity is visualized as zones of clearance within the gels and quantitated using densitometry.

To analyze NGAL-like-dependent protection, NGALHy1 or NGALHy2 is diluted in gelatinase buffer and mixed with MMP9 in different molar ratios ranging from 10:1 to 1:20 and incubated at 37° C. for varying times from 0.5 to 2 h. Aliquots are collected at each timepoint and degradation of MMP9 is monitored by substrate electrophoresis. This assay can also be performed using anti-NGAL-like antibodies.

Investigation of the MMP-NGAL-like interaction is performed in cell culture as well (see Yan et al., J. Biol. Chem. 276:37258-37265 (2001), herein incorporated by reference). Stably transfected cells expressing NGALHy1 or NGALHy2, such as MDA-MB0231 breast carcinoma cells, are incubated with serum-free media at 90% confluency for 20 h. Conditioned media is harvested, clarified and electrophoresed to detect MMP9 activity. Steady-state mRNA levels of MMP9, tissue inhibitor of metalloproteinases-1 (TIMP-1) and a housekeeping gene glyceraldehyde-3-phosphate dehydrogenase (GAPDH) are determined by quantitative real-time PCR analysis (Simpson, et al., Molec. Vision 6:178-183 (2000), herein incorporated by reference) to determine the relative copy number of MMP9 mRNA expressed per cell. Thus, a comparison of MMP9 protein and mRNA levels can be made to determine the effect of NGAL-like polypeptides on MMP9 activity.

### Example 45

### Apoptosis Assay

Apoptosis is the controlled process by which cells under a programmed cell death. Apoptosis is studied by analysis of dead or dying cells and one of the methods to to identify dying or apoptotic cells by TUNEL assayacridine orange and LysoTracker staining. The TUNEL, acrdine orange and LysoTracker staining assays are performed as described in Hersh et al (Proc. Natl. Acad. Sci. USA. 99:4355-4360 (2002), incorporated herein by reference).

### Example 46

### Endocytosis Assays

Endocytosis is a process by which the cells internalize proteins or lipids from the extracellular space to the cytoplasm. This process is characterized by several steps which include binding of the protein or lipid to a receptor, interalization of the bound material and transfer to sorting endosomes followed by transfer of the protein or lipid from sorting endosomes to lysosomes. These steps are studied by a variety of different assays that are trace the movement of lipids or proteins that are tagged with a radiolabeled or fluorescent marker. Detection of the radiolabeled or fluorescent marker is done at every step of the endocytosis pathway by fluorescence microscopy or by measuring the radioactivity associated with whole cells or isolated fractions of the cells containing the plasma membrane, endosomes, lysosomes, golgi complex etc. A combination of the assays is used to study endocytosis as described in Chen et al. (Proc. Natl. Acad. Sci USA, 95:6373-6378 (1998), incorporated herein by reference).

### Example 47

### Expression Levels of Peroxidasin-Like mRNA in Various Tumor Cell Lines

Expression of peroxidasin-like mRNA is determined in various tumor cell lines, including lymphoma, leukemia, melanoma, breast cancer, ovarian cancer, lung cancer, brain cancer, etc., and tumor tissues. Poly-A messenger RNA is isolated from the cell lines and subjected to quantitative, real-time PCR analysis (Simpson, et al., Molec. Vision. 6: 178-183 (2000), herein incorporated by reference) to determine the relative copy number of peroxidasin-like mRNA expressed per cell in each line. Elongation factor 1 mRNA expression is used as a positive control and normalization factors in all samples.

Expression of peroxidasin-like mRNA is determined in various healthy and tumor tissues. Poly-A mRNA is isolated from various tissues and subjected to quantitative, real-time PCR analysis, as described above, to determine the relative expression of peroxidasin-like mRNA in the sample.

### Example 48

### In Vitro Antibody-Dependent Cytotoxicity Assay

The ability of a peroxidasin-like protein-specific antibody to induce antibody-dependent cell-mediated cytoxicity (ADCC) is determined in vitro. ADCC is performed using the CytoTox 96 Non-Radioactive Cytoxicity Assay (Promega; Madison, Wis.) (Hornick et al., Blood 89:4437-4447, (1997)) as well as effector and target cells. Peripheral blood mononuclear cells (PBMC) or neutrophilic polymorphonuclear leukocytes (PMN) are two examples of effector cells that can be used in this assay. PBMC are isolated from healthy human donors by Ficoll-Paque gradient centrifugation, and PMN are purified by centrifugation through a discontinuous percoll gradient (70% and 62%) followed by hypotonic lysis to remove residual erythrocytes. RA1 B cell lymphoma cells (for example) are used as target cells.

RA1 cells are suspended in RPMI 1640 medium supplemented with 2% fetal bovine serum and plated in 96-well V-bottom microtitier plates at 2×10⁴ cells/well. peroxidasin-like protein-specific antibody is added in triplicate to individual wells at 1 μg/ml, and effector cells are added at various effector:target cell ratios (12.5:1 to 50:1). The plates are incubated for 4 hours at 37° C. The supernatants are then harvested, lactate dehydrogenase release determined, and percent specific lysis calculated using the manufacture's protocols.

### Example 49

### Toxin-Conjugated Peroxidasin-Like Protein-Specific Antibodies

Antibodies to peroxidasin-like protein are conjugated to toxins and the effect of such conjugates in animal models of cancer is evaluated. Chemotherapeutic agents, such as calicheamycin and carboplatin, or toxic peptides, such as ricin toxin, are used in this approach. Antibody-toxin conjugates are used to target cytotoxic agents specifically to cells bearing the antigen. The antibody-toxin binds to these antigen-bearing cells, becomes internalized by receptor-mediated endocytosis, and subsequently destroys the targeted cell. In this case, the antibody-toxin conjugate targets peroxidasin-like protein-expressing cells, such as B cell lymphomas, and deliver the cytotoxic agent to the tumor resulting in the death of the tumor cells.

One such example of a toxin that may be conjugated to an antibody is carboplatin. The mechanism by which this toxin is conjugated to antibodies is described in Ota et al., Asia-Oceania J. Obstet. Gynaecol. 19: 449-457 (1993). The cytotoxicity of carboplatin-conjugated peroxidasin-like protein-specific antibodies is evaluated in vitro, for example, by incubating peroxidasin-like protein-expressing target cells (such as the RA1 B cell lymphoma cell line) with various concentrations of conjugated antibody, medium alone, carboplatin alone, or antibody alone. The antibody-toxin conjugate specifically targets and kills cells bearing the peroxidasin-like protein antigen, whereas, cells not bearing the antigen, or cells treated with medium alone, carboplatin alone, or antibody alone, show no cytotoxicity.

The antitumor efficacy of carboplatin-conjugated peroxidasin-like protein-specific antibodies is demonstrated in in vivo murine tumor models. Five to six week old, athymic nude mice are engrafted with tumors subcutaneously or through intravenous injection. Mice are treated with the peroxidasin-like protein-carboplatin conjugate or with a non-specific antibody-carboplatin conjugate. Tumor xenografts in the mouse bearing the peroxidasin-like protein antigen are targeted and bound to by the peroxidasin-like protein-carboplatin conjugate. This results in tumor cell killing as evidenced by tumor necrosis, tumor shrinkage, and increased survival of the treated mice.

Other toxins are conjugated to peroxidasin-like protein-specific antibodies using methods known in the art. An example of a toxin conjugated antibody in human clinical trials is CMA-676, an antibody to the CD33 antigen in AML which is conjugated with calicheamicin toxin (Larson, Semin. Hematol. 38(Suppl 6):24-31 (2001)).

### Example 50

### Radioimmunotherapy Using Peroxidasin-Like Protein-Specific Antibodies

Animal models are used to assess the effect of antibodies specific to peroxidasin-like protein as vectors in the delivery of radionuclides in radioimmunotherapy to treat lymphoma, hematological malignancies, and solid tumors. Human tumors are propagated in 5-6 week old athymic nude mice by injecting a carcinoma cell line or tumor cells subcutaneously. Tumor-bearing animals are injected intravenously with radio-labeled anti-peroxidasin-like protein antibody (labeled with 30-40 μCi of ¹³¹I, for example) (Behr, et al., Int. J. Cancer 77: 787-795 (1988)). Tumor size is measured before injection and on a regular basis (i.e. weekly) after injection and compared to tumors in mice that have not received treatment. Anti-tumor efficacy is calculated by correlating the calculated mean tumor doses and the extent of induced growth retardation. To check tumor and organ histology, animals are sacrificed by cervical dislocation and autopsied. Organs are fixed in 10% formalin, embedded in paraffin, and thin sectioned. The sections are stained with hematoxylin-eosin.

### Example 51

### Immunotherapy Using Peroxidasin-Like Protein-Specific Antibodies

Animal models are used to evaluate the effect of peroxidasin-like protein-specific antibodies as targets for antibody-based immunotherapy using monoclonal antibodies. Human myeloma cells are injected into the tail vein of 5-6 week old nude mice whose natural killer cells have been eradicated. To evaluate the ability of peroxidasin-like protein-specific antibodies in preventing tumor growth, mice receive an intraperitoneal injection with peroxidasin-like protein-specific antibodies either 1 or 15 days after tumor inoculation followed by either a daily dose of 20 μg or 100 μg once or twice a week, respectively (Ozaki, et al., Blood 90:3179-3186 (1997)). Levels of human IgG (from the immune reaction caused by the human tumor cells) are measured in the murine sera by ELISA.

The effect of peroxidasin-like protein-specific antibodies on the proliferation of myeloma cells is examined in vitro using a ³H-thymidine incorporation assay (Ozaki et al., supra). Cells are cultured in 96-well plates at 1×10⁵ cells/ml in 100 μl/well and incubated with various amounts of peroxidasin-like protein antibody or control IgG (up to 100 μg/ml) for 24 h. Cells are incubated with 0.5 μCi ³H-thymidine (New England Nuclear, Boston, Mass.) for 18 h and harvested onto glass filters using an automatic cell harvester (Packard, Meriden, Conn.). The incorporated radioactivity is measured using a liquid scintillation counter.

The cytotoxicity of the peroxidasin-like protein monoclonal antibody is examined by the effect of complements on myeloma cells using a ⁵¹Cr-release assay (Ozaki et al., supra). Myeloma cells are labeled with 0.1 mCi ⁵¹Cr-sodium chromate at 37° C. for 1 h. ⁵¹Cr-labeled cells are incubated with various concentrations of peroxidasin-like protein monoclonal antibody or control IgG on ice for 30 min. Unbound antibody is removed by washing with medium. Cells are distributed into 96-well plates and incubated with serial dilutions of baby rabbit complement at 37° C. for 2 h. The supernatants are harvested from each well and the amount of ⁵¹Cr released is measured using a gamma counter. Spontaneous release of ⁵¹Cr is measured by incubating cells with medium alone, whereas maximum ⁵¹Cr release is measured by treating cells with 1% NP-40 to disrupt the plasma membrane. Percent cytotoxicity is measured by dividing the difference of experimental and spontaneous ⁵¹Cr release by the difference of maximum and spontaneous ⁵¹Cr release.

Antibody-dependent cell-mediated cytotoxicity (ADCC) for the peroxidasin-like protein monoclonal antibody is measured using a standard 4 h ⁵¹Cr-release assay (Ozaki et al., supra). Splenic mononuclear cells from SCID mice are used as effector cells and cultured with or without recombinant interleukin-2 (for example) for 6 days. ⁵¹ Cr-labeled target myeloma cells (1×10⁴ cells) are placed in 96-well plates with various concentrations of anti-peroxidasin-like protein monoclonal antibody or control IgG. Effector cells are added to the wells at various effector to target ratios (12.5:1 to 50:1). After 4 h, culture supernatants are removed and counted in a gamma counter. The percentage of cell lysis is determined as above.

### Example 52

### Peroxidasin-Like Protein-Specific Antibodies as Immunosuppressants

Animal models are used to assess the effect of peroxidasin-like protein-specific antibodies to suppress autoimmune diseases, such as arthritis or other inflammatory conditions, or rejection of organ transplants. Immunosuppression is tested by injecting mice with horse red blood cells (HRBCs) and assaying for the levels of HRBC-specific antibodies (Yang, et al., Int. Immunopharm. 2:389-397 (2002)). Animals are divided into five groups, three of which are injected with anti-TLR9 antibodies for 10 days, and 2 of which receive no treatment. Two of the experimental groups and one control group are injected with either Earle's balanced salt solution (EBSS) containing 5-10×10⁷ HRBCs or EBSS alone. Anti-peroxidasin-like protein antibody treatment is continued for one group while the other groups receive no antibody treatment. After 6 days, all animals are bled by retro-orbital puncture, followed by cervical dislocation and spleen removal. Splenocyte suspensions are prepared and the serum is removed by centrifugation for analysis.

Immunosupression is measured by the number of B cells producing HRBC-specific antibodies. The Ig isotype (for example, IgM, IgG1, IgG2, etc.) is determined using the IsoDetect™ Isotyping kit (Stratagene, La Jolla, Calif.). Once the Ig isotype is known, murine antibodies against HRBCs are measured using an ELISA procedure. 96-well plates are coated with HRBCs and incubated with the anti-HRBC antibody-containing sera isolated from the animals. The plates are incubated with alkaline phosphatase-labeled secondary antibodies and color development is measured on a microplate reader (SPECTRAmax 250, Molecular Devices) at 405 nm using p-nitrophenyl phosphate as a substrate.

Lymphocyte proliferation is measured in response to the T and B cell activators concanavalin A and lipopolysaccharide, respectively (Jiang, et al., J. Immunol. 154:3138-3146 (1995). Mice are randomly divided into 2 groups, 1 receiving anti-peroxidasin-like protein antibody therapy for 7 days and 1 as a control. At the end of the treatment, the animals are sacrificed by cervical dislocation, the spleens are removed, and splenocyte suspensions are prepared as above. For the ex vivo test, the same number of splenocytes are used, whereas for the in vivo test, the anti-peroxidasin-like protein antibody is added to the medium at the beginning of the experiment. Cell proliferation is also assayed using the ³H-thymidine incorporation assay described above (Ozaki, et al., Blood 90: 3179 (1997)).

### Example 53

### Cytokine Secretion in Response to Peroxidasin-Like Protein Peptide Fragments

Assays are carried out to assess activity of fragments of the peroxidasin-like protein, such as the Ig domain, to stimulate cytokine secretion and to stimulate immune responses in NK cells, B cells, T cells, and myeloid cells. Such immune responses can be used to stimulate the immune system to recognize and/or mediate tumor cell killing or suppression of growth. Similarly, this immune stimulation can be used to target bacterial or viral infections. Alternatively, fragments of the peroxidasin-like protein that block activation through the peroxidasin-like protein receptor may be used to block immune stimulation in natural killer (NK), B, T, and myeloid cells.

Fusion proteins containing fragments of the peroxidasin-like protein, such as the Ig domain (peroxidasin-like-Ig), are made by inserting a CD33 leader peptide, followed by a peroxidasin-like protein domain fused to the Fc region of human IgG1 into a mammalian expression vector, which is stably transfected into NS-1 cells, for example. The fusion proteins are secreted into the culture supernatant, which is harvested for use in cytokine assays, such as interferon-γ (IFN-γ) secretion assays (Martin, et al., J. Immunol. 167:3668-3676 (2001)).

PBMCs are activated with a suboptimal concentration of soluble CD3 and various concentrations of purified, soluble anti-peroxidasin-like protein monoclonal antibody or control IgG. For peroxidasin-like protein-Ig cytokine assays, anti-human Fc Ig at 5 or 20 μg/ml is bound to 96-well plates and incubated overnight at 4° C. Excess antibody is removed and either peroxidasin-like protein-Ig or control Ig is added at 20-50 μg/ml and incubated for 4 h at room temperature. The plate is washed to remove excess fusion protein before adding cells and anti-CD3 to various concentrations. Supernatants are collected after 48 h of culture and IFN-γ levels are measured by sandwich ELISA, using primary and biotinylated secondary anti-human IFN-γ antibodies as recommended by the manufacturer.

### Example 54

### Diagnostic Methods Using Peroxidasin-Like Protein-Specific Antibodies to Detect Peroxidasin-Like Protein Expression

Expression of peroxidasin-like protein in tissue samples (normal or diseased) is detected using anti-peroxidasin-like protein antibodies. Samples are prepared for immunohistochemical (IHC) analysis by fixing the tissue in 10% formalin embedding in paraffin, and sectioning using standard techniques. Sections are stained using the peroxidasin-like protein-specific antibody followed by incubation with a secondary horse radish peroxidase (HRP)-conjugated antibody and visualized by the product of the HRP enzymatic reaction.

Expression of peroxidasin-like protein on the surface of cells within a blood sample is detected by flow cytometry. Peripheral blood mononuclear cells (PBMC) are isolated from a blood sample using standard techniques. The cells are washed with ice-cold PBS and incubated on ice with the peroxidasin-like protein-specific polyclonal antibody for 30 min. The cells are gently pelleted, washed with PBS, and incubated with a fluorescent anti-rabbit antibody for 30 min. on ice. After the incubation, the cells are gently pelleted, washed with ice cold PBS, and resuspended in PBS containing 0.1% sodium azide and stored on ice until analysis. Samples are analyzed using a FACScalibur flow cytometer (Becton Dickinson) and CELLQuest software (Becton Dickinson). Instrument setting are determined using FACS-Brite calibration beads (Becton-Dickinson).

Tumors expressing peroxidasin-like protein are imaged using peroxidasin-like protein-specific antibodies conjugated to a radionuclide, such as ¹²³I, and injected into the patient for targeting to the tumor followed by X-ray or magnetic resonance imaging.

### Example 55

### Tumor Imaging Using Peroxidasin-Like Protein-Specific Antibodies

Peroxidasin-like protein-specific antibodies are used for imaging peroxidasin-like protein-expressing cells in vivo. Six-week-old athymic nude mice are irradiated with 400 rads from a cesium source. Three days later the irradiated mice are inoculated with 4×10⁷ RA1 cells and 4×10⁶ human fetal lung fibroblast feeder cells subcutaneously in the thigh. When the tumors reach approximately 1 cm in diameter, the mice are injected intravenously with an inoculum containing 100 μCi/10 μg of ¹³¹I-labeled peroxidasin-like protein-specific antibody. At 1, 3, and 5 days postinjection, the mice are anesthetized with a subcutaneous injection of 0.8 mg sodium pentobarbital. The immobilized mice are then imaged in a prone position with a Spectrum 91 camera equipped with a pinhole collimator (Raytheon Medical Systems; Melrose Park, Ill.) set to record 5,000 to 10,000 counts using the Nuclear MAX Plus image analysis software package (MEDX Inc.; Wood Dale, Ill.) (Hornick, et al., Blood 89:4437-4447 (1997)).

1. An isolated polynucleotide comprising a nucleotide sequence selected from the group consisting of SEQ ID NO: 1-4, 6, 14, 16, 25-27, 29, 157-159, 161, 183-185, 187, 214, 216, 240, 242, 271, 273, 300-301, 303, 322, 324, 345-347, 349, 353-354, 356, 377, 379, 405-407, 409, 418-419,421, 441443, 485-486, 488, 503, 504, 506, 514-515, 517, 526-527, 529, 547, 549, 556, 558, 570-571,573,577-578,580,587,589, 601, 603, 606, 608, 611, 613, 617, 619, 621, 623, 625, 627, 629, or 631 or the mature protein coding portion thereof.

2. An isolated polynucleotide encoding a polypeptide with biological activity, wherein said polynucleotide hybridizes to the polynucleotide of claim 1 under stringent hybridization conditions (0.5 M NaHPO₄, 7% sodium dodecyl sulfate (SDS), 1 mM EDTA at 65° C.).

3. The polynucleotide of claim 1 wherein said polynucleotide is DNA.

4. An isolated polynucleotide which comprises the complement of any one of the polynucleotides of claim 1.

5. A vector comprising the polynucleotide of claim 1.

6. An expression vector comprising the polynucleotide of claim 1.

7. A host cell genetically engineered to comprise the polynucleotide of claim 1.

8. A host cell genetically engineered to comprise the polynucleotide of claim 1 operatively associated with a regulatory sequence that modulates expression of the polynucleotide in the host cells.

9. An isolated polypeptide, wherein the polypeptide is selected from the group consisting of:
(a) a polypeptide encoded by any one of the polynucleotides of claim 1; and
(b) a polypeptide encoded by a polynucleotide hybridizing under stringent conditions with any one of SEQ ID NO: 5, 7-13, 15, 17-24, 28, 30-156, 160, 162-182, 186, 188-213, 215, 217-239, 241, 243-270, 272, 274-299, 302, 304-321, 323, 325-344, 348, 350-352, 355, 357-376, 378, 380-401, 408, 410-414, 415, 420, 422-439, 444-480, 482-484, 487, 489-501, 505, 507-512, 516, 518-524, 528, 530-539, 542, 544-546, 548, 550-553, 557, 559-567, 572, 574, 576, 579, 581-584, 588, 590, 596, 602, 604-605, 607, 609-610, 612, 614-615, 618, 620, 622, 624, 626, 628, 630, 632, or 634-653.

10. An isolated polypeptide comprising an amino acid sequence selected from the group consisting of any one of the polypeptides of SEQ ID NO: 5, 7-13, 15, 17-24, 28, 30-156, 160, 162-182, 186, 188-213, 215, 217-239, 241, 243-270, 272, 274-299, 302, 304-321, 323, 325-344, 348, 350-352, 355, 357-376, 378, 380-401, 408, 410-414, 415, 420, 422-439, 444-480, 482-484, 487, 489-501, 505, 507-512, 516, 518-524, 528, 530-539, 542, 544-546, 548, 550-553, 557, 559-567, 572, 574, 576, 579, 581-584, 588, 590, 596, 602, 604-605, 607, 609-610, 612, 614-615, 618, 620, 622, 624, 626, 628, 630, 632, or 634-653.

11. A composition comprising the polypeptide of claim 9 or 10 and a carrier.

12. An antibody directed against the polypeptide of claim 9 or 10.

13. A method for detecting the polynucleotide of claim 1 in a sample, comprising the steps of:
(a) contacting the sample with polynucleotide probe that specifically hybridizes to the polynucleotide under conditions which permit formation of a probe/polynucleotide complex; and
(b) detecting the presence of a probe/polynucleotide complex, wherein the presence of the complex indicates the presence of a polynucleotide.

14. A method for detecting the polynucleotide of claim 1 in a sample, comprising the steps of:
(a) contacting the sample under stringent hybridization conditions with nucleic acid primers that anneal to the polynucleotide of claim 1 under such conditions; and
(b) amplifying the polynucleotide or fragment thereof, so that if the polynucleotide or fragment is amplified, the polynucleotide is detected.

15. The method of claim 14, wherein the polynucleotide is an RNA molecule that encodes the polypeptide of claim 9 or 10, and the method further comprises reverse transcribing an annealed RNA molecule into a cDNA polynucleotide.

16. A method of detecting the presence of the polypeptide of claim 9 or 10 having the amino acid sequence of any one of SEQ ID NO: 5, 7-13, 15, 17-24, 28, 30-156, 160, 162-182, 186, 188-213, 215, 217-239, 241, 243-270, 272, 274-299, 302, 304-321, 323, 325-344, 348, 350-352, 355, 357-376, 378, 380-401, 408, 410-414, 415, 420, 422-439, 444-480, 482-484, 487, 489-501, 505, 507-512, 516, 518-524, 528, 530-539, 542, 544-546, 548, 550-553, 557, 559-567, 572, 574, 576, 579, 581-584, 588, 590, 596, 602, 604-605, 607, 609-610, 612, 614-615, 618, 620, 622, 624, 626, 628, 630, 632, or 634-653 or a fragment thereof in a cell, tissue or fluid sample comprising:
(a) contacting said cell, tissue or fluid sample with an antibody or fragment of claim 10 under conditions which permit the formation of an antibody/polypeptide complex; and
(b) detecting the presence of an antibody/polypeptide complex, wherein the presence of the antibody/polypeptide complex indicates the presence of any of the polypeptides of claim 10.

17. A method for identifying a compound that binds to a polypeptide of any one of SEQ ID NO: 5, 7-13, 15, 17-24, 28, 30-156, 160, 162-182, 186, 188-213, 215, 217-239, 241, 243-270, 272, 274-299, 302, 304-321, 323, 325-344, 348, 350-352, 355, 357-376, 378, 380-401, 408, 410-414, 415, 420, 422439, 444480, 482-484, 487, 489-501, 505, 507-512, 516, 518-524, 528, 530-539, 542, 544-546, 548, 550-553, 557, 559-567, 572, 574, 576, 579, 581-584,588,590,596, 602, 604-605,607,609-610,612,614-615,618,620,622, 624, 626, 628, 630, 632, or 634-653 comprising:
(a) contacting a compound with the polypeptide of any of SEQ ID NO: 5, 7-13, 15, 17-24, 28, 30-156, 160, 162-182, 186, 188-213, 215, 217-239, 241, 243-270, 272, 274-299, 302, 304-321, 323, 325-344, 348, 350-352, 355, 357-376, 378, 380-401, 408, 410-414, 415, 420, 422-439, 444-480, 482-484, 487, 489-501, 505, 507-512, 516, 518-524, 528, 530-539, 542, 544-546, 548, 550-553, 557, 559-567, 572, 574, 576, 579, 581-584, 588, 590, 596, 602, 604-605, 607, 609-610, 612, 614-615, 618, 620, 622, 624, 626, 628, 630, 632, or 634-653 for a time sufficient to form a polynucleotide/compound complex; and
(b) detecting the complex, so that if a polypeptide/compound complex is detected, a compound that binds to any one of SEQ ID NO: 5, 7-13, 15, 17-24, 28, 30-156, 160, 162-182, 186, 188-213, 215, 217-239, 241, 243-270, 272, 274-299, 302, 304-321, 323, 325-344, 348, 350-352, 355, 357-376, 378, 380-401, 408, 410-414, 415, 420, 422-439, 444-480, 482-484, 487, 489-501, 505, 507-512, 516, 518-524, 528, 530-539, 542, 544-546, 548, 550-553, 557, 559-567, 572, 574, 576, 579, 581-584, 588, 590, 596, 602, 604-605, 607, 609 610, 612, 614-615, 618, 620, 622, 624, 626, 628, 630, 632, or 634-653 is identified.

18. A method for identifying a compound that binds to any one of the polypeptides of SEQ ID NO: 5, 7-13, 15, 17-24, 28, 30-156, 160, 162-182, 186, 188-213, 215, 217-239, 241, 243-270, 272, 274-299, 302, 304-321, 323, 325-344, 348, 350-352, 355, 357-376, 378, 380-401, 408, 410-414, 415, 420, 422-439, 444-480, 482-484, 487, 489-501, 505, 507-512, 516, 518-524, 528, 530-539,542,544-546,548,550-553, 557, 559-567, 572, 574, 576, 579, 581-584, 588, 590, 596, 602, 604-605, 607, 609-610, 612, 614-615, 618, 620, 622, 624, 626, 628, 630, 632, or 634-653, comprising:
(a) contacting a compound with the polypeptide of any one of SEQ ID NO: 5, 7-13, 15, 17-24, 28, 30-156, 160, 162-182, 186, 188-213, 215, 217-239, 241, 243-270, 272, 274-299, 302, 304-321, 323, 325-344, 348, 350-352, 355, 357-376, 378, 380-401, 408, 410-414, 415, 420, 422-439, 444-480, 482-484, 487, 489-501, 505, 507-512, 516, 518-524, 528, 530-539, 542, 544-546, 548, 550-553, 557, 559-567, 572, 574, 576, 579, 581-584, 588, 590, 596, 602, 604-605, 607, 609-610, 612, 614-615, 618, 620, 622, 624, 626, 628, 630, 632, or 634-653, in a cell, for a time sufficient to form a polypeptide/compound complex, wherein the complex drives the expression of a reporter gene sequence in the cell; and
(b) detecting the complex by detecting reporter gene sequence expression, so that if a polypeptide/compound complex is detected, a compound that binds to any one of the polypeptides of SEQ ID NO: 5, 7-13, 15, 17-24, 28, 30-156, 160, 162-182, 186, 188-213, 215, 217-239, 241, 243-270, 272, 274-299, 302, 304-321, 323, 325-344, 348, 350-352, 355, 357-376, 378, 380401, 408, 410-414, 415, 420, 422-439, 444-480, 482-484, 487, 489-501, 505, 507-512, 516, 518-524, 528, 530-539, 542, 544-546, 548, 550-553, 557, 559-567, 572, 574, 576, 579, 581-584, 588, 590, 596, 602, 604-605, 607, 609-610, 612, 614-615, 618, 620, 622, 624, 626, 628, 630, 632, or 634-653 is identified.

19. A method of producing the polypeptides of claim 9 or 10, comprising:
(a) culturing the host cell of claim 7 or 8 for a period of time sufficient to express the polypeptide; and
(b) isolating the polypeptide from the cell or culture media in which the cell is grown.

20. A kit comprising any one of the polypeptides of claim 9 or 10.

21. A nucleic acid array comprising the polynucleotide of claim 1 attached to a surface.

22. The polypeptide of claim 9 or 10 wherein the polypeptide is provided on a polypeptide array.

23. A method for modifying the proliferation of neural cells, comprising the step of administering a composition to said cells in an amount effective to modify the proliferation of said cells, wherein said composition is an NgRHy polypeptide.

24. The method of claim 21, wherein said modifying is inducing the proliferation of neural cells.

25. The method of claim 21, wherein said modifying is inhibiting the proliferation of neural cells.
